(19) United States
(12) Patent Application Publication
(10) Pub. No.: US 2025/0382359 A1
(43) Pub. Date: Dec. 18, 2025

(54) TARGETING GDF15-GFRAL PATHWAY

(71) Applicants: BYOMass Inc., Sudbury, MA (US); Adimab, LLC, Lebanon, NH (US)

(72) Inventors: Vivienne Margaret Jackson, Sudbury, MA (US); Nels P. Nielson, Lebanon, NH (US)

(21) Appl. No.: 18/722,143

(22) PCT Filed: Dec. 21, 2022
(86) PCT No.: PCT/US22/53715

§ 371 Date: Jun. 20, 2024

Related U.S. Application Data
(60) Provisional application No. 63/292,923, filed on Dec. 22, 2021.
(60) Provisional application No. 63/396,881, filed on Aug. 10, 2022.

(51) Int. Cl. C07K 16/22; A61K 39/00; A61P 1/08; A61P 3/04; G01N 33/68

(52) U.S. Cl. CPC C07K 16/22; A61P 1/08; A61P 3/04; G01N 33/6863; A61K 2039/505; C07K 2317/526; C07K 2317/565; C07K 2317/76; C07K 2317/92; G01N 2333/475; G01N 2800/52

(57) ABSTRACT

Provided herein are methods of treating and/or preventing certain types and/or instances of nausea and/or emesis with which it had not previously been considered to be associated, by targeting the GDF15-GFRAL pathway.

### CROSS-REFERENCE TO RELATED APPLICATIONS

This application claims priority to U.S. provisional applications 63/292,923 filed on Dec. 22, 2021, and 63/396,881 filed on Aug. 10, 2022, the entire contents of each of which are hereby incorporated by reference.

### BACKGROUND

Nausea and/or emesis may occur as a chronic or recurrent condition and can lead to a significant decline in quality of life. Nausea often accompanies emesis, but doesn't always lead to emesis. There are situations when severe nausea may be present without emesis and less frequently, when emesis may be present without preceding nausea. The causes of nausea and/or emesis are broad, thus, treatment/therapies are varied.

### SUMMARY

Nausea and/or emesis are encountered in a variety of medical settings. For some types or instances of nausea and/or emesis, underlying causes may be understood and treatments available. However, for many types and/or instances of nausea and/or emesis a need in the art for effective treatments remains.

Among other things, the present disclosure provides insights relating to causes of and/or treatments for certain types and/or instances of nausea and/or emesis.

In certain embodiments, the present disclosure identifies the GDF15-GFRAL Pathway as potentially contributing to, or at least correlating with, certain types and/or instances of nausea and/or emesis with which it had not previously been considered to be associated. For example, in some embodiments, the present disclosure teaches that Hyperemesis Gravidarum (HG), Cyclic Vomiting Syndrome (CVS), Cannabinoid Hyperemesis Syndrome (CHS), Mitochondrial Disorders (MIDs), and/or Migraine Associated Nausea/Vomiting (MAN/V) and/or subjects that suffer from HG, CVS, CHS, MIDs, and/or MAN/V, may benefit from therapeutic targeting of the GDF15-GFRAL Pathway. Alternatively or additionally, in some embodiments, the present disclosure teaches that markers of GDF15-GFRAL Pathway activity (such as, for example, level and/or activity of GDF15), may be useful to identify subjects who will benefit from therapy targeting this Pathway.

In some embodiments, the present disclosure provides technologies for treatment of one or more of HG, CVS, CHS, MIDs, and/or MAN/V, by administration of a GDF15-GFRAL-Pathway-targeted therapy. In some embodiments, a GDF15-GFRAL-Pathway-targeted therapy may be or comprise administration of a composition that comprises or delivers a GDF15-GFRAL Pathway Modulating Agent.

In some embodiments, the present disclosure provides a method of reducing and/or preventing nausea, a method of preventing and/or reducing weight loss, a method for preventing and/or reducing vomiting, a method for preventing and/or reducing loss of appetite, or a method for preventing restricted fetal growth, in a patient suffering from HG. In some embodiments of any of the methods disclosed herein, a method further comprises administering an agent or composition targeting the GDF15-GFRAL pathway to a cell, tissue or subject. In some embodiments, a subject suffering from HG is pregnant.

In some embodiments, the present disclosure provides a method for preventing nausea, a method of reducing nausea, a method of preventing weight loss, a method of reducing weight loss, a method for preventing vomiting, a method of reducing vomiting, a method for preventing loss of appetite, or a method of reducing loss of appetite for a patient suffering from CVS. In some embodiments of any of the methods disclosed herein, a method further comprises administering an agent or composition targeting the GDF15-GFRAL pathway to a cell, tissue or subject.

In some embodiments, the present disclosure provides a method of preventing nausea, a method for reducing nausea, a method of preventing weight loss, a method of reducing weight loss, a method of preventing vomiting, a method of reducing vomiting, a method of preventing loss of appetite, or a method of reducing loss of appetite for a patient suffering from CHS. In some embodiments of any of the methods disclosed herein, a provided such method comprises administering an agent or composition targeting the GDF15-GFRAL pathway to a cell, tissue or subject.

In some embodiments, the present disclosure provides a method of preventing nausea, a method for reducing nausea, a method of preventing weight loss, a method of reducing weight loss, a method of preventing vomiting, a method of reducing vomiting, a method of preventing loss of appetite, or a method of reducing loss of appetite for a patient suffering from MIDs. In some embodiments of any of the methods disclosed herein, a provided such method comprises administering an agent or composition targeting the GDF15-GFRAL pathway to a cell, tissue or subject.

In some embodiments, the present disclosure provides a method for preventing nausea, a method of reducing nausea, a method of preventing weight loss, a method of reducing weight loss, a method of preventing vomiting, a method of reducing vomiting, a method for reducing loss of appetite, or a method of reducing loss of appetite for a patient suffering from MAN/V. In some embodiments of any of the methods disclosed herein, a method further comprises administering an agent or composition targeting the GDF15-GFRAL pathway to a cell, tissue or subject.

In some embodiments, administration of a GDF15-GFRAL Pathway Modulating Agent or pharmaceutical composition comprising the same reduces a level and/or activity of the GDF15-GFRAL pathway relative to a comparator. In some embodiments, a level of free and/or active GDF15 is reduced. In some embodiments, interaction of GFRAL with GDF15 and/or RET is reduced. In some embodiments, GFRAL and/or RET activation is reduced.

In some embodiments, a GDF15-GFRAL Pathway Modulating Agent comprises a GDF15 Antibody Agent, a GFRAL Antibody Agent, a RET Antibody Agent or an Antibody Agent that modulates a GDF15-GFRAL Pathway.

In some embodiments, an Antibody Agent comprises a heavy chain comprising at least one CH domain. In some embodiments, an Antibody Agent comprises a heavy chain comprising a CH1 domain, a CH2 domain, a CH3 domain, or a combination thereof.

In some embodiments, an Antibody Agent comprises a heavy chain comprising a CH3 domain. In some embodiments, a CH3 domain comprises a leucine at position 428 and/or an alanine at position 434.

In some embodiments, an Antibody Agent comprises a heavy chain comprising a CH2 domain and a CH3 domain, e.g., an Fc domain. In some embodiments, an Antibody Agent comprises an Fc domain comprising a mutation, e.g., as disclosed herein.

In some embodiments, an Fc domain comprises a mutation that has reduced binding to a neonatal Fc receptor (FcRn).

In some embodiments, an Fc domain comprises a LAGA mutation, a FEGG mutation, an AAGG mutation, an AAGA mutation, a LALA mutation or a combination thereof.

In some embodiments, an Fc domain comprises a I253A mutation, a H310A mutation, a H435R mutation, a H435A mutation or a combination thereof.

In some embodiments of any of the methods disclosed herein, when the GDF15-GFRAL Pathway Modulating Agent is an Antibody Agent comprising an Fc mutation which reduces (e.g., ablates) binding to FcRn, administration of an Antibody Agent to a pregnant subject reduces (e.g., prevents) placental transfer of an Antibody Agent, as compared to administration of an otherwise similar Antibody Agent without an Fc mutation which reduces (e.g., ablates) binding to FcRn.

In some embodiments of any of the methods disclosed herein, a subject is a mammal. In some embodiments, a subject is a human, e.g., an adult or a child. In some embodiments, a subject is a dog. In some embodiments, a subject is a cat.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIGS. 1A-1B show binding affinity of two exemplary GDF15 antibody agents (Clone A and Clone C) to biotinylated GDF15 measured with a surface plasmon resonance assay. FIG. 1A is a graph showing data for biotinylated human GDF15 Fc bound to the chip with 9 nM, 3 nM, 1 nM, 0.33 nM, or 0.11 nM Fab of Clone A in solution. The data shows a Kd of 41.7 pM. FIG. 1B is a graph showing data for biotinylated human GDF15 Fc bound to the chip with 9 nM, 3 nM, 1 nM, 0.33 nM, or 0.11 nM Fab of Clone C in solution. The data shows a Kd of 17 pM.

FIGS. 2A-2D show binding affinity of Clone A to GDF15. FIG. 2A shows BioLayer Interferometry data for Clone A IgG bound to the sensor tip with 100 nM Human GDF-15 Fc in solution [Avid] as the analyte. A good theoretical fit to the data (thin line) is shown indicating 1:1 binding yielding a K_{D} of 263 pM. FIG. 2B shows BioLayer Interferometry data for Human GDF15 Fc bound to the sensor tip with 100 nM Fab of Clone A in solution [Monovalent]. A good theoretical fit to the data (thin line) indicating 1:1 binding is shown yielding a K_{D} of 1005 pM. FIG. 2C shows BioLayer Interferometry data with Cyno GDF15 Fc bound to the sensor tip with 100 nM monovalent Fab of Clone A in solution as the analyte. A good theoretical fit to the data (thin line) indicating 1:1 binding is shown yielding a K_{D} of 536 pM. FIG. 2D shows BioLayer Interferometry data with Mouse GDF15 Fc bound to the sensor tip and 100 nM monovalent Fab of Clone A in solution. The data shows no binding of Clone A to mouse GDF15 Fc.

FIGS. 3A-3D show binding affinity of Clone C to GDF15. FIG. 3A shows BioLayer Interferometry data for Clone C IgG bound to the sensor tip with 100 nM Human GDF15 Fc in solution [Avid] as the analyte. A good theoretical fit to the data (thin line) is shown indicating 1:1 binding yielding a K_{D} of 254 pM. FIG. 3B shows BioLayer Interferometry data for Human GDF15 Fc bound to the sensor tip with 100 nM Fab of Clone Cin solution [Monovalent]. A good theoretical fit to the data (thin line) indicating 1:1 binding is shown yielding a K_{D} of 731 pM. FIG. 3C shows BioLayer Interferometry data with Cyno GDF15 Fc bound to the sensor tip with 100 nM monovalent Fab of Clone C in solution as the analyte. A good theoretical fit to the data (thin line) indicating 1:1 binding is shown yielding a K_{D} of 360 pM. FIG. 3D shows BioLayer Interferometry data with Mouse GDF15 Fc bound to the sensor tip and 100 nM monovalent Fab of Clone Cin solution, with a K_{D} of 156 nM.

FIGS. 4A and 4B show pharmacokinetic properties of an exemplary GDF15 antibody agent. FIG. 4A shows the mean serum concentration-time profile of an exemplary GDF15 antibody agent dosed intravenously in Male C57BL/6 Mice at a dose of 1 mg/Kg (N=3/timepoint). FIG. 4B shows the mean serum concentration-time profile of an exemplary GDF15 antibody agent dosed subcutaneously in Male C57BL/6 Mice at a dose of 10 mg/Kg (N=3/timepoint). Blood samples were collected from the animals at 0, 1, 2, 6, 8, 24, 48, 144, 132, 312, 480 and 648 hours post-administration and GDF15 antibody levels were measured by affinity capture Liquid Chromatography-Mass Spectrometry (LC-MS).

FIGS. 5A-5C show pharmacokinetic properties of exemplary GDF15 antibody agents in primates. The graphs provide the mean concentration-time profile of exemplary GDF15 antibody agents dosed intravenously (FIGS. 5A-5B) or subcutaneously (FIG. 5C) in Male naïve cynomolgus monkeys (N=3/timepoint) at a dose of 5 mg/Kg. Samples were collected at 0 day, 2 and 8 hours), 1 day, 2 days, 3 days 7 days, 10 days, 14 days, 21 days, 28 days and 35 days post-administration and anti-GDF15 antibody levels were measured with an ELISA. FIG. 5A depicts data with intravenous administration of Clone C, FIG. 5B depicts data with intravenous administration of Clone B, and FIG. 5C depicts data with subcutaneous administration of Clone C.

FIGS. 6A-6B show reversal of weight loss with GDF15 antibody agents. FIG. 6A shows reversal of weight loss with an exemplary GDF15 antibody agent. Mice were administered an AAV vector expressing GDF15 to induce weight loss. 21 days after AAV GDF15 administration and once the animals showed >10% loss of weight, animals were separated into groups and treated as indicated in the graph. Mice treated with 20 mg/kg SC of a single dose of the exemplary GDF15 antibody agent shows significant reversal in weight loss as observed with the increase in body weight compared to the controls. FIG. 6B demonstrates that an exemplary GDF15 antibody agent (Clone C) reversed GDF-15-induced weight loss in mice with multiple dosing. Healthy mice overexpressing human GDF-15 elicited 10% weight loss. Mice dosed with Clone C at a dose of 10 mg/Kg, S.C. reversed GDF-15-induced weight loss (n=5/group) which is sustained. Arrows denote mAb injections.

FIG. 7 is a graph depicting increase in plasma GDF15 levels in animals administered Adriamycin which is a non-platinum based chemotherapy.

FIGS. 8A-8D show inhibition of GDF15-GFRAL axis with GDF15 antibodies. FIG. 8A is a GDF15 concentration response graph showing an increase in Luciferase assay. FIG. 8B. is a graph showing the GDF15 antibodies IC50 normalised data from the luciferase assay. The percent response was determined in 2 nM GDF15 (approx EC80 value) stimulated cells. FIG. 8C is a GDF15 concentration response graph showing an increase in pERK activity. FIG. 8D is a graph showing the GDF15 antibodies IC50 normalized data from the phosphorylated ERK (pERK) assay. The percent response was determined in 850 pM GDF (approx EC80 value) stimulated cells.

FIG. 9 shows protein homology analysis of GDF15 and related TGFbeta superfamily members. The area of homology between the proteins is indicated with a blue bar labeled “Potential epitope”. The cysteines in GDF15 are labeled with their position and the position of their paired cysteine. Color is added to assist in visualizing the di-sulfide bond pairings.

FIG. 10 depicts the position of predicted binding epitope for GDF15 antibodies that have weak affinity to Activin A, Activin B and GDF-10 and its position in relation to GFRAL binding domains. The epitope does not appear to be close to the area that directly interacts with the GFRAL binding pocket.

FIGS. 11A-11B show suppression of pica activity in rats in response to chemotherapy with the administration of GDF15 antibody agents. Rats were fed regular chow (Altromin 1324) and tap water. During a 2-week habituation period, rats were exposed to chow and kaolin (Kaolin Research diet, US) and feeding containers of both diets switched every 2 days. Body weight and food intake (chow and Kaolin were recorded separately) were recorded daily from day-7. At Day-3 animals were randomized based on body weight into 4 groups (n=12-14 per group): 1) Vehicle (IP)+IgG1 (20 mg/kg), 2) Cisplatin (6 mg/kg, IP)+IgG1 (20 mg/kg, SC), 3) Cisplatin (6 mg/kg, IP)+Clone C (20 mg/kg, SC), 4) Cisplatin (6 mg/kg, IP)+Clone I (20 mg/kg, SC). At Day-1, 24 hours prior to cisplatin administration, animals are dosed with a single dose of antibody. At Day 1, 24 hours after first dose, animals were dosed with a single dose of vehicle or cisplatin, and kaolin intake recorded. Data are expressed as mean±standard error of the mean. FIG. 11A shows suppression of pica activity with administration of GDF15 antibody agent Clone C and FIG. 11B shows suppression of pica activity with administration of GDF15 antibody agent Clone I.

FIGS. 12A-12B show Fc region variant comprising a AAGA mutation and binding to FcRn. FIG. 12A: shows binding of GDF15 antibody agent clone B to FcRn at pH 6.0 (top) and no binding at pH 7.4 (bottom). FIG. 12B: shows binding of GDF15 antibody agent clone C to FcRn at pH 6.0 (top) and no binding at pH 7.4 (bottom).

FIG. 13 shows prevention of a reduction in food intake with an exemplary GDF15 antibody agent. To assess the efficacy of GDF15 antibody agent Clone C in preventing human GDF-15-induced reduction in food intake in mice, acute food intake studies were performed by administering recombinant human GDF-15 (hGDF-15, 4 nmol/Kg) to healthy mice. hGDF-15 suppressed food intake by 22% over 8 hours. GDF15 antibody agent Clone C (10 mg/Kg, S.C.) alone had a minimal effect on food intake but prevented hGDF-15-induced reduction (n=8, P<0.01).

FIGS. 14A-14D show a reversal of tumor-induced weight loss in mice with an exemplary GDF15 antibody agent. Female SCID mice subcutaneously implanted with human HT-1080 tumor cells elicited involuntary weight loss (A; black squares). Administration of GDF15 antibody agent Clone B (FIG. 14A) or GDF15 antibody agent Clone C (FIG. 14B) (10 mg/Kg S.C., n=8-12; purple triangles) fully reversed the tumor-induced weight loss. Administration of GDF15 antibody agent Clone B (FIG. 14C) or GDF15 antibody agent Clone C (FIG. 14D) had no adverse effect on tumor volume. Arrow depicts mAb injection. NTB=Non Tumor Bearing; TB=Tumor Bearing.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

GDF15: The term “GDF15”, as used herein refers to Growth Differentiation Factor 15 which is a member of the TGFbeta superfamily. Amino acid sequences for full-length GDF15, and/or for nucleic acids that encode it can be found in a public database such as GenBank, UniProt and Swiss-Prot. For example, the amino acid sequence of human GDF15 (SEQ ID NO: 183, for which residues 1-29 represent the signal peptide, residues 30-194 represent propeptide, and residues 195-308 represent mature polypeptide; position 70 has been identified as a glycosylation site; intrachain disulfide bonds have been reported between residues 203/210, 211/274, 240/305, 244/307; and residue 273 has been described as a site for an interchain disulfide bond) can be found as UniProt/Swiss-Prot Accession No. Q99988 and the nucleic acid sequence (SEQ ID NO: 190) encoding human GDF15 can be found at Accession No. NM_004864.3. GDF15 is also known, for example, as macrophage inhibiting cytokine 1 (MIC-1), prostate derived factor (PDF), placental bone morphogenetic protein (PLAB), NSAID-activated gene 1 (NAG-1), and placental transforming growth factor beta. (PTGFB). Those skilled in the art will appreciate that sequences presented in SEQ ID NOs: 183 and 190 are exemplary, and certain variations (including, for example, conservative substitutions in SEQ ID NO: 183, codon-optimized variants of SEQ ID NO: 190, etc) are understood to also be or encode human GDF15; additionally, those skilled in the art will appreciate that homologs and orthologs of human GDF15 are known and/or knowable through the exercise or ordinary skill, for example, based on degree of sequence identity, presence of one or more characteristic sequence elements, and/or one or more shared activities.

GDF15 polypeptide: The phrase “GDF15 polypeptide”, is used herein to refer to polypeptides that share significant sequence identity and/or at least one characteristic sequence element with an appropriate reference polypeptide such as, for example: (a) human GDF15, for example, as set forth in SEQ ID NO: 183; (b) cyno GDF15, for example as set forth in SEQ ID NO: 184; (c) dog GDF15, for example as set forth in SEQ ID NO: 185; and/or (d) cat GDF15 for example as set forth in SEQ ID NO: 186. In some embodiments, a GDF15 polypeptide is or comprises a fragment of a parental GDF15 polypeptide (e.g., of SEQ ID NO: 183 or a homolog, ortholog, or variant [e.g., a functional variant] thereof). In some embodiments, a GDF15 polypeptide shares at least one characteristic sequence element with a reference GDF15 polypeptide (e.g., of SEQ ID NO: 183 or a homolog, ortholog, or variant [e.g., a functional variant] thereof). Alternatively or additionally, in some embodiments, a GDF15 polypeptide shares significant amino acid sequence identity with a relevant reference polypeptide (e.g., of SEQ ID NO: 183 or a homolog, ortholog, or variant [e.g., a functional variant] thereof). For example, in some embodiments, a GDF15 polypeptide shares at least 50% with a reference GDF15. In some embodiments, a GDF15 polypeptide is characterized by an ability to activate a receptor that binds GDF15, e.g., a GFRAL receptor; in some such embodiments, such ability is comparable to that of an appropriate reference GDF15 (e.g., of SEQ ID NO: 183 or a homolog, ortholog, or variant [e.g., a functional variant] thereof). For example, in some embodiments, a GDF15 polypeptide activates a GFRAL receptor with a binding affinity that is reasonably comparable to that of an appropriate reference GDF15 (e.g., of SEQ ID NO: 183 or a homolog, ortholog, or variant [e.g., a functional variant] thereof); in some embodiments, a GDF15 polypeptide is characterized in that it competes with an appropriate reference GDF15 (e.g., of SEQ ID NO: 183 or a homolog, ortholog, or variant [e.g., a functional variant] thereof) for binding and/or activation of a GFRAL receptor; in some such embodiments, such competition is observed over a range of concentrations (e.g., which range may, for example, extend over 2 fold, 3 fold, 4 fold, 5 fold, 10 fold, or more). In some embodiments, a GDF15 polypeptide is or comprises a polypeptide with at least 50% identity to SEQ ID NO: 183.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system, for example to achieve delivery of an agent that is, or is included in or otherwise delivered by, the composition. Those of ordinary skill in the art will be aware of a variety of routes that may, in appropriate circumstances, be utilized for administration to a subject, for example an animal or a human. In some embodiments, an animal is a domestic animal, such as a companion animal, e.g., a dog or a cat; in some embodiments, an animal is an animal used in agriculture (e.g., farming [e.g., a cow, a sheep or a horse]) or for recreation. For example, in some embodiments, administration may be systemic or local. Those skilled in the art will be aware of appropriate administration routes for use with particular therapies described herein, for example which include bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may be by injection (e.g., intramuscular, intravenous, or subcutaneous injection). In some embodiments, injection may involve bolus injection, drip, perfusion, or infusion. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Adult: As used herein, the term “adult” refers to a human eighteen years of age or older. In some embodiments, a human adult has a weight within the range of about 90 pounds to about 250 pounds.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological-setting). In some embodiments, affinity is assessed relative to a reference (e.g., that has a known affinity above a particular threshold [a “positive control” reference] or that has a known affinity below a particular threshold [a “negative control” reference “]. In some embodiments, affinity may be assessed relative to a contemporaneous reference; in some embodiments, affinity may be assessed relative to a historical reference. Typically, when affinity is assessed relative to a reference, it is assessed under comparable conditions.

Affinity matured” (or “affinity matured antibody”): as used herein, refers to an antibody with one or more alterations in one or more CDRs thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al., BioTechnology 10:779-783 (1992) describes affinity maturation by V_{H} and V_{L} domain shuffling. Random mutagenesis of CDR and/or framework residues is described by: Barbas et al. Proc. Nat. Acad. Sci. U.S.A 91:3809-3813 (1994); Schier et al., Gene 169:147-155 (1995); Yelton et al., J. Immunol. 155:1994-2004 (1995); Jackson et al., J. Immunol. 154 (7): 3310-9 (1995); and Hawkins et al., J. Mol. Biol. 226:889-896 (1992).

Agent: As used herein, the term “agent”, may refer to a physical entity or phenomenon. In some embodiments, an agent may be characterized by a particular feature and/or effect. In some embodiments, an agent may be a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Agonist: Those skilled in the art will appreciate that the term “agonist” may be used to refer to an agent, condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered). Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Animal: as used herein refers to a member of the animal kingdom. In some embodiments, “animal” refers to humans; unless otherwise specified, in many embodiments, a human may be of either gender and/or at any stage of development. In some embodiments, “animal” refers to non-human animals; unless otherwise specified, in many embodiments, a non-human animal may be of any gender and/or at any stage of development. In certain embodiments, a non-human animal is a mammal (e.g., a rodent, a mouse, a rat, a rabbit, a monkey, a dog, a cat, a sheep, cattle, a primate, and/or a pig). In some embodiments, an animal may be, for example, a mammals, a bird, a reptile, an amphibian, a fish, an insect, a worm, etc., In some embodiments, an animal may be a transgenic animal, genetically engineered animal, and/or a clone.

Antagonist: Those skilled in the art will appreciate that the term “antagonist”, as used herein, may be used to refer to an agent, condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. In some embodiments, antibodies produced and/or utilized in accordance with the present disclosure include one or more modifications on an Fc domain, e.g., an effector null mutation, e.g., a LALA, LAGA, FEGG, AAGG, or AAGA mutation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of dog, cat, mouse, rabbit, primate, or human antibodies. In some embodiments, antibody sequence elements are human, humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies, alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies, etc. In some embodiments, relevant formats may be or include: Adnectins®; Affibodies®; Affilins®; Anticalins®; Avimers®; BiTERs; cameloid antibodies; Centyrins®; ankyrin repeat proteins or DARPINs®; dual-affinity re-targeting (DART) agents; Fynomers®; shark single domain antibodies such as IgNAR; immune mobilizing monoclonal T cell receptors against cancer (ImmTACs); KALBITOR®s; MicroProteins; Nanobodies® minibodies; masked antibodies (e.g., Probodies®); Small Modular ImmunoPharmaceuticals (“SMIPs™”); single chain or Tandem diabodies (TandAb®); TCR-like antibodies; Trans-bodies®; TrimerX®; VHHs. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of dog, cat, mouse, rabbit, primate, or human antibodies. In some embodiments, an antibody agent may include one or more sequence elements that are human, humanized, primatized, chimeric, etc, as is known in the art. In some embodiments, an antibody agent may include one or more complementarity determining regions that are human and/or one or more constant region sequences that are characteristic of human antibodies. In many embodiments, the term “antibody agent” is used to refer to one or more of the art-known or developed constructs or formats for utilizing antibody structural and functional features in alternative presentation. For example, in some embodiments, an antibody agent utilized in accordance with the present disclosure is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide comprising an antigen binding specificity fused to an Fc; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINS®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 96%, 96%, 97%, 98%, 99%, or 100% sequence identity with the reference CDR. In some embodiments, an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody fragment: As used herein, an “antibody fragment” refers to a portion of an antibody or antibody agent as described herein, and typically refers to a portion that includes an antigen-binding portion or variable region thereof. An antibody fragment may be produced by any means. For example, in some embodiments, an antibody fragment may be enzymatically or chemically produced by fragmentation of an intact antibody or antibody agent. Alternatively, in some embodiments, an antibody fragment may be recombinantly produced (i.e., by expression of an engineered nucleic acid sequence. In some embodiments, an antibody fragment may be wholly or partially synthetically produced. In some embodiments, an antibody fragment (particularly an antigen-binding antibody fragment) may have a length of at least about 50, 60, 70, 80, 90, 100, 110, 120, 130, 140, 150, 160, 170, 180, 190 amino acids or more, in some embodiments at least about 200 amino acids.

Antibody polypeptide: As used herein, the term “antibody polypeptide” refers to a polypeptide(s) that includes characteristic sequence element(s) (e.g., one or more CDRs, or a set of CDRs such as each of a CDR1, 2, and 3 as found in reference antibody chain and/or one or more FR regions and/or a set of FR regions, such as, for example, a complete variable region of a heavy or light chain of a reference antibody) of an antibody; in many embodiments, an antibody polypeptide includes sufficient such sequence element(s) that it binds to an epitope (e.g., an epitope bound by a reference antibody including the characteristic sequence element). In some embodiments, an antibody polypeptide is a full-length antibody or heavy or light chain thereof. In some embodiments, an antibody polypeptide is or comprises a complete heavy and/or light chain variable region of a reference antibody; in some such embodiments, an antibody polypeptide includes characteristic antibody sequence element(s) sufficient to confer specific binding to a relevant epitope—i.e., so that the antibody polypeptide includes at least one binding site. In some embodiments, an “antibody polypeptide” may include a binding domain which is homologous or largely homologous (e.g., shows significant sequence homology and/or in some embodiments significant sequence identity) to an immunoglobulin-binding domain. In some embodiments, an antibody polypeptide shows at least 99% identity with an immunoglobulin binding domain. In some embodiments, an “antibody polypeptide” has a binding domain that shows at least 70%, 80%, 85%, 90%, or 95% identity with an immuoglobulin binding domain, for example a reference immunoglobulin binding domain. In some embodiments, an “antibody polypeptide” may have an amino acid sequence identical to that of an antibody, or chain, or variable region thereof (or combination of variable region(s)) that is found in a natural source. In some embodiments, an antibody polypeptide may be prepared by, for example, isolation from a natural source or antibody library, recombinant production in or with a host system, chemical synthesis, etc., or combinations thereof. In some embodiments, an antibody polypeptide is an antibody agent as described herein.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: Those skilled in the art will appreciate that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

CDR-grafted antibody: as used herein, refers to an antibody whose amino acid sequence comprises heavy and light chain variable region sequences from one species but in which the sequences of one or more of the CDR regions of V_{H} and/or V_{L} are replaced with CDR sequences of another species, such as antibodies having murine V_{H} and V_{L} regions in which one or more of the murine CDRs (e.g., CDR3) has been replaced with human CDR sequences. Likewise, a “CDR-grafted antibody” may also refer to antibodies having human V_{H} and V_{L} regions in which one or more of the human CDRs (e.g., CDR3) has been replaced with mouse CDR sequences.

Child: As used herein, the term “child” refers to a human between 1 day and 18 years of age. In some embodiments, a child may be an infant (e.g., may be less than or equal to about 12 months, 11 months, 10 months, 9 months, 8 months, 7 months, 6 months, 5 months, 4 months, 3 months, 2 months or 1 month old); in some embodiments, a child may be older than an infant. In some embodiments, a child may be a toddler (e.g., about 1 to about 3 years old); in some embodiments, a child may be younger than or older than a toddler. In some embodiments, a child may be a teen (e.g., between about 12 and about 18 years old); in some embodiments, a child may be younger than a teen (and/or older or younger than a toddler or older than an infant). Body weight can vary widely across ages and specific children, with a typical range being 4 pounds to 150 pounds.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

Effector function: as used herein refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

Effector cell: as used herein refers to a cell of the immune system that expresses one or more Fc receptors and mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Epitope: as used herein, includes any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Framework” or “framework region: as used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Functional: As used herein, a “functional” biological molecule is a biological molecule in a form in which it exhibits a property and/or activity by which it is characterized.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

High affinity binding: The term “high affinity binding”, as used herein refers to a high degree of tightness with which a particular ligand binds to its partner. Affinities can be measured by any available method, including those known in the art. In some embodiments, binding is considered to be high affinity if the K_{d} is about 500 pM or less (e.g., below about 400 pM, about 300 pM, about 200 pM, about 100 pM, about 90 pM, about 80 pM, about 70 pM, about 60 pM, about 50 pM, about 40 pM, about 30 pM, about 20 pM, about 10 pM, about 5 pM, about 4 pM, about 3 pM, about 2 pM, etc.) in binding assays. In some embodiments, binding is considered to be high affinity if the affinity is stronger (e.g., the K_{d} is lower) for a polypeptide of interest than for a selected reference polypeptide. In some embodiments, binding is considered to be high affinity if the ratio of the K_{d} for a polypeptide of interest to the K_{d} for a selected reference polypeptide is 1:1 or less (e.g., 0.9:1, 0.8:1, 0.7:1, 0.6:1, 0.5:1, 0.4:1, 0.3:1, 0.2:1, 0.1:1, 0.05:1, 0.01:1, or less). In some embodiments, binding is considered to be high affinity if the K_{d} for a polypeptide of interest is about 100% or less (e.g., about 99%, about 98%, about 97%, about 96%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, about 5%, about 4%, about 3%, about 2%, about 1% or less) of the K_{d} for a selected reference polypeptide.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Human: In some embodiments, a human is an embryo, a fetus, an infant, a child, a teenager, an adult, or a senior citizen.

Humanized: as is known in the art, the term “humanized” is commonly used to refer to antibodies (or antibody components) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

K_{D}: as used herein, refers to the dissociation constant of a binding agent (e.g., an antibody or binding component thereof) from a complex with its partner (e.g., the epitope to which the antibody or binding component thereof binds).

Low affinity binding: The term “low affinity binding”, as used herein refers to a low degree of tightness with which a particular ligand binds to its partner. As described herein, affinities can be measured by any available method, including methods known in the art. In some embodiments, binding is considered to be low affinity if the K_{d} is about 501 pM or more (e.g., above about 501 pM, 600 pM, 700 pM, 800 pM, 900 pM, 1 nM, 1.1. nM, 1.2 nM, 1.3 nM, 1.4 nM, 1.5 nM, etc.) In some embodiments, binding is considered to be low affinity if the affinity is the same or lower (e.g., the K_{d} is about the same or higher) for a polypeptide of interest than for a selected reference polypeptide. In some embodiments, binding is considered to be low affinity if the ratio of the K_{d} for a polypeptide of interest to the K_{d} for a selected reference polypeptide is 1:1 or more (e.g., 1.1:1, 1.2:1, 1.3:1, 1.4:1, 1.5:1, 1.6:1, 1.7:1, 1.8:1, 1.9:1, 2:1, 3:1, 4:1, 5:1, 10:1 or more). In some embodiments, binding is considered to be low affinity if the K_{d} for a polypeptide of interest is 100% or more (e.g., 100%, 105%, 110%, 115%, 120%, 125%, 130%, 135%, 140%, 145%, 150%, 155%, 160%, 165%, 170%, 175%, 180%, 185%, 190%, 195%, 200%, 300%, 400%, 500%, 1000%, or more) of the K_{d} for a selected reference polypeptide.

Modulator: The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level. In some embodiments, a modulator can bind at a target entity active site. In some embodiments, a modulator can bind at a target entity allosteric site (e.g., can act as, for example, a non-competitive antagonists or an inverse agonists), in that response to a stimulus is reduced in its presence as compared with otherwise comparable conditions when the modulator is absent.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in a particular form (e.g., in a solid form or a liquid form), and/or may be specifically adapted for, for example: oral administration (for example, as a drenche [aqueous or non-aqueous solutions or suspensions], tablet, capsule, bolus, powder, granule, paste, etc, which may be formulated specifically for example for buccal, sublingual, or systemic absorption); parenteral administration (for example, by subcutaneous, intramuscular, intravenous or epidural injection as, for example, a sterile solution or suspension, or sustained-release formulation, etc); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent: Those skilled in the art will appreciate that, in the context of therapeutic treatments”, the term “prevent” refers to delay of onset (in some embodiments indefinitely) and/or reduction in incidence (e.g., frequency) and/or severity of a particular symptom or characteristic of a disease, disorder or condition. Typically, a method of “preventing” involves administering therapy prior to onset of (or prior to increase in) a particular incident or episode of such symptom or characteristic.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: as used herein refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al., Basic local alignment search tool, J. Mol. Biol., 215(3): 403-410, 1990; Altschul et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al, (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having not more than a small number (e.g., 3, 2, or 1) an amino acid sequence changes relative to that of a reference CDR. In some embodiments, a CDR that is substantially identical to a reference CDR differs from that reference CDR by one or more amino acid changes at the end of the reference CDR; in some such embodiments, the relevant CDR is identical to the reference CDR other than at one or both ends. As is known in the art, CDR elements typically have a length within a range of a few amino acids (e.g., 3, 4, 5, 6, or 7) to about 20 or 30 amino acids (see, for example, Collis et al. J. Mol. Biol. 325:337, 2003, incorporated herein by reference); thus, in some embodiments, a CDR may be considered to be substantially identical to a reference CDR when it shares at least about 80% (or less for a shorter CDR), at least about 85%, at least about 90%, at least about 95%, at least about 98%, at least about 99%, or at least about 100% identity with the reference CDR.

Substantial sequence homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids., and/or as having “polar” or “non-polar” side chains Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215 (3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Treat: As used herein, the term “treat,” “treatment,” or “treating” is used to refer to one or more of partial or complete alleviation, amelioration, relief, inhibition, prevention, delay of onset of, reduction in severity of and/or reduction in frequency (e.g., incidence) of one or more symptoms or features of a disease, disorder, and/or condition. In some embodiments, treatment may be prophylactic; for example may be administered to a subject who does not exhibit signs of a disease, disorder, and/or condition. In some embodiments, treatment may be administered to a subject who exhibits only early signs of the disease, disorder, and/or condition, and may, for example, decrease risk of developing pathology associated with the disease, disorder, and/or condition and/or delay onset and/or decrease rate of development or worsening of one or more features of a disease, disorder and/or condition.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors, e.g., that are statistically correlated with increased risk of development of the relevant disease, disorder, and/or condition. Thus, in some embodiments, treatment may be prophylactic; in some embodiments, treatment may be therapeutic.

Variant: The term “variant”, as used herein, refers to a molecule or entity (e.g., that are or comprise a nucleic acid, protein, or small molecule) that shows significant structural identity with a reference molecule or entity but differs structurally from the reference molecule or entity, e.g., in the presence or absence or in the level of one or more chemical moieties as compared to the reference molecule or entity. In some embodiments, a variant also differs functionally from its reference molecule or entity. In many embodiments, whether a particular molecule or entity is properly considered to be a “variant” of a reference is based on its degree of structural identity with the reference molecule. As will be appreciated by those skilled in the art, a biological or chemical reference molecule in typically characterized by certain characteristic structural elements. A variant, by definition, is a distinct molecule or entity that shares one or more such characteristic structural elements but differs in at least one aspect from the reference molecule or entity. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

### GDF15

Growth differentiation factor 15 (GDF15 or GDF-15) is a secreted protein, its monomer is 12 kDa that forms a 25 kDa disulfide-linked homodimer and is a member of the transforming growth factor beta (TGFbeta) superfamily. GDF15 is also known as macrophage inhibiting cytokine 1 (MIC-1), prostate derived factor (PDF), placental bone morphogenetic protein (PLAB), NSAID-activated gene 1 (NAG-1), and placental transforming growth factor beta. (PTGFB). In healthy individuals, GDF15 expression is most prominent in the placenta and prostrate. GDF15 shows low to absent constitutive expression in many cell types, but GDF15 expression can be upregulated in response to stimuli, e.g., inflammation, malignancy and/or exposure to therapeutics.

Recent reports demonstrate that increased levels of GDF15 can induce nausea and emesis, weight loss, delays in gastric emptying, and a loss of appetite (Hale et.al., Mol Metab. 2021 April; 46:101117; and Borner et.al., Cell Metab. 2020 Feb. 4; 31 (2): 351-362.e5). Increased level of GDF15 has been associated with various pathophysiological functions, including cancer cachexia, renal and heart failure, atherosclerosis and metabolism. For example, hyperemesis gravidarum has been associated with elevated levels of GDF15 levels in pregnancy (Fejzo et al 2018, Fejzo et al 2019, Mullin et al 2020). Additionally, patients carrying certain GDF15 variants associated with overexpression of GDF15 may also have an increased risk of hyperemesis gravidarum and recurrence. In some embodiments, a relevant variant may be associated with a single nucleotide polymorphism (“SNP”) such as, for example, rs105422.

A recent report by Breen et al 2020 disclosed elevation of GDF15 in cancer patients treated with a platinum-based chemotherapy and that increased GDF15 was associated with weight loss, nausea and anorexia in animal models. Increased plasma GDF15 was also shown to be associated with weight loss in cancer patients with cachexia (see, e.g., US 2020/0055930A1, WO2005/099746; WO2009/021293, WO2014/100689, and WO2016/049470, the entire contents of each of which are hereby incorporated by reference in their entireties).

A human GDF15 polypeptide sequence having UniProt accession number Q99988 is provided herein as SEQ ID NO: 183:

MPGQELRTVNGSQMLLVLLVLSWLPHGGALSLAEASRASFPGPSE
LHSEDSRFRELRKRYEDLLTRLRANQSWEDSNTDLVPAPAVRILT
PEVRLGSGGHLHLRISRAALPEGLPEASRLHRALFRLSPTASRSW
DVTRPLRRQLSLARPQAPALHLRLSPPPSQSDQLLAESSSARPQL
ELHLRPQAARGRRRARARNGDHCPLGPGRCCRLHTVRASLEDLGW
ADWVLSPREVQVTMCIGACPSQFRAANMHAQIKTSLHRLKPDTVP
APCCVPASYNPMVLIQKTDTGVSLQTYDDLLAKDCHCI

The amino acid sequence of human GDF15 (SEQ ID NO:183) includes a signal peptide (residues1-29); a propeptide (residues 30-194) and a mature polypeptide (residues 195-308).

Human GDF15 signal peptide (SEQ ID NO: 187)

MPGQELRTVN GSQMLLVLLV LSWLPHGGA

Human GDF15 propeptide (SEQ ID NO: 188)

LSLAEASRASF PGPSELHSED SRFRELRKRY EDLLTRLRAN
QSWEDSNTDL VPAPAVRILT PEVRLGSGGH LHLRISRAAL
PEGLPEASRL HRALFRLSPT ASRSWDVTRP LRRQLSLARP
QAPALHLRLS PPPSQSDQLL AESSSARPQL ELHLRPQAAR
GRRR

Human GDF15 mature polypeptide (SEQ ID NO: 189)

ARARNG DHCPLGPGRC CRLHTVRASL EDLGWADWVL
SPREVQVTMC IGACPSQFRA ANMHAQIKTS LHRLKPDTVP
APCCVPASYN PMVLIQKTDT GVSLQTYDDL LAKDCHCI

GDF15 is also expressed by other mammals besides humans and exemplary sequences are provided below:

Cyno GDF15 having UniProt accession number G7PWZ3 (SEQ ID NO: 184)

MPGQELKTLNGSQMLLVLLVLLWPPHGGAVSLAEASRASFPGPSD
LHSEDSRFRELRKRYEDLLTRLRANQSWEDSNTDLIQAPEVRILT
PEVRLGSGGHLHLRISRAVLPEGLPEACRIHRALFRLSPTASRSR
DVTRPLRRQLRLARPQAPALHLRLSPPPSQSDQLLVKSSSSRPQL
ALHLRPRASRGRRRARARNGDRCPLGPGRCCRLHTVHASLEDLGW
ADWVLSPREVQVTMCIGACPSQFREANMHAQIKMNLHRLKPDTVP
APCCVPASYNPMVLIQKTDTGVSLQTYDDLLAKDCHCV

Canis familiaris (dog) GDF 15 having UniProt accession number F1PDK9 (SEQ ID NO: 185)

MPGQGPAPAHCSPMLVILVMLSWLPSGGALSLAQEHLPAFPGPSD
PHSSTDVSRIQELRKRYEHLQTKLRLNQGWADSNPDLVPATRVRI
LTPKLRLGPRGHLHLRIARADLTAGLPAASRLHRALLRLSPTEPS
SWDVTRPLQRQLSRVGSRTPTLRLRLLPRWDRSRALPSARPQLEL
HWRPRAARGRRNAHAHARDGCPLGEGRCCRLQSLRASLQDLGWAN
WVVAPRELDVRMCVGACPSQFRSANTHAQMQARLHGLNPDAAPAP
CCVPASYEPVVLMHQDSDGRVSLTPFDDLVAKDCHCV

Felis catus (cat) GDF15 having UniProt accession number M3WC01 (SEQ ID NO: 186)

SVQNSASTGMPGPGPTPPMLLMLLMLLMLCWLPSGGALSLAQEHL
PAFPGPSEARSGTDVSRFEEFRKLYEHLQTRLRLNQSWEDSNPDR
VISEAQVRILTPKLRLGLGGHLHLRIARADLTKGLPASFRLHRAL
LRLSPTELSSWDVTRPLRRQLSLGGSGRDRSPAALPSSARPQLEL
HWRPRAARGRRNAHARSKDDCPLGAGRCCRLQSLRASLEDLGWAS
WVVAPRELDVRMCIGACPSQFRSANTHAQMQARLHGLNPDATPAP
CCVPARYEPVVLMHQDSDGRVSLTPFDDLVAKDCHCL

### GDF15-GFRAL Pathway

GDF15 can bind to and activate glial cell-derived neurotrophic factor receptor alpha-like (GFRAL). GFRAL is primarily expressed in the hindbrain (e.g., the area postrema and nucleus tractus solitaries) and is an orphan member of the GFR-alpha family (see, e.g., Hsu et al., 2017, Nature 550:255-259; Yang et al., 2017, Nature Med. 23 (10): 1158; and Emmerson et al., 2017, Nature Med. 23 (10): 1215). Studies of GDF15 binding to GFRAL demonstrated that this binding activates a GFRAL-mediated signaling pathway whereby a receptor tyrosine kinase, RET, is activated and acts as a co-receptor of GFRAL. RET, in turn, mediates downstream phosphorylation of ERK (PERK), ribosomal protein S6 (pS6), AKT, MAPK, and phospholipase C gamma 1 (PLC-gamma1) among others.

Activation of GFRAL by GDF15 occurs in the regions of the brainstem (e.g., the area postrema and nucleus tractus solitaries) that play a role in controlling appetite and emesis. Systemically administered GDF15 was indirectly shown to increase neuronal activity in the area postrema (see, Johnen et al., 2007, Nature Med. 13 (11): 1133-40). Importantly, ablation of the area postrema and regions of the hindbrain important in appetite regulation in mice resulted in a loss of GDF15 function to decrease food intake and reduce body weight (see, Tsai et al., 2014, PLOS One. 9 (6): e100370).

Intracerebroventricular (ICV) injections of small amounts of GDF15 in mice resulted in decreased food intake, possible because acute and chronic activation of GDF15-GFRAL pathway can result in visceral malaise and subsequently weight loss. (see, Sabatini et al., 2021, PNAS 18 (8): e2021357118.; Borner et al., 2020, Cell Rep. 31 (3): 107543.) Lastly, GDF15 neutralizing antibody attenuated chemotherapy agents-induced emesis and anorexia in nonhuman primates (see, Breen et al., 2020, Cell Metab. 32 (6): 938-950.).

### GDF15-GFRAL Pathway Modulating Agents

The present disclosure provides insights relating to agents (i.e., GDF15-GFRAL Pathway Modulating Agents) in whose presence level and/or activity of the GDF15-GFRAL Pathway is altered. In some embodiments, a relevant agent interacts directly with a component of the GDF15-GFRAL Pathway (e.g., with GDF15, with GFRAL, and/or with RET). In some embodiments, a relevant agent may not interact directly with such component (e.g., with GDF15, with GFRAL, and/or with RET), but may alter level and/or activity of the component (e.g., with GDF15, with GFRAL, and/or with RET). In particular, the present disclosure provides insights that such agents are useful in the treatment of certain diseases, disorders and/or conditions (e.g., where a role for the GDF15-GFRAL Pathway was not previously appreciated). For example, in embodiments, the present disclosure provides methods of treating one or more of Cyclic Vomiting Syndrome (CVS), Cannabinoid Hyperemesis Syndrome (CHS), and/or Migraine Associated Nausea/Vomiting (MAN/V) with GDF15-GFRAL Pathway Modulating Agents.

Those skilled in the art, reading the present disclosure, will appreciate that, in different embodiments, useful GDF15-GFRAL Pathway Modulating Agents may be agents of different chemical classes (e.g., nucleic acids, such as anti-sense oligonucleotides, polypeptides, such as antibody agents, small molecules, etc).

In some embodiments, a GDF15-GFRAL Pathway Modulating Agent may bind to one or more of GDF15, GFRAL, and RET and/or may disrupt interaction there between.

In some embodiments, a GDF15-GFRAL Pathway Modulating Agent may bind directly to GDF15. In some embodiments, a useful such agent may show preferential binding to GDF15 relative to one or more TGFbeta family members other than GDF15

In some embodiments, a GDF15-GFRAL Pathway Modulating Agent may show preferential binding to GFRAL. In some embodiments, a useful such agent may specifically bind to GFRAL with high affinity.

In some embodiments, a GDF15-GFRAL Pathway Modulating Agent may show preferential binding to RET. In some embodiments, a useful such agent may specifically bind to RET with high affinity.

In some embodiments, a GDF15-GFRAL Pathway Modulating Agent may reduce activation of GFRAL and/or activation of RET.

### Polypeptide Agents

In some embodiments, a GDF15-GFRAL Pathway Modulation Agent may be or comprise a polypeptide.

In some embodiments, a useful such polypeptide agent is or comprises a polypeptide that binds to one or more of GDF15 and/or GFRAL and/or RET and disrupts (e.g., competes) interaction there between. In some such examples, such polypeptide may be or comprises an Antibody Agent (e.g., an antibody, or antigen binding portion thereof) that binds specifically to GDF15 and/or to GFRAL and/or RET. Certain Antibody Agent that bind to GDF15 and/or GFRAL have been described (see, for example U.S. 62/231,484, WO2016069921, WO2017172260).

In some such examples, such polypeptide may be or comprises a dominant negative GDF15 or GFRAL or RET variant (e.g., a fragment of one that binds the other but lacks other functionality present in full-length protein).

Alternatively or additionally, in some embodiments, a useful polypeptide agent may be or comprise an agent that regulates expression and/or level of one or more components of the GDF15-GFRAL Pathway (e.g., of GDF15 and/or of GFRAL and/or RET). For example, in some embodiments, a useful polypeptide agent may modulate one or more of transcription, processing (e.g., capping, polyadenylation, splicing, localization (e.g., cytoplasmic transport and/or sequestration), translation, etc of a GDF15 and/or of a GFRAL gene and/or of RET gene.

In some embodiments, a useful polypeptide agent competes an interaction (e.g., “squelching” or sequestering an interaction component) important to the GDF15-GFRAL Pathway. In some such embodiments, such interaction is between GDF15 and/or GFRAL and/or RET.

### GDF15 Antibody Agents

Various antibodies targeting GDF15 are known (see, for example, see Table 1 and 2). Of particular interest for certain embodiments of the present disclosure are antibody agents as disclosed in U.S. Application No. 62/231,484. In some embodiments, such antibodies, or other agents that include antigen-binding portions thereof, are utilized as GDF15-GFRAL Pathway Modulating Agents as described herein.

Disclosed herein is use of GDF15-GFRAL Pathway Modulating Agents to treat and/or prevent certain types and/or instances of nausea and/or emesis with which it had not previously been considered to be associated. In some embodiments, a GDF15-GFRAL Pathway Modulating Agents may bind, e.g., specifically bind, to GDF15, e.g., with high affinity. In some embodiments, a GDF15-GFRAL Pathway Modulating Agents may be or comprise an Antibody Agent that targets GDF15 or GFRAL. In some embodiments, use of a GDF15 Antibody Agent, may be effective in the plasma and/or multiple tissue compartments, where GDF15 can act on its target cells, e.g., a cell expressing a receptor that binds to GDF15.

In some embodiments, a GDF15 target cell is or comprises a cell expressing a GDF15 receptor, e.g., GFRAL. In some embodiments, use of a GDF15 Antibody Agent can modulate a GDF15-GFRAL Pathway to inhibit one or more activities of GDF15 or to reduce a level of GDF15, e.g., reduce a level of free and/or active GDF15. In some embodiments, a GDF15 Antibody Agent binds to GDF15 and inhibits an activity and/or reduces a level of GDF15, e.g., reduce a level of free and/or active GDF15. In some embodiments, a GDF15 Antibody Agent binds to GDF15 and prevents binding of GDF15 to a receptor, e.g., GFRAL. In some embodiments, binding of a GDF15 Antibody Agents to GDF15 prevents activation of a GDF15 receptor, e.g., GFRAL and/or RET.

In some embodiments, a GDF15 target cell is or comprises a cell expressing a GDF15 receptor. In some embodiments a GDF15 target cell is within the brainstem; in some embodiments a GDF15 target cell is outside of a brain, e.g., in circulation or in a tissue other than a brain. In some embodiments, use of a GDF15 Antibody Agents inhibits an activity and/or reduces a level of GDF15, e.g., reduce a level of free and/or active GDF15. In some embodiments, a GDF15 Antibody Agents binds to GDF15 and prevents binding of GDF15 to a GDF15 receptor.

In some embodiments, a GDF15 Antibody Agents binds to any or all forms of GDF15, e.g., intracellular GDF15, soluble GDF15, extracellular matrix (ECM)-bound GDF15, mature GDF15, pro-protein GDF15 (e.g., preprocessed) and/or active GDF15.

In some embodiments, use of a GDF15 Antibody Agents reduces a level of GDF15 (e.g., free and/or active GDF15). In some embodiments, a level of circulating GDF15 is reduced. In some embodiments, a level of free and/or active GDF15 is reduced. In some embodiments, a level of free and active GDF15 is reduced.

In some embodiments, a level of GDF15 (e.g., free and/or active GDF15) is reduced relative to a comparator. In some embodiments, a comparator comprises a cell, tissue or subject which has not been contacted with a GDF15 Antibody Agents disclosed herein.

In some embodiments, a level of GDF15 (e.g., free and/or active GDF15) is reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% or about 100%. In some embodiments, a level of GDF15 (e.g., free and/or active GDF15) is reduced by about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, about 50% to about 100%, about 55% to about 100%, about 60% to about 100%, about 65% to about 100%, about 70% to about 100%, about 75% to about 100%, about 80% to about 100%, about 90% to about 100%, or about 95% to about 100%.

In some embodiments, a level of GDF15 (e.g., free and/or active GDF15) is reduced by about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, or about 5% to about 10%.

In some embodiments, a GDF15 activity comprises one or more, or all, or any combination of the following: (a) decreasing food intake; (b) decreasing appetite; (c) decreasing body weight; (d) increasing weight loss; (e) decreasing fat mass; (f) decreasing lean mass; (g) increasing loss of fat mass, (h) preventing weight gain; (i) increasing loss of lean muscle mass, (j) increasing fatigue; (k) decreasing pro-inflammation; (l) decreasing immune cell infiltration in tumor; (m) increasing metastases; (n) decreasing efficacy of immunotherapy (e.g., immune checkpoint inhibitor therapy); (o) increasing cellular senescence; (p) binding to a receptor, e.g., GFRAL; (q) increasing downstream signaling mediated by RET; (r) increasing phosphorylation of ERK; (s) increasing phosphorylation of ribosomal protein S6; (t) increasing RET-mediated activation of the MAPK signaling pathway; (u) increasing RET activation of the AKT-signaling pathway; (v) increasing activation of the PLC-D 1 signaling pathway; (w) increasing nausea, vomiting and/or emesis; (x) decreasing T cell adhesion to endothelial cells (e.g., inhibiting LFA1-ICAM interaction) or (y) increasing stimulation of the Hypothalamic-pituitary adrenal axis, e.g., as assessed by increased growth hormone (GH), adrenocorticotropic hormone (ACTH), corticosterone/cortisol, or a combination thereof.

Disclosed herein is use of GDF15 antibody agents that can modulate an activity of GDF15. In some embodiments, a GDF15 Antibody Agents specifically binds GDF15 and modulates one or more, or all, or any combination of detectable GDF15 activities such that the antibody agent: (a) increases food intake; (b) increases appetite; (c) increases body weight; (d) decreases weight loss; (e) increases fat mass; (f) increases lean mass; (g) decreases loss of fat mass, (h) promotes weight gain; (i) decreases loss of lean muscle mass, (j) decreases fatigue; (k) increases pro-inflammation; (l) increases immune cell infiltration in tumor; (m) decreases metastases; (n) increases efficacy of immunotherapy (e.g., immune checkpoint inhibitor therapy); (o) decreases cellular senescence; (p) inhibits binding of GDF15 to a receptor, e.g., GFRAL; (q) decreases downstream signaling mediated by RET; (r) decreases phosphorylation of ERK; (s) decreases phosphorylation of ribosomal protein S6; (t) decreases RET-mediated activation of the MAPK signaling pathway; (u) decreases RET activation of the AKT-signaling pathway; (v) decreases activation of the PLC-D 1 signaling pathway, (w) decreases nausea, vomiting and/or emesis; (x) increases T cell adhesion to endothelial cells (e.g., inhibiting LFA1-ICAM interaction); or (y) decreases stimulation of the Hypothalamic-pituitary adrenal axis as assessed by decreased growth hormone (GH), adrenocorticotropic hormone (ACTH) and corticosterone/cortisol.

One with skill in the art will appreciate that, in some embodiments, an activity of GDF15, e.g., as described herein can be assessed using one or more art recognized assays. For example, as disclosed in Example 4 herein, an assay using cells co-expressing GFRAL and RET can be used to evaluate GDF15 activity. Several assays can be used to measure activation of the MAPK pathway following stimulation with GDF15, e.g., a luciferase-based gene reporter system, or phospho-protein assays (e.g., assessing phospho-ERK1/2).

In some embodiments, a GDF15 Antibody Agents may show preferential binding to GDF15 relative to one or more TGFbeta family members other than GDF15. In some such embodiments, preferential binding may be assessed, for example, by simultaneously contacting GDF15 Antibody Agents with GDF15 and one or more other TGFbeta family members. Alternatively or additionally, in some embodiments, preferential binding may be assessed relative to an appropriate reference GDF15 Antibody Agent (e.g., as described in one or more of WO2014049087, WO21544855, WO2017055613, US 2020/0055930 A1, or U.S. Pat. No. 9,175,076) and, e.g., may reflect a higher level of binding to GDF15 relative to the one or more other TGFbeta family member than is observed with the reference antibody.

In some embodiments, a GDF15 Antibody Agent preferentially binds to GDF15. In some embodiments, GDF15 Antibody Agents does not bind to one or more members of the TGFbeta super family other than GDF15. In some embodiments, GDF15 Antibody Agent does not bind to GDNF, GDF8, GDF10, GDF11, BMP9, BMP 10, Activin A, Activin B or a combination thereof.

In some embodiments, a GDF15 Antibody Agent disclosed herein preferentially binds to GDF15. In some embodiments, a GDF15 Antibody Agent disclosed herein binds to one or more members of the TGFbeta super family in addition to GDF15. In some embodiments, a GDF15 Antibody Agent disclosed herein binds to GDF15 and one or more of: Activin A, Activin B, or GDF10. In some embodiments, a GDF15 Antibody Agent disclosed herein binds to GDF15 and Activin A. In some embodiments, a GDF15 Antibody Agent disclosed herein binds to GDF15 and Activin B. In some embodiments, a GDF15 Antibody Agent disclosed herein binds to GDF15 and GDF10. In some embodiments, a GDF15 Antibody Agent disclosed herein binds to GDF15, Activin A and Activin B. In some embodiments, a GDF15 Antibody Agent disclosed herein binds to GDF15, Activin A, Activin B and GDF10.

In some embodiments, a GDF15 Antibody Agent which binds to GDF15 and Activin A does not modulate an activity and/or level of Activin A, e.g., when characterized in an assay that evaluates an Activin A activity and/or level.

In some embodiments, a GDF15 Antibody Agent which binds to GDF15 and Activin B does not modulate an activity and/or level of Activin B, e.g., when characterized in an assay that evaluates an Activin B activity and/or level.

Those skilled in the art, reading the present disclosure will appreciate that, in some embodiments, an antibody agent provided by the present disclosure comprises: (i) an intact IgA, IgG, IgD, IgE or IgM antibody; (ii) an antibody fragment (e.g., an antibody variable region, containing both heavy and light chain sequences, e.g., a Fab); (iii) a single domain antibody (e.g., a light chain antibody or a heavy chain antibody); (iv) a single chain antibody (e.g., a single chain Fv, a camelid antibody, etc); (v) an antibody-drug conjugate; (vi) a bi- or other multispecific antibody; (vii) a polypeptide comprising antigen binding specificity fused to an Fc region; etc.

Those skilled in the art, reading the present disclosure will further appreciate that provided contributions are not limited to intact antibodies or fragments thereof (e.g., including both heavy and light chain sequences).

For example, those skilled in the art will appreciate that individual light chains and/or individual heavy chains, or variable region sequences thereof, as described herein (e.g., as exemplified herein, for example as presented in Table 1) may be useful in combination with other light chains and/or heavy chains. In some embodiments, a single light chain described herein (or variable region sequences thereof) may be utilized together with two (or more) different heavy chains (e.g., which may be or comprise heavy chains exemplified herein), or variable region sequences thereof, in a “common light chain” bispecific format. In some embodiments, exemplified light and heavy chains (e.g., variable region sequences thereof) may be “mixed and matched” with one another in antibody agents provided by the present disclosure (e.g., antibody agents that specifically bind to GDF15 and/or have one or more other structural and/or functional properties as described herein.

Still further, those skilled in the art will appreciate that the present disclosure provides useful heavy and light chain antibody sequences, specifically including useful variable region sequences, including for example useful CDR and/or framework (FR) sequences.

In some embodiments, the present disclosure provides methods of using polypeptides (which may, for example, be, or be included in, an antibody agent that binds specifically to GDF15) including one or more CDR and/or FR sequences as set forth in Table 1 or 2. In some embodiments, the present disclosure provides polypeptides including two or more CDR elements from Table 1 or 2, and in particular the present disclosure provides polypeptides including three or six CDR elements from Table 1 or 2.

In some embodiments, the present disclosure provides use of polypeptides (which may, for example, be, or be included in, an antibody agent that binds specifically to GDF15) including one LC CDR1, one LC CDR2, and one LC CDR3 from Table 1; in some such embodiments, two or three of the CDRs are from the same LC in Table 1.

In some embodiments, the present disclosure provides polypeptides (which may, for example, be, or be included in, an antibody agent that binds specifically to GDF15) including one HC CDR1, one HC CDR2, and one HC CDR3 from Table 2; in some such embodiments, two or three of the CDRs are from the same HC in Table 2.

In some embodiments, the present disclosure provides polypeptides (which may, for example, be, or be included in, an antibody agent that binds specifically to GDF15) including one each of a LC CDR1, a LC CDR2, a LC CDR3, a HC CDR1, a HC CDR2, and a HC CDR3 from Table 1 or 2; in some such embodiments, two or more CDRs, and in some embodiments all LC CDRs, all HC CDRS, or both, are from the same antibody in Table 1 or 2.

Those skilled in the art will further appreciate that, in some embodiments, useful polypeptides as described herein that include one or more CDRs from Table 1 or 2 may include a heavy or light chain CDR set (i.e., each of a CDR1, a CDR2, and a CDR3) that includes one or two CDRs from a first antibody chain (i.e., LC or HC) in Table 1 or 2, and at least one from a second antibody chain (e.g., of the same type) in Table 1 or 2. Alternatively or additionally, in some embodiments, those skilled in the art will appreciate that useful polypeptides as described herein that include one or more CDRs from Table 1 or 2 may include a heavy or light chain CDR set (i.e., each of a CDR1, a CDR2, and a CDR3) that includes at least one CDR from a first antibody chain (i.e., LC or HC) in Table 1 or 2 and at least one other CDR that differs from its corresponding CDR in the relevant chain in Table 1 or 2. In some such embodiments, the differing CDR(s) will differ at not more than 3, not more than 2, or not more than 1 positions relative to the corresponding CDR(s); alternatively or additionally, in some such embodiments, the differing CDR(s) will differ only at terminal residue(s) relative to the corresponding CDR(s).

In some embodiments, a GDF15 antibody agent disclosed herein which binds to GDF15 comprises a LC CDR1, a LC CDR2 and a LC CDR3 provided in Table 1. In some embodiments, the presence of a LC CDR1, a LC CDR2 and a LC CDR3 is sufficient to confer binding and/or is otherwise useful in an antibody agent disclosed herein (i.e., that specifically binds to GDF15). In some embodiments, an antibody agent comprising a LC CDR1, a LC CDR2 and a LC CDR3 can be in any format disclosed herein. For example, in some embodiments, an antibody comprising a LC CDR1, a LC CDR2 and a LC CDR3 can be a single chain antibody, and is capable of binding GDF15.

In some embodiments, a GDF15 Antibody Agent which binds to GDF15 comprises a HC CDR1, a HC CDR2 and a HC CDR3 is sufficient to confer binding and/or is otherwise useful in an antibody agent disclosed herein to GDF15. In some embodiments, an Antibody Agent comprising a HC CDR1, a HC CDR2 and a HC CDR3 can be in any format disclosed herein. For example, in some embodiments, an Antibody Agent comprising a HC CDR1, a HC CDR2 and a HC CDR3 can be a single chain antibody, and is capable of binding GDF15.

In some embodiments, a GDF15 antibody agent which binds to GDF15 comprises a set of any three LC CDRs (e.g., LC CDR1, LC CDR2 and LC CDR3) provided in Table 1, and a set of any three HC CDRs (e.g., HC CDR1, HC CDR2 and HC CDR3) provided in Table 2. In some embodiments, the presence of a set of any three LC CDRs and a set of any three HC CDRs is sufficient to confer binding of any antibody agent to GDF15. In some embodiments, such a GDF15 antibody agent can be a fragment (e.g., an scFv, a Fab or other fragments), or an intact antibody, or a polypeptide comprising antigen binding specificity fused to an Fc.

In some embodiments, disclosed herein is use of a GDF15 Antibody Agent that competes (e.g., when tested in a standard competition assay) for binding to human GDF15 with a different GDF15 Antibody Agent, e.g., a GDF15 Antibody Agent disclosed in US 2020/0055930A1, U.S. Pat. Nos. 10,174,119; and 9,175,076. In some embodiments, a GDF15 Antibody Agent disclosed herein competes for binding to human GDF15 with a different GDF15 Antibody Agent when assessed at more than one concentration (e.g., over a concentration range of at least 2-, 4-, 6-, 8-, 10-fold or more).

In some embodiments, disclosed herein is use of GDF15 Antibody Agents that do not compete (e.g., when tested in a standard competition assay) for binding to human GDF15 with a different GDF15 Antibody Agent, e.g., a GDF15 antibody disclosed in US 2020/0055930A1, U.S. Pat. Nos. 10,174,119; and 9,175,076.

In some embodiments, disclosed herein is use of GDF15 Antibody Agents that bind to a sterically overlapping (e.g., partially or completely overlapping) epitope as a GDF15 Antibody Agent disclosed in US 2020/0055930A1, U.S. Pat. Nos. 10,174,119; 9,175,076 or U.S. Pat. No. 10,604,565.

### Light Chain (e.g., Light Chain Variable Region) Polypeptides (LC Polypeptides)

The present disclosure provides use of polypeptides comprising light chain (LC) sequences (e.g., light chain variable region sequence(s)), that, for example, may be useful in an Antibody Agent as described herein targeting GDF15 and/or GFRAL to treat and/or prevent certain types and/or instances of nausea and/or emesis with which it had not previously been considered to be associated. In some such embodiments, such provided polypeptides are useful and/or included in such antibody agents as described herein. In some embodiments, a LC polypeptide comprises at least one LC CDR provided in Table 1 or a sequence with at least 85% identity thereto. In some embodiments, a LC polypeptide comprises one, two or three LC CDRs (e.g., a LC CDR1, a LC CDR2 and/or a LC CDR3). In some embodiments, a LC polypeptide comprises a LC CDR1. In some embodiments, a LC polypeptide comprises a LC CDR2. In some embodiments, a LC polypeptide comprises a LC CDR3. In some embodiments, a LC polypeptide comprises a LC CDR1, a LC CDR2 and a LC CDR3.

In some embodiments, a LC polypeptide having a LC CDR1, a LC CDR2 and a LC CDR3, e.g., in a GDF15 Antibody Agent and/or a GFRAL Antibody Agent, is capable of binding (e.g., specifically binding) to GDF15.

In some embodiments, a LC polypeptide further comprises one or more framework regions, and/or a constant region.

In some embodiments, a LC polypeptide comprises a light chain constant region and/or a heavy chain constant region. In some embodiments, a LC polypeptide comprises a light chain constant region or a portion thereof, (e.g., a lambda light chain constant region or a variant or portion thereof; or a kappa light chain constant region or a variant or a portion thereof).

In some embodiments, a light chain kappa constant region comprises the sequence of SEQ ID NO: 175, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 175.

(SEQ ID NO: 175)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC

In some embodiments, a LC polypeptide comprises the sequence of SEQ ID NO: 175, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 175.

In some embodiments, a LC polypeptide further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, a LC polypeptide comprises: (i) an LC CDR1 sequence provided in Table 1, e.g., any one of SEQ ID NOs: 92, 101, 117, 125, 129, 137, or 212; (ii) a sequence with at least 85%, 86%, 87%, 88%, 89%, 90%, 91%, 92%, 93%, 94%, 95%, 96%, 97%, 98%, 99% identity to an LC CDR1 sequence provided in Table 1, e.g., any one of SEQ ID NOs: 92, 101, 117, 125, 129, 137, or 212; or (iii) a sequence having at least 5, 10, or 20 alterations (e.g., substitutions, deletions or insertions (e.g., conservative substitutions)) relative to an LC CDR1 sequence provided in Table 1, e.g., any one of SEQ ID NOs: 92, 101, 117, 125, 129, 137, or 212.

In some embodiments, a LC polypeptide comprises: (i) an LC CDR2 sequence provided in Table 1, e.g., any one of SEQ ID NOs: 93, 102 or 130; (ii) a sequence with at least 85%, 86%, 87%, 88%, 89%, 90%, 91%, 92%, 93%, 94%, 95%, 96%, 97%, 98%, 99% identity to an LC CDR2 sequence provided in Table 1 e.g., any one of SEQ ID NOs: 93, 102 or 130; or (iii) a sequence having at least 5, 10, or 20 alterations (e.g., substitutions, deletions or insertions (e.g., conservative substitutions)) relative to an LC CDR2 sequence provided in Table 1, e.g., any one of SEQ ID NOs: 93, 102 or 130.

In some embodiments, a LC polypeptide comprises: (i) an LC CDR3 sequence provided in Table 1, e.g., any one of SEQ ID NOs: 94, 103, 110, 118, 126, 131, 138, 204, 208 or 217; (ii) a sequence with at least 85%, 86%, 87%, 88%, 89%, 90%, 91%, 92%, 93%, 94%, 95%, 96%, 97%, 98%, 99% identity to an LC CDR3 sequence provided in Table 1, e.g., any one of SEQ ID NOs: 94, 103, 110, 118, 126, 131, 138, 204, 208 or 217; or (iii) a sequence having at least 5, 10, or 20 alterations (e.g., substitutions, deletions or insertions (e.g., conservative substitutions)) relative to an LC CDR3 sequence provided in Table 1, e.g., any one of SEQ ID NOs: 94, 103, 110, 118, 126, 131, 138, 204, 208 or 217.

In some embodiments, a LC polypeptide comprises (i) an LC CDR1, LC CDR2, and LC CDR3 sequence provided in Table 1; (ii) a sequence with at least 85%, 86%, 87%, 88%, 89%, 90%, 91%, 92%, 93%, 94%, 95%, 96%, 97%, 98%, 99% identity to an LC CDR1, LC CDR2, and LC CDR3 sequence provided in Table 1; or (iii) a sequence having at least 5, 10, or 20 alterations (e.g., substitutions, deletions or insertions (e.g., conservative substitutions)) relative to an LC CDR1, LC CDR2, and LC CDR3 sequence provided in Table 1.

In some embodiments, a LC polypeptide comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, 99% identity thereto or a having at least 5, 10, or 20 alterations (e.g., substitutions, deletions or insertions (e.g., conservative substitutions)) relative to SEQ ID NO: 92; (ii) an LC CDR2 of SEQ ID NO: 93, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 93; and/or (iii) an LC CDR3 of SEQ ID NO: 94, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 94.

In some embodiments, a LC polypeptide comprises: (i) an LC CDR1 of SEQ ID NO: 101, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 101; (ii) an LC CDR2 of SEQ ID NO: 102, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 102; and/or (iii) an LC CDR3 of SEQ ID NO: 103, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 103.

In some embodiments, a LC polypeptide comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 92; (ii) an LC CDR2 of SEQ ID NO: 93, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 93; and/or (iii) an LC CDR3 of SEQ ID NO: 110, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 110.

In some embodiments, a LC polypeptide comprises: (i) an LC CDR1 of SEQ ID NO: 117, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 117; (ii) an LC CDR2 of SEQ ID NO: 93, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 93; and/or (iii) an LC CDR3 of SEQ ID NO: 118, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 118.

In some embodiments, a LC polypeptide comprises: (i) an LC CDR1 of SEQ ID NO: 125, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 125; (ii) an LC CDR2 of SEQ ID NO: 102, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 102; and/or (iii) an LC CDR3 of SEQ ID NO: 126, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 126.

In some embodiments, a LC polypeptide comprises: (i) an LC CDR1 of SEQ ID NO: 129, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 129; (ii) an LC CDR2 of SEQ ID NO: 130, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 130; and/or (iii) an LC CDR3 of SEQ ID NO: 131, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 131.

In some embodiments, a LC polypeptide comprises: (i) an LC CDR1 of SEQ ID NO: 137, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 137; (ii) an LC CDR2 of SEQ ID NO: 102, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 102; and/or (iii) an LC CDR3 of SEQ ID NO: 138, or a sequence with at least 85%, 86%, 87%, 88%, 89%, 90%, 91%, 92%, 93%, 94%, 95%, 96%, 97%, 98%, 99% identity thereto or a sequence having at least 5, 10, or 20 alterations (e.g., substitutions, deletions or insertions (e.g., conservative substitutions)) relative to SEQ ID NO: 138.

In some embodiments, a LC polypeptide comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; (ii) an LC CDR2 of SEQ ID NO: 93 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and/or (iii) an LC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

In some embodiments, a LC polypeptide comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; (ii) an LC CDR2 of SEQ ID NO: 93 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and/or (iii) an LC CDR3 of SEQ ID NO: 208, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 208.

In some embodiments, a LC polypeptide comprises: (i) an LC CDR1 of SEQ ID NO: 212, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 212; (ii) an LC CDR2 of SEQ ID NO: 102 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and/or (iii) an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103.

In some embodiments, a LC polypeptide comprises: (i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101; (ii) an LC CDR2 of SEQ ID NO: 102 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and/or (iii) an LC CDR3 of SEQ ID NO: 217, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 217.

In some embodiments, a LC polypeptide further comprises one or more framework regions (FR), e.g., as described herein. In some embodiments, a LC polypeptide comprises one, two, three or four FRs, e.g., as described herein. In some embodiments, a FR comprises a LC FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a LC FR sequence as described herein, or a sequence having at least 5, 10 or 20 alterations relative to a LC FR sequence as described herein.

In some embodiments, a LC polypeptide comprises: (i) a FR sequence provided in Table 1; (ii) a sequence with at least 92%, 93%, 94%, 95%, 96%, 97%, 98%, or 99% identity to a FR sequence provided in Table 1; or (iii) a sequence having at least 5, 10, or 20 alterations (e.g., substitutions, deletions or insertions (e.g., conservative substitutions)) compared to a FR sequence provided in Table 1.

In some embodiments, a LC polypeptide comprises a LC FR1 provided in Table 1, a sequence with at least 92%, 93%, 94%, 95%, 96%, 97%, 98%, or 99% identity to a LC FR1 sequence provided in Table 1, or a sequence having at least 5, 10, or 20 alterations (e.g., substitutions, deletions or insertions (e.g., conservative substitutions)) compared to a LC FR1 sequence provided in Table 1.

In some embodiments, a LC polypeptide comprises a LC FR2 provided in Table 1, a sequence with at least 92%, 93%, 94%, 95%, 96%, 97%, 98%, or 99% identity to a LC FR2 sequence provided in Table 1, or a sequence having at least 5, 10, or 20 alterations (e.g., substitutions, deletions or insertions (e.g., conservative substitutions)) compared to a LC FR2 sequence provided in Table 1.

In some embodiments a LC polypeptide comprises a LC FR3 provided in Table 1, a sequence with at least 92%, 93%, 94%, 95%, 96%, 97%, 98%, or 99% identity to a LC FR3 sequence provided in Table 1, or a sequence having at least 5, 10, or 20 alterations (e.g., substitutions, deletions or insertions (e.g., conservative substitutions)) compared to a LC FR3 sequence provided in Table 1.

In some embodiments, a LC polypeptide comprises a LC FR4 provided in Table 1, a sequence with at least 92%, 93%, 94%, 95%, 96%, 97%, 98%, or 99% identity to a LC FR4 sequence provided in Table 1, or a sequence having at least 5, 10, or 20 alterations (e.g., substitutions, deletions or insertions (e.g., conservative substitutions)) compared to a LC FR4 sequence provided in Table 1.

In some embodiments, a LC polypeptide comprises a LC CDR1, a LC CDR2 and LC CDR3 provided in Table 1 or a sequence with at least 85% identity thereto, and a LC FR1, LC FR2, LC FR3 and a LC FR4 of a provided in Table 1 or a sequence with at least 92% identity thereto.

In some embodiments, a LC polypeptide comprises the sequence of SEQ ID NO: 99, or a sequence with at least 85%, 86%, 87%, 88%, 89%, 90%, 91%, 92%, 93%, 94%, 95%, 96%, 97%, 98%, 99% identity thereto, or a sequence having at least 5, 10, or 20 alterations (e.g., substitutions, deletions or insertions (e.g., conservative substitutions)) relative to SEQ ID NO: 99.

In some embodiments, a LC polypeptide comprises the sequence of SEQ ID NO: 107, or a sequence with at least 85%, 86%, 87%, 88%, 89%, 90%, 91%, 92%, 93%, 94%, 95%, 96%, 97%, 98%, 99% identity thereto, or a sequence having at least 5, 10, or 20 alterations (e.g., substitutions, deletions or insertions (e.g., conservative substitutions)) relative to SEQ ID NO: 107.

In some embodiments, a LC polypeptide comprises the sequence of SEQ ID NO: 115, or a sequence with at least 85%, 86%, 87%, 88%, 89%, 90%, 91%, 92%, 93%, 94%, 95%, 96%, 97%, 98%, 99% identity thereto, or a sequence having at least 5, 10, or 20 alterations (e.g., substitutions, deletions or insertions (e.g., conservative substitutions)) relative to SEQ ID NO: 115.

In some embodiments, a LC polypeptide comprises the sequence of SEQ ID NO: 123, or a sequence with at least 85%, 86%, 87%, 88%, 89%, 90%, 91%, 92%, 93%, 94%, 95%, 96%, 97%, 98%, 99% identity thereto, or a sequence having at least 5, 10, or 20 alterations (e.g., substitutions, deletions or insertions (e.g., conservative substitutions)) relative to SEQ ID NO: 123.

In some embodiments, a LC polypeptide comprises the sequence of SEQ ID NO: 127, or a sequence with at least 85%, 86%, 87%, 88%, 89%, 90%, 91%, 92%, 93%, 94%, 95%, 96%, 97%, 98%, 99% identity thereto, or a sequence having at least 5, 10, or 20 alterations (e.g., substitutions, deletions or insertions (e.g., conservative substitutions)) relative to SEQ ID NO: 127.

In some embodiments, a LC polypeptide comprises the sequence of SEQ ID NO: 135, or a sequence with at least 85%, 86%, 87%, 88%, 89%, 90%, 91%, 92%, 93%, 94%, 95%, 96%, 97%, 98%, 99% identity thereto, or a sequence having at least 5, 10, or 20 alterations (e.g., substitutions, deletions or insertions (e.g., conservative substitutions)) relative to SEQ ID NO: 135.

In some embodiments, a LC polypeptide comprises the sequence of SEQ ID NO: 139, or a sequence with at least 85%, 86%, 87%, 88%, 89%, 90%, 91%, 92%, 93%, 94%, 95%, 96%, 97%, 98%, 99% identity thereto, or a sequence having at least 5, 10, or 20 alterations (e.g., substitutions, deletions or insertions (e.g., conservative substitutions)) relative to SEQ ID NO: 139.

In some embodiments, a LC polypeptide comprises the sequence of SEQ ID NO: 205, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 205.

In some embodiments, a LC polypeptide comprises the sequence of SEQ ID NO: 209, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 209.

In some embodiments, a LC polypeptide comprises the sequence of SEQ ID NO: 214, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 214.

In some embodiments, a LC polypeptide comprises the sequence of SEQ ID NO: 218, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 218.

In some embodiments, a LC polypeptide comprises an LC sequence provided in Table 1, e.g., any one of SEQ ID NOs: 159, 163, 164, 166, 169, 171, 173 or 206, 210, 215 or 219; or a sequence with at least 85%, 86%, 87%, 88%, 89%, 90%, 91%, 92%, 93%, 94%, 95%, 96%, 97%, 98%, 99% identity thereto; or a sequence having at least 5, 10, or 20 alterations (e.g., substitutions, deletions or insertions (e.g., conservative substitutions)) relative to any one of SEQ ID NOs: 159, 163, 164, 166, 169, 171, 173 or 206, 210, 215 or 219.

Exemplary useful LC polypeptides which may be included in GDF15 Antibody Agents are disclosed in Table 1 below.

TABLE 1
Light chain polypeptide and nucleic acid sequences

| | SEQ ID | | |
|---|---|---|---|
| CLONE | NO | Feature | SEQUENCE |
| A | 92 | LC CDR1 | RSSQSLLHSNGYNYLD |
| VL germline | 93 | LC CDR2 | LGSNRAS |
| VK2-28 | 94 | LC CDR3 | MQTLEAPPT |
| | 95 | LC FR1 | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 99 | VL amino acid | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | EAPPTFGGGTKVEIK |
| | 159 | LC | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | EAPPTFGGGTKVEIKRTVAAPSVFIFPPSDEQL |
| | | | KSGTASVVCLLNNFYPREAKVQWKVDNALQ |
| | | | SGNSQESVTEQDSKDSTYSLSSTLTLSKADYE |
| | | | KHKVYACEVTHQGLSSPVTKSFNRGEC |
| | 100 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGCAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGCAGACACTCGAAGCCCCTCCTACT |
| | | | TTTGGCGGAGGGACCAAGGTTGAGATCAAA |
| B | 92 | LC CDR1 | RSSQSLLHSNGYNYLD |
| VL germline | 93 | LC CDR2 | LGSNRAS |
| VK2-28 | 94 | LC CDR3 | MQTLEAPPT |
| | 95 | LC FRI | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 99 | VL amino acid | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | EAPPTFGGGTKVEIK |
| | 159 | LC | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | EAPPTFGGGTKVEIKRTVAAPSVFIFPPSDEQL |
| | | | KSGTASVVCLLNNFYPREAKVQWKVDNALQ |
| | | | SGNSQESVTEQDSKDSTYSLSSTLTLSKADYE |
| | | | KHKVYACEVTHQGLSSPVTKSFNRGEC |
| | 100 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGCAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGCAGACACTCGAAGCCCCTCCTACT |
| | | | TTTGGCGGAGGGACCAAGGTTGAGATCAAA |
| C | 92 | LC CDR1 | RSSQSLLHSNGYNYLD |
| VL germline | 93 | LC CDR2 | LGSNRAS |
| VK2-28 | 94 | LC CDR3 | MQTLEAPPT |
| | 95 | LC FR1 | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 99 | VL amino acid | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | EAPPTFGGGTKVEIK |
| | 159 | LC | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | EAPPTFGGGTKVEIKRTVAAPSVFIFPPSDEQL |
| | | | KSGTASVVCLLNNFYPREAKVQWKVDNALQ |
| | | | SGNSQESVTEQDSKDSTYSLSSTLTLSKADYE |
| | | | KHKVYACEVTHQGLSSPVTKSFNRGEC |
| | 100 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGCAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGCAGACACTCGAAGCCCCTCCTACT |
| | | | TTTGGCGGAGGGACCAAGGTTGAGATCAAA |
| D | 92 | LC CDR1 | RSSQSLLHSNGYNYLD |
| VL germline | 93 | LC CDR2 | LGSNRAS |
| VK2-28 | 94 | LC CDR3 | MQTLEAPPT |
| | 95 | LC FR1 | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 99 | VL amino acid | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | EAPPTFGGGTKVEIK |
| | 159 | LC | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | EAPPTFGGGTKVEIKRTVAAPSVFIFPPSDEQL |
| | | | KSGTASVVCLLNNFYPREAKVQWKVDNALQ |
| | | | SGNSQESVTEQDSKDSTYSLSSTLTLSKADYE |
| | | | KHKVYACEVTHQGLSSPVTKSFNRGEC |
| | 100 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGCAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGCAGACACTCGAAGCCCCTCCTACT |
| | | | TTTGGCGGAGGGACCAAGGTTGAGATCAAA |
| E | 101 | LC CDR1 | RASQGISSWLA |
| VL germline | 102 | LC CDR2 | AASSLQS |
| VK1-12 | 103 | LC CDR3 | QQANSFPIT |
| | 104 | LC FR1 | DIQMTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 107 | VL amino acid | DIQMTQSPSSVSASVGDRVTITCRASQGISSWL |
| | | | AWYQQKPGKAPKLLIYAASSLQSGVPSRFSGS |
| | | | GSGTDFTLTISSLQPEDFATYYCQQANSFPITF |
| | | | GGGTKVEIK |
| | 163 | LC | DIQMTQSPSSVSASVGDRVTITCRASQGISSWL |
| | | | AWYQQKPGKAPKLLIYAASSLQSGVPSRFSGS |
| | | | GSGTDFTLTISSLQPEDFATYYCQQANSFPITF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 108 | VL DNA | GACATCCAGATGACCCAGTCTCCATCTTCCG |
| | | | TGTCTGCATCTGTAGGAGACAGAGTCACCA |
| | | | TCACTTGTCGGGCGAGTCAGGGTATTAGCA |
| | | | GCTGGTTAGCCTGGTATCAGCAGAAACCAG |
| | | | GGAAAGCCCCTAAGCTCCTGATCTATGCTGC |
| | | | ATCCAGTTTGCAAAGTGGGGTCCCATCAAG |
| | | | GTTCAGCGGCAGTGGATCTGGGACAGATTT |
| | | | CACTCTCACCATCAGCAGCCTGCAGCCTGA |
| | | | AGATTTTGCAACTTACTATTGTCAACAGGCT |
| | | | AACAGTTTCCCTATCACCTTTGGCGGAGGGA |
| | | | CCAAGGTTGAGATCAAA |
| F | 92 | LC CDR1 | RSSQSLLHSNGYNYLD |
| VL germline | 93 | LC CDR2 | LGSNRAS |
| VK2-28 | 110 | LC CDR3 | MQALQTPIT |
| | 95 | LC FRI | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 115 | VL amino acid | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQAL |
| | | | QTPITFGGGTKVEIK |
| | 164 | LC | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQAL |
| | | | QTPITFGGGTKVEIKRTVAAPSVFIFPPSDEQLK |
| | | | SGTASVVCLLNNFYPREAKVQWKVDNALQS |
| | | | GNSQESVTEQDSKDSTYSLSSTLTLSKADYEK |
| | | | HKVYACEVTHQGLSSPVTKSFNRGEC |
| | 116 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGCAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGCAAGCTCTACAAACTCCTATCACC |
| | | | TTTGGCGGAGGGACCAAGGTTGAGATCAAA |
| G | 92 | LC CDR1 | RSSQSLLHSNGYNYLD |
| VL germline | 93 | LC CDR2 | LGSNRAS |
| VK2-28 | 110 | LC CDR3 | MQALQTPIT |
| | 95 | LC FR1 | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 115 | VL amino acid | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQAL |
| | | | QTPITFGGGTKVEIK |
| | 164 | LC | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQAL |
| | | | QTPITFGGGTKVEIKRTVAAPSVFIFPPSDEQLK |
| | | | SGTASVVCLLNNFYPREAKVQWKVDNALQS |
| | | | GNSQESVTEQDSKDSTYSLSSTLTLSKADYEK |
| | | | HKVYACEVTHQGLSSPVTKSFNRGEC |
| | 116 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGCAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGCAAGCTCTACAAACTCCTATCACC |
| | | | TTTGGCGGAGGGACCAAGGTTGAGATCAAA |
| H | 117 | LC CDR1 | RSSQSLLYSNGYNYLD |
| VL germline | 93 | LC CDR2 | LGSNRAS |
| VK2-28 | 118 | LC CDR3 | MQAIGVPLT |
| | 95 | LC FR1 | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 123 | VL amino acid | DIVMTQSPLSLPVTPGEPASISCRSSQSLLYSNG |
| | | | YNYLDWYLQKPGQSPQLLIYLGSNRASGVPD |
| | | | RFSGSGSGTDFTLKISRVEAEDVGVYYCMQAI |
| | | | GVPLTFGGGTKVEIK |
| | 166 | LC | DIVMTQSPLSLPVTPGEPASISCRSSQSLLYSNG |
| | | | YNYLDWYLQKPGQSPQLLIYLGSNRASGVPD |
| | | | RFSGSGSGTDFTLKISRVEAEDVGVYYCMQAI |
| | | | GVPLTFGGGTKVEIKRTVAAPSVFIFPPSDEQL |
| | | | KSGTASVVCLLNNFYPREAKVQWKVDNALQ |
| | | | SGNSQESVTEQDSKDSTYSLSSTLTLSKADYE |
| | | | KHKVYACEVTHQGLSSPVTKSFNRGEC |
| | 124 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGTAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGCAGGCAATAGGCGTCCCTCTCACT |
| | | | TTTGGCGGAGGGACCAAGGTTGAGATCAAA |
| I | 101 | LC CDR1 | RASQGISSWLA |
| VL germline | 102 | LC CDR2 | AASSLQS |
| VK1-12 | 103 | LC CDR3 | QQANSFPIT |
| | 104 | LC FR1 | DIQMTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 107 | VL amino acid | DIQMTQSPSSVSASVGDRVTITCRASQGISSWL |
| | | | AWYQQKPGKAPKLLIYAASSLQSGVPSRFSGS |
| | | | GSGTDFTLTISSLQPEDFATYYCQQANSFPITF |
| | | | GGGTKVEIK |
| | 163 | LC | DIQMTQSPSSVSASVGDRVTITCRASQGISSWL |
| | | | AWYQQKPGKAPKLLIYAASSLQSGVPSRFSGS |
| | | | GSGTDFTLTISSLQPEDFATYYCQQANSFPITF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 108 | VL DNA | GACATCCAGATGACCCAGTCTCCATCTTCCG |
| | | | TGTCTGCATCTGTAGGAGACAGAGTCACCA |
| | | | TCACTTGTCGGGCGAGTCAGGGTATTAGCA |
| | | | GCTGGTTAGCCTGGTATCAGCAGAAACCAG |
| | | | GGAAAGCCCCTAAGCTCCTGATCTATGCTGC |
| | | | ATCCAGTTTGCAAAGTGGGGTCCCATCAAG |
| | | | GTTCAGCGGCAGTGGATCTGGGACAGATTT |
| | | | CACTCTCACCATCAGCAGCCTGCAGCCTGA |
| | | | AGATTTTGCAACTTACTATTGTCAACAGGCT |
| | | | AACAGTTTCCCTATCACCTTTGGCGGAGGGA |
| | | | CCAAGGTTGAGATCAAA |
| J | 101 | LC CDR1 | RASQGISSWLA |
| VL germline | 102 | LC CDR2 | AASSLQS |
| VK1-12 | 103 | LC CDR3 | QQANSFPIT |
| | 104 | LC FRI | DIQMTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 107 | VL amino acid | DIQMTQSPSSVSASVGDRVTITCRASQGISSWL |
| | | | AWYQQKPGKAPKLLIYAASSLQSGVPSRFSGS |
| | | | GSGTDFTLTISSLQPEDFATYYCQQANSFPITF |
| | | | GGGTKVEIK |
| | 163 | LC | DIQMTQSPSSVSASVGDRVTITCRASQGISSWL |
| | | | AWYQQKPGKAPKLLIYAASSLQSGVPSRFSGS |
| | | | GSGTDFTLTISSLQPEDFATYYCQQANSFPITF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 108 | VL DNA | GACATCCAGATGACCCAGTCTCCATCTTCCG |
| | | | TGTCTGCATCTGTAGGAGACAGAGTCACCA |
| | | | TCACTTGTCGGGCGAGTCAGGGTATTAGCA |
| | | | GCTGGTTAGCCTGGTATCAGCAGAAACCAG |
| | | | GGAAAGCCCCTAAGCTCCTGATCTATGCTGC |
| | | | ATCCAGTTTGCAAAGTGGGGTCCCATCAAG |
| | | | GTTCAGCGGCAGTGGATCTGGGACAGATTT |
| | | | CACTCTCACCATCAGCAGCCTGCAGCCTGA |
| | | | AGATTTTGCAACTTACTATTGTCAACAGGCT |
| | | | AACAGTTTCCCTATCACCTTTGGCGGAGGGA |
| | | | CCAAGGTTGAGATCAAA |
| K | 125 | LC CDR1 | RASQGIDSWLA |
| VL germline | 102 | LC CDR2 | AASSLQS |
| VK1-12 | 126 | LC CDR3 | QQVHSFPIT |
| | 104 | LC FR1 | DIQMTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 127 | VL amino acid | DIQMTQSPSSVSASVGDRVTITCRASQGIDSW |
| | | | LAWYQQKPGKAPKLLIYAASSLQSGVPSRFSG |
| | | | SGSGTDFTLTISSLQPEDFATYYCQQVHSFPITF |
| | | | GGGTKVEIK |
| | 169 | LC | DIQMTQSPSSVSASVGDRVTITCRASQGIDSW |
| | | | LAWYQQKPGKAPKLLIYAASSLQSGVPSRFSG |
| | | | SGSGTDFTLTISSLQPEDFATYYCQQVHSFPITF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 128 | VL DNA | GACATCCAGATGACCCAGTCTCCATCTTCCG |
| | | | TGTCTGCATCTGTAGGAGACAGAGTCACCA |
| | | | TCACTTGTCGGGCGAGTCAGGGTATTGACA |
| | | | GCTGGTTAGCCTGGTATCAGCAGAAACCAG |
| | | | GGAAAGCCCCTAAGCTCCTGATCTATGCTGC |
| | | | ATCCAGTTTGCAAAGTGGGGTCCCATCAAG |
| | | | GTTCAGCGGCAGTGGATCTGGGACAGATTT |
| | | | CACTCTCACCATCAGCAGCCTGCAGCCTGA |
| | | | AGATTTTGCAACTTATTACTGTCAGCAGGTA |
| | | | CACAGTTTCCCTATCACTTTTGGCGGAGGGA |
| | | | CCAAGGTTGAGATCAAA |
| L | 125 | LC CDR1 | RASQGIDSWLA |
| VL germline | 102 | LC CDR2 | AASSLQS |
| VK1-12 | 126 | LC CDR3 | QQVHSFPIT |
| | 104 | LC FR1 | DIQMTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 127 | VL amino acid | DIQMTQSPSSVSASVGDRVTITCRASQGIDSW |
| | | | LAWYQQKPGKAPKLLIYAASSLQSGVPSRFSG |
| | | | SGSGTDFTLTISSLQPEDFATYYCQQVHSFPITF |
| | | | GGGTKVEIK |
| | 169 | LC | DIQMTQSPSSVSASVGDRVTITCRASQGIDSW |
| | | | LAWYQQKPGKAPKLLIYAASSLQSGVPSRFSG |
| | | | SGSGTDFTLTISSLQPEDFATYYCQQVHSFPITF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 128 | VL DNA | GACATCCAGATGACCCAGTCTCCATCTTCCG |
| | | | TGTCTGCATCTGTAGGAGACAGAGTCACCA |
| | | | TCACTTGTCGGGCGAGTCAGGGTATTGACA |
| | | | GCTGGTTAGCCTGGTATCAGCAGAAACCAG |
| | | | GGAAAGCCCCTAAGCTCCTGATCTATGCTGC |
| | | | ATCCAGTTTGCAAAGTGGGGTCCCATCAAG |
| | | | GTTCAGCGGCAGTGGATCTGGGACAGATTT |
| | | | CACTCTCACCATCAGCAGCCTGCAGCCTGA |
| | | | AGATTTTGCAACTTATTACTGTCAGCAGGTA |
| | | | CACAGTTTCCCTATCACTTTTGGCGGAGGGA |
| | | | CCAAGGTTGAGATCAAA |
| M | 129 | LC CDR1 | RASQSVSSNLA |
| VL germline | 130 | LC CDR2 | GASTRAT |
| VK3-15 | 131 | LC CDR3 | QQYNNWPIT |
| | 132 | LC FR1 | EIVMTQSPATLSVSPGERATLSC |
| | 133 | LC FR2 | WYQQKPGQAPRLLIY |
| | 134 | LC FR3 | GIPARFSGSGSGTEFTLTISSLQSEDFAVYYC |
| | 98 | LC FR4 | FGGGTKVEIK |
| | 135 | VL amino acid | EIVMTQSPATLSVSPGERATLSCRASQSVSSNL |
| | | | AWYQQKPGQAPRLLIYGASTRATGIPARFSGS |
| | | | GSGTEFTLTISSLQSEDFAVYYCQQYNNWPITF |
| | | | GGGTKVEIK |
| | 171 | LC | EIVMTQSPATLSVSPGERATLSCRASQSVSSNL |
| | | | AWYQQKPGQAPRLLIYGASTRATGIPARFSGS |
| | | | GSGTEFTLTISSLQSEDFAVYYCQQYNNWPITF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 136 | VL DNA | GAAATAGTGATGACGCAGTCTCCAGCCACC |
| | | | CTGTCTGTGTCTCCAGGGGAAAGAGCCACC |
| | | | CTCTCCTGCAGGGCCAGTCAGAGTGTTAGC |
| | | | AGCAACTTAGCCTGGTACCAGCAGAAACCT |
| | | | GGCCAGGCTCCCAGGCTCCTCATCTATGGTG |
| | | | CATCCACCAGGGCCACTGGTATCCCAGCCA |
| | | | GGTTCAGTGGCAGTGGGTCTGGGACAGAGT |
| | | | TCACTCTCACCATCAGCAGCCTGCAGTCTGA |
| | | | AGATTTTGCAGTTTATTACTGTCAGCAGTAT |
| | | | AATAACTGGCCTATCACCTTTGGCGGAGGG |
| | | | ACCAAGGTTGAGATCAAA |
| N | 129 | LC CDR1 | RASQSVSSNLA |
| VL germline | 130 | LC CDR2 | GASTRAT |
| VK3-15 | 131 | LC CDR3 | QQYNNWPIT |
| | 132 | LC FRI | EIVMTQSPATLSVSPGERATLSC |
| | 133 | LC FR2 | WYQQKPGQAPRLLIY |
| | 134 | LC FR3 | GIPARFSGSGSGTEFTLTISSLQSEDFAVYYC |
| | 98 | LC FR4 | FGGGTKVEIK |
| | 135 | VL amino acid | EIVMTQSPATLSVSPGERATLSCRASQSVSSNL |
| | | | AWYQQKPGQAPRLLIYGASTRATGIPARFSGS |
| | | | GSGTEFTLTISSLQSEDFAVYYCQQYNNWPITF |
| | | | GGGTKVEIK |
| | 171 | LC | EIVMTQSPATLSVSPGERATLSCRASQSVSSNL |
| | | | AWYQQKPGQAPRLLIYGASTRATGIPARFSGS |
| | | | GSGTEFTLTISSLQSEDFAVYYCQQYNNWPITF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 136 | VL DNA | GAAATAGTGATGACGCAGTCTCCAGCCACC |
| | | | CTGTCTGTGTCTCCAGGGGAAAGAGCCACC |
| | | | CTCTCCTGCAGGGCCAGTCAGAGTGTTAGC |
| | | | AGCAACTTAGCCTGGTACCAGCAGAAACCT |
| | | | GGCCAGGCTCCCAGGCTCCTCATCTATGGTG |
| | | | CATCCACCAGGGCCACTGGTATCCCAGCCA |
| | | | GGTTCAGTGGCAGTGGGTCTGGGACAGAGT |
| | | | TCACTCTCACCATCAGCAGCCTGCAGTCTGA |
| | | | AGATTTTGCAGTTTATTACTGTCAGCAGTAT |
| | | | AATAACTGGCCTATCACCTTTGGCGGAGGG |
| | | | ACCAAGGTTGAGATCAAA |
| O | 137 | LC CDR1 | RASQGISRWLA |
| VL germline | 102 | LC CDR2 | AASSLQS |
| VK1-12 | 138 | LC CDR3 | QQGASFPLT |
| | 104 | LC FR1 | DIQMTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 139 | VL amino acid | DIQMTQSPSSVSASVGDRVTITCRASQGISRWL |
| | | | AWYQQKPGKAPKLLIYAASSLQSGVPSRFSGS |
| | | | GSGTDFTLTISSLQPEDFATYYCQQGASFPLTF |
| | | | GGGTKVEIK |
| | 173 | LC | DIQMTQSPSSVSASVGDRVTITCRASQGISRWL |
| | | | AWYQQKPGKAPKLLIYAASSLQSGVPSRFSGS |
| | | | GSGTDFTLTISSLQPEDFATYYCQQGASFPLTF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 140 | VL DNA | GACATCCAGATGACCCAGTCTCCATCTTCCG |
| | | | TGTCTGCATCTGTAGGAGACAGAGTCACCA |
| | | | TCACTTGTCGGGCGAGTCAGGGTATTAGCA |
| | | | GGTGGTTAGCCTGGTATCAGCAGAAACCAG |
| | | | GGAAAGCCCCTAAGCTCCTGATCTATGCTGC |
| | | | ATCCAGTTTGCAAAGTGGGGTCCCATCAAG |
| | | | GTTCAGCGGCAGTGGATCTGGGACAGATTT |
| | | | CACTCTCACCATCAGCAGCCTGCAGCCTGA |
| | | | AGATTTTGCAACTTATTACTGTCAGCAGGGA |
| | | | GCCAGTTTCCCTCTCACTTTTGGCGGAGGGA |
| | | | CCAAGGTTGAGATCAAA |
| P | 137 | LC CDR1 | RASQGISRWLA |
| VL germline | 102 | LC CDR2 | AASSLQS |
| VK1-12 | 138 | LC CDR3 | QQGASFPLT |
| | 104 | LC FR1 | DIQMTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 139 | VL amino acid | DIQMTQSPSSVSASVGDRVTITCRASQGISRWL |
| | | | AWYQQKPGKAPKLLIYAASSLQSGVPSRFSGS |
| | | | GSGTDFTLTISSLQPEDFATYYCQQGASFPLTF |
| | | | GGGTKVEIK |
| | 173 | LC | DIQMTQSPSSVSASVGDRVTITCRASQGISRWL |
| | | | AWYQQKPGKAPKLLIYAASSLQSGVPSRFSGS |
| | | | GSGTDFTLTISSLQPEDFATYYCQQGASFPLTF |
| | | | GGGTKVEIKRTVAAPSVFIFPPSDEQLKSGTAS |
| | | | VVCLLNNFYPREAKVQWKVDNALQSGNSQE |
| | | | SVTEQDSKDSTYSLSSTLTLSKADYEKHKVYA |
| | | | CEVTHQGLSSPVTKSFNRGEC |
| | 140 | VL DNA | GACATCCAGATGACCCAGTCTCCATCTTCCG |
| | | | TGTCTGCATCTGTAGGAGACAGAGTCACCA |
| | | | TCACTTGTCGGGCGAGTCAGGGTATTAGCA |
| | | | GGTGGTTAGCCTGGTATCAGCAGAAACCAG |
| | | | GGAAAGCCCCTAAGCTCCTGATCTATGCTGC |
| | | | ATCCAGTTTGCAAAGTGGGGTCCCATCAAG |
| | | | GTTCAGCGGCAGTGGATCTGGGACAGATTT |
| | | | CACTCTCACCATCAGCAGCCTGCAGCCTGA |
| | | | AGATTTTGCAACTTATTACTGTCAGCAGGGA |
| | | | GCCAGTTTCCCTCTCACTTTTGGCGGAGGGA |
| | | | CCAAGGTTGAGATCAAA |
| Q | 92 | LC CDR1 | RSSQSLLHSNGYNYLD |
| | 93 | LC CDR2 | LGSNRAS |
| | 204 | LC CDR3 | MQTLELPPT |
| | 95 | LC FR1 | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 205 | VL amino acid | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | ELPPTFGGGTKVEIK |
| | 206 | LC | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMQTL |
| | | | ELPPTFGGGTKVEIK |
| | | | RTVAAPSVFIFPPSDEQLKSGTASVVCLLNNFY |
| | | | PREAKVQWKVDNALQSGNSQESVTEQDSKDS |
| | | | TYSLSSTLTLSKADYEKHKVYACEVTHQGLSS |
| | | | PVTKSFNRGEC |
| | 207 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGCAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGCAGACACTCGAACTGCCTCCTACT |
| | | | TTTGGCGGAGGGACCAAGGTTGAGATCAAA |
| R | 92 | LC CDR1 | RSSQSLLHSNGYNYLD |
| | 93 | LC CDR2 | LGSNRAS |
| | 208 | LC CDR3 | MSTLELPPT |
| | 95 | LC FR1 | DIVMTQSPLSLPVTPGEPASISC |
| | 96 | LC FR2 | WYLQKPGQSPQLLIY |
| | 97 | LC FR3 | GVPDRFSGSGSGTDFTLKISRVEAEDVGVYYC |
| | 98 | LC FR4 | FGGGTKVEIK |
| | 209 | VL amino acid | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMSTL |
| | | | ELPPTFGGGTKVEIK |
| | 210 | LC | DIVMTQSPLSLPVTPGEPASISCRSSQSLLHSNG |
| | | | YNYLDWYLQKPGQSPQLLIYLGSNRASGVPD |
| | | | RFSGSGSGTDFTLKISRVEAEDVGVYYCMSTL |
| | | | ELPPTFGGGTKVEIK |
| | | | RTVAAPSVFIFPPSDEQLKSGTASVVCLLNNFY |
| | | | PREAKVQWKVDNALQSGNSQESVTEQDSKDS |
| | | | TYSLSSTLTLSKADYEKHKVYACEVTHQGLSS |
| | | | PVTKSFNRGEC |
| | 211 | VL DNA | GATATTGTGATGACTCAGTCTCCACTCTCCC |
| | | | TGCCCGTCACCCCTGGAGAGCCGGCCTCCAT |
| | | | CTCCTGCAGGTCTAGTCAGAGCCTCCTGCAT |
| | | | AGTAATGGATACAACTATTTGGATTGGTACC |
| | | | TGCAGAAGCCAGGGCAGTCTCCACAGCTCC |
| | | | TGATCTATTTGGGTTCTAATCGGGCCTCCGG |
| | | | GGTCCCTGACAGGTTCAGTGGCAGTGGATC |
| | | | AGGCACAGATTTTACACTGAAAATCAGCAG |
| | | | AGTGGAGGCTGAGGATGTTGGGGTTTATTA |
| | | | CTGCATGAGTACACTCGAATTGCCTCCTACT |
| | | | TTTGGCGGAGGGACCAAGGTTGAGATCAAA |
| S | 212 | LC CDR1 | RASQGISPWLA |
| | 102 | LC CDR2 | AASSLQS |
| | 103 | LC CDR3 | QQANSFPIT |
| | 213 | LC FR1 | DIQLTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 214 | VL amino acid | DIQLTQSPSSVSASVGDRVTITCRASQGISPWL |
| | | | AWYQQKPGKAPKLLIYAASSLQSGVPSRFSGS |
| | | | GSGTDFTLTISSLQPEDFATYYCQQANSFPITF |
| | | | GGGTKVEIK |
| | 215 | LC | DIQLTQSPSSVSASVGDRVTITCRASQGISPWL |
| | | | AWYQQKPGKAPKLLIYAASSLQSGVPSRFSGS |
| | | | GSGTDFTLTISSLQPEDFATYYCQQANSFPITF |
| | | | GGGTKVEIK |
| | | | RTVAAPSVFIFPPSDEQLKSGTASVVCLLNNFY |
| | | | PREAKVQWKVDNALQSGNSQESVTEQDSKDS |
| | | | TYSLSSTLTLSKADYEKHKVYACEVTHQGLSS |
| | | | PVTKSFNRGEC |
| | 216 | VL DNA | GACATCCAGTTGACCCAGTCTCCATCTTCCG |
| | | | TGTCTGCATCTGTAGGAGACAGAGTCACCA |
| | | | TCACTTGTCGGGCGAGTCAGGGTATTAGCCC |
| | | | CTGGTTAGCCTGGTATCAGCAGAAACCAGG |
| | | | GAAAGCCCCTAAGCTCCTGATCTATGCTGCA |
| | | | TCCAGTTTGCAAAGTGGGGTCCCATCAAGG |
| | | | TTCAGCGGCAGTGGATCTGGGACAGATTTC |
| | | | ACTCTCACCATCAGCAGCCTGCAGCCTGAA |
| | | | GATTTTGCAACTTACTATTGTCAACAGGCTA |
| | | | ACAGTTTCCCTATCACCTTTGGCGGAGGGAC |
| | | | CAAGGTTGAGATCAAA |
| T | 101 | LC CDR1 | RASQGISSWLA |
| | 102 | LC CDR2 | AASSLQS |
| | 217 | LC CDR3 | QQASQFPIT |
| | 104 | LC FR1 | DIQMTQSPSSVSASVGDRVTITC |
| | 105 | LC FR2 | WYQQKPGKAPKLLIY |
| | 106 | LC FR3 | GVPSRFSGSGSGTDFTLTISSLQPEDFATYYC |
| | 98 | LC FR4 | FGGGTKVEIK |
| | 218 | VL amino acid | DIQMTQSPSSVSASVGDRVTITCRASQGISSWL |
| | | | AWYQQKPGKAPKLLIYAASSLQSGVPSRFSGS |
| | | | GSGTDFTLTISSLQPEDFATYYCQQASQFPITF |
| | | | GGGTKVEIK |
| | 219 | LC | DIQMTQSPSSVSASVGDRVTITCRASQGISSWL |
| | | | AWYQQKPGKAPKLLIYAASSLQSGVPSRFSGS |
| | | | GSGTDFTLTISSLQPEDFATYYCQQASQFPITF |
| | | | GGGTKVEIK |
| | | | RTVAAPSVFIFPPSDEQLKSGTASVVCLLNNFY |
| | | | PREAKVQWKVDNALQSGNSQESVTEQDSKDS |
| | | | TYSLSSTLTLSKADYEKHKVYACEVTHQGLSS |
| | | | PVTKSFNRGEC |
| | 220 | VL DNA | GACATCCAGATGACCCAGTCTCCATCTTCCG |
| | | | TGTCTGCATCTGTAGGAGACAGAGTCACCA |
| | | | TCACTTGTCGGGCGAGTCAGGGTATTAGCA |
| | | | GCTGGTTAGCCTGGTATCAGCAGAAACCAG |
| | | | GGAAAGCCCCTAAGCTCCTGATCTATGCTGC |
| | | | ATCCAGTTTGCAAAGTGGGGTCCCATCAAG |
| | | | GTTCAGCGGCAGTGGATCTGGGACAGATTT |
| | | | CACTCTCACCATCAGCAGCCTGCAGCCTGA |
| | | | AGATTTTGCAACTTATTACTGTCAACAGGCT |
| | | | AGTCAGTTCCCTATCACCTTTGGCGGAGGGA |
| | | | CCAAGGTTGAGATCAAA |

### Heavy Chain (e.g., Heavy Chain Variable Region) Polypeptides (HC Polypeptides)

The present disclosure provides uses of polypeptides comprising heavy chain (HC) sequences (e.g., heavy chain variable region sequence(s)) that, for example, may be useful in an Antibody Agent as described herein targeting GDF15, and/or GFRAL, to treat and/or prevent certain types and/or instances of nausea and/or emesis with which it had not previously been considered to be associated. In some such embodiments, such provided polypeptides are useful and/or included in such antibody agents as described herein.

In some embodiments, a HC polypeptide comprises at least one HC CDR of a GDF15 Antibody Agent as provided in Table 2 or a sequence with at least 85% identity thereto. In some embodiments, a HC polypeptide comprises one, two or three HC CDRs (e.g., a HC CDR1, a HC CDR2 and/or a HC CDR3). In some embodiments, a HC polypeptide comprises a HC CDR1. In some embodiments, a HC polypeptide comprises a HC CDR2. In some embodiments, a HC polypeptide comprises a HC CDR3. In some embodiments, a HC polypeptide comprises a HC CDR1, a HC CDR2 and a HC CDR3.

In some embodiments, a HC polypeptide comprising a HC CDR1, a HC CDR2 and a HC CDR3 is capable of binding (e.g., specifically binding) to GDF15 and/or GFRAL.

In some embodiments, a HC polypeptide (e.g., a GDF15 Antibody Agent or a GFRAL Antibody Agent) further comprises one or more framework regions, and/or a heavy chain constant region, or a portion or a variant thereof (e.g., a CH1, CH2 and/or CH3 region). In some embodiments, a HC polypeptide comprises a CH1, a CH2 or a CH3 or a combination thereof. In some embodiments, a HC polypeptide comprises a CH2 and CH3, e.g., an Fc domain.

In some embodiments, a Fc domain comprises a mammalian Fc domain. In some embodiments, a Fc domain comprises a dog, a cat, a mouse, a rat, a rabbit, a primate or a human Fc domain. In some embodiments, a Fc domain comprises a human Fc domain. In some embodiments, a Fc domain comprises a dog Fc domain. In some embodiments, a Fc domain comprises a cat Fc domain. In some embodiments, an Fc domain is chosen from an Fc domain of an immunoglobulin isotype. In some embodiments, an immunoglobulin isotype comprises IgA, IgD, IgG, IgM, or IgE. In some embodiments, an Fc domain comprises an Fc domain of an IgG, e.g., a human IgG. In some embodiments, an IgG is or comprises IgG1, lgG2, lgG3, or lgG4.

In some embodiments, an Fc region is a wildtype Fc region, e.g., a wildtype human Fc region. In some embodiments, an Fc region comprises a variant, e.g., an Fc region comprising an addition, substitution, or deletion of at least one amino acid residue in an Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein C1q, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, a HC polypeptide (e.g., a GDF15 Antibody Agent or a GFRAL Antibody Agent) comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region. In some embodiments, a GDF15 Antibody Agent comprising a variant Fc region has reduced affinity to a human Fc receptor, e.g., FcγR I, FcγR II and/or FcγR III. Exemplary Fc region variants are disclosed in Saunders K. O., (2019) Frontiers in Immunology; vol 10, Article 296, the entire contents of which is hereby incorporated by reference. For example, a Fc region variant is or comprises a modification provided in Table 3 of Saunders K O (2019). In some embodiments, a Fc region variant comprises Leu234Ala/Leu235Ala (LALA) mutation, a Leu235Glu (LE) mutation, a Ser228Pro/Leu235Glu (SPLE) mutation, Leu234Ala/Leu235Ala/Pro239Gly (LALA-PG) mutation, Pro 331 Ser/Leu234Glu/Leu235Phe (TM), Asp265Ala (DA) mutation, Leu235Ala/Gly237Ala (LAGA) mutation, or a combination thereof.

In some embodiments, a HC polypeptide (e.g., a GDF15 Antibody Agent or a GFRAL Antibody Agent) comprises a Leu234Ala/Leu235Ala (LALA) mutation.

In some embodiments, a HC polypeptide (e.g., a GDF15 Antibody Agent or a GFRAL Antibody Agent) comprises a Leu235Ala/Gly237Ala (LAGA) mutation.

In some embodiments, a Fc region variant (e.g., in a GDF15 Antibody Agent or a GFRAL Antibody Agent) comprises a mutation relative to a wildtype Fc region, e.g., a IgG1 FcR wildtype region. In some embodiments, the hinge and CH2 sequence of an IgG1 FcR wildtype region comprises the sequence of: CPPCPAPELLGGPSVFLFPPK (SEQ ID NO: 176).

In some embodiments, a Fc region variant (e.g., in a GDF15 Antibody Agent or a GFRAL Antibody Agent) comprises a LAGA mutation, e.g., as shown in bold in SEQ ID NO: 177: CPPCPAPELAGAPSVFLFPPK. In some embodiments, a HC polypeptide comprises an Fc region having a LAGA mutation, e.g., as provided in SEQ ID NO: 177.

In some embodiments, a Fc region variant (e.g., in a GDF15 Antibody Agent or a GFRAL Antibody Agent) comprises a FEGG mutation, e.g., as shown in bold in SEQ ID NO: 178: CPPCPAPEFEGGPSVFLFPPK. In some embodiments, a HC polypeptide comprises an Fc region having a FEGG mutation, e.g., as provided in SEQ ID NO: 178.

In some embodiments, a Fc region variant (e.g., in a GDF15 Antibody Agent or a GFRAL Antibody Agent) comprises a AAGG mutation, e.g., as shown in bold in SEQ ID NO: 179: CPPCPAPEAAGGPSVFLFPPK. In some embodiments, a HC polypeptide comprises an Fc region having a AAGG mutation, e.g., as provided in SEQ ID NO: 179.

In some embodiments, a Fc region variant (e.g., in a GDF15 Antibody Agent or a GFRAL Antibody Agent) comprises a AAGA mutation, e.g., as shown in bold in SEQ ID NO: 180: CPPCPAPEAAGAPSVFLFPPK. In some embodiments, an AAGA mutation is also referred to as Leu234Ala/Leu235Ala/Glu237Ala (LALAGA). In some embodiments, a HC polypeptide comprises an Fc region having an AAGA mutation, e.g., as provided in SEQ ID NO: 180.

In some embodiments, a Fc region variant (e.g., in a GDF15 Antibody Agent or a GFRAL Antibody Agent) comprises a I253A mutation, a H310A mutation, a H435R mutation, a H435A mutation or any combination thereof.

In some embodiments, a Fc region variant (e.g., in a GDF15 Antibody Agent or a GFRAL Antibody Agent) comprising an Fc mutation (e.g., as described herein) has reduced binding (e.g., no binding) to a neonatal Fc receptor (FcRn), e.g., when compared to an otherwise similar Fc region without an Fc mutation. In some embodiments, a GDF15 antibody agent comprising an Fc region having an Fc mutation has reduced binding (e.g., no binding) to FcRn and reduced placental transfer, compared to an otherwise similar GDF15 antibody agent with an Fc region without an Fc mutation (e.g., as described herein).

In some embodiments, a Fc region variant (e.g., in a GDF15 Antibody Agent or a GFRAL Antibody Agent) comprising a LAGA mutation, a FEGG mutation, an AAGG mutation, an AAGA mutation, a LALA mutation or a combination thereof, has reduced binding (e.g., no binding) to a neonatal Fc receptor (FcRn), e.g., when compared to an otherwise similar Fc region without a LAGA mutation, a FEGG mutation, an AAGG mutation, an AAGA mutation, a LALA mutation or a combination thereof. In some embodiments, a GDF15 antibody agent comprising an Fc region having a LAGA mutation, a FEGG mutation, an AAGG mutation, an AAGA mutation, a LALA mutation or a combination thereof has reduced binding (e.g., no binding) to FcRn and reduced placental transfer, compared to an otherwise similar GDF15 antibody agent with an Fc region without a LAGA mutation, a FEGG mutation, an AAGG mutation, an AAGA mutation, a LALA mutation or a combination thereof.

In some embodiments, a Fc region variant (e.g., in a GDF15 Antibody Agent or a GFRAL Antibody Agent) comprising a I253A mutation, a H310A mutation, a H435R mutation, a H435A mutation or any combination thereof, has reduced binding (e.g., no binding) to a neonatal Fc receptor (FcRn), e.g., when compared to an otherwise similar Fc region without a I253A mutation, a H310A mutation, a H435R mutation, or a H435A mutation or any combination thereof. In some embodiments, a GDF15 antibody agent comprising an Fc region having a I253A mutation, a H310A mutation, a H435R mutation, a H435A mutation, or any combination thereof has reduced binding (e.g., no binding) to FcRn and reduced placental transfer, compared to an otherwise similar GDF15 antibody agent with an Fc region without a I253A mutation, a H310A mutation, a H435R mutation, or a H435A mutation or any combination thereof.

In some embodiments, a HC polypeptide further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, a HC polypeptide comprises a CH3 domain or a variant thereof. In some embodiments, a CH3 variant is characterized in that when introduced in a HC polypeptide, a half-life of the HC polypeptide is extended without reducing other desirable characteristics, such as neutralization potency, effector function, and/or developability. In some embodiments, a HC polypeptide having a CH3 variant has an extended half-life compared to an otherwise similar HC polypeptide without a CH3 variant.

In some embodiments, a CH3 variant has an addition, substitution, or deletion of at least one amino acid residue compared to a reference CH3 domain, e.g., a wild-type CH3 domain.

In some embodiments, a CH3 variant has an amino acid residue at position 428 which differs from a reference CH3 domain, e.g., a wild-type CH3 domain. In some embodiments, a CH3 variant has an amino acid residue at position 434 which differs from a reference CH3 domain, e.g., a wild-type CH3 domain. In some embodiments, a CH3 variant has amino acid residues at positions 428 and 434 which differ from a reference CH3 domain, e.g., a wild-type CH3 domain.

In some embodiments, a CH3 variant has a leucine at position 428.

In some embodiments, a CH3 variant has an alanine at position 434.

In some embodiments, a CH3 variant has a leucine at position 428 and an alanine at position 434.

In some embodiments, a HC polypeptide comprising a CH3 variant is characterized in that when administered to a subject, increased antibody dependent cellular cytotoxicity (ADCC) and/or antibody-dependent cellular phagocytosis (ADCP) is observed as compared to a subject administered a HC polypeptide without a CH3 variant. In some embodiments, increased ADCC is characterized by one or more of: increased surface expression of CD107α on natural killer (NK) cells, increased interferon γ (IFNγ) production by NK cells or increased tumor necrosis factor α (TNFα) production by NK cells. In some embodiments, increased ADCP is characterized by one or more of: co-localization of target cells and macrophages utilizing microscopy or flow cytometry; and the inclusion of a pH-sensitive dye to differentiate between cell-associated and internalized target cells.

In some embodiments, a HC polypeptide comprising a CH3 variant has improved developability as compared to a HC polypeptide without a CH3 variant. In some embodiments, improving the developability of a HC polypeptide comprising a CH3 variant comprises increasing expression, increasing solubility, increasing covalent integrity, increasing conformational stability, increasing colloidal stability, decreasing poly-specificity, and/or decreasing immunogenicity of a HC polypeptide comprising a CH3 variant relative to a HC polypeptide without a CH3 variant.

In some embodiments, disclosed herein are preparations of an antibody agent comprising a human constant region comprising a variant CH3 domain, wherein the antibody agent is characterized in that the neutralization potency and/or effector function of the antibody agent is comparable to that of an antibody agent comprising a parent CH3 domain, and/or wherein the antibody agent is characterized in that the developability of the antibody agent is increased relative to that of an antibody agent comprising a reference (e.g., parent) CH3 domain, wherein the variant CH3 domain differs from a parent CH3 domain at positions 428 and 434, and wherein the variant CH3 domain comprises a leucine at position 428 and an alanine at position 434. In some embodiments, the developability of the antibody agent comprises high level expression, high solubility, covalent integrity, conformational stability, colloidal stability, low poly-specificity, and/or low immunogenicity

According to IMGT, the CH3 domain is the amino acid positions (or simply referred to as “positions” herein) 341-446 (EU numbering). The term “CH3 domain” is used in a broad sense herein to refer to a heavy chain region comprising at least seven consecutive amino acid positions of the heavy chain positions 341-446 (EU numbering)). A CH3 domain reference sequence, corresponding to the amino acid positions 341-446 according to EU numbering, is provided herein as SEQ ID NO: 221, which is an exemplary amino acid sequence of a wild-type (WT) CH3 domain.

Exemplary CH3 domain reference sequence:

(SEQ ID NO: 221)
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPG.

In some embodiments, a HC polypeptide comprises: (i) a HC CDR1 sequence provided in Table 2, e.g., SEQ ID NO: 1, SEQ ID NO: 10, SEQ ID NO: 14, SEQ ID NO: 18, SEQ ID NO: 22, SEQ ID NO: 31, SEQ ID NO: 40, SEQ ID NO: 49, SEQ ID NO: 56, SEQ ID NO: 63, SEQ ID NO: 68, SEQ ID NO: 73, SEQ ID NO: 78, SEQ ID NO: 82 or SEQ ID NO: 88; (ii) a sequence with at least 85%, 86%, 87%, 88%, 89%, 90%, 91%, 92%, 93%, 94%, 95%, 96%, 97%, 98%, 99% identity to a HC CDR1 sequence provided in Table 2, e.g., SEQ ID NO: 1, SEQ ID NO: 10, SEQ ID NO: 14, SEQ ID NO: 18, SEQ ID NO: 22, SEQ ID NO: 31, SEQ ID NO: 40, SEQ ID NO: 49, SEQ ID NO: 56, SEQ ID NO: 63, SEQ ID NO: 68, SEQ ID NO: 73, SEQ ID NO: 78, SEQ ID NO: 82 or SEQ ID NO: 88; or (iii) a sequence having at least 5, 10, or 20 substitutions compared to a HC CDR1 sequence provided in Table 2, e.g., SEQ ID NO: 1, SEQ ID NO: 10, SEQ ID NO: 14, SEQ ID NO: 18, SEQ ID NO: 22, SEQ ID NO: 31, SEQ ID NO: 40, SEQ ID NO: 49, SEQ ID NO: 56, SEQ ID NO: 63, SEQ ID NO: 68, SEQ ID NO: 73, SEQ ID NO: 78, SEQ ID NO: 82 or SEQ ID NO: 88.

In some embodiments, a HC polypeptide comprises: (i) a HC CDR2 sequence provided in Table 2, e.g., SEQ ID NO: 2, SEQ ID NO: 11, SEQ ID NO: 15, SEQ ID NO: 19, SEQ ID NO: 23, SEQ ID NO: 32, SEQ ID NO: 50, SEQ ID NO: 57, SEQ ID NO: 60, SEQ ID NO: 64, SEQ ID NO: 69, SEQ ID NO: 74, SEQ ID NO: 79, SEQ ID NO: 83 or SEQ ID NO: 200; (ii) a sequence with at least 85%, 86%, 87%, 88%, 89%, 90%, 91%, 92%, 93%, 94%, 95%, 96%, 97%, 98%, 99% identity to a HC CDR2 sequence provided in Table 2, e.g., SEQ ID NO: 2, SEQ ID NO: 11, SEQ ID NO: 15, SEQ ID NO: 19, SEQ ID NO: 23, SEQ ID NO: 32, SEQ ID NO: 50, SEQ ID NO: 57, SEQ ID NO: 60, SEQ ID NO:64, SEQ ID NO: 69, SEQ ID NO: 74, SEQ ID NO: 79, SEQ ID NO: 83 or SEQ ID NO: 200; or (iii) a sequence having at least 5, 10, or 20 substitutions compared to a HC CDR2 sequence provided in Table 2, e.g., SEQ ID NO: 2, SEQ ID NO: 11, SEQ ID NO: 15, SEQ ID NO: 19, SEQ ID NO: 23, SEQ ID NO: 32, SEQ ID NO: 50, SEQ ID NO: 57, SEQ ID NO: 60, SEQ ID NO:64, SEQ ID NO: 69, SEQ ID NO: 74, SEQ ID NO: 79, SEQ ID NO: 83 or SEQ ID NO: 200.

In some embodiments, a HC polypeptide comprises: (i) a HC CDR3 sequence provided in Table 2, e.g., SEQ ID NO: 3, SEQ ID NO: 191, SEQ ID NO: 192, SEQ ID NO: 193, SEQ ID NO: 24, SEQ ID NO: 33, SEQ ID NO: 42, SEQ ID NO: 51, SEQ ID NO: 65, SEQ ID NO: 70, SEQ ID NO:75, SEQ ID NO:84, SEQ ID NO:89; (ii) a sequence with at least 85%, 86%, 87%, 88%, 89%, 90%, 91%, 92%, 93%, 94%, 95%, 96%, 97%, 98%, 99% identity to a HC CDR3 sequence provided in Table 2, e.g., SEQ ID NO: 3, SEQ ID NO: 191, SEQ ID NO: 192, SEQ ID NO: 193, SEQ ID NO: 24, SEQ ID NO: 33, SEQ ID NO: 42, SEQ ID NO: 51, SEQ ID NO: 65, SEQ ID NO: 70, SEQ ID NO:75, SEQ ID NO:84, SEQ ID NO:89; or (iii) a sequence having at least 5, 10, or 20 substitutions compared to a HC CDR3 sequence provided in Table 2, e.g., SEQ ID NO: 3, SEQ ID NO: 191, SEQ ID NO: 192, SEQ ID NO: 193, SEQ ID NO: 24, SEQ ID NO: 33, SEQ ID NO: 42, SEQ ID NO: 51, SEQ ID NO: 65, SEQ ID NO: 70, SEQ ID NO: 75, SEQ ID NO:84, SEQ ID NO:89.

In some embodiments, a HC polypeptide comprises: (i) an HC CDR1, HC CDR2, and HC CDR3 sequence provided in Table 1; (ii) a sequence with at least 85%, 86%, 87%, 88%, 89%, 90%, 91%, 92%, 93%, 94%, 95%, 96%, 97%, 98%, or 99% identity to an HC CDR1, HC CDR2, and HC CDR3 sequence provided in Table 1; (iii) a sequence having at least 5, 10, or 20 substitutions relative to an HC CDR1, HC CDR2, and HC CDR3 sequence provided in Table 1.

In some embodiments, a HC polypeptide comprises: (i) an HC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; (ii) an HC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or (iii) an HC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3.

In some embodiments, a HC polypeptide comprises: (i) an HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10; (ii) an HC CDR2 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; and/or (iii) an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

In some embodiments, a HC polypeptide comprises: (i) an HC CDR1 of SEQ ID NO: 14, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 14; (ii) an HC CDR2 of SEQ ID NO: 15, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 15; and/or (iii) an HC CDR3 of SEQ ID NO: 192, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 192.

In some embodiments, a HC polypeptide comprises: (i) an HC CDR1 of SEQ ID NO: 18, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 18; (ii) an HC CDR2 of SEQ ID NO: 19, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 19; and/or (iii) an HC CDR3 of SEQ ID NO: 193, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 193.

In some embodiments, a HC polypeptide comprises: (i) an HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; (ii) an HC CDR2 of SEQ ID NO: 23, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 23; and/or (iii) an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

In some embodiments, a HC polypeptide comprises: (i) an HC CDR1 of SEQ ID NO: 31, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 31; (ii) an HC CDR2 of SEQ ID NO: 32, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 32; and/or (iii) an HC CDR3 of SEQ ID NO: 33, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 33.

In some embodiments, a HC polypeptide comprises: (i) an HC CDR1 of SEQ ID NO: 40, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 40; (ii) an HC CDR2 of SEQ ID NO: 32, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 32; and/or (iii) an HC CDR3 of SEQ ID NO: 42, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 42.

In some embodiments, a HC polypeptide comprises: (i) an HC CDR1 of SEQ ID NO: 49, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 49; (ii) an HC CDR2 of SEQ ID NO: 50, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 50; and/or (iii) an HC CDR3 of SEQ ID NO: 51, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 51.

In some embodiments, a HC polypeptide comprises: (i) an HC CDR1 of SEQ ID NO: 56, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 56; (ii) an HC CDR2 of SEQ ID NO: 57, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 57; and/or (iii) an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

In some embodiments, a HC polypeptide comprises: (i) an HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; (ii) an HC CDR2 of SEQ ID NO: 60, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 60; and/or (iii) an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

In some embodiments, a HC polypeptide comprises: (i) an HC CDR1 of SEQ ID NO: 63 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 63; (ii) an HC CDR2 of SEQ ID NO: 64, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 64; and/or (iii) an HC CDR3 of SEQ ID NO: 65, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 65.

In some embodiments, a HC polypeptide comprises: (i) an HC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68; (ii) an HC CDR2 of SEQ ID NO: 69, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and/or (iii) an HC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70.

In some embodiments, a HC polypeptide comprises: (i) an HC CDR1 of SEQ ID NO: 73, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 73; (ii) an HC CDR2 of SEQ ID NO: 74, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 74; and/or (iii) an HC CDR3 of SEQ ID NO: 75, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 75.

In some embodiments, a HC polypeptide comprises: (i) an HC CDR1 of SEQ ID NO: 78, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 78; (ii) an HC CDR2 of SEQ ID NO: 79, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 79; and/or (iii) an HC CDR3 of SEQ ID NO: 75, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 75.

In some embodiments, a HC polypeptide comprises: (i) an HC CDR1 of SEQ ID NO: 82, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 82; (ii) an HC CDR2 of SEQ ID NO: 83, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 83; and/or (iii) an HC CDR3 of SEQ ID NO: 84, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 84.

In some embodiments, a HC polypeptide comprises: (i) an HC CDR1 of SEQ ID NO: 88, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 88; (ii) an HC CDR2 of SEQ ID NO: 57, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 57; and/or (iii) an HC CDR3 of SEQ ID NO: 89, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 89.

In some embodiments, a HC polypeptide comprises: (i) an HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10; (ii) an HC CDR2 of SEQ ID NO: 200, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 200; and/or (iii) an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

In some embodiments, a HC polypeptide further comprises one or more framework regions (FR), e.g., as described herein. In some embodiments, such a HC polypeptide comprises one, two, three or four FRs, e.g., as described herein. In some embodiments, a FR comprises a HC FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a HC FR sequence as described herein, or a sequence having at least 5, 10 or 20 alterations relative to a HC FR sequence as described herein.

In some embodiments, a HC polypeptide comprises: (i) a FR sequence provided in Table 2; (ii) a sequence with at least 92%, 93%, 94%, 95%, 96%, 97%, 98%, or 99% identity to a FR sequence provided in Table 2; or (iii) a sequence having at least 5, 10, or 20 alterations (e.g., substitutions, deletions or insertions (e.g., conservative substitutions)) compared to a FR sequence provided in Table 2.

In some embodiments, a HC polypeptide comprises a HC FR1 provided in Table 2, a sequence with at least 92%, 93%, 94%, 95%, 96%, 97%, 98%, or 99% identity to a HC FR1 sequence provided in Table 2, or a sequence having at least 5, 10, or 20 alterations (e.g., substitutions, deletions or insertions (e.g., conservative substitutions)) compared to a HC FR1 sequence provided in Table 2.

In some embodiments, a HC polypeptide comprises a HC FR2 provided in Table 2, a sequence with at least 92%, 93%, 94%, 95%, 96%, 97%, 98%, or 99% identity to a HC FR2 sequence provided in Table 2, or a sequence having at least 5, 10, or 20 alterations (e.g., substitutions, deletions or insertions (e.g., conservative substitutions)) compared to a HC FR2 sequence provided in Table 2.

In some embodiments a HC polypeptide comprises a HC FR3 provided in Table 2, a sequence with at least 92%, 93%, 94%, 95%, 96%, 97%, 98%, or 99% identity to a HC FR3 sequence provided in Table 2, or a sequence having at least 5, 10, or 20 alterations (e.g., substitutions, deletions or insertions (e.g., conservative substitutions)) compared to a HC FR3 sequence provided in Table 2.

In some embodiments, a HC polypeptide comprises a HC FR4 provided in Table 2, a sequence with at least 92%, 93%, 94%, 95%, 96%, 97%, 98%, or 99% identity to a HC FR4 sequence provided in Table 2, or a sequence having at least 5, 10, or 20 alterations (e.g., substitutions, deletions or insertions (e.g., conservative substitutions)) compared to a HC FR4 sequence provided in Table 2.

In some embodiments, a HC polypeptide comprises a HC CDR1, a HC CDR2 and HC CDR3 provided in Table 2 or a sequence with at least 85% identity thereto, and a HC FR1, HC FR2, HCFR3 and a HC FR4 provided in Table 2 or a sequence with at least 92% identity thereto.

In some embodiments, a HC polypeptide comprises the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8.

In some embodiments, a HC polypeptide comprises the sequence of SEQ ID NO: 12, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 12.

In some embodiments, a HC polypeptide comprises the sequence of SEQ ID NO: 16, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 16.

In some embodiments, a HC polypeptide comprises the sequence of SEQ ID NO: 20, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 20.

In some embodiments, a HC polypeptide comprises the sequence of SEQ ID NO: 29, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 29.

In some embodiments, a HC polypeptide comprises the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38.

In some embodiments, a HC polypeptide comprises the sequence of SEQ ID NO: 47, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 47.

In some embodiments, a HC polypeptide comprises the sequence of SEQ ID NO: 54, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 54.

In some embodiments, a HC polypeptide comprises the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58.

In some embodiments, a HC polypeptide comprises the sequence of SEQ ID NO: 61, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 61.

In some embodiments, a HC polypeptide comprises the sequence of SEQ ID NO: 66, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 66.

In some embodiments, a HC polypeptide comprises the sequence of SEQ ID NO: 71, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 71.

In some embodiments, a HC polypeptide comprises the sequence of SEQ ID NO: 76, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 76.

In some embodiments, a HC polypeptide comprises the sequence of SEQ ID NO: 80, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 80.

In some embodiments, a HC polypeptide comprises the sequence of SEQ ID NO: 86, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 86.

In some embodiments, a HC polypeptide comprises the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90.

In some embodiments, a HC polypeptide comprises the sequence of SEQ ID NO: 201, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 201.

In some embodiments, a HC polypeptide comprises a HC amino acid sequence provided in Table 2, e.g., any one of SEQ ID NOs: 143, 144, 145, 146, 147, 148, 149, 150, 151, 152, 153, 154, 155, 156, 157, 158 or 202; or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to a HC amino acid sequence provided in Table 2, e.g., any one of SEQ ID NOs: 143, 144, 145, 146, 147, 148, 149, 150, 151, 152, 153, 154, 155, 156, 157, 158 or 202.

In some embodiments, a HC polypeptide comprises a terminal lysine, e.g., as provided in Table 2. In some embodiments, a HC polypeptide does not comprise a terminal lysine.

In some embodiments, a HC polypeptide comprises the amino acid of SEQ ID NO: 143, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143.

In some embodiments, a HC polypeptide comprises the amino acid of SEQ ID NO: 143 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143 without a terminal lysine.

In some embodiments, a HC polypeptide comprises the amino acid of SEQ ID NO: 144, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144.

In some embodiments, a HC polypeptide comprises the amino acid of SEQ ID NO: 144 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144 without a terminal lysine.

In some embodiments, a HC polypeptide comprises the amino acid of SEQ ID NO: 145, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 145.

In some embodiments, a HC polypeptide comprises the amino acid of SEQ ID NO: 145 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 145 without a terminal lysine.

In some embodiments, a HC polypeptide comprises the amino acid of SEQ ID NO: 146 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146.

In some embodiments, a HC polypeptide comprises the amino acid of SEQ ID NO: 146 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146 without a terminal lysine.

In some embodiments, a HC polypeptide comprises the amino acid of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147.

In some embodiments, a HC polypeptide comprises the amino acid of SEQ ID NO: 147 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147 without a terminal lysine.

In some embodiments, a HC polypeptide comprises the amino acid of SEQ ID NO: 148, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 148.

In some embodiments, a HC polypeptide comprises the amino acid of SEQ ID NO: 148 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 148 without a terminal lysine.

In some embodiments, a HC polypeptide comprises the amino acid of SEQ ID NO: 149, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 149.

In some embodiments, a HC polypeptide comprises the amino acid of SEQ ID NO: 149 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 149 without a terminal lysine.

In some embodiments, a HC polypeptide comprises the amino acid of SEQ ID NO: 150, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 150.

In some embodiments, a HC polypeptide comprises the amino acid of SEQ ID NO: 150 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 150 without a terminal lysine.

In some embodiments, a HC polypeptide comprises the amino acid of SEQ ID NO: 151, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 151.

In some embodiments, a HC polypeptide comprises the amino acid of SEQ ID NO: 151 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 151 without a terminal lysine.

In some embodiments, a HC polypeptide comprises the amino acid of SEQ ID NO: 152, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 152.

In some embodiments, a HC polypeptide comprises the amino acid of SEQ ID NO: 152 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 152 without a terminal lysine.

In some embodiments, a HC polypeptide comprises the amino acid of SEQ ID NO: 153, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 153.

In some embodiments, a HC polypeptide comprises the amino acid of SEQ ID NO: 153 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 153 without a terminal lysine.

In some embodiments, a HC polypeptide comprises the amino acid of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154.

In some embodiments, a HC polypeptide comprises the amino acid of SEQ ID NO: 154 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154 without a terminal lysine.

In some embodiments, a HC polypeptide comprises the amino acid of SEQ ID NO: 155, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155.

In some embodiments, a HC polypeptide comprises the amino acid of SEQ ID NO: 155 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155 without a terminal lysine.

In some embodiments, a HC polypeptide comprises the amino acid of SEQ ID NO: 156, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 156.

In some embodiments, a HC polypeptide comprises the amino acid of SEQ ID NO: 156 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 156 without a terminal lysine.

In some embodiments, a HC polypeptide comprises the amino acid of SEQ ID NO: 157, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 157.

In some embodiments, a HC polypeptide comprises the amino acid of SEQ ID NO: 157 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 157 without a terminal lysine.

In some embodiments, a HC polypeptide comprises the amino acid of SEQ ID NO: 158, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 158.

In some embodiments, a HC polypeptide comprises the amino acid of SEQ ID NO: 158 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 158 without a terminal lysine.

In some embodiments, a HC polypeptide comprises the amino acid of SEQ ID NO: 202, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 202.

In some embodiments, a HC polypeptide comprises the amino acid of SEQ ID NO: 202 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 202 without a terminal lysine.

Exemplary useful HC polypeptides which may be included in GDF15 Antibody Agents are disclosed in Table 2 below.

TABLE 2
Heavy chain polypeptide and nucleic acid sequences

| | SEQ ID | | |
|---|---|---|---|
| CLONE | NO | FEATURE | SEQUENCE |
| A | 1 | HC CDR1 | GSITSGGSYWS |
| VH germline | 2 | HC CDR2 | AIVYSGSTYYNPSLKS |
| VH4-31 | 3 | HC CDR3 | ARGVEYQHFDY |
| | 4 | HC FR1 | QVQLQESGPGLVKPSQTLSLTCTVSG |
| | 5 | HC FR2 | WIRQHPGKGLEWIG |
| | 6 | HC FR3 | RVTISVDTSKNQFSLKLSSVTAADTAVYYC |
| | 7 | HC FR4 | WGQGTLVTVSS |
| | 8 | VH amino | QVQLQESGPGLVKPSQTLSLTCTVSGGSITSGG |
| | | acid | SYWSWIRQHPGKGLEWIGAIVYSGSTYYNPSL |
| | | | KSRVTISVDTSKNQFSLKLSSVTAADTAVYYCA |
| | | | RGVEYQHFDYWGQGTLVTVSS |
| | 143 | HC | QVQLQESGPGLVKPSQTLSLTCTVSGGSITSGG |
| | | | SYWSWIRQHPGKGLEWIGAIVYSGSTYYNPSL |
| | | | KSRVTISVDTSKNQFSLKLSSVTAADTAVYYCA |
| | | | RGVEYQHFDYWGQGTLVTVSS ASTKGPSVFP |
| | | | LAPSSKSTSG GTAALGCLVK DYFPEPVTVS |
| | | | WNSGALTSGV HTFPAVLQSS GLYSLSSVVT |
| | | | VPSSSLGTQT YICNVNHKPS NTKVDKKVEP |
| | | | KSCDKTHTCP PCPAPEAAGA PSVFLFPPKP |
| | | | KDTLMISRTP EVTCVVVDVS HEDPEVKFNW |
| | | | YVDGVEVHNA KTKPREEQYN STYRVVSVLT |
| | | | VLHQDWLNGK EYKCKVSNKA LPAPIEKTIS |
| | | | KAKGQPREPQ VYTLPPSREE MTKNQVSLTC |
| | | | LVKGFYPSDI AVEWESNGQP ENNYKTTPPV |
| | | | LDSDGSFFLY SKLTVDKSRW QQGNVFSCSV |
| | | | MHEALHNHYT QKSLSLSPGK |
| | 9 | VH DNA | CAGGTGCAGCTGCAGGAGTCGGGCCCAGGAC |
| | | | TGGTGAAGCCTTCACAGACCCTGTCCCTCAC |
| | | | CTGTACTGTCTCTGGTGGCTCCATCACCAGTG |
| | | | GTGGTTCCTACTGGAGCTGGATCCGCCAGCA |
| | | | CCCAGGGAAGGGCCTGGAGTGGATTGGGGCC |
| | | | ATCGTCTACAGTGGGAGCACCTACTACAACC |
| | | | CGTCCCTCAAGAGTCGAGTTACCATATCAGT |
| | | | AGACACGTCTAAGAACCAGTTCTCCCTGAAG |
| | | | CTGAGCAGTGTGACTGCCGCGGACACGGCGG |
| | | | TGTACTACTGCGCCAGAGGGGTGGAATATCA |
| | | | GCATTTCGATTACTGGGGACAGGGTACATTG |
| | | | GTCACCGTCTCCTCA |
| B | 10 | HC CDR1 | GSISSGGIYWT |
| VH germline | 11 | HC CDR2 | AIAYSGSTYYNPSLKS |
| VH4-31 | 191 | HC CDR3 | ARGIEYQHFDY |
| | 4 | HC FR1 | QVQLQESGPGLVKPSQTLSLTCTVSG |
| | 5 | HC FR2 | WIRQHPGKGLEWIG |
| | 6 | HC FR3 | RVTISVDTSKNQFSLKLSSVTAADTAVYYC |
| | 7 | HC FR4 | WGQGTLVTVSS |
| | 12 | VH amino | QVQLQESGPGLVKPSQTLSLTCTVSGGSISSGGI |
| | | acid | YWTWIRQHPGKGLEWIGAIAYSGSTYYNPSLK |
| | | | SRVTISVDTSKNQFSLKLSSVTAADTAVYYCAR |
| | | | GIEYQHFDYWGQGTLVTVSS |
| | 144 | HC | QVQLQESGPGLVKPSQTLSLTCTVSGGSISSGGI |
| | | | YWTWIRQHPGKGLEWIGAIAYSGSTYYNPSLK |
| | | | SRVTISVDTSKNQFSLKLSSVTAADTAVYYCAR |
| | | | GIEYQHFDYWGQGTLVTVSS ASTKGPSVFP |
| | | | LAPSSKSTSG GTAALGCLVK DYFPEPVTVS |
| | | | WNSGALTSGV HTFPAVLQSS GLYSLSSVVT |
| | | | VPSSSLGTQT YICNVNHKPS NTKVDKKVEP |
| | | | KSCDKTHTCP PCPAPEAAGA PSVFLFPPKP |
| | | | KDTLMISRTP EVTCVVVDVS HEDPEVKFNW |
| | | | YVDGVEVHNA KTKPREEQYN STYRVVSVLT |
| | | | VLHQDWLNGK EYKCKVSNKA LPAPIEKTIS |
| | | | KAKGQPREPQ VYTLPPSREE MTKNQVSLTC |
| | | | LVKGFYPSDI AVEWESNGQP ENNYKTTPPV |
| | | | LDSDGSFFLY SKLTVDKSRW QQGNVFSCSV |
| | | | MHEALHNHYT QKSLSLSPGK |
| | 13 | VH DNA | CAGGTGCAGCTGCAGGAGTCGGGCCCAGGAC |
| | | | TGGTGAAGCCTTCACAGACCCTGTCCCTCAC |
| | | | CTGTACTGTCTCTGGTGGCTCCATCAGCAGTG |
| | | | GTGGTATCTACTGGACCTGGATCCGCCAGCA |
| | | | CCCAGGGAAGGGCCTGGAGTGGATTGGGGCC |
| | | | ATCGCCTACAGTGGGAGCACCTACTACAACC |
| | | | CGTCCCTCAAGAGTCGAGTTACCATATCAGT |
| | | | AGACACGTCTAAGAACCAGTTCTCCCTGAAG |
| | | | CTGAGCAGTGTGACTGCCGCGGACACGGCGG |
| | | | TGTACTACTGCGCCAGAGGGATTGAATATCA |
| | | | GCATTTCGATTACTGGGGACAGGGTACATTG |
| | | | GTCACCGTCTCCTCA |
| C | 14 | HC CDR1 | GSISSGGYYWS |
| VH germline | 15 | HC CDR2 | AIVYSGHTYYNPSLKS |
| VH4-31 | 192 | HC CDR3 | ARGVEYQHFSL |
| | 4 | HC FR1 | QVQLQESGPGLVKPSQTLSLTCTVSG |
| | 5 | HC FR2 | WIRQHPGKGLEWIG |
| | 6 | HC FR3 | RVTISVDTSKNQFSLKLSSVTAADTAVYYC |
| | 7 | HC FR4 | WGQGTLVTVSS |
| | 16 | VH amino | QVQLQESGPGLVKPSQTLSLTCTVSGGSISSGG |
| | | acid | YYWSWIRQHPGKGLEWIGAIVYSGHTYYNPSL |
| | | | KSRVTISVDTSKNQFSLKLSSVTAADTAVYYCA |
| | | | RGVEYQHFSLWGQGTLVTVSS |
| | 145 | HC | QVQLQESGPGLVKPSQTLSLTCTVSGGSISSGG |
| | | | YYWSWIRQHPGKGLEWIGAIVYSGHTYYNPSL |
| | | | KSRVTISVDTSKNQFSLKLSSVTAADTAVYYCA |
| | | | RGVEYQHFSLWGQGTLVTVSS ASTKGPSVFP |
| | | | LAPSSKSTSG GTAALGCLVK DYFPEPVTVS |
| | | | WNSGALTSGV HTFPAVLQSS GLYSLSSVVT |
| | | | VPSSSLGTQT YICNVNHKPS NTKVDKKVEP |
| | | | KSCDKTHTCP PCPAPEAAGA PSVFLFPPKP |
| | | | KDTLMISRTP EVTCVVVDVS HEDPEVKFNW |
| | | | YVDGVEVHNA KTKPREEQYN STYRVVSVLT |
| | | | VLHQDWLNGK EYKCKVSNKA LPAPIEKTIS |
| | | | KAKGQPREPQ VYTLPPSREE MTKNQVSLTC |
| | | | LVKGFYPSDI AVEWESNGQP ENNYKTTPPV |
| | | | LDSDGSFFLY SKLTVDKSRW QQGNVFSCSV |
| | | | MHEALHNHYT QKSLSLSPGK |
| | 17 | VH DNA | CAGGTGCAGCTGCAGGAGTCGGGCCCAGGAC |
| | | | TGGTGAAGCCTTCACAGACCCTGTCCCTCAC |
| | | | CTGTACTGTCTCTGGTGGCTCCATCAGCAGTG |
| | | | GTGGTTACTACTGGAGCTGGATCCGCCAGCA |
| | | | CCCAGGGAAGGGCCTGGAGTGGATTGGGGCC |
| | | | ATCGTCTACAGTGGGCACACCTACTACAACC |
| | | | CGTCCCTCAAGAGTCGAGTTACCATATCAGT |
| | | | AGACACGTCTAAGAACCAGTTCTCCCTGAAG |
| | | | CTGAGCAGTGTGACTGCCGCGGACACGGCGG |
| | | | TGTACTACTGCGCCAGAGGGGTGGAATATCA |
| | | | GCATTTCAGTCTTTGGGGACAGGGTACATTG |
| | | | GTCACCGTCTCCTCA |
| D | 18 | HC CDR1 | GSINSGGYYWT |
| VH germline | 19 | HC CDR2 | AIYYSGSTVYNPSLKS |
| VH4-31 | 193 | HC CDR3 | ARGVEYQHFNE |
| | 4 | HC FR1 | QVQLQESGPGLVKPSQTLSLTCTVSG |
| | 5 | HC FR2 | WIRQHPGKGLEWIG |
| | 6 | HC FR3 | RVTISVDTSKNQFSLKLSSVTAADTAVYYC |
| | 7 | HC FR4 | WGQGTLVTVSS |
| | 20 | VH amino | QVQLQESGPGLVKPSQTLSLTCTVSGGSINSGG |
| | | acid | YYWTWIRQHPGKGLEWIGAIYYSGSTVYNPSL |
| | | | KSRVTISVDTSKNQFSLKLSSVTAADTAVYYCA |
| | | | RGVEYQHFNEWGQGTLVTVSS |
| | 146 | HC | QVQLQESGPGLVKPSQTLSLTCTVSGGSINSGG |
| | | | YYWTWIRQHPGKGLEWIGAIYYSGSTVYNPSL |
| | | | KSRVTISVDTSKNQFSLKLSSVTAADTAVYYCA |
| | | | RGVEYQHFNEWGQGTLVTVSS ASTKGPSVFP |
| | | | LAPSSKSTSG GTAALGCLVK DYFPEPVTVS |
| | | | WNSGALTSGV HTFPAVLQSS GLYSLSSVVT |
| | | | VPSSSLGTQT YICNVNHKPS NTKVDKKVEP |
| | | | KSCDKTHTCP PCPAPEAAGA PSVFLFPPKP |
| | | | KDTLMISRTP EVTCVVVDVS HEDPEVKFNW |
| | | | YVDGVEVHNA KTKPREEQYN STYRVVSVLT |
| | | | VLHQDWLNGK EYKCKVSNKA LPAPIEKTIS |
| | | | KAKGQPREPQ VYTLPPSREE MTKNQVSLTC |
| | | | LVKGFYPSDI AVEWESNGQP ENNYKTTPPV |
| | | | LDSDGSFFLY SKLTVDKSRW QQGNVFSCSV |
| | | | MHEALHNHYT QKSLSLSPGK |
| | 21 | VH DNA | CAGGTGCAGCTGCAGGAGTCGGGCCCAGGAC |
| | | | TGGTGAAGCCTTCACAGACCCTGTCCCTCAC |
| | | | CTGTACTGTCTCTGGTGGCTCCATCAACAGTG |
| | | | GTGGTTACTACTGGACCTGGATCCGCCAGCA |
| | | | CCCAGGGAAGGGCCTGGAGTGGATTGGGGCC |
| | | | ATCTATTACAGTGGGAGCACCGTCTACAACC |
| | | | CGTCCCTCAAGAGTCGAGTTACCATATCAGT |
| | | | AGACACGTCTAAGAACCAGTTCTCCCTGAAG |
| | | | CTGAGCAGTGTGACTGCCGCGGACACGGCGG |
| | | | TGTACTACTGCGCCAGAGGGGTGGAATATCA |
| | | | GCATTTCAATGAGTGGGGACAGGGTACATTG |
| | | | GTCACCGTCTCCTCA |
| E | 22 | HC CDR1 | YTFTSYGIN |
| VH germline | 23 | HC CDR2 | WITAYSGDTNYAQKLQG |
| VH1-18 | 24 | HC CDR3 | ARGPGQYADVDV |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 28 | HC FR4 | WGQGTMVTVSS |
| | 147 | HO | QVQLVQSGAEVKKPGASVKVSCKASGYTFTSY |
| | | | GINWVRQAPGQGLEWMGWITAYSGDTNYAQ |
| | | | KLQGRVTMTTDTSTSTAYMELRSLRSDDTAVY |
| | | | YCARGPGQYADVDVWGQGTMVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 29 | VH amino | QVQLVQSGAEVKKPGASVKVSCKASGYTFTSY |
| | | acid | GINWVRQAPGQGLEWMGWITAYSGDTNYAQ |
| | | | KLQGRVTMTTDTSTSTAYMELRSLRSDDTAVY |
| | | | YCARGPGQYADVDVWGQGTMVTVSS |
| | 30 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTACCTCCT |
| | | | ATGGTATCAACTGGGTGCGACAGGCCCCTGG |
| | | | ACAAGGGCTTGAGTGGATGGGATGGATCACC |
| | | | GCTTACAGTGGTGACACAAACTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCCAGAGGACCTGGACAGTATGC |
| | | | AGACGTAGACGTATGGGGTCAGGGTACAATG |
| | | | GTCACCGTCTCCTCA |
| F | 31 | HC CDR1 | GTFGGYAIS |
| VH germline | 32 | HC CDR2 | GIIPPYATANYAQKFQG |
| VH1-69 | 33 | HC CDR3 | ARGVGTTHLDV |
| | 34 | HC FR1 | QVQLVQSGAEVKKPGSSVKVSCKASG |
| | 26 | HC FR2 | WVRQAPGQGLEWMG |
| | 36 | HC FR3 | RVTITADESTSTAYMELSSLRSEDTAVYYC |
| | 28 | HC FR4 | WGQGTMVTVSS |
| | 38 | VH amino | QVQLVQSGAEVKKPGSSVKVSCKASGGTFGGY |
| | | acid | AISWVRQAPGQGLEWMGGIIPPYATANYAQKF |
| | | | QGRVTITADESTSTAYMELSSLRSEDTAVYYCA |
| | | | RGVGTTHLDVWGQGTMVTVSS |
| | 148 | HC | QVQLVQSGAEVKKPGSSVKVSCKASGGTFGGY |
| | | | AISWVRQAPGQGLEWMGGIIPPYATANYAQKF |
| | | | QGRVTITADESTSTAYMELSSLRSEDTAVYYCA |
| | | | RGVGTTHLDVWGQGTMVTVSS ASTKGPSVFP |
| | | | LAPSSKSTSG GTAALGCLVK DYFPEPVTVS |
| | | | WNSGALTSGV HTFPAVLQSS GLYSLSSVVT |
| | | | VPSSSLGTQT YICNVNHKPS NTKVDKKVEP |
| | | | KSCDKTHTCP PCPAPEAAGA PSVFLFPPKP |
| | | | KDTLMISRTP EVTCVVVDVS HEDPEVKFNW |
| | | | YVDGVEVHNA KTKPREEQYN STYRVVSVLT |
| | | | VLHQDWLNGK EYKCKVSNKA LPAPIEKTIS |
| | | | KAKGQPREPQ VYTLPPSREE MTKNQVSLTC |
| | | | LVKGFYPSDI AVEWESNGQP ENNYKTTPPV |
| | | | LDSDGSFFLY SKLTVDKSRW QQGNVFSCSV |
| | | | MHEALHNHYT QKSLSLSPGK |
| | 39 | VH DNA | CAGGTGCAGCTGGTGCAGTCTGGGGCTGAGG |
| | | | TGAAGAAGCCTGGGTCCTCGGTGAAGGTCTC |
| | | | CTGCAAGGCTTCTGGAGGCACCTTCGGAGGA |
| | | | TATGCTATCAGCTGGGTGCGACAGGCCCCTG |
| | | | GACAAGGGCTTGAGTGGATGGGAGGGATCAT |
| | | | CCCTCCATACGCAACAGCAAACTACGCACAG |
| | | | AAGTTCCAGGGCAGAGTCACGATTACCGCGG |
| | | | ACGAATCCACGAGCACAGCCTACATGGAGCT |
| | | | GAGCAGCCTGAGATCTGAGGACACGGCGGTG |
| | | | TACTACTGCGCCAGAGGGGTGGGAACTACCC |
| | | | ATCTAGACGTATGGGGTCAGGGTACAATGGT |
| | | | CACCGTCTCCTCA |
| G | 40 | HC CDR1 | GTFQGYAIS |
| VH germline | 32 | HC CDR2 | GIIPPYATANYAQKFQG |
| VH1-69 | 42 | HC CDR3 | ARGVGELHLDV |
| | 34 | HC FR1 | QVQLVQSGAEVKKPGSSVKVSCKASG |
| | 44 | HC FR2 | WARQAPGQGLEWMG |
| | 36 | HC FR3 | RVTITADESTSTAYMELSSLRSEDTAVYYC |
| | 28 | HC FR4 | WGQGTMVTVSS |
| | 47 | VH amino | QVQLVQSGAEVKKPGSSVKVSCKASGGTFQGY |
| | | acid | AISWARQAPGQGLEWMGGIIPPYATANYAQKF |
| | | | QGRVTITADESTSTAYMELSSLRSEDTAVYYCA |
| | | | RGVGELHLDVWGQGTMVTVSS |
| | 149 | HC | QVQLVQSGAEVKKPGSSVKVSCKASGGTFQGY |
| | | | AISWARQAPGQGLEWMGGIIPPYATANYAQKF |
| | | | QGRVTITADESTSTAYMELSSLRSEDTAVYYCA |
| | | | RGVGELHLDVWGQGTMVTVSS ASTKGPSVFP |
| | | | LAPSSKSTSG GTAALGCLVK DYFPEPVTVS |
| | | | WNSGALTSGV HTFPAVLQSS GLYSLSSVVT |
| | | | VPSSSLGTQT YICNVNHKPS NTKVDKKVEP |
| | | | KSCDKTHTCP PCPAPEAAGA PSVFLFPPKP |
| | | | KDTLMISRTP EVTCVVVDVS HEDPEVKFNW |
| | | | YVDGVEVHNA KTKPREEQYN STYRVVSVLT |
| | | | VLHQDWLNGK EYKCKVSNKA LPAPIEKTIS |
| | | | KAKGQPREPQ VYTLPPSREE MTKNQVSLTC |
| | | | LVKGFYPSDI AVEWESNGQP ENNYKTTPPV |
| | | | LDSDGSFFLY SKLTVDKSRW QQGNVFSCSV |
| | | | MHEALHNHYT QKSLSLSPGK |
| | 48 | VH DNA | CAGGTGCAGCTGGTGCAGTCTGGGGCTGAGG |
| | | | TGAAGAAGCCTGGGTCCTCGGTGAAGGTCTC |
| | | | CTGCAAGGCTTCTGGAGGCACCTTCCAAGGA |
| | | | TATGCTATCAGCTGGGCGCGACAGGCCCCTG |
| | | | GACAAGGGCTTGAGTGGATGGGAGGGATCAT |
| | | | CCCTCCATACGCAACAGCAAACTACGCACAG |
| | | | AAGTTCCAGGGCAGAGTCACGATTACCGCGG |
| | | | ACGAATCCACGAGCACAGCCTACATGGAGCT |
| | | | GAGCAGCCTGAGATCTGAGGACACGGCGGTG |
| | | | TACTACTGCGCCAGAGGGGTGGGAGAGTTGC |
| | | | ATCTAGACGTATGGGGTCAGGGTACAATGGT |
| | | | CACCGTCTCCTCA |
| H | 49 | HC CDR1 | GTFSSYYIS |
| VH germline | 50 | HC CDR2 | GIAPISGTASYAQKFQG |
| VH1-69 | 51 | HC CDR3 | ARGLAYDGYFDL |
| | 34 | HC FR1 | QVQLVQSGAEVKKPGSSVKVSCKASG |
| | 26 | HC FR2 | WVRQAPGQGLEWMG |
| | 36 | HC FR3 | RVTITADESTSTAYMELSSLRSEDTAVYYC |
| | 53 | HC FR4 | WGRGTLVTVSS |
| | 54 | VH amino | QVQLVQSGAEVKKPGSSVKVSCKASGGTFSSY |
| | | acid | YISWVRQAPGQGLEWMGGIAPISGTASYAQKF |
| | | | QGRVTITADESTSTAYMELSSLRSEDTAVYYCA |
| | | | RGLAYDGYFDLWGRGTLVTVSS |
| | 150 | HC | QVQLVQSGAEVKKPGSSVKVSCKASGGTFSSY |
| | | | YISWVRQAPGQGLEWMGGIAPISGTASYAQKF |
| | | | QGRVTITADESTSTAYMELSSLRSEDTAVYYCA |
| | | | RGLAYDGYFDLWGRGTLVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 55 | VH DNA | CAGGTGCAGCTGGTGCAGTCTGGGGCTGAGG |
| | | | TGAAGAAGCCTGGGTCCTCGGTGAAGGTCTC |
| | | | CTGCAAGGCTTCTGGAGGCACCTTCAGCAGC |
| | | | TACTACATCAGCTGGGTGCGACAGGCCCCTG |
| | | | GACAAGGGCTTGAGTGGATGGGAGGGATCG |
| | | | CCCCTATCTCCGGTACAGCATCCTACGCACA |
| | | | GAAGTTCCAGGGCAGAGTCACGATTACCGCG |
| | | | GACGAATCCACGAGCACAGCCTACATGGAGC |
| | | | TGAGCAGCCTGAGATCTGAGGACACGGCGGT |
| | | | GTACTACTGCGCCAGAGGGTTGGCATATGAT |
| | | | GGCTACTTCGACCTATGGGGGAGGGGTACCT |
| | | | TGGTCACCGTCTCCTCA |
| I | 56 | HC CDR1 | YTFTDEGIN |
| VH germline | 57 | HC CDR2 | WITAYSGDTVYAQKLQG |
| VH1-18 | 24 | HC CDR3 | ARGPGQYADVDV |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 28 | HC FR4 | WGQGTMVTVSS |
| | 58 | VH amino | QVQLVQSGAEVKKPGASVKVSCKASGYTFTDE |
| | | acid | GINWVRQAPGQGLEWMGWITAYSGDTVYAQ |
| | | | KLQGRVTMTTDTSTSTAYMELRSLRSDDTAVY |
| | | | YCARGPGQYADVDVWGQGTMVTVSS |
| | 151 | HC | QVQLVQSGAEVKKPGASVKVSCKASGYTFTDE |
| | | | GINWVRQAPGQGLEWMGWITAYSGDTVYAQ |
| | | | KLQGRVTMTTDTSTSTAYMELRSLRSDDTAVY |
| | | | YCARGPGQYADVDVWGQGTMVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 59 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTACCGACG |
| | | | AAGGTATCAACTGGGTGCGACAGGCCCCTGG |
| | | | ACAAGGGCTTGAGTGGATGGGATGGATCACC |
| | | | GCTTACAGTGGTGACACAGTCTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCCAGAGGACCTGGACAGTATGC |
| | | | AGACGTAGACGTATGGGGTCAGGGTACAATG |
| | | | GTCACCGTCTCCTCA |
| J | 22 | HC CDR1 | YTFTSYGIN |
| VH germline | 60 | HC CDR2 | WIDAYSGSTNYAQKLQG |
| VH1-18 | 24 | HC CDR3 | ARGPGQYADVDV |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 28 | HC FR4 | WGQGTMVTVSS |
| | 61 | VH amino | QVQLVQSGAEVKKPGASVKVSCKASGYTFTSY |
| | | acid | GINWVRQAPGQGLEWMGWIDAYSGSTNYAQK |
| | | | LQGRVTMTTDTSTSTAYMELRSLRSDDTAVYY |
| | | | CARGPGQYADVDVWGQGTMVTVSS |
| | 152 | HC | QVQLVQSGAEVKKPGASVKVSCKASGYTFTSY |
| | | | GINWVRQAPGQGLEWMGWIDAYSGSTNYAQK |
| | | | LQGRVTMTTDTSTSTAYMELRSLRSDDTAVYY |
| | | | CARGPGQYADVDVWGQGTMVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 62 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTACCAGCT |
| | | | ATGGTATCAACTGGGTGCGACAGGCCCCTGG |
| | | | ACAAGGGCTTGAGTGGATGGGATGGATCGAC |
| | | | GCTTACAGTGGTTCCACAAACTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCCAGAGGACCTGGACAGTATGC |
| | | | AGACGTAGACGTATGGGGTCAGGGTACAATG |
| | | | GTCACCGTCTCCTCA |
| K | 63 | HC CDR1 | YTFTNEGIS |
| VH germline | 64 | HC CDR2 | WISAYTADTFYAQKLQG |
| VH1-18 | 65 | HC CDR3 | ARTRHDTSGLRFDY |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 7 | HC FR4 | WGQGTLVTVSS |
| | 66 | VH amino | QVQLVQSGAEVKKPGASVKVSCKASGYTFTNE |
| | | acid | GISWVRQAPGQGLEWMGWISAYTADTFYAQK |
| | | | LQGRVTMTTDTSTSTAYMELRSLRSDDTAVYY |
| | | | CARTRHDTSGLRFDYWGQGTLVTVSS |
| | 153 | HC | QVQLVQSGAEVKKPGASVKVSCKASGYTFTNE |
| | | | GISWVRQAPGQGLEWMGWISAYTADTFYAQK |
| | | | LQGRVTMTTDTSTSTAYMELRSLRSDDTAVYY |
| | | | CARTRHDTSGLRFDYWGQGTLVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 67 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTACCAACG |
| | | | AAGGTATCAGCTGGGTGCGACAGGCCCCTGG |
| | | | ACAAGGGCTTGAGTGGATGGGATGGATCTCC |
| | | | GCTTACACCGCAGACACATTCTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCCAGAACTAGACACGACACCTC |
| | | | CGGACTAAGATTCGACTACTGGGGACAGGGT |
| | | | ACATTGGTCACCGTCTCCTCA |
| L | 68 | HC CDR1 | YTFASYDII |
| VH germline | 69 | HC CDR2 | WISALSGDTVYAQKLQG |
| VH1-18 | 70 | HC CDR3 | ARTRHDTSGIRFDY |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 7 | HC FR4 | WGQGTLVTVSS |
| | 71 | VH amino | QVQLVQSGAEVKKPGASVKVSCKASGYTFASY |
| | | acid | DIIWVRQAPGQGLEWMGWISALSGDTVYAQK |
| | | | LQGRVTMTTDTSTSTAYMELRSLRSDDTAVYY |
| | | | CARTRHDTSGIRFDYWGQGTLVTVSS |
| | 154 | HC | QVQLVQSGAEVKKPGASVKVSCKASGYTFASY |
| | | | DIIWVRQAPGQGLEWMGWISALSGDTVYAQK |
| | | | LQGRVTMTTDTSTSTAYMELRSLRSDDTAVYY |
| | | | CARTRHDTSGIRFDYWGQGTLVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 72 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTGCCAGCT |
| | | | ATGACATCATCTGGGTGCGACAGGCCCCTGG |
| | | | ACAAGGGCTTGAGTGGATGGGATGGATCAGC |
| | | | GCTCTCAGTGGTGACACAGTCTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCCAGAACTAGACACGACACCTC |
| | | | CGGAATTCGGTTCGACTACTGGGGACAGGGT |
| | | | ACATTGGTCACCGTCTCCTCA |
| M | 73 | HC CDR1 | YTFDSYGIN |
| VH germline | 74 | HC CDR2 | WISAYSGDTAYAQELQG |
| VH1-18 | 75 | HC CDR3 | VRDTQYGGSPY |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 7 | HC FR4 | WGQGTLVTVSS |
| | 76 | VH amino | QVQLVQSGAEVKKPGASVKVSCKASGYTFDSY |
| | | acid | GINWVRQAPGQGLEWMGWISAYSGDTAYAQE |
| | | | LQGRVTMTTDTSTSTAYMELRSLRSDDTAVYY |
| | | | CVRDTQYGGSPYWGQGTLVTVSS |
| | 155 | HC | QVQLVQSGAEVKKPGASVKVSCKASGYTFDSY |
| | | | GINWVRQAPGQGLEWMGWISAYSGDTAYAQE |
| | | | LQGRVTMTTDTSTSTAYMELRSLRSDDTAVYY |
| | | | CVRDTQYGGSPYWGQGTLVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 77 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTGACAGCT |
| | | | ATGGTATCAACTGGGTGCGACAGGCCCCTGG |
| | | | ACAAGGGCTTGAGTGGATGGGATGGATCTCC |
| | | | GCTTACAGTGGTGACACAGCCTATGCACAGG |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGTTCGGGATACTCAGTATGGTGG |
| | | | TAGCCCATACTGGGGACAGGGTACATTGGTC |
| | | | ACCGTCTCCTCA |
| N | 78 | HC CDR1 | YTFTDYGIF |
| VH germline | 79 | HC CDR2 | WITAYSGDTHYAQKLQG |
| VH1-18 | 75 | HC CDR3 | VRDTQYGGSPY |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 7 | HC FR4 | WGQGTLVTVSS |
| | 80 | VH amino | QVQLVQSGAEVKKPGASVKVSCKASGYTFTD |
| | | acid | YGIFWVRQAPGQGLEWMGWITAYSGDTHYAQ |
| | | | KLQGRVTMTTDTSTSTAYMELRSLRSDDTAVY |
| | | | YCVRDTQYGGSPYWGQGTLVTVSS |
| | 156 | HC | QVQLVQSGAEVKKPGASVKVSCKASGYTFTD |
| | | | YGIFWVRQAPGQGLEWMGWITAYSGDTHYAQ |
| | | | KLQGRVTMTTDTSTSTAYMELRSLRSDDTAVY |
| | | | YCVRDTQYGGSPYWGQGTLVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 81 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTACCGACT |
| | | | ATGGTATCTTCTGGGTGCGACAGGCCCCTGG |
| | | | ACAAGGGCTTGAGTGGATGGGATGGATCACC |
| | | | GCTTACAGTGGTGACACACACTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGTGCGTGATACTCAGTATGGTGG |
| | | | TAGCCCATACTGGGGACAGGGTACATTGGTC |
| | | | ACCGTCTCCTCA |
| O | 82 | HC CDR1 | YTFDSLGMS |
| VH germline | 83 | HC CDR2 | YISTYSGDTVYAQKLQG |
| VH1-18 | 84 | HC CDR3 | ARESGQQVSALDP |
| | 85 | HC FR1 | QVQLVQSGAEVKKPGATVKVSCKASG |
| | 26 | HC FR2 | WVRQAPGQGLEWMG |
| | 27 | HC FR3 | RVTMTTDTSTSTAYMELRSLRSDDTAVYYC |
| | 7 | HC FR4 | WGQGTLVTVSS |
| | 86 | VH amino | QVQLVQSGAEVKKPGATVKVSCKASGYTFDSL |
| | | acid | GMSWVRQAPGQGLEWMGYISTYSGDTVYAQ |
| | | | KLQGRVTMTTDTSTSTAYMELRSLRSDDTAVY |
| | | | YCARESGQQVSALDPWGQGTLVTVSS |
| | 157 | HC | QVQLVQSGAEVKKPGATVKVSCKASGYTFDSL |
| | | | GMSWVRQAPGQGLEWMGYISTYSGDTVYAQ |
| | | | KLQGRVTMTTDTSTSTAYMELRSLRSDDTAVY |
| | | | YCARESGQQVSALDPWGQGTLVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 87 | VH DNA | CAGGTTCAGCTGGTGCAGTCTGGAGCTGAGG |
| | | | TGAAGAAGCCTGGGGCCACGGTGAAGGTCTC |
| | | | CTGCAAGGCTTCTGGTTACACCTTTGACAGCT |
| | | | TAGGTATGAGCTGGGTGCGACAGGCCCCTGG |
| | | | ACAAGGGCTTGAGTGGATGGGATATATCAGC |
| | | | ACGTACAGTGGTGACACAGTATATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCACGAGAGTCTGGCCAGCAGGT |
| | | | TTCCGCGCTCGACCCTTGGGGCCAGGGCACC |
| | | | CTGGTCACCGTCTCCTCA |
| P | 88 | HC CDR1 | YTFDSYGIH |
| VH germline | 57 | HC CDR2 | WITAYSGDTVYAQKLQG |
| VH1-18 | 89 | HC CDR3 | ARESGQTSSALGP |
| | 85 | HC FR1 | QVQLVQSGAEVKKPGATVKVSCKASG |
| | 26 | HC FR2 | WVRQAPGQGLEWMG |
| | 27 | HC FR3 | RVTMTTDTSTSTAYMELRSLRSDDTAVYYC |
| | 7 | HC FR4 | WGQGTLVTVSS |
| | 90 | VH amino | QVQLVQSGAEVKKPGATVKVSCKASGYTFDS |
| | | acid | YGIHWVRQAPGQGLEWMGWITAYSGDTVYA |
| | | | QKLQGRVTMTTDTSTSTAYMELRSLRSDDTAV |
| | | | YYCARESGQTSSALGPWGQGTLVTVSS |
| | 158 | HC | QVQLVQSGAEVKKPGATVKVSCKASGYTFDS |
| | | | YGIHWVRQAPGQGLEWMGWITAYSGDTVYA |
| | 91 | | QKLQGRVTMTTDTSTSTAYMELRSLRSDDTAV |
| | | | YYCARESGQTSSALGPWGQGTLVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | | VH DNA | CAGGTTCAGCTGGTGCAGTCTGGAGCTGAGG |
| | | | TGAAGAAGCCTGGGGCCACAGTGAAGGTCTC |
| | | | CTGCAAGGCTTCTGGTTACACCTTTGACAGCT |
| | | | ATGGTATCCACTGGGTGCGACAGGCCCCTGG |
| | | | ACAAGGGCTTGAGTGGATGGGATGGATCACC |
| | | | GCTTACAGTGGTGACACAGTCTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCCAGAGAGTCTGGACAAACAAG |
| | | | CTCTGCCCTGGGGCCATGGGGACAGGGTACA |
| | | | TTGGTCACCGTCTCCTCA |
| Q | 10 | HC CDR1 | GSISSGGIYWT |
| | 11 | HC CDR2 | AIAYSGSTYYNPSLKS |
| | 191 | HC CDR3 | ARGIEYQHFDY |
| | 4 | HC FR1 | QVQLQESGPGLVKPSQTLSLTCTVSG |
| | 5 | HC FR2 | WIRQHPGKGLEWIG |
| | 6 | HC FR3 | RVTISVDTSKNQFSLKLSSVTAADTAVYYC |
| | 7 | HC FR4 | WGQGTLVTVSS |
| | 12 | VH amino | QVQLQESGPGLVKPSQTLSLTCTVSGGSISSGGI |
| | | acid | YWTWIRQHPGKGLEWIGAIAYSGSTYYNPSLK |
| | | | SRVTISVDTSKNQFSLKLSSVTAADTAVYYCAR |
| | | | GIEYQHFDYWGQGTLVTVSS |
| | 144 | HC | QVQLQESGPGLVKPSQTLSLTCTVSGGSISSGGI |
| | | | YWTWIRQHPGKGLEWIGAIAYSGSTYYNPSLK |
| | | | SRVTISVDTSKNQFSLKLSSVTAADTAVYYCAR |
| | | | GIEYQHFDYWGQGTLVTVSS ASTKGPSVFP |
| | | | LAPSSKSTSG GTAALGCLVK DYFPEPVTVS |
| | | | WNSGALTSGV HTFPAVLQSS GLYSLSSVVT |
| | | | VPSSSLGTQT YICNVNHKPS NTKVDKKVEP |
| | | | KSCDKTHTCP PCPAPEAAGA PSVFLFPPKP |
| | | | KDTLMISRTP EVTCVVVDVS HEDPEVKFNW |
| | | | YVDGVEVHNA KTKPREEQYN STYRVVSVLT |
| | | | VLHQDWLNGK EYKCKVSNKA LPAPIEKTIS |
| | | | KAKGQPREPQ VYTLPPSREE MTKNQVSLTC |
| | | | LVKGFYPSDI AVEWESNGQP ENNYKTTPPV |
| | | | LDSDGSFFLY SKLTVDKSRW QQGNVFSCSV |
| | | | MHEALHNHYT QKSLSLSPGK |
| | 13 | VH DNA | CAGGTGCAGCTGCAGGAGTCGGGCCCAGGAC |
| | | | TGGTGAAGCCTTCACAGACCCTGTCCCTCAC |
| | | | CTGTACTGTCTCTGGTGGCTCCATCAGCAGTG |
| | | | GTGGTATCTACTGGACCTGGATCCGCCAGCA |
| | | | CCCAGGGAAGGGCCTGGAGTGGATTGGGGCC |
| | | | ATCGCCTACAGTGGGAGCACCTACTACAACC |
| | | | CGTCCCTCAAGAGTCGAGTTACCATATCAGT |
| | | | AGACACGTCTAAGAACCAGTTCTCCCTGAAG |
| | | | CTGAGCAGTGTGACTGCCGCGGACACGGCGG |
| | | | TGTACTACTGCGCCAGAGGGATTGAATATCA |
| | | | GCATTTCGATTACTGGGGACAGGGTACATTG |
| | | | GTCACCGTCTCCTCA |
| R | 10 | HC CDR1 | GSISSGGIYWT |
| | 200 | HC CDR2 | AIAYSGSTYYNPSLES |
| | 191 | HC CDR3 | ARGIEYQHFDY |
| | 4 | HC FR1 | QVQLQESGPGLVKPSQTLSLTCTVSG |
| | 5 | HC FR2 | WIRQHPGKGLEWIG |
| | 6 | HC FR3 | RVTISVDTSKNQFSLKLSSVTAADTAVYYC |
| | 7 | HC FR4 | WGQGTLVTVSS |
| | 201 | VH amino | QVQLQESGPGLVKPSQTLSLTCTVSGGSISSGGI |
| | | acid | YWTWIRQHPGKGLEWIGAIAYSGSTYYNPSLE |
| | | | SRVTISVDTSKNQFSLKLSSVTAADTAVYYCAR |
| | | | GIEYQHFDYWGQGTLVTVSS |
| | 202 | HC | QVQLQESGPGLVKPSQTLSLTCTVSGGSISSGGI |
| | | | YWTWIRQHPGKGLEWIGAIAYSGSTYYNPSLE |
| | | | SRVTISVDTSKNQFSLKLSSVTAADTAVYYCAR |
| | | | GIEYQHFDYWGQGTLVTVSS ASTKGPSVFP |
| | | | LAPSSKSTSG GTAALGCLVK DYFPEPVTVS |
| | | | WNSGALTSGV HTFPAVLQSS GLYSLSSVVT |
| | | | VPSSSLGTQT YICNVNHKPS NTKVDKKVEP |
| | | | KSCDKTHTCP PCPAPEAAGA PSVFLFPPKP |
| | | | KDTLMISRTP EVTCVVVDVS HEDPEVKFNW |
| | | | YVDGVEVHNA KTKPREEQYN STYRVVSVLT |
| | | | VLHQDWLNGK EYKCKVSNKA LPAPIEKTIS |
| | | | KAKGQPREPQ VYTLPPSREE MTKNQVSLTC |
| | | | LVKGFYPSDI AVEWESNGQP ENNYKTTPPV |
| | | | LDSDGSFFLY SKLTVDKSRW QQGNVFSCSV |
| | | | MHEALHNHYT QKSLSLSPGK |
| | 203 | VH DNA | CAGGTGCAGCTGCAGGAGTCGGGCCCAGGAC |
| | | | TGGTGAAGCCTTCACAGACCCTGTCCCTCAC |
| | | | CTGTACTGTCTCTGGTGGCTCCATCAGCAGTG |
| | | | GTGGTATCTACTGGACCTGGATCCGCCAGCA |
| | | | CCCAGGGAAGGGCTTGGAGTGGATTGGGGCC |
| | | | ATCGCCTACAGTGGGAGCACCTACTACAACC |
| | | | CGTCCCTCGAGAGTCGAGTTACCATATCAGT |
| | | | AGACACGTCTAAGAACCAGTTCTCCCTGAAG |
| | | | CTGAGCAGTGTGACTGCCGCGGACACGGCGG |
| | | | TGTACTACTGCGCCAGAGGGATTGAATATCA |
| | | | GCATTTCGATTACTGGGGACAGGGTACATTG |
| | | | GTCACCGTCTCCTCA |
| S | 22 | HC CDR1 | YTFTSYGIN |
| | 23 | HC CDR2 | WITAYSGDTNYAQKLQG |
| | 24 | HC CDR3 | ARGPGQYADVDV |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 28 | HC FR4 | WGQGTMVTVSS |
| | 29 | VH amino | QVQLVQSGAEVKKPGASVKVSCKASGYTFTSY |
| | | acid | GINWVRQAPGOGLEWMGWITAYSGDTNYAQ |
| | | | KLQGRVTMTTDTSTSTAYMELRSLRSDDTAVY |
| | | | YCARGPGQYADVDVWGQGTMVTVSS |
| | 147 | HC | QVQLVQSGAEVKKPGASVKVSCKASGYTFTSY |
| | | | GINWVRQAPGQGLEWMGWITAYSGDTNYAQ |
| | | | KLQGRVTMTTDTSTSTAYMELRSLRSDDTAVY |
| | | | YCARGPGQYADVDVWGQGTMVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 30 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTACCTCCT |
| | | | ATGGTATCAACTGGGTGCGACAGGCCCCTGG |
| | | | ACAAGGGCTTGAGTGGATGGGATGGATCACC |
| | | | GCTTACAGTGGTGACACAAACTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCCAGAGGACCTGGACAGTATGC |
| | | | AGACGTAGACGTATGGGGTCAGGGTACAATG |
| | | | GTCACCGTCTCCTCA |
| T | 22 | HC CDR1 | YTFTSYGIN |
| | 23 | HC CDR2 | WITAYSGDTNYAQKLQG |
| | 24 | HC CDR3 | ARGPGQYADVDV |
| | 25 | HC FR1 | QVQLVQSGAEVKKPGASVKVSCKASG |
| | 26 | HC FR2 | WVRQAPGQGLEWMG |
| | 27 | HC FR3 | RVTMTTDTSTSTAYMELRSLRSDDTAVYYC |
| | 28 | HC FR4 | WGQGTMVTVSS |
| | 29 | VH amino | QVQLVQSGAEVKKPGASVKVSCKASGYTFTSY |
| | | acid | GINWVRQAPGQGLEWMGWITAYSGDTNYAQ |
| | | | KLQGRVTMTTDTSTSTAYMELRSLRSDDTAVY |
| | | | YCARGPGQYADVDVWGQGTMVTVSS |
| | 147 | HC | QVQLVQSGAEVKKPGASVKVSCKASGYTFTSY |
| | | | GINWVRQAPGQGLEWMGWITAYSGDTNYAQ |
| | | | KLQGRVTMTTDTSTSTAYMELRSLRSDDTAVY |
| | | | YCARGPGQYADVDVWGQGTMVTVSS |
| | | | ASTKGPSVFP LAPSSKSTSG GTAALGCLVK |
| | | | DYFPEPVTVS WNSGALTSGV HTFPAVLQSS |
| | | | GLYSLSSVVT VPSSSLGTQT YICNVNHKPS |
| | | | NTKVDKKVEP KSCDKTHTCP PCPAPEAAGA |
| | | | PSVFLFPPKP KDTLMISRTP EVTCVVVDVS |
| | | | HEDPEVKFNW YVDGVEVHNA KTKPREEQYN |
| | | | STYRVVSVLT VLHQDWLNGK EYKCKVSNKA |
| | | | LPAPIEKTIS KAKGQPREPQ VYTLPPSREE |
| | | | MTKNQVSLTC LVKGFYPSDI AVEWESNGQP |
| | | | ENNYKTTPPV LDSDGSFFLY SKLTVDKSRW |
| | | | QQGNVFSCSV MHEALHNHYT QKSLSLSPGK |
| | 30 | VH DNA | CAGGTTCAGCTGGTGCAGTCTGGAGCTGAGG |
| | | | TGAAGAAGCCTGGGGCCTCAGTGAAGGTCTC |
| | | | CTGCAAGGCTTCTGGTTACACCTTTACCTCCT |
| | | | ATGGTATCAACTGGGTGCGACAGGCCCCTGG |
| | | | ACAAGGGCTTGAGTGGATGGGATGGATCACC |
| | | | GCTTACAGTGGTGACACAAACTATGCACAGA |
| | | | AGCTCCAGGGCAGAGTCACCATGACCACAGA |
| | | | CACATCCACGAGCACAGCCTACATGGAGCTG |
| | | | AGGAGCCTGAGATCTGACGACACGGCGGTGT |
| | | | ACTACTGCGCCAGAGGACCTGGACAGTATGC |
| | | | AGACGTAGACGTATGGGGTCAGGGTACAATG |
| | | | GTCACCGTCTCCTCA |

### GDF15 Antibody Agents Comprising a Light Chain Polypeptide and a Heavy Chain Polypeptide

In some embodiments, provided herein is use of a GDF15 Antibody Agent, e.g., a GDF15 Antibody Agent polypeptide, comprising a light chain comprising a variable region comprising one, two or three LC CDRs and a heavy chain comprising a variable region comprising one, two or three HC CDRs to treat and/or prevent certain types and/or instances of nausea and/or emesis with which it had not previously been considered to be associated. In some embodiments, a GDF15 Antibody Agent comprises a light chain comprising a LC CDR1, a LC CDR2 and a LC CDR3, and a heavy chain comprising a HC CDR1, a HC CDR2 and HC CDR3.

In some embodiments, a GDF15 Antibody Agent comprising a LC CDR1, a LC CDR2 and a LC CDR3, and a heavy chain comprising a HC CDR1, a HC CDR2 and HC CDR3 is able to specifically bind to GDF15, e.g., human, cyno, dog, cat or mouse GDF15.

In some embodiments, a GDF15 Antibody Agent comprises one, two, or three LC CDRs provided in Table 1 or a sequence with at least 85%, 86%, 87%, 88%, 89%, 90%, 91%, 92%, 93%, 94%, 95%, 96%, 97%, 98%, or 99% thereto; and one, two, or three HC CDRs provided in Table 2, or a sequence with at least 85%, 86%, 87%, 88%, 89%, 90%, 91%, 92%, 93%, 94%, 95%, 96%, 97%, 98%, or 99% thereto.

In some embodiments, a GDF15 Antibody Agent comprises: (a) a light chain comprising: (i) an LC CDR1 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR1 provided in Table 1; (ii) an LC CDR2 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR2 provided in Table 1; and/or (iii) an LC CDR3 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR3 provided in Table 1; and (b) a heavy chain comprising: (i) a HC CDR1 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR1 provided in Table 2; (ii) a HC CDR2 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR2 provided in Table 2; and/or (iii) a HC CDR3 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a HC CDR3 provided in Table 2.

In some embodiments, a GDF15 Antibody Agent comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and (ii) a HC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; a HC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and a HC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3.

In some embodiments, a GDF15 Antibody Agent comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and (ii) a HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10; a HC CDR2 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; and a HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

In some embodiments, a GDF15 Antibody Agent comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and (ii) a HC CDR1 of SEQ ID NO: 14, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 14; a HC CDR2 of SEQ ID NO: 15, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 15; and a HC CDR3 of SEQ ID NO: 192, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 192.

In some embodiments, a GDF15 Antibody Agent comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and (ii) a HC CDR1 of SEQ ID NO: 18, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 18; a HC CDR2 of SEQ ID NO: 19, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 19; and a HC CDR3 of SEQ ID NO: 193, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 193.

In some embodiments, a GDF15 Antibody Agent comprises: (i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; and (ii) a HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; a HC CDR2 of SEQ ID NO: 23, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 23; and a HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

In some embodiments, a GDF15 Antibody Agent comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 110, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 110; and (ii) a HC CDR1 of SEQ ID NO: 31, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 31; a HC CDR2 of SEQ ID NO: 32, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 32; and a HC CDR3 of SEQ ID NO: 33, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 33.

In some embodiments, a GDF15 Antibody Agent comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 110, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 110; and (ii) a HC CDR1 of SEQ ID NO: 40, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 40; a HC CDR2 of SEQ ID NO: 32, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 32; and a HC CDR3 of SEQ ID NO: 42, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 42.

In some embodiments, a GDF15 Antibody Agent comprises: (i) an LC CDR1 of SEQ ID NO: 117, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 117; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 118, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 118; and (ii) a HC CDR1 of SEQ ID NO: 49, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 49; an HC CDR2 of SEQ ID NO: 50, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 50; and an HC CDR3 of SEQ ID NO: 51, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 51.

In some embodiments, a GDF15 Antibody Agent comprises: (i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; and (ii) a HC CDR1 of SEQ ID NO: 56, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 56; an HC CDR2 of SEQ ID NO: 57, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 57; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

In some embodiments, a GDF15 Antibody Agent comprises: (i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; and (ii) a HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; an HC CDR2 of SEQ ID NO: 60, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 60; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

In some embodiments, a GDF15 Antibody Agent comprises: (i) an LC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 126, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 126; and (ii) a HC CDR1 of SEQ ID NO: 63, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 63; an HC CDR2 of SEQ ID NO: 64, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 64; and an HC CDR3 of SEQ ID NO: 65, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 65.

In some embodiments, a GDF15 Antibody Agent comprises: (i) an LC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 126, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 126; and (ii) a HC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68; an HC CDR2 of SEQ ID NO: 69, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and an HC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70.

In some embodiments, a GDF15 Antibody Agent comprises: (i) an LC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129; an LC CDR2 of SEQ ID NO: 130, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and an LC CDR3 of SEQ ID NO: 131, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 131; and (ii) a HC CDR1 of SEQ ID NO: 73, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 73; an HC CDR2 of SEQ ID NO: 74, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 74; and an HC CDR3 of SEQ ID NO: 75, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 75.

In some embodiments, a GDF15 Antibody Agent comprises: (i) an LC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129; an LC CDR2 of SEQ ID NO: 130, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and an LC CDR3 of SEQ ID NO: 131, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 131; and (ii) a HC CDR1 of SEQ ID NO: 78, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 78; an HC CDR2 of SEQ ID NO: 79, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 79; and an HC CDR3 of SEQ ID NO: 75, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 75.

In some embodiments, a GDF15 Antibody Agent comprises: (i) an LC CDR1 of SEQ ID NO: 137, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 137; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 138, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 138; and (ii) a HC CDR1 of SEQ ID NO: 82, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 82; an HC CDR2 of SEQ ID NO: 83, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 83; and an HC CDR3 of SEQ ID NO: 84, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 84.

In some embodiments, a GDF15 Antibody Agent comprises: (i) an LC CDR1 of SEQ ID NO: 137, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 137; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 138, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 138; and (ii) a HC CDR1 of SEQ ID NO: 88, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 88; an HC CDR2 of SEQ ID NO: 57, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 57; and an HC CDR3 of SEQ ID NO: 89, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 89.

In some embodiments, a GDF15 antibody agent comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204; and (ii) a HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10; an HC CDR2 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; and an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

In some embodiments, a GDF15 antibody agent comprises: (i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 208, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 208; and (ii) a HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10; an HC CDR2 of SEQ ID NO: 200, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 200; and an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

In some embodiments, a GDF15 antibody agent comprises: (i) an LC CDR1 of SEQ ID NO: 212, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 212; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; and (ii) a HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; an HC CDR2 of SEQ ID NO: 23, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 23; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

In some embodiments, a GDF15 antibody agent comprises: (i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 217, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 217; and (ii) a HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; an HC CDR2 of SEQ ID NO: 23, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 23; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

In some embodiments, a GDF15 Antibody Agent comprises a light chain polypeptide (LC polypeptide) as described herein.

In some embodiments, a GDF15 Antibody Agent comprises a heavy chain polypeptide (HC polypeptide) as described herein. In some embodiments, a HC polypeptide in a GDF15 antibody does not include a terminal lysine.

In some embodiments, a GDF15 Antibody Agent comprises a light chain polypeptide (LC polypeptide) as described herein and a heavy chain polypeptide (HC polypeptide) as described herein. In some embodiments, a HC polypeptide in a GDF15 Antibody Agent does not include a terminal lysine.

In some embodiments, a GDF15 Antibody Agent comprises a light chain comprising a variable region (VL) comprising three LC CDRs and one or more framework regions (e.g., as described herein); and a heavy chain comprising a variable region (VH) comprising three HC CDRs and one or more framework regions (e.g., as described herein).

In some embodiments, a VL and/or a VH of a GDF15 Antibody Agent further comprises one or more framework regions (FR), e.g., as described herein. In some embodiments, a VL and/or a VH of a GDF15 Antibody Agent comprises one, two, three or four FRs, e.g., as described herein. In some embodiments, a FR comprises a FR from a human mature antibody, a human germline sequence, a non-human framework (e.g., a rodent framework); or a non-human framework that has been modified, e.g., to remove antigenic or cytotoxic determinants, e.g., deimmunized, or partially humanized, or a sequence with at least 85% identity to a LC FR sequence as described herein, or a sequence having at least 5, 10 or 20 alterations relative to a LC FR sequence as described herein.

In some embodiments, a VL and/or a VH of a GDF15 Antibody Agent comprises: (i) a FR sequence provided in Table 1 or Table 2; (ii) a sequence with at least 92%, 93%, 94%, 95%, 96%, 97%, 98%, or 99% identity to a FR sequence provided in Table 1 or 2; or (iii) a sequence having at least 5, 10, or 20 alterations (e.g., substitutions, deletions or insertions (e.g., conservative substitutions)) compared to a FR sequence provided in Table 1 or 2.

In some embodiments, a VL and/or a VH of a GDF15 Antibody Agent comprises a FR1 provided in Table 1 or 2, a sequence with at least 92%, 93%, 94%, 95%, 96%, 97%, 98%, or 99% identity to a FR1 sequence provided in Table 1 or 2, or a sequence having at least 5, 10, or 20 alterations (e.g., substitutions, deletions or insertions (e.g., conservative substitutions)) compared to a FR1 sequence provided in Table 1 or 2.

In some embodiments, a VL and/or a VH of a GDF15 Antibody Agent comprises a FR2 provided in Table 1 or 2, a sequence with at least 92%, 93%, 94%, 95%, 96%, 97%, 98%, or 99% identity to a FR2 sequence provided in Table 1 or 2, or a sequence having at least 5, 10, or 20 alterations (e.g., substitutions, deletions or insertions (e.g., conservative substitutions)) compared to a FR2 sequence provided in Table 1 or 2.

In some embodiments a VL and/or a VH of a GDF15 Antibody Agent comprises a FR3 provided in Table 1 or 2, a sequence with at least 92%, 93%, 94%, 95%, 96%, 97%, 98%, or 99% identity to a FR3 sequence provided in Table 1 or 2, or a sequence having at least 5, 10, or 20 alterations (e.g., substitutions, deletions or insertions (e.g., conservative substitutions)) compared to a FR3 sequence provided in Table 1 or 2.

In some embodiments, a VL and/or a VH of a GDF15 Antibody Agent comprises a FR4 provided in Table 1 or 2, a sequence with at least 92%, 93%, 94%, 95%, 96%, 97%, 98%, or 99% identity to a FR4 sequence provided in Table 1 or 2, or a sequence having at least 5, 10, or 20 alterations (e.g., substitutions, deletions or insertions (e.g., conservative substitutions)) compared to a FR4 sequence provided in Table 1 or 2.

In some embodiments, a GDF15 Antibody Agent comprises a VL comprising 3 LC CDRs and a LC FR1, LC FR2, LCFR3 and a LC FR4 of a GDF15 Antibody Agent provided in Table 1 or a sequence with at least 92% identity thereto; and/or a VH comprising 3 HC CDRs and a HC FR1, HC FR2, HC FR3 and a HC FR4 of a GDF15 Antibody Agent provided in Table 2 or a sequence with at least 92% identity thereto.

In some embodiments, a GDF15 Antibody Agent comprises a VL sequence provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to a VL sequence provided in Table 1; and a VH sequence provided in Table 2 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to a VH sequence provided in Table 2.

In some embodiments, a GDF15 Antibody Agent comprises: (i) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8.

In some embodiments, a GDF15 Antibody Agent comprises the sequence SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 12, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 12.

In some embodiments, a GDF15 Antibody Agent comprises the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 16, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 16.

In some embodiments, a GDF15 Antibody Agent comprises the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 20, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 20.

In some embodiments, a GDF15 Antibody Agent comprises the sequence of SEQ ID NO: 107, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 107; and the sequence of SEQ ID NO: 29, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 29.

In some embodiments, a GDF15 Antibody Agent comprises the sequence of SEQ ID NO: 115, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 115; and the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38.

In some embodiments, a GDF15 Antibody Agent comprises the sequence of SEQ ID NO: 115, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 115; and the sequence of SEQ ID NO: 47, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 47.

In some embodiments, a GDF15 Antibody Agent comprises the sequence of SEQ ID NO: 123, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 123; and the sequence of SEQ ID NO: 54, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 54.

In some embodiments, a GDF15 Antibody Agent comprises the sequence of SEQ ID NO: 107, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 107; and the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58.

In some embodiments, a GDF15 Antibody Agent comprises the sequence of SEQ ID NO: 107, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 107; and the sequence of SEQ ID NO: 61, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 61.

In some embodiments, a GDF15 Antibody Agent comprises the sequence of SEQ ID NO: 127, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 127; and the sequence of SEQ ID NO: 66, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 66.

In some embodiments, a GDF15 Antibody Agent comprises the sequence of SEQ ID NO: 127, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 127; and the sequence of SEQ ID NO: 71, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 71.

In some embodiments, a GDF15 Antibody Agent comprises the sequence of SEQ ID NO: 135, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 135; and the sequence of SEQ ID NO: 76, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 76.

In some embodiments, a GDF15 Antibody Agent comprises the sequence of SEQ ID NO: 135, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 135; and the sequence of SEQ ID NO: 80, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 80.

In some embodiments, a GDF15 Antibody Agent comprises the sequence of SEQ ID NO: 139, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 139; and the sequence of SEQ ID NO: 86, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 86.

In some embodiments, a GDF15 Antibody Agent comprises the sequence of SEQ ID NO: 139, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 139; and the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90.

In some embodiments, a GDF15 antibody agent comprises the sequence of SEQ ID NO: 205, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 205; and the sequence of SEQ ID NO: 12, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 12.

In some embodiments, a GDF15 antibody agent comprises the sequence of SEQ ID NO: 209, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 209; and the sequence of SEQ ID NO: 201, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 201.

In some embodiments, a GDF15 antibody agent comprises the sequence of the sequence of SEQ ID NO: 214, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 214; and the sequence of SEQ ID NO: 29, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 29.

In some embodiments, a GDF15 antibody agent comprises the sequence of the sequence of SEQ ID NO: 218, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 218; and the sequence of SEQ ID NO: 29, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 29.

In some embodiments, a GDF15 Antibody Agent comprises: a light chain comprising three LC CDRs, one or more framework regions (e.g., as described herein) and a constant region; and a heavy chain comprising three HC CDRs, one or more framework regions (e.g., as described herein), and at least one constant region.

In some embodiments, a light chain constant region comprises a light chain kappa or a light chain lambda constant region.

In some embodiments, a light chain kappa constant region comprises the sequence of SEQ ID NO: 175, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 175.

(SEQ ID NO: 175)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, a heavy chain constant region comprises a CH1, CH2 and/or CH3. In some embodiments, at least one constant region comprises an Fc domain. In some embodiments, an Fc domain comprises a mammalian Fc domain. In some embodiments, an Fc domain comprises a dog, a cat, a mouse, a rat, a rabbit, a primate or a human Fc domain. In some embodiments, an Fc domain is chosen from an Fc domain of an immunoglobulin isotype. In some embodiments, an immunoglobulin isotype comprises IgA, IgD, IgG, IgM, or IgE. In some embodiments, an Fc domain comprises an Fc domain of an IgG, e.g., a human IgG. In some embodiments, an IgG is or comprises IgG1, lgG2, lgG3, or lgG4.

In some embodiments, a GDF15 Antibody Agent comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein C1q, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, a GDF15 Antibody Agent comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region. In some embodiments, a GDF15 Antibody Agent comprising a variant Fc region has reduced affinity to a human Fc receptor, e.g., FcγR I, FcγR II and/or FcγR III. Exemplary Fc region variants are disclosed in Saunders K. O., (2019) Frontiers in Immunology; vol 10, Article 296, the entire contents of which is hereby incorporated by reference. For example, a Fc region variant is or comprises a modification provided in Table 3 of Saunders K O (2019). In some embodiments, a Fc region variant comprises Leu234Ala/Leu235Ala (LALA) mutation, a Leu235Glu (LE) mutation, a Ser228Pro/Leu235Glu (SPLE) mutation, Leu234Ala/Leu235Ala/Pro239Gly (LALA-PG) mutation, Pro 331 Ser/Leu234Glu/Leu235Phe™, Asp265Ala (DA) mutation, Leu235Ala/Gly237Ala (LAGA) mutation, or a combination thereof.

In some embodiments, a GDF15 Antibody Agent comprises a Leu234Ala/Leu235Ala (LALA) mutation.

In some embodiments, a GDF15 Antibody Agent comprises a Leu235Ala/Gly237Ala (LAGA) mutation.

In some embodiments, a Fc region variant comprises a mutation relative to a wildtype Fc region, e.g., a IgG1 FcR wildtype region. In some embodiments, the hinge and CH2 sequence of an IgG1 FcR wildtype region comprises the sequence of:

(SEQ ID NO: 176)
CPPCPAPELLGGPSVFLFPPK

In some embodiments, a Fc region variant comprises a LAGA mutation, e.g., as shown in bold in SEQ ID NO: 177: CPPCPAPELAGAPSVFLFPPK. In some embodiments, a GDF15 Antibody Agent comprises an Fc region having a LAGA mutation, e.g., as provided in SEQ ID NO: 177.

In some embodiments, a Fc region variant comprises a FEGG mutation, e.g., as shown in bold in SEQ ID NO: 178: CPPCPAPEFEGGPSVFLFPPK. In some embodiments, a GDF15 Antibody Agent comprises an Fc region having a FEGG mutation, e.g., as provided in SEQ ID NO: 178.

In some embodiments, a Fc region variant comprises a AAGG mutation, e.g., as shown in bold in SEQ ID NO: 179: CPPCPAPEAAGGPSVFLFPPK. In some embodiments, a GDF15 Antibody Agent comprises an Fc region having a AAGG mutation, e.g., as provided in SEQ ID NO: 179.

In some embodiments, a Fc region variant comprises a AAGA mutation, e.g., as shown in bold in SEQ ID NO: 180: CPPCPAPEAAGAPSVFLFPPK. In some embodiments, an AAGA mutation is also referred to as Leu234Ala/Leu235Ala/Glu237Ala (LALAGA). In some embodiments, a GDF15 Antibody Agent comprises an Fc region having an AAGA mutation, e.g., as provided in SEQ ID NO: 180.

In some embodiments, a Fc region variant comprises a I253A mutation, a H310A mutation, a H435R mutation, a H435A mutation or any combination thereof.

In some embodiments, a GDF15 Antibody Agent comprising a Fc region having an Fc mutation (e.g., as described herein) has reduced binding (e.g., no binding) to a neonatal Fc receptor (FcRn), e.g., when compared to an otherwise similar GDF15 Antibody Agent having an Fc region without an Fc mutation. In some embodiments, a GDF15 antibody agent comprising a Fc region having an Fc mutation has reduced binding (e.g., no binding) to FcRn and reduced placental transfer, compared to an otherwise similar GDF15 antibody agent having an Fc region without an Fc mutation (e.g., as described herein).

In some embodiments, an Fc region variant comprising a LAGA mutation, a FEGG mutation, an AAGG mutation, an AAGA mutation, a LALA mutation or a combination thereof, has reduced binding (e.g., no binding) to a neonatal Fc receptor (FcRn), e.g., when compared to an otherwise similar Fc region without a LAGA mutation, a FEGG mutation, an AAGG mutation, an AAGA mutation, a LALA mutation or a combination thereof. In some embodiments, a GDF15 antibody agent comprising an Fc region having a LAGA mutation, a FEGG mutation, an AAGG mutation, an AAGA mutation, a LALA mutation or a combination thereof has reduced binding (e.g., no binding) to FcRn and reduced placental transfer, compared to an otherwise similar GDF15 antibody agent with an Fc region without a LAGA mutation, a FEGG mutation, an AAGG mutation, an AAGA mutation, a LALA mutation or a combination thereof.

In some embodiments, an Fc region variant comprising a I253A mutation, a H310A mutation, a H435R mutation, a H435A mutation or any combination thereof, has reduced binding (e.g., no binding) to a neonatal Fc receptor (FcRn), e.g., when compared to an otherwise similar Fc region without a I253A mutation, a H310A mutation, a H435R mutation, or a H435A mutation or any combination thereof. In some embodiments, a GDF15 antibody agent comprising an Fc region having a I253A mutation, a H310A mutation, a H435R mutation, a H435A mutation, or any combination thereof has reduced binding (e.g., no binding) to FcRn and reduced placental transfer, compared to an otherwise similar GDF15 antibody agent with an Fc region without a I253A mutation, a H310A mutation, a H435R mutation, or a H435A mutation or any combination thereof.

In some embodiments, a GDF15 Antibody Agent comprises an IgG1 Fc region comprising the sequence of SEQ ID NO: 181, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 181.

(SEQ ID NO: 181)
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPEAAGA
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSREE
MTKNQVSLTC LVKGFYPSDI AVEWESNGQP ENNYKTTPPV
LDSDGSFFLY SKLTVDKSRW QQGNVFSCSV MHEALHNHYT
QKSLSLSPG

In some embodiments, a GDF15 Antibody Agent comprises an IgG1 Fc region comprising the sequence of SEQ ID NO: 182, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 182.

(SEQ ID NO: 182)
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPEAAGA
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSREE
MTKNQVSLTC LVKGFYPSDI AVEWESNGQP ENNYKTTPPV
LDSDGSFFLY SKLTVDKSRW QQGNVFSCSV MHEALHNHYT
QKSLSLSPGK

In some embodiments, a GDF15 Antibody Agent further comprises a half-life extender. In some embodiments, a half-life extender is or comprises albumin, e.g., human serum albumin. In some embodiments, a half-life extender comprises a modification that increases binding to neonatal Fc receptor (FcRn).

In some embodiments, a GDF15 Antibody Agent comprises a heavy chain (HC) provided in Table 2 (or a sequence having at least 85% identity thereto) and a light chain (HC) provided in Table 1 (or a sequence having at least 85% identity thereto).

In some embodiments, a GDF15 antibody agent comprises the sequence of SEQ ID NO: 143, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159.

In some embodiments, a GDF15 antibody agent comprises the sequence of SEQ ID NO: 143 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143 without a terminal lysine; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159.

In some embodiments, a GDF15 antibody agent comprises the sequence of SEQ ID NO: 144, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159.

In some embodiments, a GDF15 antibody agent comprises the sequence of SEQ ID NO: 144 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144 without a terminal lysine; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;

In some embodiments, a GDF15 antibody agent comprises the sequence of SEQ ID NO: 145, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 145; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159.

In some embodiments, a GDF15 antibody agent comprises the sequence of SEQ ID NO: 145 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 145 without a terminal lysine; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159.

In some embodiments, a GDF15 antibody agent comprises the sequence of SEQ ID NO: 146, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159.

In some embodiments, a GDF15 antibody agent comprises the sequence of SEQ ID NO: 146 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146 without a terminal lysine; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159.

In some embodiments, a GDF15 antibody agent comprises the sequence of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163.

In some embodiments, a GDF15 antibody agent comprises the sequence of SEQ ID NO: 147 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147 without a terminal lysine; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163.

In some embodiments, a GDF15 antibody agent comprises the sequence of SEQ ID NO: 148, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 148; and the sequence of SEQ ID NO: 164, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 164.

In some embodiments, a GDF15 antibody agent comprises the sequence of SEQ ID NO: 148 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 148 without a terminal lysine; and the sequence of SEQ ID NO: 164, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 164.

In some embodiments, a GDF15 antibody agent comprises the sequence of SEQ ID NO: 149, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 149; and the sequence of SEQ ID NO: 164, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 164.

In some embodiments, a GDF15 antibody agent comprises the sequence of SEQ ID NO: 149 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 149 without a terminal lysine; and the sequence of SEQ ID NO: 164, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 164.

In some embodiments, a GDF15 antibody agent comprises the sequence of SEQ ID NO: 150, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 150; and the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166.

In some embodiments, a GDF15 antibody agent comprises the sequence of SEQ ID NO: 150 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 150 without a terminal lysine; and the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166.

In some embodiments, a GDF15 antibody agent comprises the sequence of SEQ ID NO: 151, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 151; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163.

In some embodiments, a GDF15 antibody agent comprises the sequence of SEQ ID NO: 151 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 151 without a terminal lysine; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163.

In some embodiments, a GDF15 antibody agent comprises the sequence of SEQ ID NO: 152, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 152; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163.

In some embodiments, a GDF15 antibody agent comprises the sequence of SEQ ID NO: 152 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 152 without a terminal lysine; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163.

In some embodiments, a GDF15 antibody agent comprises the sequence of SEQ ID NO: 153, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 153; and the sequence of SEQ ID NO: 169, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 169.

In some embodiments, a GDF15 antibody agent comprises the sequence of SEQ ID NO: 153 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 153 without a terminal lysine; and the sequence of SEQ ID NO: 169, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 169.

In some embodiments, a GDF15 antibody agent comprises the sequence of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154; and the sequence of SEQ ID NO: 169, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 169.

In some embodiments, a GDF15 antibody agent comprises the sequence of SEQ ID NO: 154 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154 without a terminal lysine; and the sequence of SEQ ID NO: 169, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 169.

In some embodiments, a GDF15 antibody agent comprises the sequence of SEQ ID NO: 155, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155; and the sequence of SEQ ID NO: 171, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 171.

In some embodiments, a GDF15 antibody agent comprises the sequence of SEQ ID NO: 155 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155 without a terminal lysine; and the sequence of SEQ ID NO: 171, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 171.

In some embodiments, a GDF15 antibody agent comprises the sequence of SEQ ID NO: 156, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 156; and the sequence of SEQ ID NO: 171, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 171.

In some embodiments, a GDF15 antibody agent comprises the sequence of SEQ ID NO: 156 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 156 without a terminal lysine; and the sequence of SEQ ID NO: 171, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 171.

In some embodiments, a GDF15 antibody agent comprises the sequence of SEQ ID NO: 157, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 157; and the sequence of SEQ ID NO: 173, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 173.

In some embodiments, a GDF15 antibody agent comprises the sequence of SEQ ID NO: 157 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 157 without a terminal lysine; and the sequence of SEQ ID NO: 173, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 173.

In some embodiments, a GDF15 antibody agent comprises the sequence of SEQ ID NO: 158, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 158; and the sequence of SEQ ID NO: 173, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 173.

In some embodiments, a GDF15 antibody agent comprises the sequence of SEQ ID NO: 158 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 158 without a terminal lysine; and the sequence of SEQ ID NO: 173, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 173.

In some embodiments, a GDF15 antibody agent comprises the sequence of SEQ ID NO: 144, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144; and the sequence of SEQ ID NO: 206, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206.

In some embodiments, a GDF15 antibody agent comprises the sequence of SEQ ID NO: 144 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144 without a terminal lysine; and the sequence of SEQ ID NO: 206, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206

In some embodiments, a GDF15 antibody agent comprises the sequence of SEQ ID NO: 202, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 202; and the sequence of SEQ ID NO: 210, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 210.

In some embodiments, a GDF15 antibody agent comprises the sequence of SEQ ID NO: 202 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 202 without a terminal lysine; and the sequence of SEQ ID NO: 210, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 210.

In some embodiments, a GDF15 antibody agent comprises the sequence of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147; and the sequence of SEQ ID NO: 215, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 215.

In some embodiments, a GDF15 antibody agent comprises the sequence of SEQ ID NO: 147 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147 without a terminal lysine; and the sequence of SEQ ID NO: 215, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 215.

In some embodiments, a GDF15 antibody agent comprises the sequence of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147; and the sequence of SEQ ID NO: 219, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 219.

In some embodiments, a GDF15 antibody agent comprises the sequence of SEQ ID NO: 147 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147 without a terminal lysine; and the sequence of SEQ ID NO: 219, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 219.

### Bispecific or Multispecific GDF15 Antibody Agents

Disclosed herein, among other things, are use of bispecific or multispecific GDF15 Antibody Agents and compositions comprising the same to treat and/or prevent certain types and/or instances of nausea and/or emesis with which it had not previously been considered to be associated. In some embodiments, a bispecific GDF15 Antibody Agent comprises a first binding specificity for GDF15 and a second binding specificity for a second antigen. In some embodiments, a second antigen is other than GDF15. In some embodiments, a second antigen is a member of the TGFbeta superfamily.

In some embodiments of a GDF15 bispecific Antibody Agent, a single chain GDF15 polypeptide (e.g., a GDF15 light chain polypeptide or a GDF15 heavy chain polypeptide) provides binding specificity to GDF15.

In some embodiments, a GDF15 bispecific Antibody Agent comprises a light chain (LC) polypeptide comprising a LC CDR1, a LC CDR2, and a LC CDR3, e.g., as provided in Table 1). In some embodiments, a GDF15 bispecific Antibody Agent comprising a LC polypeptide with a LC CDR1, a LC CDR2, and a LC CDR3 binds to GDF15.

In some embodiments, a GDF15 bispecific Antibody Agent comprises a heavy chain (HC) polypeptide comprising a HC CDR1, a HC CDR2, and a HC CDR3, e.g., as provided in Table 2). In some embodiments, a GDF15 bispecific Antibody Agent comprising a HC polypeptide with a HC CDR1, a HC CDR2, and a HC CDR3 binds to GDF15.

In some embodiments, a GDF15 bispecific Antibody Agent comprises a GDF15 Antibody Agent comprising a heavy chain (HC) comprising a HC CDR1, a HC CDR2, and a HC CDR3, e.g., as provided in Table 2; and a light chain (LC) comprising a LC CDR1, a LC CDR2, and a LC CDR3, e.g., as provided in Table 1.

In some embodiments, a GDF15 bispecific Antibody Agent is or comprises: a heterodimer, a Crossmab, a DVD-Ig, a 2 in 1 IgG, an IgG-sc-FV, an scFv-scFv, a BiTE, a DART, a diabody, a Fab-scFv fusion, a Fab-Fab fusion, a tandem antibody, or any other art recognized formats for an antibody having dual-specificity.

### Nucleic Acids Encoding GDF15 Antibody Agents and or Provided Polypeptides

The present disclosure, among other things, provides nucleic acids encoding GDF15 Antibody Agents described herein, or polypeptides provided herein (e.g., LC polypeptides and/or HC polypeptides) to treat and/or prevent certain types and/or instances of nausea and/or emesis with which it had not previously been considered to be associated. The present disclosure includes nucleic acids encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein. Such nucleic acids may be present in a vector. Such nucleic acids may be present in the genome of a cell, e.g., a cell of a subject in need of treatment or a cell for production of an antibody, e.g. a mammalian cell for production of a GDF15 Antibody Agent described herein, or polypeptides provided herein (e.g., LC polypeptides and/or HC polypeptides).

Nucleic acids encoding GDF15 Antibody Agents, or polypeptides provided herein (e.g., LC polypeptides and/or HC polypeptides) may be modified to include codons that are optimized for expression in a particular cell type or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acids encoding a GDF15 Antibody Agent described herein, or polypeptides provided herein (e.g., LC polypeptides and/or HC polypeptides), in whole or in part, may include an altered sequence to optimize codon usage for a particular cell type (e.g., a eukaryotic or prokaryotic cell). For example, a coding sequence for a humanized heavy (or light) chain variable region as described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host. Selection of vectors and methods to construct them are commonly known to persons of ordinary skill in the art (see, e.g., “Recombinant DNA Part D,” Methods in Enzymology, Vol. 153, Wu and Grossman, eds., Academic Press (1987)).

Conventionally used techniques including, for example, electrophoresis, calcium phosphate precipitation, DEAE-dextran transfection, or lipofection, may be used to introduce a foreign nucleic acid (e.g., DNA or RNA) into a prokaryotic or eukaryotic host cell. Desirably, a vector may include regulatory sequences, such as transcription and/or translation initiation and/or termination codons, which are specific to the type of host (e.g., bacterium, fungus, plant, or animal) into which a vector is to be introduced, as appropriate and taking into consideration whether a vector is DNA or RNA. In some embodiments, a vector comprises regulatory sequences that are specific to a genus of a host cell. In some embodiments, a vector comprises regulatory sequences that are specific to a species of a host.

In addition to a replication system and an inserted nucleic acid, a nucleic acid construct can include one or more marker genes, which allow for selection of transformed or transfected hosts. Exemplary marker genes include, e.g., biocide resistance (e.g., resistance to antibiotics or heavy metals) or complementation in an auxotrophic host to provide prototrophy.

An expression vector can comprise a native or nonnative promoter operably linked to an isolated or purified nucleic acid as described above. Selection of promoters, e.g., strong, weak, inducible, tissue-specific, and/or developmental-specific, is within the skill of one in the art. Similarly, combining a nucleic acid as described above with a promoter is also within the skill of one in the art.

Suitable vectors include those designed for propagation and expansion and/or for expression. For example, a cloning vector may be selected from the pUC series, the pBluescript series (Stratagene, LaJolla, Calif.), the pET series (Novagen, Madison, Wis.), the pGEX series (Pharmacia Biotech, Uppsala, Sweden), the pcDNA3 series (Invitrogen) or the pEX series (Clontech, Palo Alto, Calif.). Bacteriophage vectors, such as λGT10, λGT11, λZapII (Stratagene), λEMBL4, and λNM1149, may be used. Examples of plant expression vectors that can be used include pBI110, pBI101.2, pBI101.3, pBI121, or pBIN19 (Clontech). Examples of animal expression vectors that can be used include pEUK-C1, pMAM, or pMAMneo (Clontech). The TOPO cloning system (Invitrogen, Carlsbad, Calif.) also can be used in accordance with the manufacturer's recommendations.

Additional sequences can be added to such cloning and/or expression sequences to optimize their function in cloning and/or expression, to aid in isolation of a nucleic acid encoding a GDF15 Antibody Agent described herein, or to improve introduction of a nucleic acid into a cell. Use of cloning vectors, expression vectors, adapters, and linkers is well known in the art (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding a GDF15 Antibody Agent described herein, or polypeptides provided herein (e.g., LC polypeptides and/or HC polypeptides), under control of transcriptional and/or translational control signals. These methods may include in vitro recombinant DNA and synthetic techniques and in vivo recombination (see, e.g., Ausubel, supra; or Sambrook, supra).

### Nucleic Acid Agents

In some embodiments, a GDF15-GFRAL Pathway Modulating Agent may be or comprise a nucleic acid.

To give but a few examples, in some embodiments, a useful such agent may be or comprise a nucleic acid that encodes (or that is the complement of a nucleic acid that encodes) a polypeptide agent as described herein.

In some embodiments, a useful nucleic acid agent may be or comprise an antisense nucleic acid (e.g., an antisense oligonucleotide) that may target a GDF15 or GFRAL or RET transcript for degradation (e.g., by recruiting RNAse H or other degradation machinery), modification (e.g., cleavage or chemical modification of one or more residues), localization (e.g., sequestration) or other mechanism that reduces its level, processing (e.g., capping, polyadenylation, splicing, cytoplasmic transport) and/or expression (i.e., translation), etc. In some embodiments, such agents may be assessed and/or utilized as described herein to treat one or more of CVS, CHS and/or MAN/V.

### Small Molecule Agents

In some embodiments, a GDF15-GFRAL Pathway Modulating Agent may be or comprise a small molecule.

In some embodiments, a useful such small molecule agent is or comprises a small molecule that binds to one or both of GDF15 and/or GFRAL and/or RET and disrupts (e.g., competes) interaction there between. In some such examples, such small molecule may be or comprises an inhibitor or negative allosteric modulator that binds specifically to GDF15 and/or GFRAL and/or RET. In some embodiments, a small molecule may bind to GDF15 (e.g., GDF15 inhibitor and/or negative allosteric modulator). In some embodiments, a small molecule may bind to GFRAL (e.g., GFRAL antagonist and/or negative allosteric modulator). In some embodiments, a small molecule may bind to RET (e.g., RET kinase inhibitor and/or negative allosteric modulator) In some embodiments, one or more such agents may be assessed and/or utilized as described herein to treat one or more of HG, CVS, CHS, MIDs and/or MAN/V.

### Characterization of GDF15-GFRAL Pathway Modulating Agents

The present disclosure teaches use of GDF15-GFRAL Pathway Modulating Agents for the treatment of certain diseases, disorders, or conditions. In some embodiments, agents (e.g., candidate agents) may be identified, characterized, and/or assessed for their usefulness as described herein.

In some embodiments, a GDF15-GFRAL Pathway Modulating Agent may be identified and/or characterized by, for example, its impact on expression and/or activity of GDF15, GFRAL, RET, etc. For example, in some embodiments, direct binding of such agent to a pathway component (e.g., to GDF15, to GFRAL and/or to RET, etc.), and/or ability of such agent to compete an interaction between a pathway component and a relevant interaction partner (e.g., between GDF15 and GFRAL) is assessed, optionally at a plurality of concentrations and/or optionally in comparison to an appropriate reference (e.g., absence of such agent and/or presence of a positive control agent and/or presence of a negative control agent). Alternatively or additionally, impact of such agent on expression (e.g., expression level) of a nucleic acid encoding a GDF15-GFRAL Pathway component (e.g., GDF15, GFRAL, RET, etc.) and/or of a particular form thereof (e.g., of a particular splice variant etc.) may be assessed, Still further alternatively or additionally, in some embodiments, impact of such agent on expression (e.g., expression level) of a polypeptide that is a GDF15-GFRAL Pathway component (e.g., GDF15, GFRAL, RET, etc.) and/or of a particular form thereof (e.g., of a particular post-translationally-modified form such as a glycoform and/or phosphorylated form etc.) may be assessed. Yet further alternatively or additionally, in some embodiments, impact of such agent on activity (e.g., as noted above, binding activity and/or another activity indicative of participation in the GDF15-GFRAL Pathway (including activity of the pathway itself, for example as may be revealed using a reporter or other appropriate system).

For example, in some embodiments one or more such agents may be characterized with respect to one or more characteristics disclosed herein, e.g., high binding affinity to a target of interest (e.g., GDF15, GFRAL, RET, etc.), favorable binding kinetics (e.g., to such target of interest), binding specificity (e.g., to such target of interest relative to other potential targets such as, for example, other TGFb family members), favorable pharmacokinetics, pharmacodynamics, and/or stability, manageable production and/or formulation characteristics, etc.

In some embodiments, a useful agent as described herein is characterized in that it modulates one or more, or all or a combination of detectable GDF15-GFRAL Pathway (e.g., GDF15, GFRAL, RET, etc.) activities such that the GDF15-GFRAL Pathway Modulating Agent: (a) increases food intake; (b) increases appetite; (c) increases body weight; (d) decreases weight loss; (e) increases fat mass; (f) increases lean mass; (g) decreases loss of fat mass, (h) promotes weight gain; (i) decreases loss of lean muscle mass, (i) decreases fatigue; (j) increases pro-inflammation; (k) increases immune cell infiltration in tumor; (1) decreases metastases; (m) increases efficacy of immunotherapy (e.g., immune checkpoint inhibitor therapy); (n) decreases cellular senescence; (o) inhibits binding of GDF15 to a GDF15 receptor, e.g., GFRAL; (p) decreases downstream signaling mediated by RET; (q) decreases phosphorylation of ERK; (r) decreases phosphorylation of ribosomal protein S6; (s) decreases RET-mediated activation of the MAPK signaling pathway; (t) decreases RET activation of the AKT-signaling pathway; (u) decreases activation of the PLC-D 1 signaling pathway, (v) decreases nausea, vomiting and/or emesis; (w) increases T cell adhesion to endothelial cells (e.g., inhibiting an LFA1-ICAM interaction), (x) decreases stimulation of the Hypothalamic-pituitary adrenal axis as assessed by decreased growth hormone (GH), adrenocorticotropic hormone (ACTH) and corticosterone/cortisol; or (y) decreases levels of circulating GDF15 (e.g., active and/or free GDF15).

In some embodiments, an agent useful as described herein binds to mammal (e.g., human, mouse, rat, cyno, dog, and/or cat) GDF15, GFRAL, and/or RET.

In some embodiments, an agent useful as described herein binds to human GDF15, GFRAL, and/or RET with a binding affinity (K_{D}) of about 0.2×10(−10)M to about 20×10(−10)M. In some embodiments, a useful agent as described herein binds to human GDF15, GFRAL, and/or RET with a K_{D} of about 0.2×10(−10)M, 0.4×10(−10)M, 0.6×10(−10)M, 0.8×10(−10)M, 1×10(−10)M, 1.5 ×10(−10)M, 2 ×10(−10)M, 2.1 ×10(−10)M, 2.2 ×10(−10)M, 2.3 ×10(−10)M, 2.4 ×10(−10)M, 2.5 ×10(−10)M, 2.6 ×10(−10)M, 2.7 ×10(−10)M, 2.8 ×10(−10)M, 2.9 ×10(−10)M, 3 ×10(−10)M, 3.5 ×10(−10)M, 4 ×10(−10)M, 6 ×10(−10)M, 8 ×10(−10)M, 10 ×10(−10)M, 15×10(−10)M, or 20 ×10(−10)M. In some embodiments, binding of an agent as described herein (e.g., binding of such agent to GDF15) is measured using a Surface Plasmon Resonance assay (e.g., Biacore assay) or an Octet assay.

In some embodiments, an agent useful as described herein binds to human GDF15, GFRAL, and/or RET with a binding affinity (K_{D}) of about 00.7×10(−12)M to 1000×10(−12)M with a Fab format. In some embodiments, a use Antibody Agent binds to human GDF15, GFRAL, and/or RET with a K_{D} of about 0.7×10(−12)M, about 0.8×10(−12)M, about 0.9×10(−12)M, 1×10(−12)M, about 2×10(−12)M, about 3×10(−12)M, about 4×10(−12)M, about 5×10(−12)M, about 6×10(−12)M, about 7×10(−12)M, about 8×10(−12)M, about 9×10(−12)M, about 10×10(−12)M, about 20×10(−12)M, about 30×10(−12)M, about 40×10(−12)M, about 50×10(−12)M, about 60×10(−12)M, about 70×10(−12)M, about 80×10(−12)M, about 90×10(−12)M, about 100×10(−12)M, about 200×10(−12)M, about 300×10(−12)M, about 400×10(−12)M, about 500×10(−12)M, about 600×10(−12)M, about 700×10(−12)M, about 800×10(−12)M, about 900×10(−12)M, about 1000×10(−12)M, e.g., with a Fab format. In some embodiments, an agent useful as described herein binds to human GDF15, GFRAL, and/or RET with a K_{D} of about 7.3×10(−12)M to about 599×10(−12)M, e.g., with a Fab format. In some embodiments, binding of an agent useful as described herein to GDF15, GFRAL, and/or RET is measured using a Surface Plasmon Resonance assay (e.g., Biacore assay) or an Octet assay as described herein.

In some embodiments, an agent useful as described herein binds to cyno GDF15, GFRAL, and/or RET with a binding affinity (K_{D}) of about 0.1×10-9M to about 25×10-10 M. In some embodiments, an agent useful as described herein binds to cyno GDF15 with a K_{D} of about 0.1×10-9M, 0.2×10-9M, 0.5×10-9M, 1×10-9M, 1.5×10-9M, 2×10-9M, 5×10-9M, 1 ×10-10M, 2×10-10M, 4×10-10M, 6×10-10M, 7×10-10M, 8×10-10M, 9×10-10M, 10×10-10M, 15×10-10M, 20×10-10M, or 25×10-10M. In some embodiments, an agent useful as described herein binds to cyno GDF15 with a KD of about 1.42×10-9 M to about 8.51×10-10M.

In some embodiments, an agent useful as described herein binds to mouse GDF15, GFRAL, and/or RET with a binding affinity (KD) of about 0.1×10-M to about 25×10-8M. In some embodiments, an agent useful as described herein binds to mouse GDF15 with a KD of about 0.1×10-7M, 0.2×10-7M, 0.5 ×10-7M, 1 ×10-7M, 2 ×10-7M, 4 ×10-7M, 6 ×10-7M, 8 ×10-7M, 10 ×10-7M, 15 ×10-7M, 20 ×10-7M, or 25 ×10-7M. In some embodiments, an agent useful as described herein binds to mouse GDF15, GFRAL, and/or RET with a K_{D} of about 1.57×10-7 M to about 8.3×10-8M.

In some embodiments, a binding affinity is determined with a binding affinity determining assay such as an Octet assay or a comparable assay

In some embodiments, an agent useful as described herein does not bind to or has minimal binding affinity for one or more TGFbeta super family members other than GDF15. In some embodiments, an agent useful as described herein does not bind to or has minimal binding affinity for any one or all or a combination of GDNF, GDF8, GDF10, GDF11, BMP9, BMP10, Activin A, or Activin B. In some embodiments, an agent useful as described herein does not bind to GDNF. In some embodiments, an agent useful as described herein does not bind to or has minimal binding affinity GDF8. In some embodiments, an agent useful as described herein does not bind to or has minimal binding affinity GDF10. In some embodiments, an agent useful as described herein not bind to or has minimal binding affinity GDF11.

In some embodiments, an agent useful as described herein binds to one or more members of the TGFbeta super family in addition to GDF15. In some embodiments, an agent useful as described herein to GDF15 and one or more of: Activin A, Activin B, or GDF10. In some embodiments, an agent useful as described herein binds to GDF15 and Activin A. In some embodiments, an agent useful as described herein binds to GDF15 and Activin B. In some embodiments, an agent useful as described herein binds to GDF15 and GDF10. In some embodiments, an agent useful as described herein binds to GDF15, Activin A and Activin B. In some embodiments, an agent useful as described herein binds to GDF15, Activin A, Activin B and GDF10.

In some embodiments, an agent useful as described herein which binds to GDF15 and Activin A does not modulate an activity and/or level of Activin A, e.g., when characterized in an assay that evaluates an Activin A activity and/or level.

In some embodiments, an agent useful as described herein which binds to GDF15 and Activin B does not modulate an activity and/or level of Activin B, e.g., when characterized in an assay that evaluates an Activin B activity and/or level.

In some embodiments, an agent useful as described herein is characterized by its IC50 when assessed in an appropriate assay (as understood in the art). In some embodiments, A useful agent is characterized by an IC50 value below about 100 μM, or 10 μM, or 1 uM. In some embodiments, a useful agent is characterized by an IC50 value below about 1000 nM, or 100 nM, or 10 nM. In some embodiments, a useful agent is characterized by an IC50 value that is sub-nanomolar.

### Antibody Agents or Compositions Thereof

The present disclosure teaches use of GDF15-GFRAL Pathway Modulating Agents for the treatment of certain diseases, disorders, or conditions. In some embodiments, agents (e.g., Antibody Agent) may be identified, characterized, and/or assessed for their usefulness as described herein.

In some embodiments, a useful Antibody Agent has high specificity for GDF15, GFRAL, and/or RET and low polyreactivity, e,g., as measured with a poly-specificity reagent (PSR) or a comparable reagent that measures antibody binding specificity. In some embodiments, a useful Antibody Agent has a clean PSR score of less than 0.1. In some embodiments, a useful Antibody Agent has a low PSR score of between 0.1 to 0.33. In some embodiments, a useful Antibody Agent has a low PSR score of about 0.1. In some embodiments, a useful Antibody Agent has a low PSR score of about 0.2. In some embodiments, a useful Antibody Agent has a low PSR score of about 0.22. In some embodiments, a useful Antibody Agent has a low PSR score of about 0.24. In some embodiments, a useful Antibody Agent has a low PSR score of about 0.26. In some embodiments, a useful Antibody Agent has a low PSR score of about 0.28. In some embodiments, a useful Antibody Agent has a low PSR score of about 0.3. In some embodiments, a useful Antibody Agent has a low PSR score of about 0.31. In some embodiments, a useful Antibody Agent has a low PSR score of about 0.32. In some embodiments, a useful Antibody Agent has a low PSR score of about 0.33.

In some embodiments, a useful Antibody Agent has low hydrophobicity as measured in a HIC assay or a comparable assay that measures hydrophobicity. In some embodiments, a useful Antibody Agent has a HIC retention time of less than 10.5 minutes indicating a clean to low HIC. In some embodiments, a useful Antibody Agent has a retention time of less than 10.5 minutes, less than 10 minutes, or less than 9.5 minutes. In some embodiments, a useful Antibody Agent has a retention time of about 9.4 minutes, about 9.5 minutes, about 9.6 minutes, about 9.7 minutes, about 9.8 minutes, about 9.9 minutes, about 10 minutes, about 10.1 minutes, about 10.2 minutes, about 10.3 minutes, about 10.4 minutes or about 10.5 minutes. In some embodiments, a useful Antibody Agent has a retention time of between 10.5 to 11.5 minutes indicating a medium HIC. In some embodiments, a useful Antibody Agent has a retention time of about 10.5 minutes. In some embodiments, a useful Antibody Agent has a retention time of about 10.6 minutes. In some embodiments, a useful Antibody Agent has a retention time of about 10.7 minutes. In some embodiments, a useful Antibody Agent has a retention time of about 10.8 minutes. In some embodiments, a useful Antibody Agent has a retention time of about 10.9 minutes. In some embodiments, a useful Antibody Agent has a retention time of about 11 minutes. In some embodiments, a useful Antibody Agent has a retention time of about 11.1 minutes. In some embodiments, a useful Antibody Agent has a retention time of about 11.2 minutes. In some embodiments, a useful Antibody Agent has a retention time of about 11.3 minutes. In some embodiments, a useful Antibody Agent has a retention time of about 11.4 minutes. In some embodiments, a useful Antibody Agent has a retention time of about 11.5 minutes.

In some embodiments, a useful Antibody Agent has low self-association as measured with an AC-SINS assay or a comparable assay that measures self-association. In some embodiments, a useful Antibody Agent has an AC-SINS score between 5 and 20 indicating low self-association. In some embodiments, a useful Antibody Agent has an AC-SINS score of 5. In some embodiments, a useful Antibody Agent has an AC-SINS score of 6. In some embodiments, a useful Antibody Agent has an AC-SINS score of 7. In some embodiments, a useful Antibody Agent has an AC-SINS score of 8. In some embodiments, a useful Antibody Agent has an AC-SINS score of 9. In some embodiments, a useful Antibody Agent has an AC-SINS score of 10. In some embodiments, a useful Antibody Agent has an AC-SINS score of 11. In some embodiments, a useful Antibody Agent has an AC-SINS score of 12. In some embodiments, a useful Antibody Agent has an AC-SINS score of 13. In some embodiments, a useful Antibody Agent has an AC-SINS score of 14. In some embodiments, a useful Antibody Agent has an AC-SINS score of 15. In some embodiments, a useful Antibody Agent has an AC-SINS score of 16. In some embodiments, a useful Antibody Agent has an AC-SINS score of 17. In some embodiments, a useful Antibody Agent has an AC-SINS score of 18. In some embodiments, a useful Antibody Agent has an AC-SINS score of 19. In some embodiments, a useful Antibody Agent has an AC-SINS score of 20.

In some embodiments, a useful Antibody Agent has a melting temperature (Tm) of about 65° C. to about 90° C., about 65° C. to about 85° C., about 65° C. to about 80° C., about 65° C. to about 75° C., about ° C. to about 70° C., about 70° C. to about 90° C., about 75° C. to about 90° C., about 80° C. to about 90° C. or about 85° C. to about 90° C. In some embodiments, a useful Antibody Agent has a melting temperature (Tm) of about 73° C. to about 84.5° C.

In some embodiments, an Antibody Agent is produced in a bacterial cell, e.g., E. coli.

In some embodiments, an Antibody Agent is produced in a yeast cell, e.g., S. cerevisiae or S. pombe.

In some embodiments, an Antibody Agent is produced in an insect cell, e.g., Sf9.

In some embodiments, an Antibody Agent is produced in a mammalian cell. In some embodiments, a mammalian cell is chosen from a CHO cell, a COS cell, a HEK-293 cell, an NS0 cell, a PER.C6 cell, or an Sp2.0 cell.

In some embodiments, an Antibody Agent can be produced at a concentration of about 10 mg/L to about 20,000 mg/L. In some embodiments, an Antibody Agent can be produced at a concentration of about 10 mg/L, about 20 mg/L, about 30 mg/L, about 40 mg/L, about 50 mg/L, about 60 mg/L, about 70 mg/L, about 80 mg/L, about 90 mg/L, about 100 mg/L, about 150 mg/L, about 200 mg/L, about 250 mg/L, about 300 mg/L, about 350 mg/L, about 400 mg/L, about 450 mg/L, about 500 mg/L, about 550 mg/L, about 600 mg/L, about 650 mg/L, about 700 mg/L, about 750 mg/L, about 800 mg/L, about 850 mg/L, about 900 mg/L, about 950 mg/L, about 1000 mg/L, about 2000 mg/L, about 2000 mg/L, about 3000 mg/L, about 4000 mg/L, about 5000 mg/L, about 6000 mg/L, about 7000 mg/L, about 8000 mg/L, about 9000 mg/L, about 10,000 mg/L, about 15,000 mg/L or about 20,000 mg/L. In some embodiments, an Antibody Agent can be produced at a concentration of more than 100 mg/L, more than 200 mg/L, more than 500 mg/L, more than 1000 mg/L, more than 2000 mg/L, more than 3000 mg/L, more than 4000 mg/L, more than 5000 mg/L, more than 6000 mg/L, more than 7000 mg/L, more than 8000 mg/L, more than 9000 mg/L, more than 10,000 mg/L. In some embodiments, an Antibody Agent can be produced at a concentration of about 1000 to 20,000 mg/L, about 2000 to 20,000 mg/L, about 5000 to 20,000 mg/L, about 6000 to 20,000 mg/L, about 7000 to 20,000 mg/L, about 8000 to 20,000 mg/L, about 9000 to 20,000 mg/L, 10,000 to 20,000 mg/L or about 15,000 to 20,000 mg/L.

In some embodiments, a useful Antibody Agent is characterized in that when tested in an assay that evaluates activation of a GFRAL and/or RET receptor, a useful Antibody Agent or polypeptide binds to GDF15, GFRAL, and/or RET and prevents activation of one or more signaling pathways activated by the GDF15-GFRAL Pathway. In some embodiments, a useful Antibody Agent inhibits activation of one or more signaling pathways activated by the GFRAL receptor by about 50%, about 60%, about 70%, about 80%, about 90% about 95%, about 96%, about 97% about 98% about 99% or 100%.

In some embodiments, a signaling pathway activated by a GFRAL receptor comprises a MAP kinase pathway. In some embodiments, a MAP kinase pathway activation is measured by phosphorylation of ERK. In some embodiments, a useful Antibody Agent inhibits ERK phosphorylation by about 50%, about 60%, about 70%, about 80%, about 90% about 95%, about 96%, about 97% about 98% about 99% or 100%. In some embodiments, a useful Antibody Agent decreases pERK by about 1.5-, 2-, 4-, 5-, 10-, 20-, 50-fold or more relative to a comparator (e.g., an otherwise similar cell not contacted with GDF15 Antibody Agents). In some embodiments, pERK can be measured by an assay described in Example 4.

In some embodiments, a useful Antibody Agent in an IgG format results in a PERK concentration (IC50) of about 35 pM to about 254 PM, e.g., when tested in an assay described in Example 4.

In some embodiments, a useful Antibody Agent in a Fab format results in a pERK concentration (IC50) of about 11 pM to about 46 pM, e.g., when tested in an assay described in Example 4.

In some embodiments, an Antibody Agent described herein is identified, characterized and/or producing using hybridoma technology.

Phage library based methods for identifying, characterizing, and/or producing polypeptide agents (e.g., antibody agents) are known in the art (as described in, e.g., Ladner et al. U.S. Pat. No. 5,223,409; Kang et al. International Publication No. WO 92/18619; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; each of which his hereby incorporated by reference in its entirety).

Yeast library based methods for identifying, characterizing, and/or producing polypeptide agents (e.g., antibody agents) are known in the art, e.g., as described in U.S. Pat. No. 8,691,730 and Chao G. et al (2006) Nature Protocols 1 (2): 755-68, each of which his hereby incorporated by reference in its entirety.

In some embodiments, an antibody agent described herein may be derived from another species (e.g., a species other than human). A humanized antibody is an antibody (typically produced by recombinant DNA technology), in which some or all amino acids of a human immunoglobulin light chain or heavy chain that are not required for antigen binding (e.g., constant regions and/or framework regions of variable domains) are used to substitute for the corresponding amino acids from light chain or heavy chain of the cognate, nonhuman antibody. By way of example, a humanized version of a murine antibody to a given antigen has on both heavy and light chains: (1) constant regions of a human antibody; (2) FRs from the variable domains of a human antibody; and (3) CDRs from the murine antibody. Human FRs may be selected based on their highest sequence homology to mouse FR sequence. When necessary or desirable, one or more residues in human FRs can be changed to residues at corresponding positions in a murine antibody so as to preserve binding affinity of the humanized antibody to a target. Such a change is sometimes called “back mutation.” Similarly, forward mutations may be made to revert back to murine sequence for a desired reason, e.g. stability or affinity to a target. Those skilled in the art are aware that humanized antibodies generally are less likely to elicit an immune response in humans as compared to chimeric human antibodies because the former contain considerably fewer non-human components.

Technologies for humanizing non-human antibodies are well known in the art. Suitable methods for making humanized antibodies in accordance with the present disclosure are described in, e.g., Winter EP 0 239 400; Jones et al., Nature 321:522-525 (1986); Riechmann et al., Nature 332:323-327 (1988); Verhoeyen et al., Science 239:1534-1536 (1988); Queen et al., Proc. Nat. Acad. ScL USA 86:10029 (1989); U.S. Pat. No. 6,180,370; and Orlandi et al., Proc. Natl. Acad. Sd. USA 86:3833 (1989); the disclosures of each of which are incorporated herein by reference in their entireties. Generally, transplantation of non-human (e.g., murine) CDRs onto a human antibody is achieved as follows. cDNAs encoding VH and VL are isolated from a hybridoma, and nucleic acid sequences encoding VH and VL including CDRs are determined by sequencing. Nucleic acid sequences encoding CDRs are inserted into corresponding regions of a human antibody VH or VL coding sequences and attached to human constant region gene segments of a desired isotype (e.g., γ1 for CH and kappa for CL). Humanized heavy and light chain genes are co-expressed in mammalian host cells (e.g., CHO or NSO cells) to produce soluble humanized antibody. To facilitate large-scale production of antibodies, it is often desirable to select for a high expressor using, for example, a DHFR gene or GS gene in the producer line.

In some embodiments, an Antibody Agent (e.g., an Antibody Agent such as a GDF15 Antibody Agents) described herein comprises or is a human antibody. Completely human antibodies may be particularly desirable for therapeutic treatment of human subjects. Human antibodies can be made by a variety of methods known in the art including phage display methods described above using antibody libraries derived from human immunoglobulin sequences (see, e.g., U.S. Pat. Nos. 4,444,887 and 4,716,111; and PCT publications WO 98/46645, WO 98/60433, WO 98/24893, WO 98/16664, WO 96/34096, WO 96/33735, and WO 91/10741; each of which is incorporated herein by reference in its entirety). Techniques are also available for the preparation of human monoclonal antibodies in, e.g., Cole et al., Monoclonal Antibodies and Cancer Therapy, Alan R. Riss, (1985); and Boerner et al., J. Immunol., 147 (1): 86-95, (1991), each of which is incorporated herein by reference in its entirety.

Those skilled in the art, reading the present disclosure, will appreciate that, if desired, polypeptide agents for use as described herein may be produced, for example, by culturing a host cell comprising a heterologous nucleic acid encoding GDF15 Antibody Agents polypeptide or combination thereof, under a condition wherein the GDF15 antibody polypeptide or combination thereof (e.g., a GDF15 polypeptide agent) is expressed by said host cell. In some such embodiments, the heterologous nucleic acid is or comprises a vector comprising a GDF15 antibody nucleic acid sequence.

In some embodiments, a host cell is a yeast cell, a bacterial cell, a mammalian cell or an insect cell.

In some embodiments, a host cell is a mammalian cell. In some embodiments, a mammalian cell is chosen from a CHO cell, a COS cell, a HEK-293 cell, an NS0 cell, a PER.C6 cell, or an Sp2.0 cell.

### Nucleic Acid Agents or Composition Thereof

The present disclosure teaches use of GDF15-GFRAL Pathway Modulating Agents for the treatment of certain diseases, disorders, or conditions. In some embodiments, agents (e.g., nucleic acid agents) may be identified, characterized, and/or assessed for their usefulness as described herein. Those skilled in the art are familiar with a variety of screening technologies that can be useful, in some embodiments, for identification and/or characterization of agents as described herein.

For example, in some embodiments, nucleic acid agents may be identified and/or characterized by assessing their mechanism of action. To give but a few examples, a nucleic acid agent that is or comprises an antisense oligonucleotide may be assessed by determining its impact on level and/or processing (e.g., alternative splicing) of a targeted transcript. In some embodiments, a nucleic acid agent that is or comprises a mRNA encoding one or more component and/or inhibitor (e.g., Antibody Agent) targeting the GDF15-GFRAL pathway may be assessed by determining its impact on expression and/or activity of GDF15, GFRAL, RET, etc.

### Small Molecule Agents or Composition Thereof

The present disclosure teaches use of GDF15-GFRAL Pathway Modulating Agents for the treatment of certain diseases, disorders, or conditions. In some embodiments, agents (e.g., small molecule agents) may be identified, characterized, and/or assessed for their usefulness as described herein. Those skilled in the art are familiar with a variety of screening technologies that can be useful, in some embodiments, for identification and/or characterization of agents as described herein.

For example, in some embodiments, small molecule agents may be identified and/or characterized by assessing their mechanism of action, as previously described. To give but a few examples, a small molecule agent may be assessed by determining its impact on binding to GDF15, GFRAL, and/or RET. In some embodiments, a small molecule agent may reduce activity and/or activation of GDF15, GFRAL, and/or RET measured by luciferase assays, pERK functional assays, and/or RET kinase activation assays. In some embodiment, a small molecule agent may be assessed, in-vivo, by determining its impact on behavior (e.g., food intake, taste aversion, pica, emesis, and/or weight loss).

### Diagnostic Technologies

Among other things, the present disclosure teaches that subjects suffering from or susceptible to one or more certain diseases, disorders or conditions as described herein may benefit from therapy with a GDF15-GFRAL Pathway Modulating Agent. In some embodiments, such subjects may be identified and/or characterized by detection of GDF15. For example, in some embodiments, a subject to whom therapy as described herein is administered is characterized by (e.g., has been determined to have) expression of GDF15 (e.g., above a particular threshold level). Indeed, among other things, the present disclosure provides an insight that subjects with a GDF15 level above about 1 ng/ml (e.g., when assessed in serum, blood and/or other bodily fluids, e.g., by ELISA or other appropriate methods known in the field) are likely to respond well to therapy as described herein.

Those skilled in the art are aware that a variety of technologies are available for detection or determination of GDF15 level.

Such detection methods are well known in the art and include ELISA, radioimmunoassay, immunoblot, Western blot, immunofluorescence, immunoprecipitation, and other comparable techniques. An Antibody Agent or a polypeptide (e.g., a LC polypeptide and/or a HC polypeptide), may further be provided in a diagnostic kit that incorporates one or more of these techniques to detect a protein (e.g., GDF15). Such a kit may contain other components, packaging, instructions, or other material to aid the detection of the protein and use of the kit.

Where the a GDF15-GFRAL Pathway Modulating Agent (e.g., an Antibody Agent such as a GDF15 Antibody Agent) are intended for diagnostic purposes, it may be desirable to modify them, for example, with a ligand group (such as biotin) or a detectable marker group (such as a fluorescent group, a radioisotope or an enzyme). If desired, the GDF15-GFRAL Pathway Modulating Agent (e.g., an Antibody Agent such as a GDF15 Antibody Agent) may be labeled using conventional techniques. Suitable labels include fluorophores, chromophores, radioactive atoms, electron-dense reagents, enzymes, and ligands having specific binding partners. Enzymes are typically detected by their activity. For example, horseradish peroxidase can be detected by its ability to convert tetramethylbenzidine (TMB) to a blue pigment, quantifiable with a spectrophotometer. Other suitable labels may include biotin and avidin or streptavidin, IgG and protein A, and the numerous receptor-ligand couples known in the art. Other permutations and possibilities will be readily apparent to those of ordinary skill in the art, and are considered as equivalents within the scope of the instant invention.

Accordingly, is a method comprising, assessing a level and/or activity of GDF15 in a sample from a subject, and administering a GDF15 pharmaceutical composition to the subject if the level of GDF15 is higher than a comparator.

In some embodiments, a level of GDF15 is evaluated with a GDF15-GFRAL Pathway Modulating Agent (e.g., an Antibody Agent such as a GDF15 Antibody Agent). In some embodiments, an increased level and/or activity of GDF15 is determined relative to a comparator. In some embodiments, a comparator comprises a predetermined reference sample such as a sample obtained from an otherwise similar subject who does not have a condition, disease or disorder, or a symptom of a disease or disorder.

### Pharmaceutical Compositions

The present disclosure, among other things, provides use of pharmaceutical compositions that comprise or otherwise deliver a GDF15-GFRAL Pathway Modulating Agent; typically, such pharmaceutical compositions comprise an active agent (e.g., an antibody agent or portion thereof, or a nucleic acid that encodes such antibody agent or portion thereof, etc.) one or more pharmaceutically or physiologically acceptable carriers, diluents, or excipients.

When “a therapeutically effective amount”, “an immunologically effective amount,” “an anti-immune response effective amount,” or “an immune response-inhibiting effective amount” is indicated, a precise amount of a pharmaceutical composition that comprises or delivers a GDF15-GFRAL Pathway Modulating Agent described herein can be determined by a physician with consideration, for example, of individual differences in age, weight, immune response, and condition of the patient (subject).

In some embodiments, pharmaceutical compositions described herein may comprise buffers including neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of a contaminant (e.g., an endotoxin).

In some embodiments, pharmaceutical compositions described herein may be administered in a manner appropriate to the disease, disorder, or condition to be treated or prevented. In some embodiments, quantity and/or frequency of administration may be determined by such factors as condition of a patient, and/or type and/or severity of a patient's disease, disorder, or condition, although appropriate dosages may be determined by clinical trials.

In some embodiments, a pharmaceutical composition provided by the present disclosure may be in a form such as, for example, liquid, semi-solid and solid dosage forms, such as liquid solutions (e.g., injectable and infusible solutions), dispersions or suspensions, liposomes, and suppositories. In some such embodiments, such compositions can be formulated for administration orally, intravenously, subcutaneously, intradermally, intratumorally, intranodally, intramedullary, intramuscularly, transarterially, sublingually, intranasally, topically or intraperitoneally. In some embodiments, provided pharmaceutical compositions are formulated for oral administration. In some embodiments, provided pharmaceutical compositions are formulated for intravenous administration. In some embodiments, provided pharmaceutical compositions are formulated for subcutaneous administration.

Pharmaceutical compositions described herein can be formulated for administration by using infusion techniques that are commonly known in the field (See, e.g., Rosenberg et al., New Eng. J. of Med. 319:1676, 1988, which is hereby incorporated by reference in its entirety). Pharmaceutical compositions described herein can be formulated for oral administration by using techniques that are commonly known in the field (See, e.g., Institute for Safe Medication Practices (ISMP). ISMP Acute Care Guidelines for Timely Administration of Scheduled Medications; 2011; Martyn et. al., Nurse Educ Pract. 2019 May; 37:109-114; and Fasinu et.al., Biopharm Drug Dispos. 2011 May; 32 (4): 185-209. which is hereby incorporated by reference in its entirety).

In some embodiments, pharmaceutical compositions described herein are administered in combination with (e.g., before, simultaneously, or following) an additional therapy for a symptom, disease or disorder, e.g., a SOC therapy for a symptom, disease or disorder. In some embodiments, pharmaceutical compositions described herein may be administered before or following surgery.

In some embodiments, a dosage of any aforementioned therapy to be administered to a subject will vary with a disease, disorder, or condition being treated and based on a specific subject. Scaling of dosages for human administration can be performed according to art-accepted practices.

### Dosing Regimens

Those skilled in the art will be able to determine, according to known methods, the appropriate amount, dose or dosage of a GDF15-GFRAL Pathway Modulating Agent (e.g., an Antibody Agent such as a GDF15 Antibody Agent), to administer to a patient, taking into account factors such as age, weight, general health, the route of administration, the nature of the symptom, disease or disorder requiring treatment, and the presence of other medications.

In some embodiments, a GDF15-GFRAL Pathway Modulating Agent (e.g., an Antibody Agent such as a GDF15 Antibody Agent) is administered at a fixed dose, i.e. independent of body weight. In some embodiments, a fixed dose reduces interpatient variability, e.g., efficacy and/or PK/PD parameters.

In some embodiments, a GDF15-GFRAL Pathway Modulating Agent (e.g., an Antibody Agent such as a GDF15 Antibody Agent) is administered at a fixed dose of about of 0.1 mg to about 2000 mg. In some embodiments, GDF15-GFRAL Pathway Modulating Agent (e.g., an Antibody Agent such as a GDF15 Antibody Agent) is administered at a fixed dose of about 0.1 mg, about 0.2 mg, about 0.25 mg, about 0.5 mg, about 1 mg, about 5 mg, about 10 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, or about 2000 mg. In some embodiments, a GDF15-GFRAL Pathway Modulating Agent (e.g., an Antibody Agent such as a GDF15 Antibody Agent) is administered intravenously (IV), subcutaneously (SC) or orally (OR) at a fixed dose of about 0.1 mg to about 2000 mg.

In some embodiments, a GDF15-GFRAL Pathway Modulating Agent administered at a fixed dose is administered daily, at an interval of once, twice, or three times in a day. In some embodiments, a GDF15-GFRAL Pathway Modulating Agent (e.g., an Antibody Agent such as a GDF15 Antibody Agent) administered at a fixed dose is administered daily, weekly or monthly. In some embodiments, a GDF15-GFRAL Pathway Modulating Agent (e.g., an Antibody Agent such as a GDF15 Antibody Agent) is administered at a fixed dose is administered once a week, once every 2 weeks, once every 3 weeks or once every 4 weeks. In some embodiments, a GDF15-GFRAL Pathway Modulating Agent (e.g., GDF15 Antibody Agent (e.g., an Antibody Agent such as a GDF15 Antibody Agent) is administered twice at a fixed dose once a week, once every 2 weeks, once every 3 weeks or once every 4 weeks.

In some embodiments, a dose, e.g., a fixed dose or a weight based dose, of a GDF15-GFRAL Pathway Modulating Agent (e.g., an Antibody Agent such as a GDF15 Antibody Agent) comprises one injection (e.g., SC, IM or IV injection).

In some embodiments, a dose, e.g., a fixed dose or a weight based dose, of a GDF15-GFRAL Pathway Modulating Agent (e.g., an Antibody Agent such as a GDF15 Antibody Agent) comprises more than one injection (e.g., SC, IM or IV injection). In some embodiments, a dose, e.g., a fixed dose or a weight based dose, of a GDF15-GFRAL Pathway Modulating Agent (e.g., an Antibody Agent such as a GDF15 Antibody Agent) comprises two injections. In some embodiments, more than one injections (e.g., two injections) of a dose, e.g., a fixed dose or a weight based dose, of a GDF15-GFRAL Pathway Modulating Agent (e.g., an Antibody Agent such as a GDF15 Antibody Agent) are administered simultaneously, substantially simultaneously, or consecutively. In some embodiments, more than one injections (e.g., two injections) of a dose, e.g., a fixed dose or a weight based dose, of a GDF15-GFRAL Pathway Modulating Agent (e.g., an Antibody Agent such as a GDF15 Antibody Agent) are administered within a specified duration of time, e.g., within about 1-120 minutes.

In some embodiments, a GDF15-GFRAL Pathway Modulating Agent (e.g., an Antibody Agent such as a GDF15 Antibody Agent) is administered based on body weight, e.g., in a mg/kg dosing. In some embodiments, a GDF15-GFRAL Pathway Modulating Agent (e.g., an Antibody Agent such as a GDF15 Antibody Agent) is administered at a dose of about 0.025 mg/kg to about 50 mg/kg. In some embodiments, a GDF15-GFRAL Pathway Modulating Agent (e.g., an Antibody Agent such as a GDF15 Antibody Agent) is administered at a dose of about 0.025 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg In some embodiments, a GDF15-GFRAL Pathway Modulating Agent (e.g., an Antibody Agent such as a GDF15 Antibody Agent) is administered intravenously (IV), subcutaneously (SC) or Orally (OR), for example, at a dose of about 0.025 mg/kg to about 50 mg/kg.

In some embodiments, a GDF15-GFRAL Pathway Modulating Agent (e.g., an Antibody Agent such as a GDF15 Antibody Agent) is administered at an initial dose. In some embodiments, an initial dose may be followed by one or more subsequent doses. In some embodiments, one or more subsequent dose may be administered daily, weekly, or monthly, or at other intervals in between. In some embodiments, a dosing regimen may be repeated for one or more times.

### Diseases, Disorders and Conditions

The present disclosure teaches that subjects suffering from or susceptible to one or more certain diseases, disorders or conditions as described herein may benefit from therapy with a GDF15-GFRAL Pathway Modulating Agent (e.g., an Antibody Agent such as a GDF15 Antibody Agent). In particular, the present disclosure provides an insight that certain types of nausea/emesis (specifically including types not previously associated with GDF15 and/or the GDF15-GFRAL Pathway) may benefit from treatment with a GDF15-GFRAL Pathway Modulating Agent (e.g., an Antibody Agent such as a GDF15 Antibody Agent) as described herein.

### Cyclic Vomiting Syndrome (CVS)

Cyclic vomiting syndrome (CVS) is characterized by recurrent episodes of heavy nausea, vomiting and frequently abdominal pain. (See, e.g., Blumentrath et.al., Ger Med Sci. 2017; 15: Doc06; and Hejazi et.al., Aliment Pharmacol Ther. 2011 August; 34 (3): 263-73; and Fleisher et.al., BMC Med. 2005 December; 3:20). Episodes lengths vary and can occur either randomly or regularly (See, e.g., Blumentrath et.al., Ger Med Sci. 2017; 15: Doc06). For several diagnosed with CVS, there are no definite causes (see, e.g., Blumentrath et.al., Ger Med Sci. 2017; 15: Doc06; and Hayes et.al., Clin Exp Gastroenterol. 2018; 11:77-84). CVS is difficult to diagnosis, because vomiting can be a symptom of many disorders. A patient is suspected of suffering from CVS if they experience three or more separate episodes in a year, and if they experience acute-onset episodes of vomiting each occurring at least 1 week apart (see, e.g., Hayes et.al., Clin Exp Gastroenterol. 2018; 11:77-84). They may also not experience nausea or vomiting between episodes, but may experience other, milder symptoms (see, e.g., Hayes et.al., Clin Exp Gastroenterol. 2018; 11:77-84). In some case, a more extensive diagnostic workup is required. Biochemical testing to dismiss hypothyroidism, hypercalcemia, Addison's disease, and other electrolyte and acid-base abnormalities may be required (see, e.g., Hayes et.al., Clin Exp Gastroenterol. 2018; 11:77-84). Furthermore, consideration of urine measurements of aminolevulinic acid and porphobilinogen, plasma ammonia levels, plasma amino acid, and urine organic acid quantification may be needed to exclude some rare conditions (see, e.g., Hayes et.al., Clin Exp Gastroenterol. 2018; 11:77-84).

CVS occurs in four distinct phases (e.g., the prodrome phase, vomiting phase, the recovery phase, or well phase) and for each phase treatment differs (see, e.g., Blumentrath et.al., Ger Med Sci. 2017; 15: Doc06; and Hayes et.al., Clin Exp Gastroenterol. 2018; 11:77-84). Patients experiencing the prodromal phase of CVS are treated to help stop an episode from happening (see, e.g., Blumentrath et.al., Ger Med Sci. 2017; 15: Doc06; and Hayes et.al., Clin Exp Gastroenterol. 2018; 11:77-84). Patients experiencing the vomiting phase are advised to stay in bed, sleep in a dark quiet room (see, e.g., Blumentrath et.al., Ger Med Sci. 2017; 15: Doc06; and Hayes et.al., Clin Exp Gastroenterol. 2018; 11:77-84). In severe cases, hospitalization may be necessary, since severe vomiting can increase the risk of dehydration and starvation. Patients in the recovery phase may need IV fluids and/or liquids aim to replenish glucose and/or electrolytes (see, e.g., Blumentrath et.al., Ger Med Sci. 2017; 15: Doc06; and Hayes et.al., Clin Exp Gastroenterol. 2018; 11:77-84). Patients often lose their appetite and may have trouble accepting solid foods. Lastly, patients in the well phase are prescribed medicines (e.g., anti-epileptics, anti-depressants, anti-histamines) to help prevent future episodes (see, e.g., Blumentrath et.al., Ger Med Sci. 2017; 15: Doc06; and Hayes et.al., Clin Exp Gastroenterol. 2018; 11:77-84).

Even though the main symptoms of CVS, nausea and vomiting, are associated with the GI tract, it is believed that the underlying cause of CVS may result from dysfunction in the nervous system (see, e.g., Levinthal Clin Transl Gastroenterol. 2016 October; 7 (1) e198). Additionally, there is a strong relationship between CVS and migraines. Some individuals with CVS have a family history of migraines; some suffer from migraines themselves. It has been proposed that targeting neuronal excitability may be a promising therapeutic target for CVS. (see, e.g., Levinthal, Clin Transl Gastroenterol. 2016 October; 7 (1) e198). Antidepressant, antiepileptic, and antimigraine medications have been reported to show an overall reduction or remission of CVS symptoms (see, e.g., Hayes et.al., Clin Exp Gastroenterol. 2018; 11:77-84). However, there are no standard therapies for CVS, and currently no one therapy works for all affected individuals (see, e.g., Blumentrath et.al., Ger Med Sci. 2017; 15: Doc06; and Hayes et.al., Clin Exp Gastroenterol. 2018; 11:77-84).

The present disclosure provides an insight that treatment with a GDF15-GFRAL Pathway Modulating Agent (e.g., an Antibody Agent such as a GDF15 Antibody Agent) may benefit some or all subject suffering from or susceptible to CVS. That is, without wishing to be bound by any particular theory, the present disclosure proposes that nausea and vomiting associated with CVS may be the result of elevated GDF15 levels.

The present disclosure specifically proposes that treatment with a GDF15-GFRAL Pathway Modulating Agent (e.g., an Antibody Agent such as a GDF15 Antibody Agent) may alter neuronal signaling pathways that may contribute to nausea and/or emesis and/or gastrointestinal hyperactivity in CVS. The present disclosure appreciates that it has recently been demonstrated that Calcitonin Gene-Related Peptide Receptor (CGRP) activity can cause nausea and gastrointestinal distress (see, e.g., Falkenberg et.al., Headache. 2020 May; 60 (5): 929-937). Others have proposed that CGRP antagonists might be explored as a potential new therapeutic strategy for CVS (see, e.g., Hasler et.al., Neurogastroenterol Motil. 2019 June; 31 Suppl 2 (Suppl 2): e13607; and Yu et.al., Curr Treat Options Gastroenterol. 2018 December; 16 (4): 511-527).

The present disclosure describes an alternative or complementary (e.g., combination) approach to treating CVS-using GDF15-GFRAL Pathway Modulating Agents (e.g., an Antibody Agent such as a GDF15 Antibody Agents) as described herein. The present disclosure observes that GFRAL positive neurons from the brainstem strongly innervate the parabrachial nucleus (PBN), where they target CGRP-expressing PBN neurons (CGRPPBN) (see, e.g., Sabatini et. al., PNAS. 2021 Feb. 23; 118 (8): e2021357118), and furthermore that CGRPPBN neurons relay a wide variety of aversive signals to the brain and play a major role in regulating appetite (see, e.g., Palmiter, Trends Neurosci. 2018 May; 41 (5): 280-293). Indeed, the present disclosure notes that chronic activation of CGRPPBN neurons can lead to severe anorexia and starvation (see, e.g., Palmiter, Trends Neurosci. 2018 May; 41 (5): 280-293). Importantly, infection or pathophysiologic states (rather than meal ingestion) stimulate GFRAL neurons and silencing CGRPPBN neurons can reduce aversive and anorexic effects of GDF15; it has been proposed that GFRAL neurons link non-meal-associated pathophysiologic signals to suppress nutrient uptake and absorption (see, e.g., Sabatini et. al., PNAS. 2021 Feb. 23; 118 (8): e2021357118).

In some embodiments, the GDF15-GFRAL pathway may contribute to the prodromal phase (feeling of nausea) in CVS, so that subjects in or at risk of such prodromal phase may benefit from treatment with a GDF15-GFRAL Pathway Modulating Agent (e.g., an Antibody Agent such as a GDF15 Antibody Agent) as described herein. In some embodiments, the GDF15-GFRAL pathway may contribute to the vomiting phase in CVS, so that subjects in the vomiting phase (e.g., who have experienced or are experiencing vomiting) may benefit from treatment with a GDF15-GFRAL Pathway Modulating Agent (e.g., an Antibody Agent such as a GDF15 Antibody Agent) as described herein.

Thus, the present disclosure teaches, in some embodiments, that treatment with a GDF15-GFRAL Pathway Modulating Agent (e.g., an Antibody Agent such as a GDF15 Antibody Agent) may be used as a prophylactic and/or treatment to delay onset of and/or reduce frequency and/or severity of nausea and/or emesis in CVS subjects.

In some embodiments, treatment with a GDF15-GFRAL Pathway Modulating Agent (e.g., an Antibody Agent such as a GDF15 Antibody Agent) is administered to CVS patients determined to have a GDF15 level (e.g., a GDF15 plasma level) above a particular threshold (e.g., 1 ng/ml).

### Cannabinoid Hyperemesis Syndrome (CHS)

Cannabinoid hyperemesis syndrome (CHS) is a condition that leads to repeated and severe bouts of vomiting amongst long-term users of cannabinoid (e.g., see, Sorensen et.al., J Med Toxicol. 2017 March; 13 (1): 71-87; Sun et.al., Hosp Pharm. 2013 September; 48 (8): 650-5.; Blumentrath et.al., Ger Med Sci. 2017; 15; Allen et.al., Gut. 2004 November; 53 (11): 1566-70. DOI: 10.1136/gut.2003.036350; and Darmani et.al., Pharmaceuticals (Basel). 2010 July; 3 (7): 2163-2177.). Similar to CVS, CHS also occurs in distinct phases (prodromal, hyperemetic, and recovery phases). Patients suffering from CHS exhibit similar symptoms to CVS, however there needs to be a history of long-term cannabinoid use in order to arrive at a CHS diagnosis (see, e.g., Chu F, Cascella M. Cannabinoid Hyperemesis Syndrome. [Updated 2021 Jul. 17]. In: StatPearls [Internet]. Treasure Island (FL): StatPearls Publishing; 2021 January-. Available from: https://www.ncbi.nlm.nih.gov/books/NBK549915/)

A diagnosis of CHS is currently a diagnosis of exclusion. If it is a patient's first presentation for nausea and/or emesis, other primary etiologies are normally considered before a diagnosis of CHS. Generally, repeat presentation of nausea, emesis, and/or delayed gastric emptying with chronic cannabinoid use allows for a CHS diagnosis (e.g., see, Sorensen et.al., J Med Toxicol. 2017 March; 13 (1): 71-87; and Blumentrath et.al., Ger Med Sci. 2017; 15).

Clinicians typically treat symptoms of CHS including nausea and/or emesis using anti-emetics, anti-psychotics, benzodiazepines, and/or anti-histamine (e.g., see, Sorensen et.al., J Med Toxicol. 2017 March; 13 (1): 71-87; and Blumentrath et.al., Ger Med Sci. 2017; 15). Interestingly, patients suffering from CHS have reported success relieving their nausea by taking a hot shower and/or topical capsaicin application (see, e.g., Blumentrath et.al., Ger Med Sci. 2017; Moon et.al., ACG Case Rep J. 2018; 5: e3; and Aziz et.al., Case Rep Gastrointest Med. 2020; 2020:8868385). In many cases, cessation of cannabinoid use results in long term resolution of symptoms (see, e.g., Blumentrath et.al., Ger Med Sci. 2017).

The pathophysiology underlying CHS is unclear, and for some patients cannabis cessation alone is not curative of CHS symptoms (see, e.g., Blumentrath et.al., Ger Med Sci. 2017). Although CHS and CVS show similarities in their clinical presentations, but they are clearly defined as distinct syndromes, and effort is invested in distinguishing them in order to provide desirable treatment (see, e.g., Blumentrath et.al., Ger Med Sci. 2017).

By contrast, the present disclosure proposes that subject suffering from CHS, like those suffering from CVS, might benefit from treatment with a GDF15-GFRAL Pathway Modulating Agent (e.g., an Antibody Agent such as a GDF15 Antibody Agent) as described herein. At least, the present disclosure teaches, in some embodiments, subjects suspected of or demonstrated to be suffering from CHS can be assessed to determine GDF15 level and, at least where such level is observed to be above a particular threshold (e.g., as described herein), can be treated with a GDF15-GFRAL Pathway Modulating Agent (e.g., an Antibody Agent such as a GDF15 Antibody Agent) as described herein.

That is, without wishing to be bound by any particular theory, the present disclosure provides an observation that nausea and vomiting associated with CHS may be the result of elevated GDF15 levels. In some embodiment, use of a GDF15-GFRAL Pathway Modulating Agent (e.g., an Antibody Agent such as a GDF15 Antibody Agent) may reduce GDF15 levels in CHS, thereby reducing symptoms of nausea and emesis in CHS.

In some embodiments, the GDF15-GFRAL pathway may contribute to the prodromal phase (feeling of nausea) in CVS, so that subjects in or at risk of such prodromal phase may benefit from treatment with a GDF15-GFRAL Pathway Modulating Agent (e.g., an Antibody Agent such as a GDF15 Antibody Agent) as described herein. In some embodiments, the GDF15-GFRAL pathway may contribute to the hyperemesis phase in CHS, so that subjects in the vomiting phase (e.g., who have experienced or are experiencing vomiting) may benefit from treatment with a GDF15-GFRAL Pathway Modulating Agent (e.g., an Antibody Agent such as a GDF15 Antibody Agent) as described herein. In some embodiments, the GDF15-GFRAL pathway may contribute to gastric stasis in CHS, so that subjects with decreased gastric emptying (e.g., who have experienced or are experiencing gastric stasis) may benefit from treatment with a GDF15-GFRAL Pathway Modulating Agent (e.g., an Antibody Agent such as a GDF15 Antibody Agent) as described herein.

Thus, the present disclosure teaches, in some embodiments, that treatment with a GDF15-GFRAL Pathway Modulating Agent may be used as a prophylactic and/or treatment to delay onset of and/or reduce frequency and/or severity of nausea and/or emesis in CHS subjects.

In some embodiments, treatment with a GDF15-GFRAL Pathway Modulating Agent (e.g., an Antibody Agent such as a GDF15 Antibody Agent) is administered to CHS patients determined to have a GDF15 level (e.g., a GDF15 plasma level) above a particular threshold (e.g., 1 ng/ml).

### Migraine Associated Nausea Vomiting (MAN V)

A migraine is a common neurological disease associated with a variety of symptoms. A migraine attack is characterized by debilitating frequent headaches, often accompanied by one or more additional symptoms such as photophobia, phonophobia, nausea, and/or emesis. It is estimated that approximately over 50% of patients with migraines develop nausea and/or emesis (see, e.g., Láinez et.al., Patient Relat Outcome Meas. 2013; 4:61-73.).

Nausea and/or emesis can occur during the prodromal phase of a migraine attack. Once a migraine attack has started, a main goal of migraine management, in the majority of patients, is to initiate abortive treatment as soon as possible (see, e.g., Láinez et.al., Patient Relat Outcome Meas. 2013; 4:61-73.). Commonly used therapies for migraine management include nonsteroidal anti-inflammatory drugs (NSAIDs), acetaminophen, triptans, ergots (see, e.g., Láinez et.al., Patient Relat Outcome Meas. 2013; 4:61-73.). Various reports recommend early administration of oral therapies, or selection of non-oral treatments (e.g., subcutaneously-administered therapies), particularly for subjects who have experienced (e.g., have a history of experiencing) and/or are experiencing nausea and/or emesis in association with a migraine (see, e.g., Láinez et.al., Patient Relat Outcome Meas. 2013; 4:61-73.).

The present disclosure provides an alternative strategy for treatment and/or prevention of MAN/V—i.e., by administration of GDF15-GFRAL Pathway Modulating Therapy (e.g., by administration of a GDF15-GFRAL Pathway Modulating Agent as described herein). In some embodiments, such an approach permits simultaneous or complementary administration of oral migraine therapies, e.g., without requiring particularly early administration.

The present disclosure further appreciates that certain reports have indicated that, in at least some cases, patients may experience gastric stasis. Gastric stasis can delay absorption and/or lower bioavailability of therapeutics. Without wishing to be bound by any particular theory, the present disclosure proposes that elevated GDF15 may cause or contribute to gastric stasis in at least some such cases. In some embodiments, the present disclosure teaches administration of GDF15-GFRAL Pathway Modulating Therapy, as described herein, to subjects suffering from or susceptible to gastric stasis. In some embodiments, such an approach can improve bioavailability of other therapy(ies) (e.g., that may be administered in combination with such GDF15-GFRAL Pathway Modulating Therapy and/or to which such subject may be otherwise exposed).

Certain reports have suggested that CVS can be considered to be a migraine variant (see, e.g., Láinez et.al., Patient Relat Outcome Meas. 2013; 4:61-73; Wang et.al., Am J Med Genet A. 2004 Nov. 15; 131 (1): 50-8.; and Li et.al., Gastroenterol Clin North Am. 2003 September; 32 (3): 997-1019.), including for example in light of observations that certain patients with CVS have a family history of migraines or have migraines themselves (see, e.g., Blumentrath et.al., Ger Med Sci. 2017). Also, certain medications (e.g., tricyclic antidepressants, antiepileptic drugs as prophylaxis, and triptans and antiemetics as abortive therapies) have been utilized in both contexts (see, e.g., Aurora et.al., Headache. 2021 April; 61 (4): 576-589.).

Without wishing to be bound by any particular theory, the present disclosure proposes that a relationship between CVS and migraines (and/or MAN/V) may be attributable, at least in part, to GDF15-GFRAL Pathway activity. Among other things, as described herein, the present disclosure proposes that CVS and/or MAN/V patients may benefit from GDF15-GFRAL Pathway Modulating Therapy as described herein. In some embodiments, CVS and/or MAN/V patients who show elevated GDF15 may benefit from such therapy.

Thus, the present disclosure teaches an alternative or complementary (e.g., combination) approach to treating MAN/V—using GDF15-GFRAL Pathway Modulating Agents (e.g., an Antibody Agent such as a GDF15 Antibody Agents) as described herein.

In some embodiments, the GDF15-GFRAL pathway may contribute to nausea in MAN/V, so that subjects in or at risk of such nausea may benefit from GDF15-GFRAL Pathway Modulating Therapy (e.g., by administration of a GDF15-GFRAL Pathway Modulating Agent as described herein). In some embodiments, the GDF15-GFRAL pathway may contribute to vomiting in MAN/V, so that subjects in or at risk of vomiting may benefit from treatment with a GDF15-GFRAL Pathway Modulating Agent (e.g., an Antibody Agent such as a GDF15 Antibody Agent) as described herein. In some embodiments, the GDF15-GFRAL pathway may contribute to gastric stasis in MAN/V, so that subjects with decreased gastric emptying (e.g., who have experienced or are experiencing gastric stasis) may benefit from treatment with a GDF15-GFRAL Pathway Modulating Agent (e.g., an Antibody Agent such as a GDF15 Antibody Agent) as described herein.

In some embodiments, treatment with a GDF15-GFRAL Pathway Modulating Agent may be used as a prophylactic and/or treatment to delay onset of and/or reduce frequency and/or severity of nausea and/or emesis in MAN/V subjects.

In some embodiments, treatment with a GDF15-GFRAL Pathway Modulating Agent (e.g., an Antibody Agent such as a GDF15 Antibody Agent) is administered to MAN/V patients determined to have a GDF15 level (e.g., a GDF15 plasma level) above a particular threshold (e.g., 1 ng/ml).

### Mitochondrial Disorders (MIDs)

Mitochondrial disorders (MIDs) are multisystem conditions which can affect the central nervous system (CNS), peripheral nervous system, eyes, ears, endocrine organs, heart, kidneys, bone marrow, lungs, arteries, and also the intestinal tract (Finsterer and Frank, Therap Adv Gastroenterol. 2017 January; 10 (1): 142-154). A key feature of MIDs is dysfunction of the mitochondrial respiratory chain leading to defective ATP production (Montero R et al., PLOS One. 2016; 11 (2): e0148709).

More than half of MIDs present with some GI involvement. MIDs may also mimic cyclic vomiting syndrome, e.g., as described herein. GI manifestations in MIDs include poor appetite, gastroesophageal sphincter dysfunction, constipation, dysphagia, vomiting, gastroparesis, GI pseudo-obstruction, diarrhea, or pancreatitis and hepatopathy (see Finsterer and Frank). Commonly used therapies for treating GI manifestations in MIDs may be symptomatic, causative, invasive, noninvasive, surgical or non-surgical. In a majority of cases, symptomatic treatment is used such as dietary supplements, appetite stimulation, intravenous glucose administration, and/or antidepressants.

The present disclosure provides an alternative strategy for treatment and/or prevention of MIDs—i.e., by administration of GDF15-GFRAL Pathway Modulating Therapy (e.g., by administration of a GDF15-GFRAL Pathway Modulating Agent as described herein). In some embodiments, such an approach permits simultaneous or complementary administration of standard MIDs therapies, e.g., without requiring particularly early administration.

The present disclosure further appreciates that certain reports have indicated that, in at least some cases, GDF-15 is elevated in children with MIDs and that GDF-15 can be used as a biomarker for the diagnosis of MIDs in children (See Montero R et al., PLOS One. 2016; 11 (2): e0148709). Furthermore, GDF-15 was also shown to be produced by skeletal muscle cells in response to mitochondrial dysfunction. Without wishing to be bound by any particular theory, the present disclosure proposes that elevated GDF15 may cause or contribute to MIDs, and/or GI manifestations in MIDs in at least some such cases. In some embodiments, the present disclosure teaches administration of GDF15-GFRAL Pathway Modulating Therapy, as described herein, to subjects suffering from or susceptible to MIDs (e.g., MIDs with GI manifestations). In some embodiments, such an approach can improve bioavailability of other therapy(ies) (e.g., that may be administered in combination with such GDF15-GFRAL Pathway Modulating Therapy and/or to which such subject may be otherwise exposed).

In some embodiments, the GDF15-GFRAL pathway may contribute to one or more GI manifestations in MIDs, so that subjects in or at risk of such GI manifestations may benefit from GDF15-GFRAL Pathway Modulating Therapy (e.g., by administration of a GDF15-GFRAL Pathway Modulating Agent as described herein). In some embodiments, the GDF15-GFRAL pathway may contribute to nausea in MIDs, so that subjects in or at risk of such nausea may benefit from GDF15-GFRAL Pathway Modulating Therapy (e.g., by administration of a GDF15-GFRAL Pathway Modulating Agent as described herein). In some embodiments, the GDF15-GFRAL pathway may contribute to vomiting in MIDs, so that subjects in or at risk of vomiting may benefit from treatment with a GDF15-GFRAL Pathway Modulating Agent (e.g., an Antibody Agent such as a GDF15 Antibody Agent) as described herein.

In some embodiments, treatment with a GDF15-GFRAL Pathway Modulating Agent may be used as a prophylactic and/or treatment to delay onset of and/or reduce frequency and/or severity of GI manifestations in (e.g., nausea and/or emesis) in MIDs subjects.

In some embodiments, treatment with a GDF15-GFRAL Pathway Modulating Agent (e.g., an Antibody Agent such as a GDF15 Antibody Agent) is administered to MIDs patients determined to have a GDF15 level (e.g., a GDF15 plasma level) above a particular threshold (e.g., 1 ng/ml).

### Exemplary Uses of GDF15-GFRAL Pathway Modulating Agents or Components (e.g., Polypeptide Elements or Portions) Thereof

GDF15-GFRAL Pathway Modulating Agent (e.g., an Antibody Agent such as a GDF15 Antibody Agent) are useful in a variety of contexts, including in research, diagnosis, and therapy. In some embodiments, a GDF15-GFRAL Pathway Modulating Agent (e.g., an Antibody Agent such as a GDF15 Antibody Agent) can be used as a reference agent and/or a reagent in research, e.g., to understand GDF15 biology and/or biological processes directly or indirectly related to GDF15. In some embodiments, a GDF15-GFRAL Pathway Modulating Agent (e.g., an Antibody Agent such as a GDF15 Antibody Agent) can be used as a reference agent and/or a reagent in diagnosis and/or treatment (e.g., patient selection).

In some embodiments, a GDF15-GFRAL Pathway Modulating Agent (e.g., an Antibody Agent such as a GDF15 Antibody Agent) is characterized in that when administered to a subject it reduces a level and/or activity of GDF15, e.g., as compared to before administration of GDF15-GFRAL Pathway Modulating Agent. In some embodiments, reduced GDF15 level and/or activity is assessed in a subject, e.g., via imaging, or in a sample from a subject, e.g., a tissue sample (e.g., a biopsy), or a bodily fluid sample (e.g., a blood, plasma, serum, urine, CSF, saliva or other bodily fluid).

In some embodiments, GDF15-GFRAL Pathway Modulating Agent (e.g., an Antibody Agent such as a GDF15 Antibody Agent) reduces a GDF15 level to less than 1 ng/ml. In some embodiments, GDF15-GFRAL Pathway Modulating Agent (e.g., an Antibody Agent such as a GDF15 Antibody Agent) reduces a GDF15 level to at least 1%-90% less than before administration of GDF15-GFRAL Pathway Modulating Agent (e.g., an Antibody Agent such as a GDF15 Antibody Agent).

Also are uses of GDF15-GFRAL Pathway Modulating Agent (e.g., an Antibody Agent such as a GDF15 Antibody Agent) or a composition comprising the same for ameliorating (e.g., reducing) one or more symptoms associated with certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V).

In some embodiments, use of GDF15-GFRAL Pathway Modulating Agent (e.g., an Antibody Agent such as a GDF15 Antibody Agent) or a composition comprising the same may be useful in treating and/or preventing nausea. In some embodiments, a subject having nausea has an increased level of GDF15, e.g., as compared to a subject who does not have nausea. In some embodiments, an increased level of GDF15 is a level of about 1 ng/ml or more, e.g., as evaluated in a sample from a subject, e.g., a blood, plasma, serum or urine sample.

In some embodiments GDF15-GFRAL Pathway Modulating Agent (e.g., an Antibody Agent such as a GDF15 Antibody Agent) or a composition comprising the same may be useful in treating and/or preventing vomiting. In some embodiments, a subject having vomiting has an increased level of GDF15, e.g., as compared to a subject who does not have vomiting. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing both nausea and vomiting. In some embodiments, a subject having nausea and vomiting has an increased level of GDF15, e.g., as compared to a subject who does not have nausea and vomiting. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing weight loss in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having weight loss has an increased level of GDF15, e.g., as compared to a subject who does not have weight loss. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing loss of appetite in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having loss of appetite has an increased level of GDF15, e.g., as compared to a subject who does not have loss of appetite. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing taste aversion in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having taste aversion has an increased level of GDF15, e.g., as compared to a subject who does not have taste aversion. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing fatigue in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having fatigue has an increased level of GDF15, e.g., as compared to a subject who does not have fatigue. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing muscle loss in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having muscle loss has an increased level of GDF15, e.g., as compared to a subject who does not have muscle loss. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing pain in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having pain has an increased level of GDF15, e.g., as compared to a subject who does not have pain. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing gastric stasis in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having gastric stasis has an increased level of GDF15, e.g., as compared to a subject who does not have pain. In some embodiments, an increased level of GDF15 is a level of about 1 ng/ml or more, e.g., as evaluated in a sample from a subject, e.g., a blood, plasma, serum or urine sample.

In some embodiments, GDF15-GFRAL Pathway Modulating Agent (e.g., an Antibody Agent such as a GDF15 Antibody Agent) or a composition comprising the same may be useful in treating and/or preventing a poor quality of life in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having a poor quality of life has an increased level of GDF15, e.g., as compared to a subject who does not have pain. In some embodiments, an increased level of GDF15 is a level of about 1 ng/ml or more, e.g., as evaluated in a sample from a subject, e.g., a blood, plasma, serum or urine sample.

In some embodiments, where a standard of care has been established for treatment or prevention of a particular symptom or symptom(s) (e.g., of a particular disease, disorder or condition), therapy with a provided GDF15-GFRAL Pathway Modulating Agent (e.g., an Antibody Agent such as a GDF15 Antibody Agent) is administered in combination with such standard of care; in some embodiments, therapy with a provided GDF15-GFRAL Pathway Modulating Agent (e.g., an Antibody Agent such as a GDF15 Antibody Agent) may be administered as an alternative to such standard of care.

In some embodiments, a therapy for a disease increases the level and/or activity of GDF15, e.g., compared to the level of GDF15 prior to administration of a therapy, e.g., a therapy for a disorder disclosed herein. In some embodiments, an increased level of GDF15 is a level of at least 1 ng/ml, e.g., in a sample from a subject, e.g., a blood, plasma, serum or urine sample.

In some embodiments, a therapy for a disease does not increase the level and/or activity of GDF15, e.g., compared to the level of GDF15 prior to administration of a therapy, e.g., a therapy for a disorder disclosed herein.

In some embodiments, a subject having a disease disclosed herein, a symptom of a disease disclosed herein, or a symptom associated with a therapy for a disease disclosed herein, has an increased level of GDF15 relative to a comparator. In some embodiments, a comparator is a subject who does not have a disease disclosed herein, does not have a symptom of a disease or have has a disease but has not been administered a therapy for a disease.

In some embodiments, administration of GDF15-GFRAL Pathway Modulating Agent (e.g., an Antibody Agent such as a GDF15 Antibody Agent) ameliorates a symptom of a disease or a symptom associated with (e.g., induced by) a disease disclosed herein.

In some embodiments, administration of GDF15-GFRAL Pathway Modulating Agent (e.g., an Antibody Agent such as a GDF15 Antibody Agent) to a subject having increased GDF15 reduces a level and/or activity of GDF15, e.g., relative to before administration of GDF15-GFRAL Pathway Modulating Agent (e.g., an Antibody Agent such as a GDF15 Antibody Agent). In some embodiments, GDF15-GFRAL Pathway Modulating Agent (e.g., an Antibody Agent such as a GDF15 Antibody Agent) reduces a level of GDF15 (e.g., free and/or active GDF15) to less than 1 ng/mL, e.g., as assessed in a sample from a subject, e.g., a blood, plasma, serum or urine sample.

In some embodiments, a symptom ameliorated (e.g., reduced) with use of GDF15-GFRAL Pathway Modulating Agent (e.g., an Antibody Agent such as a GDF15 Antibody Agent) is nausea. In some embodiments, nausea is associated with a disease. In some embodiments, nausea is induced by a therapy for a disease. In some embodiments, a subject having nausea has an increased level of GDF15, e.g., as compared to a subject who does not have nausea. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of GDF15-GFRAL Pathway Modulating Agent (e.g., an Antibody Agent such as a GDF15 Antibody Agent) is vomiting. In some embodiments, vomiting is associated with a disease. In some embodiments, vomiting is caused by a therapy for a disease. In some embodiments, a subject having vomiting has an increased level of GDF15, e.g., as compared to a subject who does not have vomiting. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of GDF15-GFRAL Pathway Modulating Agent (e.g., an Antibody Agent such as a GDF15 Antibody Agent) is weight loss. In some embodiments, weight loss is associated with a disease. In some embodiments, weight loss is caused by a therapy for a disease. In some embodiments, weight loss is caused by a reduction in food intake, e.g., as compared to an earlier time point in the same subject or as compared to a subject of comparable age. In some embodiments, a subject having weight loss has an increased level of GDF15, e.g., as compared to a subject who does not have weight loss. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of GDF15-GFRAL Pathway Modulating Agent (e.g., an Antibody Agent such as a GDF15 Antibody Agent) is a loss of appetite. In some embodiments, a loss of appetite is associated with a disease. In some embodiments, a loss of appetite is induced by a therapy for a disease. In some embodiments, a loss of appetite is induced by nausea. In some embodiments, a loss of appetite is not induced by nausea. In some embodiments, a subject having loss of appetite has an increased level of GDF15, e.g., as compared to a subject who does not have loss of appetite. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of GDF15-GFRAL Pathway Modulating Agent (e.g., an Antibody Agent such as a GDF15 Antibody Agent) is fatigue. In some embodiments, fatigue is associated with a disease. In some embodiments, fatigue is caused by a therapy for a disease. In some embodiments, a subject having fatigue has an increased level of GDF15, e.g., as compared to a subject who does not have fatigue. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of GDF15-GFRAL Pathway Modulating Agent (e.g., an Antibody Agent such as a GDF15 Antibody Agent) is taste aversion. In some embodiments, taste aversion is associated with a disease. In some embodiments, taste aversion is caused by a therapy for a disease. In some embodiments, a subject having taste aversion has an increased level of GDF15, e.g., as compared to a subject who does not have taste aversion. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of GDF15-GFRAL Pathway Modulating Agent (e.g., an Antibody Agent such as a GDF15 Antibody Agent) is muscle loss. In some embodiments, muscle loss is associated with a disease. In some embodiments, muscle loss is caused by a therapy for a disease. In some embodiments, a subject having muscle loss has an increased level of GDF15, e.g., as compared to a subject who does not have muscle loss. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of GDF15-GFRAL Pathway Modulating Agent (e.g., an Antibody Agent such as a GDF15 Antibody Agent) is a poor quality of life. In some embodiments, muscle loss is associated with a disease. In some embodiments, a poor quality of life is caused by a therapy for a disease. In some embodiments, a subject having a poor quality of life has an increased level of GDF15, e.g., as compared to a subject who does not have a poor quality of life. In some embodiments, an increased level of GDF15 is a level of about 1 ng/ml or more, e.g., as evaluated in a sample from a subject, e.g., a blood, plasma, serum or urine sample.

This disclosure provides methods of using GDF15-GFRAL Pathway Modulating Agents for, e.g., inhibiting GDF15 (e.g., reducing an activity and/or level of GDF15) in a cell, tissue or subject (e.g., in a subject or in a sample from a subject). In some embodiments, a cell, tissue or subject administered a GDF15-GFRAL Pathway Modulating Agent has an increased level of GDF15. In some embodiments, an increased level of GDF15 is about 1 ng/ml or more. In some embodiments, a level and/or activity of GDF15 is evaluated in a subject, e.g., via imaging, or in a sample from a subject, e.g., a tissue sample (e.g., a biopsy), or a bodily fluid sample (e.g., a blood, plasma, serum, urine, CSF, saliva or other bodily fluid).

In some embodiments, a GDF15-GFRAL Pathway Modulating Agent (e.g., an Antibody Agent such as a GDF15 Antibody Agent) is characterized in that when administered to a subject it reduces a level and/or activity of GDF15, e.g., as compared to before administration of GDF15-GFRAL Pathway Modulating Agent (e.g., an Antibody Agent such as a GDF15 Antibody Agent). In some embodiments, reduced GDF15 level and/or activity is assessed in a subject, e.g., via imaging, or in a sample from a subject, e.g., a tissue sample (e.g., a biopsy), or a bodily fluid sample (e.g., a blood, plasma, serum, urine, CSF, saliva or other bodily fluid).

In some embodiments, a GDF15-GFRAL Pathway Modulating Agent (e.g., an Antibody Agent such as a GDF15 Antibody Agent) reduces a GDF15 level to less than 1 ng/ml. In some embodiments, a GDF15-GFRAL Pathway Modulating Agent reduces a GDF15 level to at least 1%-90% less than before administration of a GDF15-GFRAL Pathway Modulating Agent (e.g., an Antibody Agent such as a GDF15 Antibody Agent).

Also disclosed herein are uses of a GDF15-GFRAL Pathway Modulating Agent (e.g., an Antibody Agent such as a GDF15 Antibody Agent) (or a composition comprising the same) for ameliorating (e.g., reducing) one or more symptoms associated with certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V).

In some embodiments, a GDF15-GFRAL Pathway Modulating Agent (e.g., an Antibody Agent such as a GDF15 Antibody Agent) or a composition comprising the same may be useful in treating and/or preventing nausea. In some embodiments, a subject having nausea has an increased level of GDF15, e.g., as compared to a subject who does not have nausea. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing vomiting. In some embodiments, a subject having vomiting has an increased level of GDF15, e.g., as compared to a subject who does not have vomiting. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing both nausea and vomiting. In some embodiments, a subject having nausea and vomiting has an increased level of GDF15, e.g., as compared to a subject who does not have nausea and vomiting. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing weight loss in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having weight loss has an increased level of GDF15, e.g., as compared to a subject who does not have weight loss. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing loss of appetite in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having loss of appetite has an increased level of GDF15, e.g., as compared to a subject who does not have loss of appetite. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing taste aversion in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having taste aversion has an increased level of GDF15, e.g., as compared to a subject who does not have taste aversion. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing fatigue in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having fatigue has an increased level of GDF15, e.g., as compared to a subject who does not have fatigue. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing muscle loss in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having muscle loss has an increased level of GDF15, e.g., as compared to a subject who does not have muscle loss. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing pain in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having pain has an increased level of GDF15, e.g., as compared to a subject who does not have pain. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing gastric stasis in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having gastric stasis has an increased level of GDF15, e.g., as compared to a subject who does not have gastric stasis. In some embodiments, an increased level of GDF15 is a level of about 1 ng/ml or more, e.g., as evaluated in a sample from a subject, e.g., a blood, plasma, serum or urine sample.

In some embodiments, a GDF15-GFRAL Pathway Modulating Agent (e.g., an Antibody Agent such as a GDF15 Antibody Agent) or a composition comprising the same may be useful in treating and/or preventing a poor quality of life in certain types and/or instances of nausea and/or emesis with which it had not previously been considered to be associated. For example, in some embodiments, treating and/or preventing symptoms of Cyclic Vomiting Syndrome (CVS), Cannabinoid Hyperemesis Syndrome (CHS) and/or Migraine Associated Nausea/Vomiting (MAN/V). In some embodiments, a subject having a poor quality of life has an increased level of GDF15, e.g., as compared to a subject who does not have a poor quality of life. In some embodiments, an increased level of GDF15 is a level of about 1 ng/ml or more, e.g., as evaluated in a sample from a subject, e.g., a blood, plasma, serum or urine sample.

In some embodiments, where a standard of care has been established for treatment or prevention of a particular symptom or symptom(s) (e.g., of a particular disease, disorder or condition), therapy with a provided a GDF15-GFRAL Pathway Modulating Agent (e.g., an Antibody Agent such as a GDF15 Antibody Agent) is administered in combination with such standard of care; in some embodiments, therapy with a provided a GDF15-GFRAL Pathway Modulating Agent (e.g., an Antibody Agent such as a GDF15 Antibody Agent) may be administered as an alternative to such standard of care.

In some embodiments, a therapy for a disease increases the level and/or activity of GDF15, e.g., compared to the level of GDF15 prior to administration of a therapy, e.g., a therapy for a disorder disclosed herein. In some embodiments, an increased level of GDF15 is a level of at least 1 ng/ml, e.g., in a sample from a subject, e.g., a blood, plasma, serum or urine sample.

In some embodiments, a therapy for a disease does not increase the level and/or activity of GDF15, e.g., compared to the level of GDF15 prior to administration of a therapy, e.g., a therapy for a disorder disclosed herein.

In some embodiments, a subject having a disease disclosed herein, a symptom of a disease disclosed herein, or a symptom associated with a therapy for a disease disclosed herein, has an increased level of GDF15 relative to a comparator. In some embodiments, a comparator is a subject who does not have a disease disclosed herein, does not have a symptom of a disease or have has a disease but has not been administered a therapy for a disease.

In some embodiments, administration of a GDF15-GFRAL Pathway Modulating Agent (e.g., an Antibody Agent such as a GDF15 Antibody Agent) ameliorates a symptom of a disease or a symptom associated with (e.g., induced by) a disease disclosed herein.

In some embodiments, administration of a GDF15-GFRAL Pathway Modulating Agent (e.g., an Antibody Agent such as a GDF15 Antibody Agent) to a subject having increased GDF15 reduces a level and/or activity of GDF15, e.g., relative to before administration of a GDF15-GFRAL Pathway Modulating Agent (e.g., an Antibody Agent such as a GDF15 Antibody Agent). In some embodiments, a GDF15-GFRAL Pathway Modulating Agent (e.g., an Antibody Agent such as a GDF15 Antibody Agent) reduces a level of GDF15 (e.g., free and/or active GDF15) to less than 1 ng/mL, e.g., as assessed in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is nausea. In some embodiments, nausea is associated with a disease. In some embodiments, nausea is induced by a therapy for a disease. In some embodiments, a subject having nausea has an increased level of GDF15, e.g., as compared to a subject who does not have nausea. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is vomiting. In some embodiments, vomiting is associated with a disease. In some embodiments, vomiting is caused by a therapy for a disease. In some embodiments, a subject having vomiting has an increased level of GDF15, e.g., as compared to a subject who does not have vomiting. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is weight loss. In some embodiments, weight loss is associated with a disease. In some embodiments, weight loss is caused by a therapy for a disease. In some embodiments, weight loss is caused by a reduction in food intake, e.g., as compared to an earlier time point in the same subject or as compared to a subject of comparable age. In some embodiments, a subject having weight loss has an increased level of GDF15, e.g., as compared to a subject who does not have weight loss. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is a loss of appetite. In some embodiments, a loss of appetite is associated with a disease. In some embodiments, a loss of appetite is induced by a therapy for a disease. In some embodiments, a loss of appetite is induced by nausea. In some embodiments, a loss of appetite is not induced by nausea. In some embodiments, a subject having loss of appetite has an increased level of GDF15, e.g., as compared to a subject who does not have loss of appetite. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is fatigue. In some embodiments, fatigue is associated with a disease. In some embodiments, fatigue is caused by a therapy for a disease. In some embodiments, a subject having fatigue has an increased level of GDF15, e.g., as compared to a subject who does not have fatigue. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is taste aversion. In some embodiments, taste aversion is associated with a disease. In some embodiments, taste aversion is caused by a therapy for a disease. In some embodiments, a subject having taste aversion has an increased level of GDF15, e.g., as compared to a subject who does not have taste aversion. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is muscle loss. In some embodiments, muscle loss is associated with a disease. In some embodiments, muscle loss is caused by a therapy for a disease. In some embodiments, a subject having muscle loss has an increased level of GDF15, e.g., as compared to a subject who does not have muscle loss. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is pain. In some embodiments, pain is associated with a disease. In some embodiments, pain is caused by a therapy for a disease. In some embodiments, a subject having pain has an increased level of GDF15, e.g., as compared to a subject who does not have pain. In some embodiments, an increased level of GDF15 is a level of about 1 ng/ml or more, e.g., as evaluated in a sample from a subject, e.g., a blood, plasma, serum or urine sample.

In some embodiments, a symptom ameliorated (e.g., reduced) with use of a GDF15-GFRAL Pathway Modulating Agent (e.g., an Antibody Agent such as a GDF15 Antibody Agent) is gastric stasis. In some embodiments, pain is associated with a disease. In some embodiments, gastric stasis is caused by a therapy for a disease. In some embodiments, a subject having gastric stasis has an increased level of GDF15, e.g., as compared to a subject who does not have gastric stasis. In some embodiments, an increased level of GDF15 is a level of about 1 ng/ml or more, e.g., as evaluated in a sample from a subject, e.g., a blood, plasma, serum or urine sample.

### Exemplary Use of an Antibody Agent

GDF15-GFRAL Pathway Modulating Agent (e.g., an Antibody Agent such as a GDF15 Antibody Agent) is useful in a variety of contexts, including in research, diagnosis, and therapy. In some embodiments, a GDF15-GFRAL Pathway Modulating Agent comprises a polypeptide. In some embodiments, a polypeptide comprises an antibody or fragment thereof.

In some embodiments, administration of a provided GDF15 Antibody Agent reduces a level of GDF15 (e.g., free and/or active GDF15) in a sample, e.g., from a subject. In some embodiments, a level of GDF15 (e.g., free and/or active GDF15) is reduced by about 5%, about 10%, about 20%, about 30%, about 40%, about 50%, about 60%, about 70%, about 80%, about 90% or about 100%.

In some embodiments, a GDF15 Antibody Agents has improved binding affinity to GDF15 as compared to GDF15 antibodies disclosed in PCT/EP2016/073520 and are expected to have improved therapeutic efficacy. For example, binding affinity (Kd) of a GDF15 Antibody Agent to GDF15 is about 7.3 pM to about 117 pM, e.g., when assessed with a plasmon resonance assay. In contrast, GDF15 antibodies disclosed in PCT/EP2016/073520 have a Kd of 790 pM (see Reference Example 1 therein).

In some embodiments, administration of a provided GDF15 antibody Agent reduces a level of GDF15 (e.g., free and/or active GDF15) in a sample, e.g., from a subject. In some embodiments, a level of GDF15 (e.g., free and/or active GDF15) is reduced by about 5%, about 10%, about 20%, about 30%, about 40%, about 50%, about 60%, about 70%, about 80%, about 90% or about 100%.

### Subjects and Indications

In some embodiments, is a method of treating a subject having a disease or disorder associated with nausea and/or emesis For example, in some embodiments, the present disclosure teaches that Cyclic Vomiting Syndrome (CVS), Cannabinoid Hyperemesis Syndrome (CHS) and/or Migraine Associated Nausea/Vomiting (MAN/V) and/or subjects that suffer from CVS, CHS, and/or MAN/V, may benefit from therapeutic targeting of the GDF15-GFRAL Pathway Modulating Agent (e.g., an Antibody Agent such as a GDF15 Antibody Agent). In some embodiments, nausea and/or emesis is associated with increased GDF15. In some embodiments, administration of a GDF15-GFRAL Pathway Modulating Agent (e.g., an Antibody Agent such as a GDF15 Antibody Agent), or a composition comprising the same to a subject having nausea reduces a severity, onset and/or frequency of nausea. In some embodiments, administration of a GDF15-GFRAL Pathway Modulating Agent (e.g., an Antibody Agent such as a GDF15 Antibody Agent), or a composition comprising the same to a subject having weight loss, reduces, prevents and/or reverses weight loss. In some embodiments, administration of a GDF15-GFRAL Pathway Modulating Agent (e.g., an Antibody Agent such as a GDF15 Antibody Agent), or a composition comprising the same to a subject having a loss of appetite, increases appetite.

In some embodiments, a disease or disorder associated with nausea is Hyperemesis gravidarum. In some embodiments, a subject having nausea is pregnant. In some embodiments, a pregnant subject having nausea has an increased level and/or activity of GDF15, e.g., as compared to a subject who is not pregnant, or a pregnant subject who does not have nausea. In some embodiments, administration of a GDF15-GFRAL Pathway Modulating Agent (e.g., an Antibody Agent such as a GDF15 Antibody Agent), to a subject having nausea, e.g., Hyperemesis gravidarum, reduces a severity, onset, and/or frequency of nausea. In some embodiments, a GDF15-GFRAL Pathway Modulating Agent (e.g., an Antibody Agent such as a GDF15 Antibody Agent) can be administered to a subject having nausea, e.g., Hyperemesis gravidarum, alone, in combination with, or as an alternative to a nausea therapy, e.g., a SOC for nausea.

In some embodiments, a subject administered a GDF15-GFRAL Pathway Modulating Agent (e.g., an Antibody Agent such as a GDF15 Antibody Agent) is pregnant. In some embodiments, a GDF15-GFRAL Pathway Modulating Agent (e.g., an Antibody Agent such as a GDF15 Antibody Agent) administered to a pregnant subject has one or more modifications to reduce binding of a GDF15-GFRAL Pathway Modulating Agent (e.g., an Antibody Agent such as a GDF15 Antibody Agent) to a neonatal Fc receptor (FcRn). In some embodiments, a GDF15-GFRAL Pathway Modulating Agent (e.g., an Antibody Agent such as a GDF15 Antibody Agent) with reduced binding to FcRn can reduce or prevent placental transfer of a GDF15-GFRAL Pathway Modulating Agent (e.g., an Antibody Agent such as a GDF15 Antibody Agent) compared to an otherwise similar GDF15-GFRAL Pathway Modulating Agent (e.g., an Antibody Agent such as a GDF15 Antibody Agent) without reduced FcRn binding.

In some embodiments, a GDF15-GFRAL Pathway Modulating Agent (e.g., an Antibody Agent such as a GDF15 Antibody Agent) with reduced binding to FcRn comprises a mutation in an Fc portion of a GDF15-GFRAL Pathway Modulating Agent (e.g., an Antibody Agent such as a GDF15 Antibody Agent), e.g., a mutation described herein. For example, a GDF15-GFRAL Pathway Modulating Agent (e.g., an Antibody Agent such as a GDF15 Antibody Agent) with reduced binding to FcRn comprising a mutation in an Fc portion has reduced binding (e.g., no binding) to a FcRn at ph7.4.

Exemplary mutations to reduced FcRn binding of antibodies are disclosed in Thorn M et al., (2012) Birth Defects Research Part B 95:363-375, which is hereby incorporated by reference. In some embodiments, a modification to reduce binding of an antibody agent to FcRn comprises a H435A mutation as described in Thorn et al., 2012.

In some embodiments, a modification to reduce binding of an antibody agent to FcRn comprises an Fc mutation, e.g., as described herein.

In some embodiments, a modification to reduce binding of an antibody agent to FcRn comprises a I253A mutation, a H310A mutation, a H435R mutation, a H435A mutation or a combination thereof.

In some embodiments, a modification to reduce binding of an antibody agent to FcRn comprises a LAGA mutation, a FEGG mutation, an AAGG mutation, an AAGA mutation, a LALA mutation or a combination thereof.

In some embodiments, a pregnant subject having nausea (e.g., Hyperemesis gravidarum), loss of appetite and/or loss of body weight is administered a GDF15-GFRAL Pathway Modulating Agent (e.g., an Antibody Agent such as a GDF15 Antibody Agent) having one or more modifications to reduce FcRn binding. In some embodiments, administration of a GDF15-GFRAL Pathway Modulating Agent (e.g., an Antibody Agent such as a GDF15 Antibody Agent) having reduced FcRn binding treats and/or prevents: nausea (e.g., Hyperemesis gravidarum), loss of appetite and/or loss of body weight in a pregnant subject. In some embodiments, a GDF15-GFRAL Pathway Modulating Agent (e.g., an Antibody Agent such as a GDF15 Antibody Agent) having reduced FcRn binding when administered to a pregnant woman has reduced (e.g., none) placental transfer compared to an otherwise similar GDF15-GFRAL Pathway Modulating Agent (e.g., an Antibody Agent such as a GDF15 Antibody Agent) without reduced FcRn binding.

In some embodiments of any of the methods or uses disclosed herein, a subject is a child. In some embodiments, a child is between 1 day and 18 years of age. In some embodiments, a child has a body weight of about 4 pounds to 150 pounds.

In some embodiments of any of the methods or uses disclosed herein, a subject is an adult. In some embodiments, an adult, is a human 18 years of age or older. In some embodiments, a human adult has a weight within the range of about 90 pounds to about 250 pounds.

### Combination Therapies

In some embodiments, a GDF15-GFRAL Pathway Modulating Agent (e.g., an Antibody Agent such as a GDF15 Antibody Agent), or a composition comprising the same is administered in combination with an additional agent, e.g., additional therapy. In some embodiments, an additional therapy comprises a therapy for a disease or disorder, e.g., a standard of care (SOC) therapy, for a symptom, disease or disorder. In some embodiments, a GDF15-GFRAL Pathway Modulating Agent (e.g., an Antibody Agent such as a GDF15 Antibody Agent) is administered before, concurrently with or after administration of an additional therapy, e.g., a SOC therapy. In some embodiments of concurrent administration of a GDF15-GFRAL Pathway Modulating Agent (e.g., an Antibody Agent such as a GDF15 Antibody Agent), or a composition comprising the same and an additional therapy, GDF15-GFRAL Pathway Modulating Agent (e.g., an Antibody Agent such as a GDF15 Antibody Agent), or a composition comprising the same is administered first followed by an additional therapy. In some embodiments of concurrent administration of a GDF15-GFRAL Pathway Modulating Agent (e.g., an Antibody Agent such as a GDF15 Antibody Agent), or a composition comprising the same and an additional therapy, an additional therapy is administered first followed by a GDF15-GFRAL Pathway Modulating Agent (e.g., an Antibody Agent such as a GDF15 Antibody Agent) disclosed herein, or a composition comprising the same.

In some embodiments, when a subject has CVS, an additional therapy, e.g., a SOC, comprises one or more of small molecule therapy, targeted therapy such as antibody therapy, immunotherapy or other therapies, e.g., as are known in the field and appreciated by one with skill in the art.

In some embodiments, when a subject has CHS, an additional therapy, e.g., a SOC, comprises one or more of small molecule therapy, topical creams, or other therapies, e.g., as are known in the field and appreciated by one with skill in the art.

In some embodiments, when a subject has MAN/V, an additional therapy, e.g., a SOC, comprises one or more of small molecule therapy, targeted therapy such as antibody therapy, immunotherapy or other therapies, e.g., as are known in the field and appreciated by one with skill in the art.

In some embodiments, when a subject has nausea, an additional therapy, e.g., a SOC comprises an anti-emetic, e.g., one or more of a 5-hydroxytryptamine-3 (5-HT3) receptor antagonist, a dopamine antagonist, a neurokinin-1 (NK-1) receptor antagonist, an antihistamine, a cannabinoid, a benzodiazepine, a CGRP antagonist, non-steroidal anti-inflammatory drug, an anticholinergic, or a steroid.

In some embodiments, when a subject has weight loss, an additional therapy, e.g., a SOC, comprises nutrition management, nutritional supplements, and/or an appetite stimulant.

In some embodiments, when a subject has loss of appetite, an additional therapy, e.g., a SOC comprises an appetite stimulant, e.g., a supplement such as Zinc, a cannabinoid, a synthetic progestin, a testosterone derivative, and/or a steroid.

### INCORPORATION BY REFERENCE

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXEMPLARY EMBODIMENTS

Embodiment 1. A method of treating and/or preventing hyperemesis gravidarum (HG) comprising administering to a subject a GDF15-GFRAL Pathway Modulating Agent.

Embodiment 2. A method of treating and/or preventing Cyclic Vomiting Syndrome (CVS) comprising administering to a subject a GDF15-GFRAL Pathway Modulating Agent.

Embodiment 3. A method of treating and/or preventing Cannabinoid Hyperemesis Syndrome (CHS) comprising administering to a subject a GDF15-GFRAL Pathway Modulating Agent.

Embodiment 4. A method of treating and/or preventing Migraine Associated Nausea/Vomiting (MAN/V) comprising administering to a subject a GDF15-GFRAL Pathway Modulating Agent.

Embodiment 5. A method of treating and/or preventing a Mitochondrial Disorder (MID) comprising administering to a subject a GDF15-GFRAL Pathway Modulating Agent.

Embodiment 6. The method of any one of the preceding embodiments, wherein:
(a) the step of modulating comprises: administering a composition that comprises or delivers a GDF15-GFRAL Pathway Modulating Agent to a subject suffering from or susceptible to HG, CVS, CHS, MID, and/or MAN/V; and/or
(b) the administering is according to a dosing regimen established to reduce severity or incidence of, and/or delay onset of (“ameliorate”) of one or more symptoms symptom associated with HG, CVS, CHS, MID, and/or MAN/V.

Embodiment 7. The method of embodiment 6, wherein the symptom is chosen from: nausea, retching, emesis, migraine, weight loss, pain, muscle loss, gastric stasis, reduced fetal growth or a combination thereof.

Embodiment 8. The method of any one of the preceding embodiments, wherein the subject has been demonstrated to show elevated GDF15 level and/or activity.

Embodiment 9. The method of embodiment 8, wherein increased GDF15 comprises a level of about 1 ng/ml or more, e.g., as evaluated in a sample from a subject, e.g., a blood, plasma, serum, cerebrospinal fluid (CSF), or urine sample.

Embodiment 10. A method of ameliorating a symptom associated with hyperemesis gravidarum (HG) comprising administering to a subject a GDF15-GFRAL Pathway Modulating Agent.

Embodiment 11. A method of ameliorating a symptom associated with Cyclic Vomiting Syndrome (CVS), comprising administering to a subject a GDF15-GFRAL Pathway Modulating Agent.

Embodiment 12. A method of ameliorating a symptom associated with Cannabinoid Hyperemesis Syndrome (CHS), comprising administering to a subject a GDF15-GFRAL Pathway Modulating Agent.

Embodiment 13. A method of ameliorating a symptom associated with Migraine Associated Nausea/Vomiting (MAN/V), comprising administering to a subject a GDF15-GFRAL Pathway Modulating Agent.

Embodiment 14 A method of ameliorating a symptom associated with a Mitochondrial Disorder (MID) comprising administering to a subject a GDF15-GFRAL Pathway Modulating Agent.

Embodiment 15. The method of any one of embodiments 10-14, wherein the symptom is chosen from: nausea, retching, emesis, migraine, weight loss, pain, gastric stasis, restricted fetal growth, or a combination thereof.

Embodiment 16. The method of any one of embodiments 10-15, wherein the subject has a condition or disorder associated with increased GDF15.

Embodiment 17. The method of any one of embodiments 1-16, wherein the subject is pregnant.

Embodiment 18. The method of embodiment 16 or 17, wherein increased GDF15 comprises a level of about 1 ng/ml or more, e.g., as evaluated in a sample from a subject, e.g., a blood, plasma, serum, cerebrospinal fluid (CSF), or urine sample.

Embodiment 19. The method of any one of the preceding embodiments, wherein the GDF15-GFRAL Pathway Modulating Agent is characterized in that when administered to the subject it reduces the level and/or activity of GDF15 relative to a comparator.

Embodiment 20. The method of embodiment 19, wherein the level of free and/or active GDF15 is reduced.

Embodiment 21. The method of embodiment 19, wherein the level of free and active GFRAL is reduced.

Embodiment 22. The method of any one of embodiments 10-21, wherein the symptom is reduced relative to a comparator.

Embodiment 23. The method of embodiment 22, wherein the comparator comprises an otherwise similar cell, tissue or subject not administered a GDF15-GFRAL Pathway Modulating Agent or administered a different GDF15-GFRAL Pathway Modulating Agent.

Embodiment 24. The method of any one of embodiments 15-23, wherein the symptom frequency and/or severity is reduced by about 1.5 fold to about 10-fold.

Embodiment 25. The method of any one of embodiments 15-24, wherein the symptom is reduced by about 5%, about 10%, about 20%, about 30%, about 40%, about 50%, about 60%, about 70%, about 80%, about 90% or about 100%.

Embodiment 26. The method of any one of the preceding embodiments, further comprising determining a level and/or activity of GDF15 in a sample from the subject.

Embodiment 27. The method of embodiment 26, wherein an increased level and/or activity of GDF15 is determined relative to a comparator.

Embodiment 28. The method of any one of the preceding embodiments, wherein a composition comprising the GDF15-GFRAL Pathway Modulating Agent is administered to the subject.

Embodiment 29. The method of embodiment 28, wherein the composition comprises a pharmaceutical composition.

Embodiment 30. The method of embodiment 29, wherein the pharmaceutical composition comprises an excipient and/or a pharmaceutically acceptable carrier.

Embodiment 31. The method of embodiment 29 or 30, wherein the pharmaceutical composition is formulated in one or more unit dosage forms.

Embodiment 32. A method comprising steps of:
determining a GDF15 expression or activity level in a subject suffering from or susceptible to CVS; and
administering GDF15-GFRAL Pathway Modulating therapy to the subject if the level is above 1 ng/mL.

Embodiment 33. A method comprising steps of:
determining a GDF15 expression or activity level in a subject suffering from or susceptible to CHS; and
administering GDF15-GFRAL Pathway Modulating therapy to the subject if the level is above 1 ng/mL.

Embodiment 34. A method comprising steps of:
determining a GDF15 expression or activity level in a subject suffering from or susceptible to MAN/V; and
administering GDF15-GFRAL Pathway Modulating therapy to the subject if the level is above 1 ng/mL.

Embodiment 35. A method comprising steps of:
determining a GDF15 expression or activity level in a subject suffering from or susceptible to MID; and
administering GDF15-GFRAL Pathway Modulating therapy to the subject if the level is above 1 ng/mL.

Embodiment 36. The method of any one of embodiments 32-35, wherein the step of administering comprises administering a composition that comprises or delivers a GDF15-GFRAL Pathway Modulating Agent to a subject suffering from or susceptible to HG, CVS, CHS, MID, and/or MAN/V.

Embodiment 37. The method of any one of embodiments 32-35, wherein the administering is according to a dosing regimen established to reduce severity or incidence of, and/or delay onset of (“ameliorate”) of one or more symptoms symptom associated with HG, CVS, CHS, MID, and/or MAN/V.

Embodiment 38. The method of any one of embodiments 32-35, wherein the subject has recently experienced or is experiencing nausea and/or emesis.

Embodiment 39. The method of any one of the preceding embodiments, wherein the GDF15-GFRAL Pathway Modulating Agent comprises nucleic acids, polypeptides, and/or small molecules.

Embodiment 40. The method of embodiment 39, wherein the GDF15-GFRAL Pathway Modulating Agent binds to genetic variants of GDF-15, GFRAL and RET associated with nausea, vomiting and emesis.

Embodiment 41. The method of any one of the preceding embodiments, wherein the GDF15-GFRAL Pathway Modulating Agent is or comprises a GDF15 Antibody Agent.

Embodiment 42. The method of embodiment 41, wherein the GDF15 Antibody Agent comprises a polypeptide that binds to human growth differentiation factor 15 (GDF15), comprising at least one light chain complementarity determining region (LC CDR) and/or at least one heavy chain complementary determining region (HC CDR).

Embodiment 43. The method of any one of embodiments 1-40, wherein the GDF15-GFRAL Pathway Modulating Agent is or comprises a GDF15 nucleic acid agent

Embodiment 44. The method of any one of embodiments 1-40, wherein the GDF15-GFRAL Pathway Modulating Agent is or comprises a GDF15 small molecule agent.

Embodiment 45. The method of embodiment 44, wherein the GDF15 small molecule agent is or comprises a GDF15 inhibitor or negative allosteric modulator.

Embodiment 46. The method of any one of embodiments 1-40, wherein the GDF15-GFRAL Pathway Modulating Agent is or comprises a GFRAL Antibody Agent.

Embodiment 47. The method of embodiment 46, wherein the GFRAL Antibody Agent comprises a polypeptide that binds to human GFRAL, comprising at least one light chain complementarity determining region (LC CDR) and/or at least one heavy chain complementary determining region (HC CDR).

Embodiment 48. The method of any one of embodiments 1-40, wherein the GDF15-GFRAL Pathway Modulating Agent is or comprises a GFRAL nucleic acid agent

Embodiment 49. The method of any one of embodiments 1-40, wherein the GDF15-GFRAL Pathway Modulating Agent is or comprises a GFRAL small molecule agent.

Embodiment 50. The method of embodiment 49, wherein the GFRAL small molecule agent is or comprises a GFRAL inhibitor or negative allosteric modulator.

Embodiment 51. The method of any one of embodiments 1-40, wherein the GDF15-GFRAL Pathway Modulating Agent is or comprises a RET Antibody Agent.

Embodiment 52. The method of embodiment 51, wherein the RET Antibody Agent comprises a polypeptide that binds to human RET comprising at least one light chain complementarity determining region (LC CDR) and/or at least one heavy chain complementary determining region (HC CDR).

Embodiment 53. The method of any one of embodiments 1-40, wherein the GDF15-GFRAL Pathway Modulating Agent is or comprises a RET nucleic acid agent

Embodiment 54. The method of any one of embodiments 1-40, wherein the GDF15-GFRAL Pathway Modulating Agent is or comprises a RET small molecule agent.

Embodiment 55. The method of embodiment 54, wherein the RET small molecule agent is or comprises a RET inhibitor or negative allosteric modulator.

Embodiment 56. The method of any one of embodiments 41-42, 46-47 or 51-52, wherein the Antibody Agent comprises:
(i) an intact IgA, IgG, IgD, IgE or IgM antibody;
(ii) an antibody fragment;
(iii) a single domain antibody;
(iv) a single chain Fv; or
(v) a polypeptide comprising antigen binding specific fused to a Fc domain.

Embodiment 57. The method of any one of embodiments 41-42, 46-47 or 51-52, wherein the Antibody Agent comprises:
(i) one, two, or three LC CDRs;
(ii) one, two or three HC CDRs; or
(iii) both (i) and (ii).

Embodiment 58. The method of embodiment 56 or 57, wherein the antibody agent comprises:
(i) an LC CDR1 sequence provided in Table 1, e.g., any one of SEQ ID NOs: 92, 101, 117, 125, 129, 137, 212;
(ii) a sequence with at least 85%, 86%, 87%, 88%, 89%, 90%, 91%, 92%, 93%, 94%, 95%, 96%, 97%, 98%, 99% identity to an LC CDR1 sequence provided in Table 1, e.g., any one of SEQ ID NOs92, 101, 117, 125, 129, 137, 212; or
(iii) a sequence having at least 5, 10, or 20 substitutions relative to an LC CDR1 sequence provided in Table 1, e.g., any one of SEQ ID NOs: 92, 101, 117, 125, 129, 137, 212.

Embodiment 59. The method of any one of embodiments 56-58, wherein the antibody agent comprises:
(i) an LC CDR2 sequence provided in Table 1, e.g., any one of SEQ ID NOs: 93, 102 or 130;
(ii) a sequence with at least 85%, 86%, 87%, 88%, 89%, 90%, 91%, 92%, 93%, 94%, 95%, 96%, 97%, 98%, 99% identity to an LC CDR2 sequence provided in Table 1 e.g., any one of SEQ ID NOs: 93, 102 or 130; or
(iii) a sequence having at least 5, 10, or 20 substitutions relative to an LC CDR2 sequence provided in Table 1, e.g., any one of SEQ ID NOs: 93, 102 or 130.

Embodiment 60. The method of any one of embodiments 56-59, wherein the antibody agent comprises:
(i) an LC CDR3 sequence provided in Table 1, e.g., any one of SEQ ID NOs: 94, 103, 110, 118, 126, 131, 138, 204, 208 or 217;
(ii) a sequence with at least 85%, 86%, 87%, 88%, 89%, 90%, 91%, 92%, 93%, 94%, 95%, 96%, 97%, 98%, 99% identity to an LC CDR3 sequence provided in Table 1, e.g., any one of SEQ ID NOs: 94, 103, 110, 118, 126, 131, 138, 204, 208 or 217; or
(iii) a sequence having at least 5, 10, or 20 substitutions relative to an LC CDR3 sequence provided in Table 1, e.g., any one of SEQ ID NOs: 94, 103, 110, 118, 126, 131,138, 204, 208 or 217.

Embodiment 61. The method of any one of embodiments 56-60, wherein the antibody agent comprises a LC CDR1, LC CDR2 and/or LC CDR3 and is capable of binding specifically to GDF15.

Embodiment 62. The method of any one of embodiments 56-61, wherein the antibody agent comprises
(i) an LC CDR1, LC CDR2, and LC CDR3 sequence provided in Table 1;
(ii) a sequence with at least 85%, 86%, 87%, 88%, 89%, 90%, 91%, 92%, 93%, 94%, 95%, 96%, 97%, 98%, 99% identity to an LC CDR1, LC CDR2, and LC CDR3 sequence provided in Table 1; or
(iii) a sequence having at least 5, 10, or 20 substitutions relative to an LC CDR1, LC CDR2, and LC CDR3 sequence provided in Table 1.

Embodiment 63. The method of any one of embodiments 56-62, wherein the antibody agent comprises
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92;
(ii) an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and/or
(iii) an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94.

Embodiment 64. The method of any one of embodiments 56-62, wherein the antibody agent comprises
(i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101;
(ii) an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and/or
(iii) an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103.

Embodiment 65. The method of any one of embodiments 56-62, wherein the antibody agent comprises
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92;
(ii) an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and/or
(iii) an LC CDR3 of SEQ ID NO: 110, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 110.

Embodiment 66. The method of any one of embodiments 56-62, wherein the antibody agent comprises
(i) an LC CDR1 of SEQ ID NO: 117, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 117;
(ii) an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and/or
(iii) an LC CDR3 of SEQ ID NO: 118, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 118.

Embodiment 67. The method of any one of embodiments 56-62, wherein the antibody agent comprises
(i) an LC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125;
(ii) an LC CDR2 of SEQ ID NO: 102 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and/or
(iii) an LC CDR3 of SEQ ID NO: 126, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 126.

Embodiment 68. The method of any one of embodiments 56-62, wherein the antibody agent comprises
(i) an LC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129;
(ii) an LC CDR2 of SEQ ID NO: 130 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and/or
(iii) an LC CDR3 of SEQ ID NO: 131, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 131.

Embodiment 69. The method of any one of embodiments 56-62, wherein the antibody agent comprises
(i) an LC CDR1 of SEQ ID NO: 137, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 137;
(ii) an LC CDR2 of SEQ ID NO: 102 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and/or
(iii) an LC CDR3 of SEQ ID NO: 138, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 138.

Embodiment 70. The method of any one of embodiments 56-62, wherein the antibody agent comprises
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92;
(ii) an LC CDR2 of SEQ ID NO: 93 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and/or
(iii) an LC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204.

Embodiment 71. The method of any one of embodiments 56-62, wherein the antibody agent comprises
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92;
(ii) an LC CDR2 of SEQ ID NO: 93 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and/or
(iii) an LC CDR3 of SEQ ID NO: 208, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 208.

Embodiment 72. The method of any one of embodiments 56-62, wherein the antibody agent comprises
(i) an LC CDR1 of SEQ ID NO: 212, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 212;
(ii) an LC CDR2 of SEQ ID NO: 102 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and/or
(iii) an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103.

Embodiment 73. The method of any one of embodiments 56-62, wherein the antibody agent comprises:
(i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101;
(ii) an LC CDR2 of SEQ ID NO: 102 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and/or
(iii) an LC CDR3 of SEQ ID NO: 217, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 217.

Embodiment 74. The method of any one of embodiments 56-73, wherein the antibody agent comprises:
(i) an HC CDR1 sequence provided in Table 2, e.g., SEQ ID NO: 1, SEQ ID NO: 10, SEQ ID NO: 14, SEQ ID NO: 18, SEQ ID NO: 22, SEQ ID NO: 31, SEQ ID NO: 40, SEQ ID NO: 49, SEQ ID NO: 56, SEQ ID NO: 63, SEQ ID NO: 68, SEQ ID NO: 73, SEQ ID NO: 78, SEQ ID NO: 82 or SEQ ID NO: 88;
(ii) a sequence with at least 85%, 86%, 87%, 88%, 89%, 90%, 91%, 92%, 93%, 94%, 95%, 96%, 97%, 98%, 99% identity to an HC CDR1 sequence provided in Table 2, e.g., SEQ ID NO: 1, SEQ ID NO: 10, SEQ ID NO: 14, SEQ ID NO: 18, SEQ ID NO: 22, SEQ ID NO: 31, SEQ ID NO: 40, SEQ ID NO: 49, SEQ ID NO: 56, SEQ ID NO: 63, SEQ ID NO: 68, SEQ ID NO: 73, SEQ ID NO: 78, SEQ ID NO: 82 or SEQ ID NO: 88; or
(iii) a sequence having at least 5, 10, or 20 substitutions compared to an HC CDR1 sequence provided in Table 2, e.g., SEQ ID NO: 1, SEQ ID NO: 10, SEQ ID NO: 14, SEQ ID NO: 18, SEQ ID NO: 22, SEQ ID NO: 31, SEQ ID NO: 40, SEQ ID NO: 49, SEQ ID NO: 56, SEQ ID NO: 63, SEQ ID NO: 68, SEQ ID NO: 73, SEQ ID NO: 78, SEQ ID NO: 82 or SEQ ID NO: 88.

Embodiment 75. The method of any one of embodiments 56-74, wherein the antibody agent comprises
(i) an HC CDR2 sequence provided in Table 2, e.g., SEQ ID NO: 2, SEQ ID NO: 11, SEQ ID NO: 15, SEQ ID NO: 19, SEQ ID NO: 23, SEQ ID NO: 32, SEQ ID NO: 50, SEQ ID NO: 57, SEQ ID NO: 60, SEQ ID NO:64, SEQ ID NO: 69, SEQ ID NO: 74, SEQ ID NO: 79, SEQ ID NO: 83 or SEQ ID NO: 200;
(ii) a sequence with at least 85%, 86%, 87%, 88%, 89%, 90%, 91%, 92%, 93%, 94%, 95%, 96%, 97%, 98%, 99% identity to an HC CDR2 sequence provided in Table 2, e.g., SEQ ID NO: 2, SEQ ID NO: 11, SEQ ID NO: 15, SEQ ID NO: 19, SEQ ID NO: 23, SEQ ID NO: 32, SEQ ID NO: 50, SEQ ID NO: 57, SEQ ID NO: 60, SEQ ID NO:64, SEQ ID NO: 69, SEQ ID NO: 74, SEQ ID NO: 79, SEQ ID NO: 83 or SEQ ID NO: 200; or
(iii) a sequence having at least 5, 10, or 20 substitutions compared to an HC CDR2 sequence provided in Table 2, e.g., SEQ ID NO: 2, SEQ ID NO: 11, SEQ ID NO: 15, SEQ ID NO: 19, SEQ ID NO: 23, SEQ ID NO: 32, SEQ ID NO: 50, SEQ ID NO: 57, SEQ ID NO: 60, SEQ ID NO:64, SEQ ID NO: 69, SEQ ID NO: 74, SEQ ID NO: 79, SEQ ID NO: 83 or SEQ ID NO: 200.

Embodiment 76. The method of any one of embodiments 56-75, wherein the antibody agent comprises
(i) an HC CDR3 sequence provided in Table 2, e.g., SEQ ID NO: 3, SEQ ID NO: 191, SEQ ID NO: 192, SEQ ID NO: 193, SEQ ID NO: 24, SEQ ID NO: 33, SEQ ID NO: 42, SEQ ID NO: 51, SEQ ID NO: 65, SEQ ID NO:70, SEQ ID NO:75, SEQ ID NO:84, SEQ ID NO: 89;
(ii) a sequence with at least 85%, 86%, 87%, 88%, 89%, 90%, 91%, 92%, 93%, 94%, 95%, 96%, 97%, 98%, 99% identity to an HC CDR3 sequence provided in Table 2, e.g., SEQ ID NO: 3, SEQ ID NO: 191, SEQ ID NO: 192, SEQ ID NO: 193, SEQ ID NO: 24, SEQ ID NO: 33, SEQ ID NO: 42, SEQ ID NO: 51, SEQ ID NO: 65, SEQ ID NO:70, SEQ ID NO:75, SEQ ID NO:84, SEQ ID NO:89; or
(iii) a sequence having at least 5, 10, or 20 substitutions compared to an HC CDR3 sequence provided in Table 2, e.g., SEQ ID NO: 3, SEQ ID NO: 191, SEQ ID NO: 192, SEQ ID NO: 193, SEQ ID NO: 24, SEQ ID NO: 33, SEQ ID NO: 42, SEQ ID NO: 51, SEQ ID NO: 65, SEQ ID NO:70, SEQ ID NO:75, SEQ ID NO:84, SEQ ID NO:89.

Embodiment 77. The method of any one of embodiments 56-76, wherein the antibody agent comprising an HC CDR1, an HC CDR2 and/or an HC CDR3 is able to specifically bind to GDF15.

Embodiment 78. The method of any one of embodiments 56-77, wherein the antibody agent comprises
(i) an HC CDR1, HC CDR2, and HC CDR3 sequence provided in Table 2;
(ii) a sequence with at least 85%, 86%, 87%, 88%, 89%, 90%, 91%, 92%, 93%, 94%, 95%, 96%, 97%, 98%, or 99% identity to an HC CDR1, HC CDR2, and HC CDR3 sequence provided in Table 2;
(iii) a sequence having at least 5, 10, or 20 substitutions relative to an HC CDR1, HC CDR2, and HC CDR3 sequence provided in Table 2.

Embodiment 79. The method of any one of embodiments 56-77, wherein the antibody agent comprises
(i) an HC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1;
(ii) an HC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and/or
(iii) an HC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3.

Embodiment 80. The method of any one of embodiments 56-77, wherein the antibody agent comprises
(i) an HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10;
(ii) an HC CDR2 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; and/or
(iii) an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

Embodiment 81. The method of any one of embodiments 56-77, wherein the antibody agent comprises
(i) an HC CDR1 of SEQ ID NO: 14, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 14;
(ii) an HC CDR2 of SEQ ID NO: 15, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 15; and/or
(iii) an HC CDR3 of SEQ ID NO: 192, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 192.

Embodiment 82. The method of any one of embodiments 56-77, wherein the antibody agent comprises
i) an HC CDR1 of SEQ ID NO: 18, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 18;
(ii) an HC CDR2 of SEQ ID NO: 19, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 19; and/or
(iii) an HC CDR3 of SEQ ID NO: 193, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 193.

Embodiment 83. The method of any one of embodiments 56-77, wherein the antibody agent comprises
(i) an HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22;
(ii) an HC CDR2 of SEQ ID NO: 23, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 23; and/or
(iii) an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

Embodiment 84. The method of any one of embodiments 56-77, wherein the antibody agent comprises
(i) an HC CDR1 of SEQ ID NO: 31, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 31;
(ii) an HC CDR2 of SEQ ID NO: 32, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 32; and/or
(iii) an HC CDR3 of SEQ ID NO: 33, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 33.

Embodiment 85. The method of any one of embodiments 56-77, wherein the antibody agent comprises
(i) an HC CDR1 of SEQ ID NO: 40, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 40;
(ii) an HC CDR2 of SEQ ID NO: 32, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 32; and/or
(iii) an HC CDR3 of SEQ ID NO: 42, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 42.

Embodiment 86 The method of any one of embodiments 56-77, wherein the antibody agent comprises
(i) an HC CDR1 of SEQ ID NO: 49, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 49;
(ii) an HC CDR2 of SEQ ID NO: 50, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 50; and/or
(iii) an HC CDR3 of SEQ ID NO: 51, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 51.

Embodiment 87. The method of any one of embodiments 56-77, wherein the antibody agent comprises
(i) an HC CDR1 of SEQ ID NO: 56, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 56;
(ii) an HC CDR2 of SEQ ID NO: 57, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 57; and/or
(iii) an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

Embodiment 88. The method of any one of embodiments 56-77, wherein the antibody agent comprises
(i) an HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22;
(ii) an HC CDR2 of SEQ ID NO: 60, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 60; and/or
(iii) an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

Embodiment 89. The method of any one of embodiments 56-77, wherein the antibody agent comprises
(i) an HC CDR1 of SEQ ID NO: 63, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 63;
(ii) an HC CDR2 of SEQ ID NO: 64, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 64; and/or
(iii) an HC CDR3 of SEQ ID NO: 65, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 65.

Embodiment 90. The method of any one of embodiments 56-77, wherein the antibody agent comprises
(i) an HC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68;
(ii) an HC CDR2 of SEQ ID NO: 69, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and/or
(iii) an HC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70.

Embodiment 91. The method of any one of embodiments 56-77, wherein the antibody agent comprises
(i) an HC CDR1 of SEQ ID NO: 73, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 73;
(ii) an HC CDR2 of SEQ ID NO: 74, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 74; and/or
(iii) an HC CDR3 of SEQ ID NO: 75, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 75.

Embodiment 92. The method of any one of embodiments 56-77, wherein the antibody agent comprises
(i) an HC CDR1 of SEQ ID NO: 78, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 78;
(ii) an HC CDR2 of SEQ ID NO: 79, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 79; and/or
(iii) an HC CDR3 of SEQ ID NO: 75, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 75.

Embodiment 93. The method of any one of embodiments 56-77, wherein the antibody agent comprises
(i) an HC CDR1 of SEQ ID NO: 82, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 82;
(ii) an HC CDR2 of SEQ ID NO: 83, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 83; and/or
(iii) an HC CDR3 of SEQ ID NO: 84, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 84.

Embodiment 94. The method of any one of embodiments 56-77, wherein the antibody agent comprises
(i) an HC CDR1 of SEQ ID NO: 88, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 88;
(ii) an HC CDR2 of SEQ ID NO: 57, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 57; and/or
(iii) an HC CDR3 of SEQ ID NO: 89, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 89.

Embodiment 95. The method of any one of embodiments 56-77, wherein the
(i) an HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10;
(ii) an HC CDR2 of SEQ ID NO: 200, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 200; and/or
(iii) an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

Embodiment 96. The method of any one of embodiments 56-95, wherein the antibody agent comprises one, two, or three LC CDRs provided in Table 1 or a sequence with at least 85%, 86%, 87%, 88%, 89%, 90%, 91%, 92%, 93%, 94%, 95%, 96%, 97%, 98%, or 99% thereto; and one, two, or three HC CDRs provided in Table 2, or a sequence with at least 85%, 86%, 87%, 88%, 89%, 90%, 91%, 92%, 93%, 94%, 95%, 96%, 97%, 98%, or 99% thereto.

Embodiment 97. The method of any one of embodiments 56-96, wherein the antibody agent comprises three LC CDRs provided in Table 1 or a sequence with at least 85%, 86%, 87%, 88%, 89%, 90%, 91%, 92%, 93%, 94%, 95%, 96%, 97%, 98%, or 99% thereto; and three HC CDRs provided in Table 2, or a sequence with at least 85%, 86%, 87%, 88%, 89%, 90%, 91%, 92%, 93%, 94%, 95%, 96%, 97%, 98%, or 99% thereto.

Embodiment 98. The method of any one of embodiments 56-97, wherein the antibody agent comprises
(a) a light chain comprising:
(i) an LC CDR1 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR1 provided in Table 1;
(ii) an LC CDR2 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR2 provided in Table 1; and/or
(iii) an LC CDR3 provided in Table 1 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an LC CDR3 provided in Table 1; and
(b) a heavy chain comprising:
(i) an HC CDR1 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an HC CDR1 provided in Table 2;
(ii) an HC CDR2 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an HC CDR2 provided in Table 2; and/or
(iii) an HC CDR3 provided in Table 2 or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to an HC CDR3 provided in Table 2.

Embodiment 99. The method of any one of embodiments 56-98, wherein the Antibody Agent comprises:
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and
(ii) an HC CDR1 of SEQ ID NO: 1, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 1; an HC CDR2 of SEQ ID NO: 2, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 2; and an HC CDR3 of SEQ ID NO: 3, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 3.

Embodiment 100. The method of any one of embodiments 56-98, wherein the Antibody Agent comprises:
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and
(ii) an HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10; an HC CDR2 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; and an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

Embodiment 101. The method of any one of embodiments 56-98, wherein the Antibody Agent comprises:
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and
(ii) an HC CDR1 of SEQ ID NO: 14, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 14; an HC CDR2 of SEQ ID NO: 15, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 15; and an HC CDR3 of SEQ ID NO: 192, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 192.

Embodiment 102. The method of any one of embodiments 56-98, wherein the Antibody Agent comprises:
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 94, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 94; and
(ii) a HC CDR1 of SEQ ID NO: 18, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 18; an HC CDR2 of SEQ ID NO: 19, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 19; and an HC CDR3 of SEQ ID NO: 193, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 193.

Embodiment 103. The method of any one of embodiments 56-98, wherein the Antibody Agent comprises:
(i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; and
(ii) an HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; an HC CDR2 of SEQ ID NO: 23, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 23; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

Embodiment 104. The method of any one of embodiments 56-98, wherein the Antibody Agent comprises:
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 110, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 110; and
(ii) an HC CDR1 of SEQ ID NO: 31, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 31; an HC CDR2 of SEQ ID NO: 32, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 32; and an HC CDR3 of SEQ ID NO: 33, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 33.

Embodiment 105. The method of any one of embodiments 56-98, wherein the Antibody Agent comprises:
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 110, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 110; and
(ii) an HC CDR1 of SEQ ID NO: 40, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 40; an HC CDR2 of SEQ ID NO: 32, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 32; and an HC CDR3 of SEQ ID NO: 42, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 42.

Embodiment 106. The method of any one of embodiments 56-98, wherein the Antibody Agent comprises:
(i) an LC CDR1 of SEQ ID NO: 117, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 117; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 118, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 118; and
(ii) an HC CDR1 of SEQ ID NO: 49, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 49; an HC CDR2 of SEQ ID NO: 50, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 50; and an HC CDR3 of SEQ ID NO: 51, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 51.

Embodiment 107. The method of any one of embodiments 56-98, wherein the Antibody Agent comprises:
(i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; and
(ii) an HC CDR1 of SEQ ID NO: 56, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 56; an HC CDR2 of SEQ ID NO: 57, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 57; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

Embodiment 108. The method of any one of embodiments 56-98, wherein the Antibody Agent comprises:
(i) an LC CDR1 of SEQ ID NO: 101, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 101; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; and
(ii) an HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; an HC CDR2 of SEQ ID NO: 60, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 60; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

Embodiment 109. The method of any one of embodiments 56-98, wherein the Antibody Agent comprises:
(i) an LC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 126, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 126; and
(ii) an HC CDR1 of SEQ ID NO: 63, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 63; an HC CDR2 of SEQ ID NO: 64, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 64; and an HC CDR3 of SEQ ID NO: 65, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 65.

Embodiment 110. The method of any one of embodiments 56-98, wherein the Antibody Agent comprises:
(i) an LC CDR1 of SEQ ID NO: 125, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 125; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 126, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 126; and
(ii) an HC CDR1 of SEQ ID NO: 68, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 68; an HC CDR2 of SEQ ID NO: 69, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 69; and an HC CDR3 of SEQ ID NO: 70, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 70.

Embodiment 111. The method of any one of embodiments 56-98, wherein the Antibody Agent comprises:
(i) an LC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129; an LC CDR2 of SEQ ID NO: 130, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and an LC CDR3 of SEQ ID NO: 131, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 131; and
(ii) an HC CDR1 of SEQ ID NO: 73, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 73; an HC CDR2 of SEQ ID NO: 74, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 74; and an HC CDR3 of SEQ ID NO: 75, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 75.

Embodiment 112. The method of any one of embodiments 56-98, wherein the Antibody Agent comprises:
(i) an LC CDR1 of SEQ ID NO: 129, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 129; an LC CDR2 of SEQ ID NO: 130, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 130; and an LC CDR3 of SEQ ID NO: 131, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 131; and
(ii) an HC CDR1 of SEQ ID NO: 78, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 78; an HC CDR2 of SEQ ID NO: 79, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 79; and an HC CDR3 of SEQ ID NO: 75, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 75.

Embodiment 113. The method of any one of embodiments 56-98, wherein the Antibody Agent comprises:
(i) an LC CDR1 of SEQ ID NO: 137, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 137; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 138, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 138; and
(ii) an HC CDR1 of SEQ ID NO: 82, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 82; an HC CDR2 of SEQ ID NO: 83, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 83; and an HC CDR3 of SEQ ID NO: 84, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 84.

Embodiment 114. The method of any one of embodiments 56-98, wherein the Antibody Agent comprises:
(i) an LC CDR1 of SEQ ID NO: 137, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 137; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 138, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 138; and
(ii) an HC CDR1 of SEQ ID NO: 88, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 88; an HC CDR2 of SEQ ID NO: 57, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 57; and an HC CDR3 of SEQ ID NO: 89, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 89.

Embodiment 115. The method of any one of embodiments 56-98, wherein the Antibody Agent comprises:
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 204, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 204; and
(ii) an HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10; an HC CDR2 of SEQ ID NO: 11, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 11; and an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

Embodiment 116. The method of any one of embodiments 56-98, wherein the Antibody Agent comprises:
(i) an LC CDR1 of SEQ ID NO: 92, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 92; an LC CDR2 of SEQ ID NO: 93, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 93; and an LC CDR3 of SEQ ID NO: 208, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 208; and
ii) an HC CDR1 of SEQ ID NO: 10, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 10; an HC CDR2 of SEQ ID NO: 200, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 200; and an HC CDR3 of SEQ ID NO: 191, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 191.

Embodiment 117. The method of any one of embodiments 56-98, wherein the Antibody Agent comprises:
(i) an LC CDR1 of SEQ ID NO: 212, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 212; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 103, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 103; and
(ii) an HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; an HC CDR2 of SEQ ID NO: 23, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 23; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

Embodiment 118. The method of any one of embodiments 56-98, wherein the Antibody Agent comprises:
88%, 89%, 90%, 91%, 92%, 93%, 94%, 95%, 96%, 97%, 98%, or 99% thereto or a sequence having at least 5, 10, or 20 substitutions relative to SEQ ID NO: 101; an LC CDR2 of SEQ ID NO: 102, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 102; and an LC CDR3 of SEQ ID NO: 217, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 217; and
(ii) an HC CDR1 of SEQ ID NO: 22, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 22; an HC CDR2 of SEQ ID NO: 23, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 23; and an HC CDR3 of SEQ ID NO: 24, or a sequence with at least 85%, 86%, 87%, 88%, 89%, 90%, 91%, 92%, 93%, 94%, 95%, 96%, 97%, 98%, or 99% thereto or a sequence having at least 5, 10, or 20 substitutions relative to SEQ ID NO: 24.

Embodiment 119. The method of any one of embodiments 56-118, wherein the Antibody Agent comprises:
(a) a light chain (LC) comprising:
(i) a variable region (VL) comprising one, two, or three LC CDRs, and at least one framework region (FR); and
(ii) a constant region (CL); and/or
(b) a heavy chain (HC) comprising:
(i) a variable region (VH) comprising one, two, three HC CDRs and at least one framework region; and
(ii) a sequence for at least one constant region (CH).

Embodiment 120. The method of any one of embodiments 56-119, wherein the Antibody Agent comprises:
(i) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99;
(ii) the sequence of SEQ ID NO: 107, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 107;
(iii) the sequence of SEQ ID NO: 115, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 115;
(iv) the sequence of SEQ ID NO: 123, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 123;
(v) the sequence of SEQ ID NO: 127, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 127;
(vi) the sequence of SEQ ID NO: 135, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 135;
(vii) the sequence of SEQ ID NO: 139, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 139;
(viii) the sequence of SEQ ID NO: 205, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 205;
(ix) the sequence of SEQ ID NO: 209, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 209;
(x) the sequence of SEQ ID NO: 214, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 214; or
(xi) the sequence of SEQ ID NO: 218, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 218.

Embodiment 121. The method of any one of embodiments 56-120, wherein the Antibody Agent comprises:
(i) the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8;
(ii) the sequence of SEQ ID NO: 12, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 12;
(iii) the sequence of SEQ ID NO: 16, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 16;
(iv) the sequence of SEQ ID NO: 20, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 20;
(v) the sequence of SEQ ID NO: 29, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 29;
(vi) the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38;
(vii) the sequence of SEQ ID NO: 47, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 47;
(viii) the sequence of SEQ ID NO: 54, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 54;
(ix) the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58;
(x) the sequence of SEQ ID NO: 61, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 61;
(xi) the sequence of SEQ ID NO: 66, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 66;
(xii) the sequence of SEQ ID NO: 71, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 71;
(xiii) the sequence of SEQ ID NO: 76, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 76;
(xiv) the sequence of SEQ ID NO: 80, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 80;
(xv) the sequence of SEQ ID NO: 86, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 86;
(xvi) the sequence of SEQ ID NO: 90 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90; or
(xvii) the sequence of SEQ ID NO: 201 or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 201.

Embodiment 122. The method of any one of embodiments 56-121, wherein the Antibody Agent comprises:
(i) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 8, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 8;
(ii) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 12, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 12;
(iii) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 16, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 16;
(iv) the sequence of SEQ ID NO: 99, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 99; and the sequence of SEQ ID NO: 20, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 20;
(v) the sequence of SEQ ID NO: 107, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 107; and the sequence of SEQ ID NO: 29, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 29;
(vi) the sequence of SEQ ID NO: 115, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 115; and the sequence of SEQ ID NO: 38, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 38;
(vii) the sequence of SEQ ID NO: 115, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 115; and the sequence of SEQ ID NO: 47, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 47;
(viii) the sequence of SEQ ID NO: 123, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 123; and the sequence of SEQ ID NO: 54, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 54;
(ix) the sequence of SEQ ID NO: 107, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 107; and the sequence of SEQ ID NO: 58, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 58;
(x) the sequence of SEQ ID NO: 107, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 107; and the sequence of SEQ ID NO: 61, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 61;
(xi) the sequence of SEQ ID NO: 127, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 127; and the sequence of SEQ ID NO: 66, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 66;
(xii) the sequence of SEQ ID NO: 127, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 127; and the sequence of SEQ ID NO: 71, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 71;
(xiii) the sequence of SEQ ID NO: 135, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 135; and the sequence of SEQ ID NO: 76, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 76;
(xiv) the sequence of SEQ ID NO: 135, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 135; and the sequence of SEQ ID NO: 80, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 80;
(xv) the sequence of SEQ ID NO: 139, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 139; and the sequence of SEQ ID NO: 86, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 86;
(xvi) the sequence of SEQ ID NO: 139, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 139; and the sequence of SEQ ID NO: 90, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 90;
(xvii) the sequence of SEQ ID NO: 205, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 205; and the sequence of SEQ ID NO: 12, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 12;
(xviii) the sequence of SEQ ID NO: 209, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 209; and the sequence of SEQ ID NO: 201, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 201;
(xix) the sequence of SEQ ID NO: 214, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 214; and the sequence of SEQ ID NO: 29, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 29; or
(xx) the sequence of SEQ ID NO: 218, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 218; and the sequence of SEQ ID NO: 29, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 29.

Embodiment 123. The method of any one of embodiments 56-122, wherein the Antibody Agent comprises:
(i) the sequence of SEQ ID NO: 143, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;
(ii) the sequence of SEQ ID NO: 144, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;
(iii) the sequence of SEQ ID NO: 145, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 145; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;
(iv) the sequence of SEQ ID NO: 146, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;
(v) the sequence of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163;
(vi) the sequence of SEQ ID NO: 148, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 148; and the sequence of SEQ ID NO: 164, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 164;
(vii) the sequence of SEQ ID NO: 149, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 149; and the sequence of SEQ ID NO: 164, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 164;
(viii) the sequence of SEQ ID NO: 150, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 150; and the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166;
(ix) the sequence of SEQ ID NO: 151, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 151; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163;
(x) the sequence of SEQ ID NO: 152, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 152; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163;
(xi) the sequence of SEQ ID NO: 153, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 153; and the sequence of SEQ ID NO: 169, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 169;
(xii) the sequence of SEQ ID NO: 154, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154; and the sequence of SEQ ID NO: 169, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 169;
(xiii) the sequence of SEQ ID NO: 155, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155; and the sequence of SEQ ID NO: 171, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 171;
(xiv) the sequence of SEQ ID NO: 156, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 156; and the sequence of SEQ ID NO: 171, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 171;
(xv) the sequence of SEQ ID NO: 157, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 157; and the sequence of SEQ ID NO: 173, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 173;
(xvi) the sequence of SEQ ID NO: 158, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 158; and the sequence of SEQ ID NO: 173, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 173;
(xvii) the sequence of SEQ ID NO: 144, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144; and the sequence of SEQ ID NO: 206, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206;
(xviii) the sequence of SEQ ID NO: 202, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 202; and the sequence of SEQ ID NO: 210, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 210;
(xix) the sequence of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147; and the sequence of SEQ ID NO: 215, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 215; or
(xx) the sequence of SEQ ID NO: 147, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147; and the sequence of SEQ ID NO: 219, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 219.

Embodiment 124. The method of any one of embodiments 56-122, wherein the Antibody Agent comprises:
(i) the sequence of SEQ ID NO: 143 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 143 without a terminal lysine; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;
(ii) the sequence of SEQ ID NO: 144 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144 without a terminal lysine; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;
(iii) the sequence of SEQ ID NO: 145 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 145 without a terminal lysine; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;
(iv) the sequence of SEQ ID NO: 146 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 146 without a terminal lysine; and the sequence of SEQ ID NO: 159, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 159;
(v) the sequence of SEQ ID NO: 147 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147 without a terminal lysine; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163;
(vi) the sequence of SEQ ID NO: 148 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 148 without a terminal lysine; and the sequence of SEQ ID NO: 164, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 164;
(vii) the sequence of SEQ ID NO: 149 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 149 without a terminal lysine; and the sequence of SEQ ID NO: 164, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 164;
(viii) the sequence of SEQ ID NO: 150 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 150 without a terminal lysine; and the sequence of SEQ ID NO: 166, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 166;
(ix) the sequence of SEQ ID NO: 151 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 151 without a terminal lysine; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163;
(x) the sequence of SEQ ID NO: 152 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 152 without a terminal lysine; and the sequence of SEQ ID NO: 163, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 163;
(xi) the sequence of SEQ ID NO: 153 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 153 without a terminal lysine; and the sequence of SEQ ID NO: 169, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 169;
(xii) the sequence of SEQ ID NO: 154 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 154 without a terminal lysine; and the sequence of SEQ ID NO: 169, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 169;
(xiii) the sequence of SEQ ID NO: 155 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 155 without a terminal lysine; and the sequence of SEQ ID NO: 171, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 171;
(xiv) the sequence of SEQ ID NO: 156 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 156 without a terminal lysine; and the sequence of SEQ ID NO: 171, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 171;
(xv) the sequence of SEQ ID NO: 157 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 157 without a terminal lysine; and the sequence of SEQ ID NO: 173, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 173; or
(xvi) the sequence of SEQ ID NO: 158 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 158 without a terminal lysine; and the sequence of SEQ ID NO: 173, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 173;
(xvii) the sequence of SEQ ID NO: 144 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 144 without a terminal lysine; and the sequence of SEQ ID NO: 206, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 206;
(xviii) the sequence of SEQ ID NO: 202 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 202 without a terminal lysine; and the sequence of SEQ ID NO: 210, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 210;
(xix) the sequence of SEQ ID NO: 147 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147 without a terminal lysine; and the sequence of SEQ ID NO: 215, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 215; or
(xx) the sequence of SEQ ID NO: 147 without a terminal lysine, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 147 without a terminal lysine; and the sequence of SEQ ID NO: 219, or a sequence with at least 85%, 86%, 87%, 88%, 89%, 90%, 91%, 92%, 93%, 94%, 95%, 96%, 97%, 98%, or 99% thereto, or a sequence having at least 5, 10, or 20 substitutions relative to SEQ ID NO: 219.

Embodiment 125. The method of any one of embodiments 119-124, wherein the at least one CH sequence comprises:

(a) an Fc domain chosen from an Fc domain of an immunoglobulin isotype;
(b) an Fc domain comprising a mutation disclosed herein; and/or
(c) a CH3 domain, optionally wherein the CH3 domain comprises a leucine at position 428 and/or an alanine at position 434.

Embodiment 126. The method of embodiment 125, wherein the Fc domain comprises a LAGA mutation, a FEGG mutation, an AAGG mutation, an AAGA mutation, a LALA mutation or a combination thereof.

Embodiment 127. The method of embodiment 125 or 126, wherein the Fc domain comprises a mutation that has reduced binding to a neonatal Fc receptor (FcRn).

Embodiment 128. The method of any one of embodiments 125-127, wherein the Fc domain comprises a I253A mutation, a H310A mutation, a H435R mutation, a H435A mutation or a combination thereof.

Embodiment 129. The method of any one of the preceding embodiments, wherein when the Antibody Agent is a GDF15 Antibody Agent, a GFRAL Antibody Agent or a RET Antibody Agent, and the Antibody Agent comprises a Fc mutation which reduces (e.g., ablates) binding to FcRn, administration of the Antibody Agent to a pregnant subject reduces (e.g., prevents) placental transfer of the Antibody Agent, as compared to administration of an otherwise similar Antibody Agent without an Fc mutation which reduces (e.g., ablates) binding to FcRn.

Embodiment 130. The method of any one of embodiments 7-129, wherein the symptom is nausea and the nausea is reduced to: a complete response; reduced or no emesis; no significant nausea (NSN), or a combination thereof.

Embodiment 131. The method of embodiment 130, wherein a complete response comprises no emesis, or no need for nausea medication, or both.

Embodiment 132. The method of any one of the preceding embodiments, wherein the subject is a mammal.

Embodiment 133. The method of embodiment 132, wherein the mammal is a human.

Embodiment 134. The method of embodiment 133, wherein the human is an adult.

Embodiment 135. The method of embodiment 133, wherein the human is a child.

Embodiment 136. The method of embodiment 32, wherein the mammal is a dog.

Embodiment 137. The method of embodiment 132, wherein the mammal is a cat.

### Examples

The following examples are provided so as to describe to the skilled artisan how to make and use methods and compositions described herein, and are not intended to limit the scope of the present disclosure.

### Example 1: Identification of GDF15 Antibodies

This Example demonstrates the identification of GDF15 Antibodies from human synthetic yeast libraries.

### Materials and Methods

### Antigen Preparation

Antigens were biotinylated using the EZ-Link Sulfo-NHS-Biotinylation Kit (Thermo Scientific, Cat #21425). The antigens were concentrated to ˜1 mg/mL and buffer exchanged into PBS before addition of 1:7.5 molar ratio biotinylation reagent. The mixture was held at 4C overnight prior to another buffer exchange to remove free biotin in the solution. Biotinylation was confirmed through streptavidin sensor binding of the labeled proteins on a ForteBio.

### Naïve Library Selections

Eight naïve human synthetic yeast libraries each of ˜109 diversity were propagated as previously described (see, e.g., Y. Xu et al, PEDS 26 (10), 663-70 (2013); WO2009036379; WO2010105256; and WO2012009568.)

For the first two rounds of selection, a magnetic bead sorting technique utilizing the Miltenyi MACS system was performed, as previously described (see, e.g., Siegel et al, J Immunol Methods 286 (1-2), 141-153 (2004).) Briefly, yeast cells (˜10¹⁰ cells/library) were incubated with 10 nM biotinylated human GDF15-Fc fusion for 30 min at 30° C. in wash buffer (phosphate-buffered saline (PBS)/0.1% bovine serum albumin (BSA)). After washing once with 40 mL ice-cold wash buffer, the cell pellet was resuspended in 20 mL wash buffer, and Streptavidin MicroBeads (500 μl) were added to the yeast and incubated for 15 min at 4° C. Next the yeast were pelleted, resuspended in 5 mL wash buffer, and loaded onto a Miltenyi LS column. After the 5 mL were loaded, the column was washed 3 times with 3 mL wash buffer. The column was then removed from the magnetic field, and the yeast were eluted with 5 mL of growth media and then grown overnight.

The following rounds of selection were performed using flow cytometry (FACS). Yeast were pelleted, washed three times with wash buffer, and incubated at 30° C. with either 10 nM biotinylated human GDF15-Fc fusion, one of 10 nM biotinylated cyno GDF15-Fc fusion or 10 nM biotinylated mouse GDF15-Fc fusion in order to obtain species cross-reactivity, or with a polyspecificity reagent (PSR) to remove non-specific antibodies from the selection. For the PSR depletion, the libraries were incubated with a 1:10 dilution of biotinylated PSR reagent as previously described (see, e.g., Y. Xu et al, PEDS 26 (10), 663-70 (2013).) Yeast were then washed twice with wash buffer and stained with goat F(ab′)2 anti-human kappa-FITC (LC-FITC) diluted 1:100 (Southern Biotech, Cat #2062-02) and either Streptavidin-AF633 (SA-633) diluted 1:500 (Life Technologies, Cat #S21375) or Extravidin-phycoerthyrin (EA-PE) diluted 1:50 (Sigma-Aldrich, Cat #E4011), secondary reagents for 15 min at 4° C. After washing twice with ice-cold wash buffer, the cell pellets were resuspended in 0.3 mL wash buffer and transferred to strainer-capped sort tubes. Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast were plated and individual colonies were picked for characterization.

### Antibody Optimization

Optimization of antibodies was performed via a light chain batch shuffle, and then by introducing diversities into the heavy chain variable region using approaches described below.

Light chain batch shuffle: Heavy chains from the naïve output were used to prepare light chain diversification libraries. Selections were performed on these libraries as described above, i.e., with one round of MACS and four rounds of FACS. In the different FACS selection rounds, the libraries were evaluated for, e.g., PSR binding, species cross-reactivity, and affinity pressure by antigen titration. Sorting was performed in order to obtain a population with the desired characteristics. Individual colonies from each terminal FACS selection round were picked for sequencing and characterization.

CDRH1 and CDRH2 selection: The CDRH3 of a single antibody was recombined into a premade library with CDRH1 and CDRH2 variants of a diversity of ˜108 and selections were performed with one round of MACS and four rounds of FACS as described in the naïve discovery. For each FACS round the libraries were looked at for PSR binding and affinity pressure, and sorting was performed in order to obtain a population with the desired characteristics. For these selections, affinity pressures were applied by preincubating the biotinylated antigen with parental IgG or Fab for 30 minutes and then applying that precomplexed mixture to the yeast library for a length of time which would allow the selection to reach an equilibrium. The higher affinity antibodies were then able to be sorted.

CDRH3 selection: Oligos were ordered from IDT which comprised the CDRH3 as well as a flanking region on either side of the CDRH3. Each oligo variegated two amino acids in the CDRH3 via NNK diversity. The CDRH3 oligos were recombined with heavy chain FR1-FR3 variable regions containing selected variants from the CDRH1 and CDRH2 selections. Selections were performed similar to previous cycles using FACS sorting for four rounds. For each FACS round the libraries were looked at for PSR binding and affinity pressure, and sorting was performed in order to obtain a population with the desired characteristics. Affinity pressures for these selections were performed as described above in the CDRH1 and CDRH2 selection.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 3.5.

### Results

This process resulted in the identification of human GDF15 Antibody Agents including clones A to T. The selected antibodies had a low PSR score as provided in Table 3. The PSR score was determined by normalizing values against a set of control IgGs whereby a clean PSR is a score of less than 0.1, a low PSR is a score between 0.1 and 0.33, a medium PSR is a score between 0.33 and 0.66 and a high PSR is a score between 0.66 and 1. The data provided in Table 3 shows that tested GDF15 Antibody Agents had low PSR scores indicating high specificity to the GDF15 antigen and minimal polyreactivity.

TABLE 3
PSR scores

| | Poly-Specificity Reagent |
|---|---|
| Clone | (PSR) Score (0-1) |
| A | 0.00 |
| B | 0.01 |
| C | 0.10 |
| D | 0.04 |
| E | 0.08 |
| F | 0.00 |
| G | 0.00 |
| H | 0.11 |
| I | 0.00 |
| J | 0.12 |
| K | 0.00 |
| L | 0.18 |
| M | 0.00 |
| N | 0.00 |
| O | 0.07 |
| P | 0.02 |
| Q | 0.16 |
| R | 0.02 |
| S | 0.12 |
| T | 0.22 |

### Example 2: Characterization of GDF15 Antibodies

This Example describes the evaluation of binding kinetics, binding specificity, stability and aggregation behavior for GDF15 Antibody Agents identified in Example 1.

### Materials and Methods

### ForteBio Octet KD Measurements

ForteBio affinity measurements were performed on an Octet HTX generally as previously described (see, e.g., Estep et al, Mabs 5 (2), 270-278 (2013)). Briefly, ForteBio affinity measurements were performed by loading IgGs on-line onto AHC sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model.

### ForteBio Octet Epitope Binning

Epitope binning was performed using a standard sandwich format cross-blocking assay. Control anti-target IgG was loaded onto AHQ sensors and unoccupied Fc-binding sites on the sensor were blocked with an irrelevant human IgG1 antibody. The sensors were then exposed to 100 nM human GDF15-Fc antigen followed by a second GDF15 antibody. Additional binding by the second antibody after antigen association indicates an unoccupied epitope (non-competitor), while no binding indicates epitope blocking (competitor).

### Size Exclusion Chromatography

A TSKgel SuperSW mAb HTP column (22855) was used for fast SEC analysis of yeast and mammalian produced mAbs at 0.4 mL/min with a cycle time of 6 min/run. 200 mM Sodium Phosphate and 250 mM Sodium Chloride was used as the mobile phase.

### Dynamic Scanning Fluorimetry (DSF)

10 μL of 20× Sypro Orange is added to 20 μL of 0.2-1 mg/mL mAb or Fab solution. A RT-PCR instrument (BioRad CFX96 RT PCR) is used to ramp the sample plate temperature from 40° C. to 95° C. at 0.5° C. increment, with 2 min equilibrate at each temperature. The negative of first derivative for the raw data is used to extract Tm.

### HIC (Hydrophobic Interaction Chromatography)

The methodology for this assay was described previously (see Estep P, et al. (2015) An alternative assay to hydrophobic interaction chromatography for high-throughput characterization of monoclonal antibodies. MAbs 7 (3): 553-561). In brief, 5 ug IgG samples (1 mg/mL) were spiked in with a mobile phase A solution (1.8 M ammonium sulfate and 0.1 M sodium phosphate at pH 6.5) to achieve a final ammonium sulfate concentration of about 1 M before analysis. A Sepax Proteomix HIC butyl-NP5 column was used with a linear gradient of mobile phase A and mobile phase B solution (0.1 M sodium phosphate, pH 6.5) over 20 min at a flow rate of 1 mL/min with UV absorbance monitoring at 280 nm.

### Surface Plasmon Resonance Kinetics Experiments

Kinetic analysis was conducted at 25° C. in HBS-EP+running buffer (10 mM HEPES pH 7.4, 150 mM NaCl, 3 mM EDTA, 0.05% Surfactant P20) using a Biacore 8K optical biosensor (Cytiva, Marlborough, MA). The sample compartment was maintained at 10° C. for the duration of the experiment. The Biacore 8K can analyze up to 8 separate interactions in a single cycle.

Each experiment cycle began with an injection (300 s at 2 μL/min) overflow cells 1 and 2 of a 1:100 solution of biotin CAPture reagent (Cytiva) in running buffer. This was followed by an injection (45 s at 10.0 μL/min) of biotinylated human GDF15 (5 nM) overflow cell 2. Upon capture of GDF15 to the sensor surface, a single concentration of Fab ranging from 0.111-81.0 nM was injected (300 s at 30 μL/min) overflow cells 1 and 2. The dissociation of the antibodies were monitored for 3420 s. Finally, an injection (120 s at 10 μL/min) of regeneration solution (6 M Guanidine-HCl in 0.25 M NaOH) overflow cells 1 and 2 prepared the sensor surface for another cycle. Several blank buffer samples were injected (300 s at 30 μL/min) overflow cells 1 and 2 throughout the duration of the experiment and used for reference surface subtraction—each were monitored for the same amount of dissociation time (3420 s) and regenerated under the same conditions as those for the Fab sample injections.

The resulting sensorgrams were cropped to include only the association and dissociation steps of the experiment. This cropped data was subsequently aligned, double reference subtracted, and then non-linear least squares fit to a 1:1 binding model using Biacore Insight Evaluation software version 3.0.11.15423. (Myszka D G. Improving biosensor analysis. J Mol Recognit. 1999; 12 (5): 279-84. pmid: 10556875).

### AC-SINS

The AC-SINS assay was performed as described previously (Sule et al, Biophysical Journal 101, 1749-1757 (2011), Liu et al, mAbs 6 (2), 483-492 (2014)). In short, gold nanoparticles (15705; Ted Pella Inc.) were coated with 80% capturing anti-human goat IgG Fc (109-005-098; Jackson ImmunoResearch) and 20% with polyclonal goat nonspecific antibody (005-000-003; Jackson ImmunoResearch). The antibodies of interest were then incubated with the particles for 2 h and the wavelength shift was measured using Molecular Devices SpectraMax M2 with SoftMax Pro6 software. The self-interacting clones show a higher wavelength shift away from the PBS sample.

### Transient CHO Expression

Transient CHO expression was performed using standard methods known in the art. In general, CHO-K1 cells grown to about 4×10*6 cells/mL were pelleted and resuspended in transfection medium. DNA plasmids (1.5 ug total DNA/mL) were incubated with PEIpro (1:2 final, PolyPlus, Cat #115-100) in transfection medium at room temperature before addition to the CHO-K1 cell suspension. Transfected cultures were fed and maintained at 32° C. or 37° C., shaking until supernatant was harvested (at day 9) for purification.

### Functional Assay Using the Activin 2B Receptor/SMAD Reporter Assay

Compound information: Antibodies provided in HEPES buffer (25 mM HEPES, 150 mM NaCl pH 7.3). It was assumed that the molecular mass of the compounds is 150,000.

Antibody treatment condition: 100 nM, 10 nM, 1 nM, 100 pM, 10 pM, 1 pM, 0.1 pM, 0.01 pM, vehicle control 1 uM single concentration.

Storage: Stocks are stored in −80° C. freezer.

Protocol: HEK-Activin 2B Receptor/SMAD reporter cell line clone 2 was plated in white 96 well plates at 10K cells per well. After overnight incubation the medium was removed and replaced with 90 μL DMEM containing 0.2% FBS, 2 mM L-glutamine and 50 mM HEPES pH7.4 and cells were incubated for 4 h. Endogenous ligands of the Activin 2B receptor i.e. Activin A, Activin B and GDF-11 were diluted in medium at 10× final concentration. A final concentration of an EC₈₀ of each was used to assess the potential inhibitory activity of mAbs. This medium was used to prepare dilutions of the mAb at 10× the final required concentration. Ligand/GDF15 mAb mixtures were incubated for 15 minutes at room temperature before adding 10 μL to the cells. Cells were incubated at 37° C. for 16 hours. Luciferase activity was detected by adding Bright Glo reagent and reading in an En Vision plate reader.

### Results:

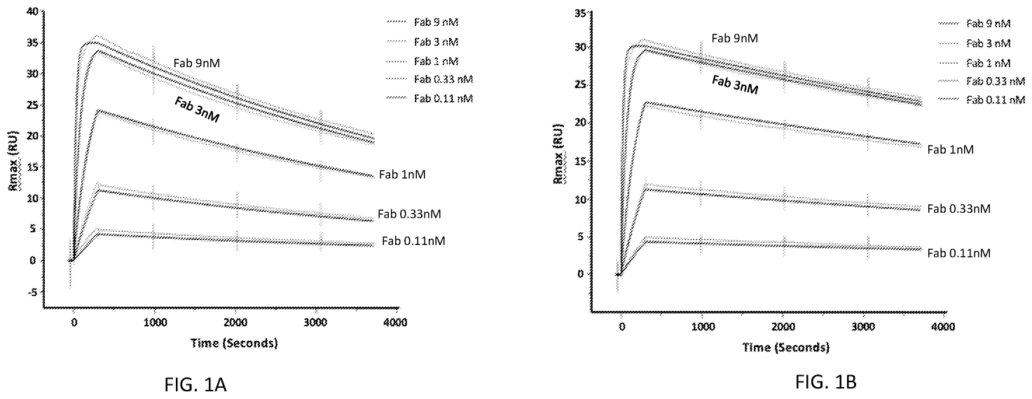

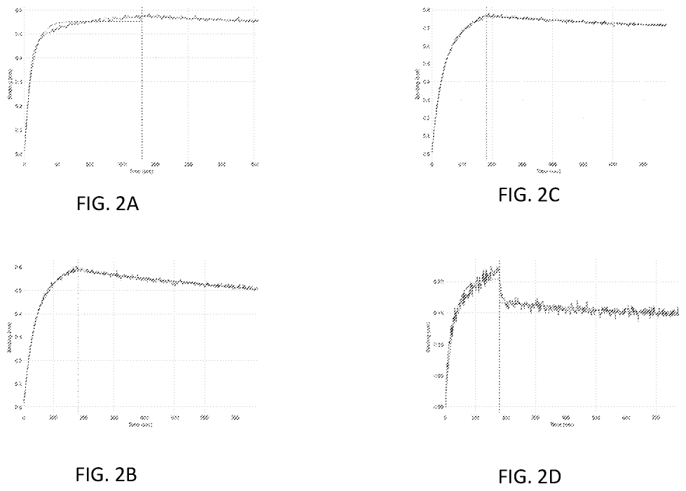

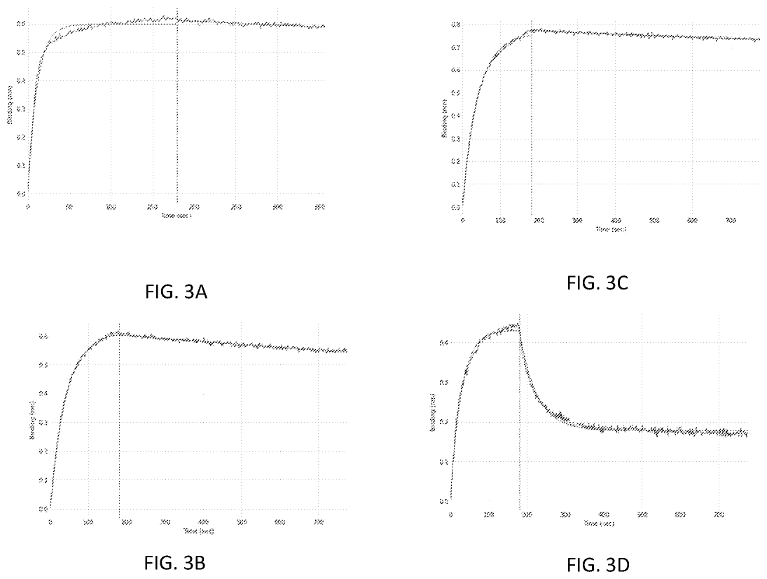

GDF15 Antibody Agents identified in Example 1 were characterized. First, binding kinetics of GDF15 Antibody Agents was assessed with a Surface Plasmon Resonance assay (Biacore). The binding kinetics of GDF15 Antibody Agents are shown in Table 4 and representative graphs for Clone A and Clone C are provided in FIGS. 1A-1B.

The data shows that GDF15 Antibody Agents are potent and bind to GDF15 with binding affinities of 7.3 pM to 599 pM.

TABLE 4
Binding kinetics of GDF15 antibodies

| | Biacore Fab K_{D} | | | |
|---|---|---|---|---|
| | biotinylated | | | |
| | Human GDF15 | | | |
| | Fc (M) | kon | koff | Rmax |
| Clone | Monovalent | (1/Ms) | (1/s) | (RU) |
| A | 4.17E−11 | 4.05E+06 | 1.69E−04 | 35.1 |
| B | 1.34E−11 | 5.42E+06 | 7.26E−05 | 33.9 |
| C | 1.71E−11 | 4.75E+06 | 8.12E−05 | 30.3 |
| D | 7.30E−12 | 1.22E+07 | 8.90E−05 | 38.5 |
| E | 3.31E−11 | 2.63E+06 | 8.71E−05 | 35.4 |
| F | 2.91E−11 | 3.77E+06 | 1.10E−04 | 34.8 |
| G | 2.29E−11 | 3.25E+06 | 7.46E−05 | 32.8 |
| H | 1.17E−10 | 6.29E+06 | 7.35E−04 | 36.2 |
| I | 6.72E−11 | 3.85E+06 | 2.59E−04 | 29.0 |
| J | 8.84E−11 | 4.57E+06 | 4.04E−04 | 29.5 |
| K | 2.30E−10 | 4.87E+06 | 1.12E−03 | 39.0 |
| L | 1.44E−10 | 3.99E+06 | 5.76E−04 | 38.5 |
| M | 5.99E−10 | 3.20E+06 | 1.91E−03 | 44.9 |
| N | 5.45E−10 | 5.84E+06 | 3.18E−03 | 37.0 |
| O | 3.41E−11 | 2.08E+06 | 7.11E−05 | 26.7 |
| P | 1.26E−10 | 1.66E+07 | 2.10E−03 | 34.8 |
| Q | 4.18E−11 | 2.32E+06 | 9.71E−05 | 71.2 |
| R | 4.94E−11 | 3.41E+06 | 1.68E−04 | 72.4 |
| S | 3.69E−11 | 1.34E+06 | 4.93E−05 | 68.4 |
| T | 4.11E−11 | 1.10E+06 | 4.52E−05 | 68.5 |

Binding affinity and epitope binning assays were performed with a ForteBio instrument as described herein. The results of the binding affinity experiments for all antibodies tested are provided in Table 5 and the results of epitope binning experiments for all antibodies tested are provided in Table 6.

TABLE 5
Binding affinity for GDF15 antibodies

| | Octet IgG | Octet Fab | Octet Fab | Octet Fab |
|---|---|---|---|---|
| | K_{D} Human | K_{D} Human | K_{D} Cyno | K_{D} Mouse |
| | GDF15 Fc | GDF15 Fc (M) | GDF15 Fc (M) | GDF15 Fc (M) |
| Clone | (M) Avid | Monovalent | Monovalent | Monovalent |
| A | 2.63E−10 | 1.05E−09 | 5.36E−10 | P.F. |
| B | 2.60E−10 | 5.48E−10 | 3.49E−10 | P.F. |
| C | 2.54E−10 | 7.31E−10 | 3.60E−10 | 1.56E−07 |
| D | 2.63E−10 | 6.95E−10 | 5.62E−10 | 1.57E−07 |
| E | 2.78E−10 | 9.21E−10 | 4.13E−10 | 3.08E−09 |
| F | 2.44E−10 | 6.98E−10 | 5.53E−10 | 2.61E−08 |
| G | 2.56E−10 | 4.40E−10 | 4.74E−10 | P.F. |
| H | 2.35E−10 | 1.24E−09 | 1.42E−09 | 5.09E−08 |
| I | 2.62E−10 | 1.25E−09 | 1.38E−09 | 1.45E−09 |
| J | 2.76E−10 | 1.28E−09 | 7.69E−10 | P.F. |
| K | 2.71E−10 | 2.11E−09 | 2.40E−09 | 3.33E−07 |
| L | 3.25E−10 | 1.58E−09 | 1.56E−09 | 2.29E−07 |
| M | 3.17E−10 | 3.88E−09 | 1.91E−09 | 3.14E−08 |
| N | 3.36E−10 | P.F. | P.F. | 2.56E−09 |
| O | .79E−10 | .16E−10 | .77E−10 | .16E−08 |
| P | 2.74E−10 | 3.68E−09 | 1.72E−09 | P.F. |
| Q | | 5.40E−10 | 3.27E−10 | 2.04E−07 |
| R | | 5.07E−10 | 3.66E−10 | 2.34E−07 |
| S | | 4.80E−10 | 2.52E−10 | 1.36E−09 |
| T | | 6.13E−10 | 3.69E−10 | 1.92E−09 |

TABLE 6
Epitope binning data for GDF15 antibodies

| | | | | | Octet IgG | Octet IgG | Octet IgG | Octet IgG |
|---|---|---|---|---|---|---|---|---|
| | Octet IgG | Octet IgG | Octet IgG | Octet IgG | K_{D} Human | K_{D} Human | K_{D} Human | K_{D} Human |
| | K_{D} GDNF | K_{D} GDF8 | K_{D} GDF10 | K_{D} GDF11 | Activin A Fc | Activin B | BMP9 | BMP10 |
| Clone | (M) Avid | (M) Avid | (M) Avid | (M) Avid | (M) Avid | (M) Avid | (M) Avid | (M) Avid |
| A | N.B. | N.B. | P.F. | N.B. | 7.34E−09 | N.B. | P.F. | P.F. |
| B | N.B. | N.B. | N.B. | N.B. | 6.49E−09 | 1.45E−07 | P.F. | P.F. |
| C | N.B. | N.B. | 5.93E−08 | N.B. | 6.48E−09 | 1.32E−07 | P.F. | N.B. |
| D | N.B. | N.B. | P.F. | N.B. | 7.87E−09 | N.B. | P.F. | N.B. |
| E | N.B. | N.B. | N.B. | N.B. | N.B. | N.B. | N.B. | N.B. |
| F | N.B. | N.B. | P.F. | N.B. | N.B. | N.B. | P.F. | N.B. |
| G | N.B. | N.B. | 3.68E−08 | N.B. | N.B. | N.B. | P.F. | P.F. |
| H | N.B. | N.B. | N.B. | N.B. | N.B. | N.B. | N.B. | N.B. |
| I | 1.45E−09 | N.B. | N.B. | N.B. | N.B. | N.B. | N.B. | N.B. |
| J | P.F. | N.B. | N.B. | N.B. | N.B. | N.B. | N.B. | N.B. |
| K | 3.33E−07 | N.B. | N.B. | N.B. | N.B. | N.B. | N.B. | N.B. |
| L | 2.29E−07 | N.B. | N.B. | N.B. | N.B. | N.B. | N.B. | N.B. |
| M | 3.14E−08 | N.B. | N.B. | N.B. | N.B. | N.B. | N.B. | N.B. |
| N | 2.56E−09 | N.B. | N.B. | N.B. | N.B. | N.B. | N.B. | N.B. |
| O | N.B. | N.B. | .67E−08 | N.B. | N.B. | N.B. | N.B. | N.B. |
| P | P.F. | N.B. | N.B. | N.B. | N.B. | N.B. | N.B. | N.B. |
| Q | N.B. | N.B. | N.B. | N.B. | P.F. | 1.72E−07 | P.F. | N.B. |
| R | N.B. | N.B. | N.B. | N.B. | P.F. | 1.88E−07 | P.F. | P.F. |
| S | N.B. | N.B. | N.B. | N.B. | N.B. | N.B. | N.B. | N.B. |
| T | N.B. | N.B. | N.B. | N.B. | N.B. | N.B. | N.B. | N.B. |

P.F. indicates a binder with a Poor Fit according to a 1:1 Binding model.
N.B. indicates a non-binder under the conditions tested.

TABLE 6A
Functional selectivity data for GDF15 antibody agents

| | Activin A- | Activin B- | GDF-11- |
|---|---|---|---|
| | ActRIIB | ActRIIB | ActRIIB |
| | SMAD | SMAD | SMAD |
| | IC50 nM | IC50 nM | IC50 nM |
| | Top Conc. | Top Conc. | Top Conc. |
| | tested 100 | tested 100 | tested 100 |
| Clone | nM | nM | nM |
| A | ND | ND | ND |
| B | ND | ND | ND |
| C | ND | ND | ND |
| D | ND | ND | ND |
| E | ND | ND | ND |
| F | ND | ND | ND |
| G | ND | ND | ND |
| H | ND | ND | ND |
| I | ND | ND | ND |
| J | ND | ND | ND |
| K | ND | ND | ND |
| L | ND | ND | ND |
| P | ND | ND | ND |

ND: Not detected
Conc: Concentration

As demonstrated in Table 5, GDF15 Antibody Agents bound to human, cyno and mouse GDF15 with a high affinity. Further, the data in Table 6 shows that GDF15 Antibody Agents are highly specific to GDF15. Even though some binding to Activin A and Activin B was observed for some GDF15 Antibody Agents (Table 6); importantly, GDF15 Antibody Agents did not functionally inhibit any one of Activin A, Activin B and GDF11 as measured in an Activin 2B Receptor/SMAD reporter assay (Table 6A).

Next, hydrophobicity of GDF15 Antibody Agents was tested using a HIC assay as described herein. Typically, hydrophobic antibodies show self-association and thus low hydrophobicity is preferred. The results of this assay for GDF15 Antibody Agents are shown in Table 7. Retention time of less than 10.5 minutes indicates a clean to low HIC, retention time of 10.5 to 11.5 indicates medium HIC and a retention time of more than 11.5 minutes indicates high HIC. The data in Table 7 demonstrates that each GDF15 Antibody Agents tested had low to medium hydrophobicity and are thus expected to have low self-association.

TABLE 7
HIC Retention Time

| | HIC Retention |
|---|---|
| Clone | Time (min) |
| A | 10.7 |
| B | 11.0 |
| C | 10.6 |
| D | 12.2 |
| E | 11.4 |
| F | 12.4 |
| G | 11.5 |
| H | 11.1 |
| I | 11.3 |
| J | 11.3 |
| K | 11.4 |
| L | 11.2 |
| M | 11.8 |
| N | 10.0 |
| 0 | 9.9 |
| P | 9.4 |
| Q | 10.8 |
| R | 10.9 |
| S | 11.3 |
| T | 11.1 |

Stability of the GDF15 Antibody Agents was assessed with a DSF assay as described herein. The data shown in Table 8 shows that the melting temperature for the Fab fragments tested was between 64.5° C. to 87.5° C.

TABLE 8
Melting temperature for Fab fragments

| Clone | Fab Tₘ by DSF (° C.) |
|---|---|
| A | 82.5 |
| B | 84.5 |
| C | 84.5 |
| D | 83.0 |
| E | 67.0 |
| F | 81.0 |
| G | 76.5 |
| H | 73.0 |
| I | 64.5 |
| J | 71.5 |
| K | 78.5 |
| L | 75.5 |
| M | 74.0 |
| N | 74.5 |
| O | 79.5 |
| P | 76.5 |
| Q | 87.5 |
| R | 84.5 |
| S | 69.0 |
| T | 69.5 |

As a final characterization, self-association of GDF15 Antibody Agents was evaluated using the AC-SINS assay described herein using a CHO mammalian expression system. Transient CHO expression was first performed using standard methods, followed by purification of the resulting antibodies. The purified antibodies were then subjected to an AC-SINS assay to evaluate self-interaction of the antibodies. The data from this experiment is provided in Table 9 and shows that GDF15 Antibody Agents tested had low AC-SINS values and thus have a low likelihood of self-associating.

TABLE 9

| | AC-SINS |
|---|---|
| Clone | Δλmax (nM) |
| A | — |
| B | 0.42 |
| C | — |
| D | 3.6 |
| E | 2.68 |
| F | 0.98 |
| G | −0.24 |
| H | 1.84 |

Taken together, the data provided in this Example indicates that human GDF15 Antibody Agents identified in Example 1 have suitable “developability” characteristics and supports the development of these antibodies as therapeutics.

### Example 3: Functional Assessment of GDF15 Antibody Agents

This Example describes GDF15 Antibody Agents can induce functional activity of the GFRAL receptor by measuring phosphorylated ERK (PERK) and luciferase expression.

### Methods

### GDF15 Antibody Screen Using GFRAL/RET PERK ELISA Assay

Antibodies: GDF15 IgG and Fab molecules and a reference antibody were assessed.

Cell line: HEK293 stable cells expressing GFRAL/RET were used. Co-expression of GFRAL and RET and GFRAL activation induce ERK phosphorylation in the cells.

Compounds tested: GDF15 IgG Antibodies and Fabs and a reference antibody were provided in PBS (10 mM Sodium phosphate, 140 mM NaCl pH 7.3). Antibody and Fab treatment conditions were as follows: 10 nM, 1 nM, 100 pM, 10 pM, 1 pM, 0.1 pM, 0.01 pM, 0.001 pM.

Protocol: GFRAL/RET stable cell line cells were seeded out in 96 well plates at 25K cells per well, induced by addition of 1 μg/mL tetracycline and incubated overnight. Cells were serum starved for 4 hours prior to stimulation (in 90 uL serum free medium containing 1 μg/mL tetracycline). Antibodies were diluted at 10× the required final concentration in serum free medium containing 10× EC80 concentration of GDF15 and incubated at room temperature. Cells were equilibrated at room temperature for 20 minutes then 10 μL of antibody/GDF15 mixture added to the cells. Cells were incubated with antibody and GDF15 for 15 minutes at RT, the medium was aspirated from the cells and plates stored at −80° C. Cell lysis and detection of pERK content was performed using the pERK ELISA (R and D systems, DYC1018B-5) according to the manufacturer's guidelines. The pERK produced in the assays was calculated by interpolation from a pERK standard curve. The IC50 concentration-response graphs were plotted in GraphPad Prism and where possible the IC50 value determined.

### GDF15 Antibody Agents Screen Using Luciferase Assay for IC50

Antibodies: GDF15 IgG and Fab molecules and a reference antibody were assessed.

Cell line: HEK293 stable cells expressing GFRAL/RET/SRE reporter. Co-expression of GFRAL and RET and GFRAL stimulation induces pERK levels in the cells. The SRE transcription factor binding sequence drives luciferase expression in response to GFRAL activation and is a measure of the pERK/MAPkinase pathway activation.

Compounds tested: IgG Antibodies and Fabs were provided in PBS (10 mM Sodium phosphate, 140 mM NaCl pH 7.3). It is assumed that the molecular mass of the IgG is 150,000 and the molecular mass of Fab is 48,000. Antibody and Fab treatment for IC50 analysis: 10 nM, 1 nM, 100 pM, 10 pM, 1 pM, 0.1 pM, 0.01 pM, 0.001 pM.

Antibody treatment for pA2 analysis: 100 nM starting concentration of GDF15 followed by semi-log serial dilution (100, 31.7, 10.1, 3.2, 1, 0.3, 0.1 nM) and vehicle was tested together with antibody at 0, 1, 0.1 and 0.01 nM.

Protocol: GFRAL/RET/SRE reporter stable cell line cells were seeded out in 96 well plates at 25K cells per well, induced by addition of 1 μg/mL tetracycline and incubated overnight. Cells were serum starved for 4 hours prior to stimulation (for IC50 testing, serum starving was carried out in 90 μl media containing 1 μg/mL tetracycline and for pA2 analysis, serum starving was performed in 80 μl media containing 1 μg/mL tetracycline).

For IC50 testing, antibodies were diluted at 10× the required final concentration in serum free medium containing 10× EC50 concentration of GDF15. The mixture was incubated at room temperature for 30 minutes and then 10 μL of antibody/GDF15 mixture was added to the cells. For pA2 analysis, antibodies were diluted at 10× the required final concentration in serum free medium and 10 μL of each dilution was applied to cells for 30 minutes. Then, 10 μL of EC50 concentration of GDF15 was applied. Cells were then incubated overnight at 37° C./5% CO2. 100 μL Bright Glo luciferase (Promega) detection reagent added to each well. Cells incubated for 10 minutes before reading luminescence. The IC50 concentration-response graphs were plotted in GraphPad Prism and where possible the IC50 value determined. For pA2 analysis the Gaddum/Schild EC50 equation in GraphPad Prism was applied.

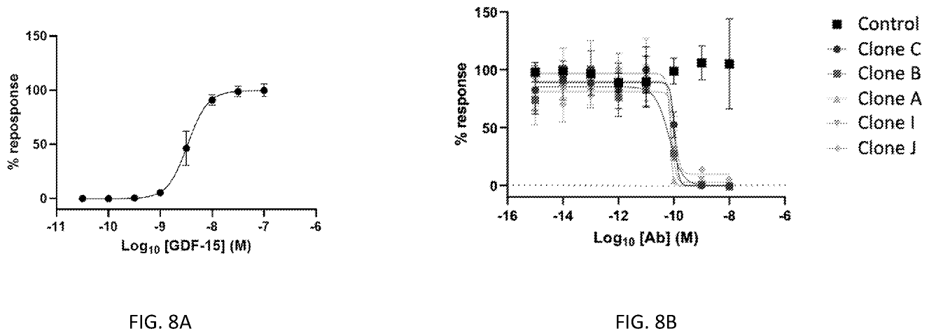

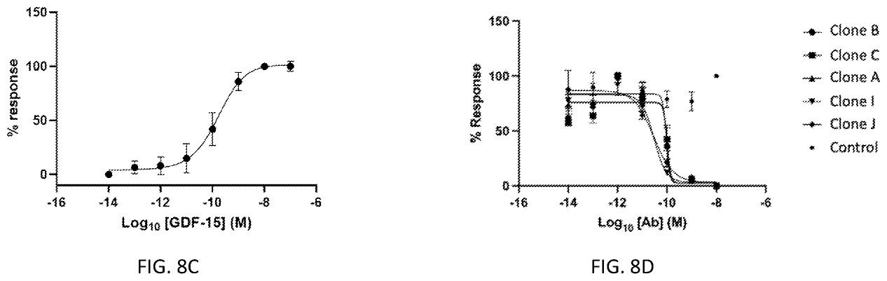

Results: All GDF15 Antibody Agents tested in this Example resulted in inhibition of phospho-ERK (pERK) and luciferase expression, as shown in Table 11 and FIGS. 8A-8D. As shown in the data, the inhibition of pERK and luciferase, both of which are surrogates of inhibition of the GDF15-GFRAL signaling axis, was almost complete (100% inhibition). This data demonstrates that all GDF15 Antibody Agents tested herein inhibit GDF15 functional activity.

TABLE 11

| | GDF15 - | | GDF15 - | | | |
|---|---|---|---|---|---|---|
| | GFRAL | | GFRAL-RET | | | |
| | GFRAL RET | | Luciferase | | GDF15 GFRAL | |
| | pERK IC50 | | IC50 | | RET pERK | |
| Clone | (pM) | | (pM) | | Fab IC50 pM | |
| A | 102.2 | pM | 75.01 | pM | 26.5 | pM |
| B | 104.2 | pM | 66.2 | pM | 30.9 | pM |
| C | 144.9 | pM | 39.6 | pM | 36.8 | pM |
| D | 50.41 | pM | 58.7 | pM | 27 | pM |
| E | 35 | pM | 190 | pM | 46.7 | pM |
| F | 34.93 | pM | 91.3 | pM | 11.2 | pM |
| G | 66.73 | pM | 97.1 | pM | 33.2 | pM |
| H | 254 | pM | 200 | pM | 24.65 | pM |
| I | 34.51 | pM | 79.3 | pM | 110.9 | pM |
| J | 30.31 | pM | 110.4 | pM | 75.1 | pM |
| K | 187.4 | pM | 812.6 | pM | 169 | pM |
| L | 13.50 | pM | 273.8 | pM | ND; 2100 nM | |
| M | 88.65 | pM | | | ND; 1370 pM | |
| N | 99.69 | pM | | | ND; 322.4 pM | |
| O | | | 150.1 | pM | | |

| | | | |
|---|---|---|---|
| P | 35.63 | pM | 701.1 pM; |
| | | | 1000 pM |

ND: Not detected

### Example 4: Expression of GDF15 Antibody Agents in a Human Cell Line

This Example describes expression of GDF15 antibody Clones B, C, E and G identified in Example 1 in a HEK293 expression system.

Methods: HEK293 cells were used to supply a quick transient expression of antibodies. Heavy and light chain of the antibody were individually cloned into an expression vector under CMV promotor. Equal amount of the heavy and light chain DNA constructs were mixed with polyethyleneimine (PEI). Transfection of HEK293 cells was performed by adding the PEI/DNA mixture to the suspension cells in exponential growth phase. The cells were then incubated at 37° C., 8% CO2 and 120 rpm for up to 7 days. The culture was harvested by centrifuging the cell culture fluid and filtering the supernatant using a 0.2 um depth filter.

SEC-HPLC was used to determine the molecular size variants in purified antibodies. Samples were diluted to 1-2 mg/mL with mobile phase and filtered through a 0.22 um cellulose acetate membrane before analysis by YMC-Pack Diol column or equivalent. After peak integration, the main peak, aggregates (HMW), and fragments (LMW) were calculated based on the area normalization method.

Results: As shown in Table 12, all antibodies tested were successful expressed and purified in the HEK293 transient expression system. High expression of Clones B, C, and G was observed, with antibody concentrations of over 200 mg/L.

TABLE 12
Antibody concentration and SEC peak

| | HEX-293 | HEK-293 |
|---|---|---|
| | expression | produced SEC |
| Clone | mg/L | Main Peak (%) |
| B | 471.8 | 97.6 |
| C | 308, 232.8 | 99.2, 100 |
| | average 270.4 | |
| E | 21, 25. | N/A |
| | Average 23 | |
| G | 233.8 | 100 |

### Example 5: Pharmacokinetics of GDF15 Antibodies

This Example describes pharmacokinetic analysis of an exemplary GDF15 antibody identified in Example 1 in mice and primates.

TABLE 13A
Methods: IV Protocol in Primates

| Category | Item | Description |
|---|---|---|
| Species | Cynomolgus | Male, naïve Cynomolgus (3-3.5 kg), |
| | Monkey | N = 3 |
| In-life | mAb, IV, | Dosing day will be named as Day 0. |
| | 5 mg/kg, | PK sampling at predose, 2 hr, 8 hr |
| | 1 mL/kg, | post dosing on Day 0, then daily on |
| | N = 3 | Day 1 (24 hr), 2, 3, 7, 10, 14, 21, |
| | | 28 and 35. 12 time points. Serial |
| | | bleeding from the same animal for |
| | | plasma only. |
| BioAnalytical | Method | Bioanalytical method development |
| | development | using generic ELISA for antibody, |
| | | AP run. |

| | |
|---|---|
| Data analysis and | WinNonlin analysis and report |
| report writing | in Excel format |

TABLE 13B
SC Protocol in Primates

| Category | Item | Description |
|---|---|---|
| Species | Cynomolgus | Male, naïve Cynomolgus (3-3.5 kg), |
| | Monkey | N = 3 |
| In-life | mAb, SC, | Dosing day will be named as Day 0. |
| | 5 mg/kg, | PK sampling at predose, 2 hr, 8 hr |
| | 1 mL/kg, | post dosing on Day 0, then daily on |
| | N = 3 | Day 1 (24 hr), 2, 3, 7, 10, 14, 21, |
| | | 28 and 35. 12 time points. Serial |
| | | bleeding from the same animal for |
| | | plasma only. |
| BioAnalytical | Method | Bioanalytical method development |
| | development | using generic ELISA for antibody, |
| | | AP run. |

| | |
|---|---|
| Data analysis and | WinNonlin analysis and report |
| report writing | in Excel format |

PK protocol in mice: Male C57BL/6 mice were dosed with an GDF15 antibody intravenously (IV, 1 mg/Kg, n=18) and subcutaneously (SC, 10 mg/Kg, SC, n=18). IV doses were delivered directly through the tail vein with an appropriately sized needle. SC doses were administered to the intrascapular region. Animals were weighed on the morning of dose administration. Blood was collected at 0, 1, 2, 6, 8, 24, 48, 144, 312, 480, and 648 hours. ˜ 0.2 mL (retro orbital) or ˜1 mL (cardiac puncture) samples were stored in tubes containing K2EDTA. Each mouse had 2 blood samples collected. Blood samples were collected from 3 mice at each specified time point with preference to the retro orbital sinus (first sample) or cardiac puncture (second or only sample) while anesthetized with inhaled Isoflurane. Clinical observations were conducted at every time point. Blood samples were kept on ice until being centrifuged at 3200 RPM for 10 minutes at ˜ 5° C. within 1 hour of collection. Plasma was directly transferred to cluster tubes and stored at −20° C.±5° C. Plasma antibody concentrations were measured using Affinity-Capture Liquid Chromatography Mass Spectrometry (LC/MS).

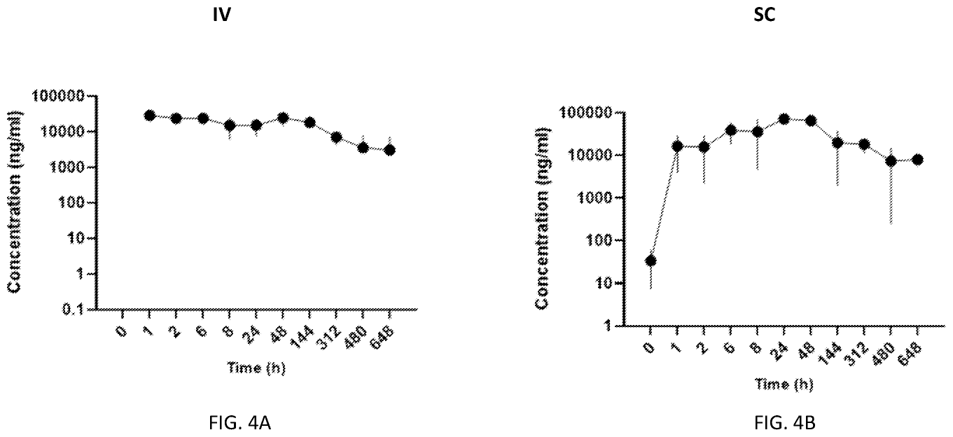

Results: FIG. 4A shows the mean serum concentration-time profile of antibody Clone C dosed intravenously at a dose of 1 mg/Kg to male C57BL/6 Mice (N=3/timepoint). The antibody concentration in serum was relatively stable for the duration of the experiment. FIG. 4B shows the mean serum concentration-time profile of antibody Clone C dosed subcutaneously at a dose of 10 mg/Kg to male C57BL/6 Mice (N=3/timepoint). The antibody concentration in serum peaked at about 1 hour post dosing and remained relatively stable for the rest of the experiment (upto 648 hours post administration).

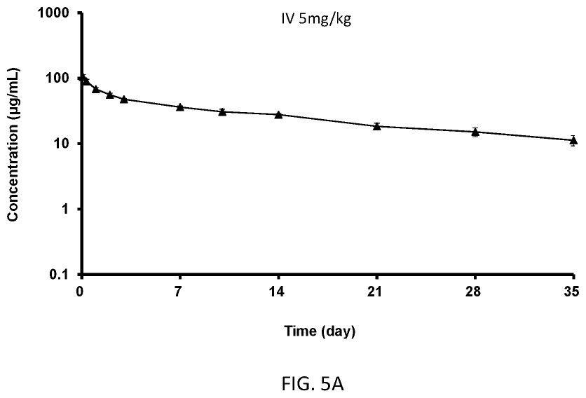

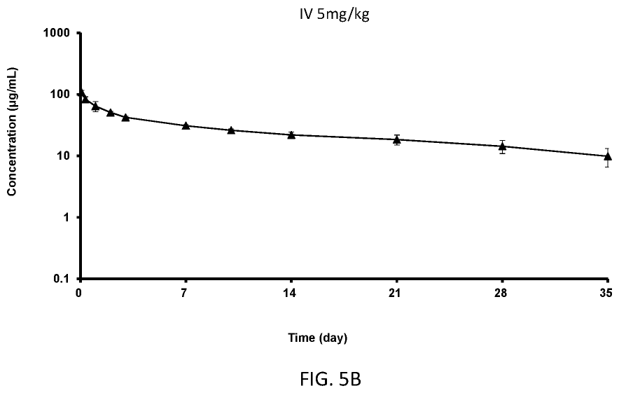

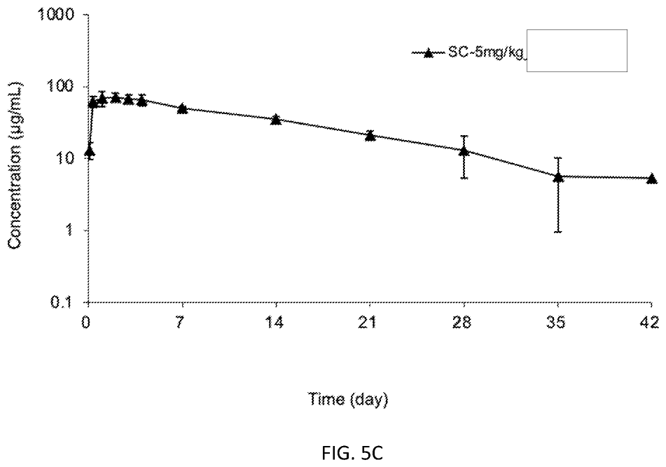

In monkeys dosed intravenously with antibody Clone B or Clone C, or subcutaneously with antibody Clone C at a dose of 5 mg/Kg, similar favorable pharmacokinetics were observed. As shown in FIGS. 5A-5B, the antibody concentration was about 100 μg/mL immediately after intravenous dosing and remained at a high concentration throughout the course of the experiment (up to 35 days). Similarly, FIG. 5C shows a high concentration of the antibody in the plasma after subcutaneous administration of Clone C.

This data shows that the exemplary GDF15 antibodies tested (Clones B and C) had favorable pharmacokinetic properties in vivo in mice and primates suggesting its usefulness as a therapeutic.

### Example 6: GDF15 Antibody Treatment Stops and Reverses Weight Loss in a Mouse Model

This Example describes the effect of a GDF15 antibody on preventing and/or reversing weight loss in mice overexpressing GDF15.

Method: AAV-GDF15-induced weight loss in lean mice Protocol

Mice C57BL/6J mice (7-8 weeks of age upon arrival) were purchased from Charles River UK (Margate, Kent). Mice were singly housed in polypropylene cages with free access to standard chow (Envigo Teklad Global 2018) and filtered tap water. Single housing was undertaken to avoid the chance of fighting in the mice (and to maximize accuracy of food intake measurements) and cages contained sawdust, red house, red tunnel, sizzlenest and nestlet. Upon arrival, mice were weighed and given wet mash to aid recovery from transport. Mice were weighed the following day and the Monday of the following week. Relative humidity was 55±15% with prolonged periods below 40% RH or above 70% RH avoided as detailed in the UK Code of Practice. Animals were maintained on a normal light/dark cycle (lights on: 07:00-19:00). Animals were acclimatized to the facility for approximately one week prior to use and will be handled prior to the onset of procedures. The experiment was performed in the light phase of the light/dark cycle.

The week after arrival, all animals began a 3-day daily handling protocol where each animal was picked up but not dosed or weighed. On Day 0 animals were allocated to dosing groups based on body weight, body composition and daily food and water intake. The allocation was undertaken by a statistician and it will be ensured that groups will be balanced as closely as possible for these variables. Dosing was undertaken on Day 1. Dosing is not time critical but started approximately 10:00 am.

Mice were dosed by the intravenous route and received either control vector (2.92×10¹³ GC/mL) or AAV8-EF1a-hGDF15 (2.92×10¹³ GC/mL); 0.1 mL. Subsequent to dosing, mice were observed and weighed daily (beginning circa 10:00 am). Food and water intake was also recorded daily for the duration of the study. The next phase of the study commenced once a 10% weight loss was evident when compared to the appropriate controls. Mice were allocated to the treatment groups based on original treatment, body weight, body composition, and food and water intake. Once animals achieved 10% weight loss, vehicle (IgG1 isotype control) or mAb 20 mg/Kg SC was administered.

During the vehicle or antibody dosing phase, mice were weighed daily and food and water intake monitored.

All animals will be sacrificed by a schedule 1 method (increasing exposure to CO2 and confirmation of death by cervical dislocation). The blood was taken by cardiac puncture into an EDTA-coated tube and stored at −80° C. prior to analysis.

Body weight, food and water intake data were analyzed by mixed linear model with the group*day interaction as a fixed factor, and animal as the random subject using an AR(1)+RE covariance structure. Body weight gains and average food and water intake (weekly and for other relevant periods of the study) were derived from contrasts from the mixed models.

Results: Increased levels of GDF15 has been shown to induce weight loss. In this Example, mice experiencing weight loss induced by overexpression of human GDF15 by means of an AAV vector, were treated with anti-GDF15 antibody Clone C to evaluate the effect of the antibody on reversing weight loss.

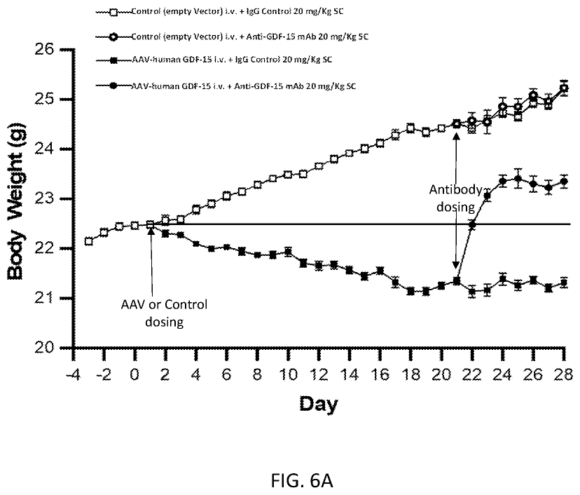

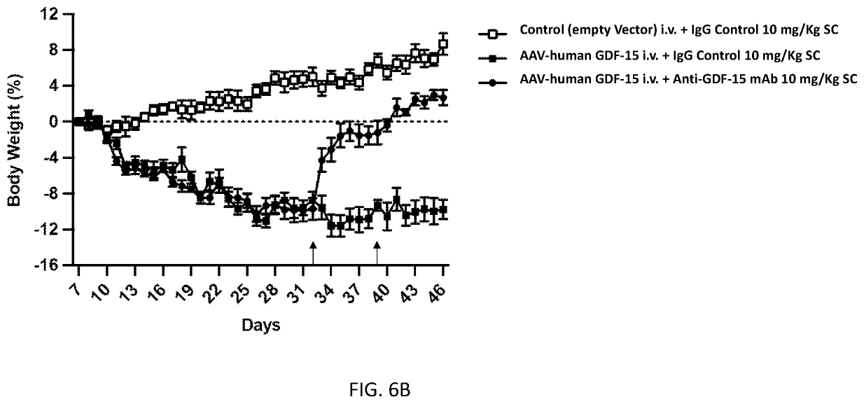

As shown in FIG. 6A, a single subcutaneous administration of 20 mg/kg Clone C antibody to mice suffering weight loss resulted in a significant reversal of weight loss as assessed by increasing body weight in mice post-administration. FIG. 6B shows reversal of GDF15 induced weight loss in mice with two subcutaneous administrations of 10 mg/kg GDF15 antibody agent Clone C. After administration of the first dose of the GDF15 antibody agent, significant reversal of weight loss (as shown by increased body weight) was observed. The body weight of the animals continued to increase upon administration of the second dose and was sustained. This data demonstrates the in vivo potency and efficacy of GDF15 antibody agents in inhibiting GDF15 mediated weight loss in mice.

In summary, the data provided herein supports the development of GDF15 antibody agents disclosed herein as therapeutic modalities to treat and/or prevent diseases or disorders, or symptoms thereof, associated with increased GDF15.

### Example 6B: Non-Platinum Based Chemotherapy Adriamycin (Doxorubicin)-Induced Weight Loss in Mice is Associated with Increased Plasma GDF15 Levels

This Example demonstrates that weight loss in mice induced by a non-platinum based chemotherapy is associated with increased GDF15 levels.

For this experiment, male BALB/c OLA mice 11-12 weeks of age (23-28 g; purchased from Envigo, UK) were on a normal light/dark cycle (lights on: 07:00-19:00 h), acclimatized to the animal unit for approximately 3 weeks and had free access to a standard rodent maintenance diet and filtered water ad libitum. All animals were housed in individually ventilated cages. The holding room was maintained at an elevated temperature of 24±2° C. and relative humidity typically at 55±15% with prolonged periods below 40% RH or above 70% RH avoided as detailed in the UK Code of Practice. As a refinement, each cage contained plenty of sizzlenest, a red house, perspex tunnel, plastic chew stick and a nestlet so that the mice could make nests and facilitate warmth.

All mice were handled as if to be dosed but not actually dosed for seven consecutive days. This handling and dosing procedure has been found to reduce the incidence of stress related effects in studies e.g. in a chronic study the body weight response to baseline dosing is more stable. Mice were allocated to the study groups on the basis of body weight by a Statistician on day-2. On Day 0, mice received either Vehicle (PBS, n=4) or Adriamycin (11, 11.5 or 11.75 mg/Kg, n=4 each) intravenously via the tail vein. Adriamycin was purchased from Tocris. Body weight was measured daily. On Day 14, animals were fasted overnight. On Day 15 all animals were weighed and then humanely killed to a timed schedule by a Schedule 1 method (exposure to an increasing concentration of CO₂ with confirmation of death by cervical dislocation). A terminal blood sample was collected into a lithium heparin tube and plasma separated by cold centrifugation (4° C.). Samples were immediately frozen on dry-ice and stored frozen (−80° C.) prior to analysis. Plasma GDF15 was measured using the Mouse/Rat GDF15 Quantikine ELISA kit from R&D Systems.

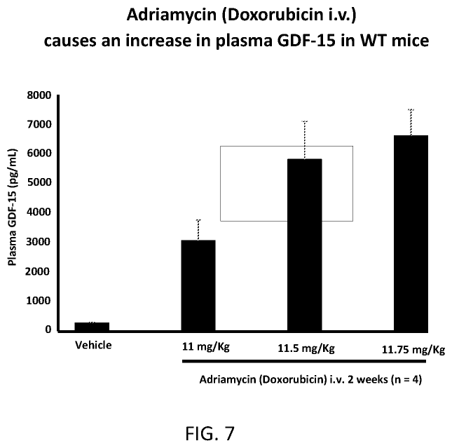

As shown in FIG. 7, treatment with Adriamycin resulted in a substantial increase in plasma GDF15 levels in the animals. The increase in GDF15 levels was dose dependent.

### Example 7: GDF15 Antibody May Reduce Pica In-Vivo

This present example demonstrates that, among other things, certain GDF15-GFRAL Pathway Modulating Agents (e.g., an Antibody Agent such as a GDF15 Antibody Agents) may be useful to treat or prevent pica in-vivo (e.g., in one or more rat models).

Male Sprague Dawley rats (Taconic) at 7-8 weeks of age are used. Animals are group-housed in cages with 4 animals/cage in the HM2 system. Body weight and food intake (chow and Kaolin are recorded separately) is recorded daily from day-7. Animals that ingest or pulverises Kaolin in the baseline period are excluded prior randomization. Day-3: Animals are randomized based on body weight (n=10-12 per group): 1) Vehicle+IgG1 control, 2) Cisplatin with IgG1 control or GDF-15 mAb, or 3) GDF15 with IgG1 control or GDF-15 mAb. All vehicles and drugs are administered IP or subcutaneously. Day-1: 24 h hours prior to cisplatin or GDF15 administration, animals are dosed with a single dose of antibody or mock dosed. Day 1: 24 hours after first dose, animals are dosed with a single dose of vehicle, cisplatin, or GDF15. Chow and Kaolin intake are recorded. Plasma GDF15 is measured by ELISA (R&D Systems).

Among other things, the present disclosure may demonstrate that rats administered a GDF15-GFRAL Pathway Modulating Agents (e.g., an Antibody Agent such as a GDF15 Antibody Agents) may exhibit reduced pica behavior compared to a control (e.g. vehicle administered rats). In some embodiments, GDF15 Antibody Agents may be used as a therapeutic modalities to treat and/or prevent diseases or disorders, or symptoms thereof, associated with increased GDF15.

### Example 8: GDF15 Levels in a Subject Suffering from Nausea and/or Emesis

This present example demonstrates that, among other things, GDF15 may be elevated in a subject suffering from nausea and/or emesis (e.g. a person suffering from chemotherapy emetics, CVS, CHS, and/or MAN/V).

Plasma sample from a subject suffering from nausea and/or emesis can be assessed for GDF15 levels. In some embodiment, a subject has been previously administered or is currently administered a non-platinum based chemotherapy (i.e., 5-Fluorouracil, epirubicin and cyclophosphamide and/or adriamycin and cyclophosphamide). In some embodiments, a subject is suffering from CVS, CHS, and/or MAN/V. In some embodiments, a subject suffering from nausea or emesis has a genetic variant in GDF15, GFRAL, and/or RET.

Among other things, the present disclosure may demonstrate that GDF15 may be elevated in a subject suffering from nausea and/or emesis. In some embodiment, elevated GDF15 levels may be used to identify a subject that may benefit from a GDF15-GFRAL Pathway Modulating Agents (e.g., an Antibody Agent such as a GDF15 Antibody Agents).

### Example 9: Method to Estimate Binding Epitopes of GDF15 Antibodies

This Example described a method to estimate binding epitopes for GDF15 Antibody Agents that may be useful disclosed herein.

As GDF15 Antibody Agents can be assessed, for example, for off-target weak binding to homologous proteins within the TGFb superfamily, e.g., to one or more of: GDNF, GDF8, GDF10, GDF11, Activin A, Activin B; BMP9 and BMP10.

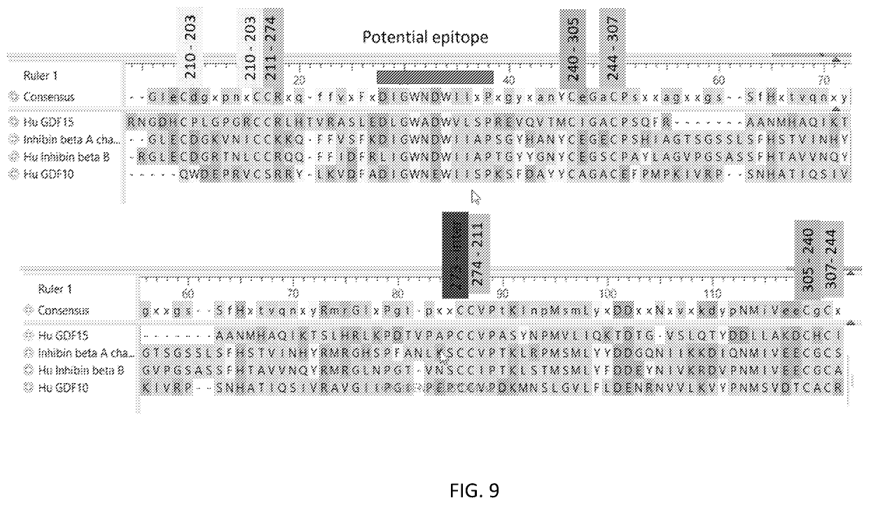

Homology analysis conducted using DNASTAR Lazergene Megalign Pro 17 to compare the protein sequences of GDF15 (https://www.uniprot.org/uniprot/Q99988) and related proteins was used to predict potential biding epitope peptide sequences (FIG. 9). For example, some GDF15 Antibody Agents may show some binding to Activin A (Inhibin beta A, https://www.uniprot.org/uniprot/P08476), Activin B (inhibin beta B, https://www.uniprot.org/uniprot/P09529) and/or GDF10 (https://www.uniprot.org/uniprot/P55107). Analysis of these four proteins demonstrated there was a single region of homology across the four proteins (see FIG. 9). A possible binding epitope covers around the region indicated by the amino acid sequence DLGWADWVLSP at the positions 222 to 232 of the transcribed polypeptide (signal peptide, propeptide, chain) and positions 25 to 33 of the mature protein.

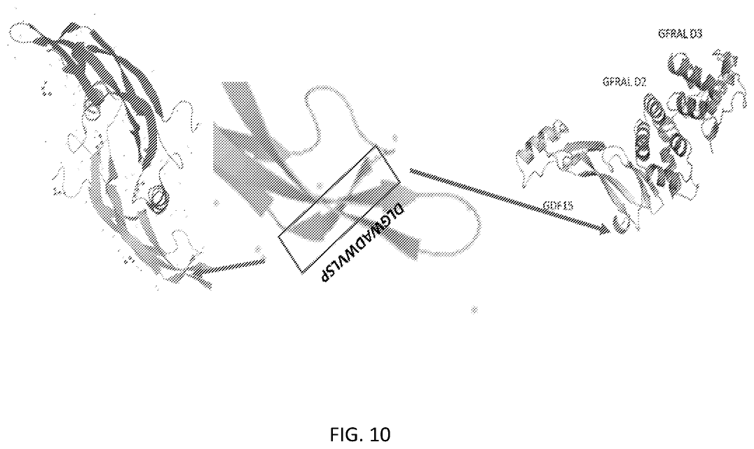

The region of homology between the proteins or the possible binding epitope is contained within an exposed Beta strand that does not appear to be close to the area that directly interacts with the GFRAL binding pocket (FIG. 10). In some embodiments, this binding epitope, would suggest that the antibodies binding within this group use steric interference to prevent GDF15 from interacting with the GFRAL binding pocket.

### Example 10: GDF15 Antibodies that Bind Particular GDF15 Forms

This present example applies teachings described herein to those situations (e.g., subjects) where GDF15 may be present in a particular form (e.g., as a particular variant).

Hyperemesis gravidarum has reported to be associated with elevated levels of GDF15 levels during pregnancy (Fejzo et al 2018, Fejzo et al 2019). Furthermore, it has been reported that patients carrying certain GDF15 variants may also have an increased risk of hyperemesis gravidarum (Fejzo et al 2018, Fejzo et al 2019). The present disclosure teaches that a GDF15-GFRAL Pathway Modulating Agents (e.g., a GDF15 antibody agents) effective against (e.g., in some embodiments specifically targeting) the relevant GDF15 variant may be used in those situations.

### Example 11: GDF15 Antibody Agents Suppress Pica Activity in Rats in Response to Chemotherapy

This example describes suppression of pica activity in rats in response to chemotherapy with the administration of GDF15 antibody agents.

Methods: Male Sprague Dawley rats (Taconic) at 7-8 weeks of age were used to assess rat pica activity in response to chemotherapy. Each animal upon arrival were uniquely identified by an implantable microchip (Pet ID Microchip, E-vet) At the same time animals were registered in a proprietary software system called Cobra. Animals were identified using the WS-2 weigh station (MBrose, Denmark) connected to a computer running the Cobra software. The Cobra software matched body weight with ID. Animals were group-housed in cages with 4 animals/cage in the HM2 system (MBRose, Denmark). The HM-2 system continuously monitored in real-time mode the feeding activity. Intake was measured in two independent channels and recorded the start and finishing time of each meal and the amount of food consumed by each animal within the selected time interval without human intervention. As rats were uniquely identified with microchips, each individual animal was identified by its microchip upon entry and exit from the food channel. Since the HM-2 system is closed off from the environment it is possible to very accurately control temperature and light/dark-cycles. Rats were fed regular chow (Altromin 1324) and tap water. The animal room environment was controlled (21±2° C.; relative humidity 50±10%) and on a 12:12 light: dark cycle, with lights off at 1 pm. Animals had access to bedding material, shelters and chewing sticks. During a 2-week habituation period, rats were exposed to chow and kaolin (Kaolin Research diet, US) and feeding containers of both diets switched every 2 days. Body weight and food intake (chow and Kaolin were recorded separately) were recorded daily from day-7. Animals that ingested or pulverized Kaolin in the baseline period were excluded prior randomization. Day-3: Animals were randomized based on body weight into 4 groups (n=12-14 per group): 1) Vehicle (IP)+IgG1 (20 mg/kg), 2) Cisplatin (6 mg/kg, IP)+IgG1 (20 mg/kg, SC), 3) Cisplatin (6 mg/kg, IP)+Clone C (20 mg/kg, SC), 4) Cisplatin (6 mg/kg, IP)+Clone I (20 mg/kg, SC). Day-1:24 hours prior to cisplatin administration, animals are dosed with a single dose of antibody. Day 1:24 hours after first dose, animals were dosed with a single dose of vehicle or cisplatin, and kaolin intake recorded. Data are expressed as mean #standard error of the mean.

Results: Rats do not vomit and instead experience pica activity which is the eating of nonnutritive substances such as kaolin (Takeda N et al (1993) Pharm. Biochem and Behavior 45 (4): 817-821). Intake of kaolin is widely used as a marker for illness from a wide variety of stimuli, including cisplatin and is considered a proxy for emesis in rats (Li S et al., (2018) Neuromodulation 21 (3): 254-260).

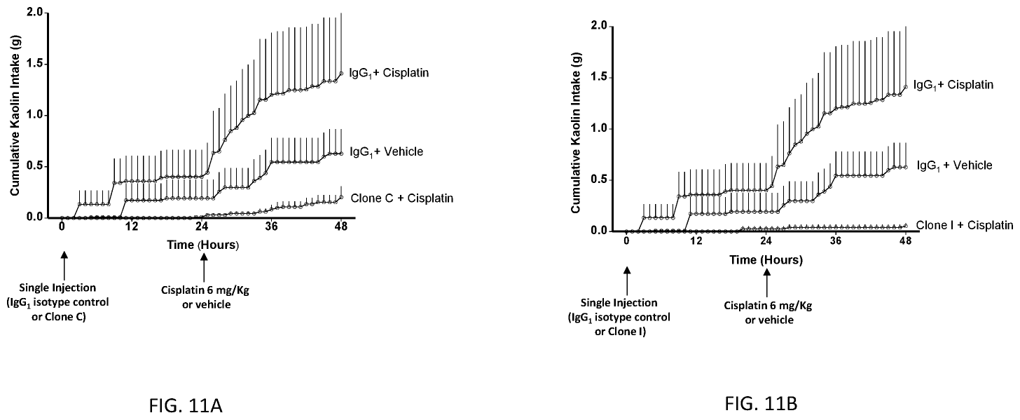

To assess the efficacy of GDF15 antibody agents in suppressing pica activity in rats, animals were dosed with a single injection of GDF15 antibody agents (Clone C or Clone I), or a control antibody. The animals were then given either Cisplatin or a vehicle control and Kaolin intake was recorded. As shown in FIG. 11A, administration of Cisplatin resulted in a significant increase in Kaolin intake in the animals (see IgG+Cisplatin group and IgG+vehicle group). In contrast, animals who were pre-treated with GDF15 antibody agent Clone C did not have an increased Kaolin intake upon Cisplatin treatment. A similar suppression of pica activity was observed with GDF15 antibody agent Clone I (FIG. 11B).

This data demonstrates the in vivo efficacy of GDF15 antibody agents in suppressing pica activity in rats. Since pica activity is a proxy for emesis in humans, this data further supports the development of GDF15 antibody agents disclosed herein as therapeutic modalities to treat and/or prevent GDF15 mediated nausea, emesis and/or related conditions or symptoms.

### Example 12: GDF15 Antibody Agents Having a AAGA Mutation in the Fc Region Display Reduced Binding to FcRn at Neutral pH

This Example describes reduced binding of GDF15 antibody agents having an AAGA mutation in the Fc region to a neonatal Fc receptor (FcRn) at pH7.4.

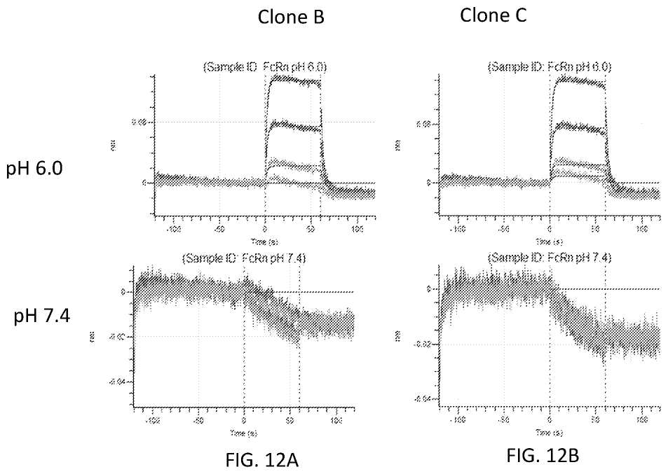

Binding of GDF15 antibody agents with a AAGA mutation in the Fc region (Clone B or Clone C) was tested at an acidic pH and a neutral pH. As shown in FIGS. 12A-12B, both antibodies bound to FcRn at pH 6 (acidic), but there was no binding of either GDF15 antibody agent to FcRn at a neutral pH of 7.4.

This data demonstrates that GDF15 antibody agents having a AAGA mutation in the Fc region do not bind FcRn. Accordingly, administration of GDF15 antibody agents with Fc mutations in the Fc region such as the AAGA mutation, to a pregnant subject is expected to result in minimal or no transfer of the antibody agent into the placenta. This data further supports the development of GDF15 antibody agents with reduced FcRn binding for treating and/or preventing GDF15-associated disorders or symptoms in subjects.

### Example 13: Prevention of GDF15 Induced Reduction in Food Intake with a GDF15 Antibody Agent

This Example describes the prevention of reduced food intake in mice induced by human GDF-15, with the administration of an exemplary GDF15 antibody agent.

Methods: Male C57BL/6J (JAX) mice (weight range 20-25 g) were ordered from Charles River. Upon arrival, animals were singly housed on a normal light/dark cycle (lights on: 07:00-19:00 h) with ad libitum access to standard pelleted diet (Envigo Teklad 2018) and filtered tap water. Mice were weighed and given wet mash to aid recovery from transport. Mice were also checked and weighed the day after arrival. The mice were housed in polypropylene cages with sawdust-coated floors, red house, red tunnel, sizzlenest and nestlet at a temperature of 21±4° C. Relative humidity was 55±15% with prolonged periods below 40% RH or above 70% RH avoided as detailed in the UK Code of Practice.

From the day after arrival, animals were habituated to a daily presentation of a wet mash diet (1 part powdered chow: 1.4 parts tap water: e.g. 0.5 L of water to 350 g of powdered chow). Animals were accustomed to these conditions for approximately nine days before experimentation (wet mash not presented at weekends). Presentation was for approximately 4 hours starting at approximately 09.30 am. On the eighth day of presentation, animals underwent a baseline test session. The mice were weighed and picked up as if to be dosed by the relevant route but not actually dosed. Maintenance diet was removed at the time of handling and wet mash was presented 30 minutes later. The weight of mash presented and the amount of mash present 2 and 4 hours later was recorded (to the nearest 0.1 g). Maintenance diet was returned after the 4 hour reading. Animals were allocated to the treatment groups on the basis of body weight and 2 hour and 4 hour mash intake by a Statistician.

Anti-GDF-15 mAb or isotype control was administered 24 hours prior to study start. GDF-15 (4 nmol/Kg in PBS, SC) administration began at approximately 09:00 and 30 minutes later wet mash was presented. At the time of dosing animals and water bottles were weighed. Maintenance diet was removed. Wet mash was weighed 1, 2 and 4 hours after presentation. The mash was then replaced with a fresh quantity of wet mash at the 4 hour time point. Wet mash was re-weighed at the 8 hour time point. Wet mash will then be removed and replaced with a known quantity of maintenance diet at the 8 hour time point. Water bottles, food pellets and animals were weighed 24 hours after compound dosing. Mash was not presented on these days. Food and water intake of the different groups or animals was measured concurrently. The animals were monitored at each reading and any overt drug-induced behavioral effects recorded.

Data and statistical analysis: Food intake (kCal) for each time interval was analyzed by analysis of covariance with the nearest available baseline time interval as a covariate. For 0-1, 1-2 and 0-2 hours, the covariate was 0-2 h. For 2-4 and 4-8 h, the covariate was 2-4 h. For 0-4 and 0-8 hours, the covariate will be 0-4 hours. Appropriate 2-sided multiple comparisons tests were used to compare each treatment group to vehicle.

Results: Increased levels of GDF-15 has been shown to reduce food intake. To evaluate the efficacy of GDF15 antibody agents in preventing a reduction of food intake induced by increased levels of GDF-15, animals were first administered a GDF15 antibody agent or a control antibody and then dosed with GDF15 or a vehicle control via subcutaneous administration.

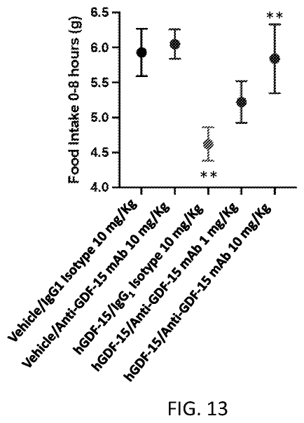

As shown in FIG. 13, increased levels of human GDF-15 resulted in a reduction in food intake (compare first and second groups on the left with third group). Pre-treatment with 1 mg/kg GDF15 antibody agent of animals dosed with human GDF-15 resulted in a smaller decrease in food intake compared to animals dosed with human GDF-15 but not administered the GDF15 antibody agent (compare middle group to fourth and fifth groups). The prevention of reduction in food intake by the GDF15 antibody agent was dose dependent as observed by an even smaller decrease in food intake in animals dosed with human GDF-15 and pre-treated with 10 mg/kg of the GDF15 antibody agent compared to animals pre-treated with 1 mg/kg the GDF15 antibody agent.

This data demonstrates the in vivo efficacy of GDF15 antibody agents in preventing a reduction of food intake induced by GDF15. This data further supports the development of GDF15 antibody agents disclosed herein as therapeutic modalities to treat and/or prevent GDF15 mediated appetite suppression and/or reduced food intake.

### Example 14: Reversal of Tumor-Induced Weight Loss with GDF15 Antibody Agents

This example describes a reversal of tumor-induced weight loss in mice with the administration of GDF15 antibody agents. The tested GDF15 antibody agents had no adverse impact on tumor volume in the HT-1080 tumor bearing cachexia mouse model described herein.

Methods: The HT-1080 cell line was acquired from the American Type Culture Collection (ATCC) and cultured according to the suppliers' recommendations and confirmed to be free of mycoplasma testing. To generate HT-1080 tumor-bearing mice, 8-12 week-old female C.B-17 Severe Combined Immune Deficiency (SCID) mice (Envigo) were acclimatized for 1 week upon receipt. Mice were inoculated subcutaneously (SC) into the flank with 200 μL volume containing 5×10⁶ cells in 1:1 RPMI 1640 (Invitrogen)/Matrigel (BD Biosciences) with. Non-tumor bearing female SCID mice were used as a comparator of control baseline body weight (n=12). Body weight, tumor volume and animal health were assessed daily. Mice were randomized based on body weight. Once tumor bearing mice achieved ˜8% weight loss, mice were treated with antibody (Clone B, Clone C or isotype control) (10 mg/Kg SC; n=12 mice/group). Mice with weight loss >18%, tumor burden >2000 mm³, open weeping tumor ulceration, severe respiratory distress, severe impaired movement or loss of righting reflex were humanly sacrificed as per IUCAC protocol. Body weight and tumor volume parameters were expressed as mean±standard error of the mean.

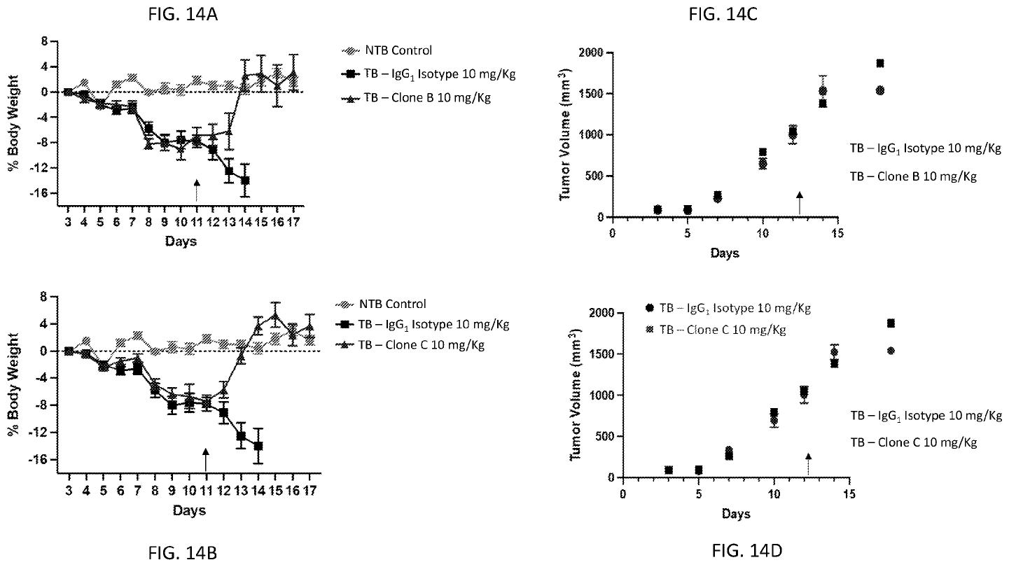

Results: In the HT-1080 tumor bearing cachexia mouse model, tumor growth is associated with weight loss in the tumor-bearing animals. To evaluate the efficacy of GDF15 antibody agents in preventing and/or reversing tumor induced weight loss, tumor-bearing animals were dosed with a single dose of GDF15 antibody agent Clone B or GDF15 antibody agent Clone C on day 11 when tumor-bearing animals had experienced about 10% weight loss. As shown in FIG. 14A, compared to tumor-bearing animals administered a control antibody, tumor-bearing animals that were treated with GDF15 antibody agent Clone B stopped experiencing weight loss after treatment (as observed by stable weight on days 11-12) and showed a reversal of the weight loss starting at around day 13. By day 14, the weight of tumor-bearing animals treated with GDF15 antibody agent Clone B was comparable to the weight of control non-tumor bearing animals. A similarly significant reversal of weight loss was observed in tumor-bearing animals treated with GDF15 antibody agent Clone C (FIG. 14B).

This data demonstrates the in vivo efficacy of GDF15 antibody agents in preventing and reversing cancer induced weight loss, and supports the development of GDF15 antibody agents as therapeutic modalities for treating and/or preventing cancer-associated weight loss.

1. A method of treating and/or preventing hyperemesis gravidarum (HG), Cyclic Vomiting Syndrome (CVS), Cannabinoid Hyperemesis Syndrome (CHS), a Mitochondrial Disorder (MID) and/or Migraine Associated Nausea/Vomiting (MAN/V), comprising administering to a subject a GDF15-GFRAL Pathway Modulating Agent.

2. The method of claim 1, wherein the GDF15-GFRAL Pathway Modulating Agent is a GDF15 Antibody Agent, A GFRAL Antibody Agent, or a RET Antibody Agent.

3. The method of claim 2, wherein the GDF15 Antibody Agent comprises:
(i) an LC CDR1 of SEQ ID NO: 92, an LC CDR2 of SEQ ID NO: 93, and an LC CDR3 of SEQ ID NO: 94; and
(ii) a HC CDR1, a HC CDR2 and a HC CDR3, wherein:
(a) the HC CDR1 is SEQ ID NO: 1, the HC CDR2 is SEQ ID NO: 2 and the HC CDR3 is SEQ ID NO: 3;
(b) the HC CDR1 is SEQ ID NO: 10, the HC CDR2 is SEQ ID NO: 11, and the HC CDR3 is SEQ ID NO: 191;
(c) the HC CDR1 is SEQ ID NO: 14, the HC CDR2 is SEQ ID NO: 15, and the HC CDR3 is SEQ ID NO: 192; or
(d) the HC CDR1 is SEQ ID NO: 18, the HC CDR2 is SEQ ID NO: 19, and the HC CDR3 is SEQ ID NO: 193.

4. The method of claim 3, wherein the GDF15 Antibody Agent comprises:
(a) a light chain (LC) comprising:
(A) a variable region (VL) comprising the sequence of SEQ ID NO: 99 or a sequence having at least 85% identity to SEQ ID NO: 99; and
(B) a constant region (CL); and
(b) a heavy chain (HC) comprising:
(A) a variable region (VH) comprising the sequence of SEQ ID NO: 8, SEQ ID NO 12, SEQ ID NO: 16 or SEQ ID NO: 20, or a sequence having at least 85% identity to SEQ ID NO: 8, SEQ ID NO 12, SEQ ID NO: 16 or SEQ ID NO: 20.

5. The method of claim 4, wherein the GDF15 Antibody Agent comprises a polypeptide comprising:
(i) the sequence of SEQ ID NO: 143, or a sequence with at least 85% identity to SEQ ID NO: 143; and the sequence of SEQ ID NO 159, or a sequence with at least 85% identity to SEQ ID NO 159;
(ii) the sequence of SEQ ID NO: 144, or a sequence with at least 85% identity to SEQ ID NO: 144; and the sequence of SEQ ID NO 159, or a sequence with at least 85% identity to SEQ ID NO 159;
(iii) the sequence of SEQ ID NO: 145, or a sequence with at least 85% identity to SEQ ID NO: 145; and the sequence of SEQ ID NO 159, or a sequence with at least 85% identity to SEQ ID NO 159; or
(iv) the sequence of SEQ ID NO: 146, or a sequence with at least 85% identity to SEQ ID NO: 146; and the sequence of SEQ ID NO 159, or a sequence with at least 85% identity to SEQ ID NO 159 treating and/or preventing Migraine Associated Nausea/Vomiting (MAN/V) comprising administering to a subject a GDF15-GFRAL Pathway Modulating Agent.

6. (canceled)

7. The method of claim 1, wherein the administering is according to a dosing regimen established to reduce severity or incidence of, and/or delay onset of one or more symptoms associated with HG, CVS, CHS, MID, and/or MAN/V.

8. The method of claim 7, wherein the one or more symptoms is chosen from: nausea, retching, emesis, migraine, weight loss, pain, muscle loss, gastric stasis, reduced fetal growth or any combination thereof.

9. The method of claim 1, wherein the subject has increased GDF15 level and/or activity.

10. The method of claim 9, wherein the increased GDF15 level comprises a level of about 1 ng/ml or more.

11. A method of ameliorating a symptom associated with hyperemesis gravidarum (HG), Cyclic Vomiting Syndrome (CVS), Cannabinoid Hyperemesis Syndrome (CHS), a Mitochondrial Disorder (MID) and/or Migraine Associated Nausea/Vomiting (MAN/V), comprising administering to a subject a GDF15-GFRAL Pathway Modulating Agent, wherein the GDF15-GFRAL Pathway Modulating Agent comprises:
(i) an LC CDR1 of SEQ ID NO: 92, an LC CDR2 of SEQ ID NO: 93, and an LC CDR3 of SEQ ID NO: 94; and
(ii) a HC CDR1, a HC CDR2 and a HC CDR3, wherein:
(a) the HC CDR1 is SEQ ID NO: 1, the HC CDR2 is SEQ ID NO: 2 and the HC CDR3 is SEQ ID NO: 3;
(b) the HC CDR1 is SEQ ID NO: 10, the HC CDR2 is SEQ ID NO: 11, and the HC CDR3 is SEQ ID NO: 191;
(c) the HC CDR1 is SEQ ID NO: 14, the HC CDR2 is SEQ ID NO: 15, and the HC CDR3 is SEQ ID NO: 192; or
(d) the HC CDR1 is SEQ ID NO: 18, the HC CDR2 is SEQ ID NO: 19, and the HC CDR3 is SEQ ID NO: 193.

12-15. (canceled)

16. The method of claim 11, wherein the symptom is chosen from: nausea, retching, emesis, migraine, weight loss, pain, gastric stasis, restricted fetal growth, or any combination thereof.

17. The method of claim 11, wherein the subject has a condition or disorder associated with increased GDF15.

18. The method of claim 1, wherein the subject is pregnant.

19. The method of claim 17, wherein the increased GDF15 level comprises a level of about 1 ng/ml or more.

20. The method of claim 11, wherein the GDF15-GFRAL Pathway Modulating Agent is characterized in that when administered to the subject it reduces the level and/or activity of GDF15 relative to a comparator.

21. The method of claim 20, further comprising determining a level and/or activity of GDF15 and/or GFRAL in a sample from the subject, wherein the level of free and/or active;
a) GDF15 is reduced relative to a comparator; and/or
b) GFRAL is reduced relative to a comparator,
wherein the comparator comprises an otherwise similar cell, tissue or subject not administered the GDF15-GFRAL Pathway Modulating Agent or administered a different GDF15-GFRAL Pathway Modulating Agent.

22-24. (canceled)

25. The method of claim 16, wherein: a) the symptom frequency and/or severity is reduced by about 1.5 fold to about 10-fold; and/or b) the symptom is reduced by about 5% or more.

26-28. (canceled)

29. The method of claim 1, wherein a composition comprising the GDF15-GFRAL Pathway Modulating Agent is administered to the subject.

30. The method of claim 29, wherein the composition comprises a pharmaceutical composition, wherein the pharmaceutical composition:
a) comprises an excipient and/or a pharmaceutically acceptable carrier; and/or
b) is formulated in one or more unit dosage forms.

31-33. (canceled)

34. The method of claim 1, wherein the GDF15-GFRAL Pathway Modulating Agent binds to genetic variants of GDF-15, GFRAL and/or RET associated with nausea, vomiting and emesis.

35-40. (canceled)

41. The method of claim 2, wherein the Antibody Agent comprises:
(i) an intact IgA, IgG, IgD, IgE or IgM antibody;
(ii) an antibody fragment;
(iii) a single chain Fv; or
(iv) a polypeptide comprising antigen binding specific fused to a Fc domain.

42-43. (canceled)

44. The method of claim 41, wherein the Antibody Agent comprises at least one heavy chain (HC) comprising at least one constant region (CH), wherein the at least one CH comprises:
(a) an Fc domain chosen from an Fc domain of an immunoglobulin isotype;
(b) an Fc domain comprising a mutation; and/or
(c) a CH3 domain, wherein the CH3 domain comprises a leucine at position 428 and/or an alanine at position 434;
and wherein the Fc domain comprises an Fc mutation selected from:
(a) a LAGA mutation, a FEGG mutation, an AAGG mutation, an AAGA mutation, a LALA mutation or a combination thereof;
(b) a mutation that has reduced binding to a neonatal Fc receptor (FcRn); and/or
(c) a I253A mutation, a H310A mutation, a H435R mutation, a H435A mutation or a combination thereof.

45-46. (canceled)

47. The method of claim 7, wherein the one or more symptoms is nausea, and wherein and the nausea is reduced to: (a) a complete response, wherein the complete response comprises no emesis, or no need for nausea medication, or both; (b) reduced or no emesis; (c) no significant nausea (NSN), or (b) any combination of (a) and (c).

48. (canceled)

49. The method of claim 1, wherein the subject is a mammal.

50-54. (canceled)


SEQUENCE LISTING (234 sequences)

SEQ ID NO: 1 - protein, 11 aa, synthetic construct
GSITSGGSYWS

SEQ ID NO: 2 - protein, 16 aa, synthetic construct
AIVYSGSTYYNPSLKS

SEQ ID NO: 3 - protein, 11 aa, synthetic construct
ARGVEYQHFDY

SEQ ID NO: 4 - protein, 26 aa, synthetic construct
QVQLQESGPGLVKPSQTLSLTCTVSG

SEQ ID NO: 5 - protein, 14 aa, synthetic construct
WIRQHPGKGLEWIG

SEQ ID NO: 6 - protein, 30 aa, synthetic construct
RVTISVDTSKNQFSLKLSSVTAADTAVYYC

SEQ ID NO: 7 - protein, 11 aa, synthetic construct
WGQGTLVTVSS

SEQ ID NO: 8 - protein, 119 aa, synthetic construct
QVQLQESGPGLVKPSQTLSLTCTVSGGSITSGGSYWSWIRQHPGKGLEWIGAIVYSGSTYYNPSLKSRVTISVDTSKNQFSLKLSSVTAADTAVYYCARGVEYQHFDYWGQGTLVTVSS

SEQ ID NO: 9 - DNA, 357 bp, synthetic construct
  source 1..357: mol_type = other DNA
caggtgcagctgcaggagtcgggcccaggactggtgaagccttcacagaccctgtccctcacctgtactgtctctggtggctccatcaccagtggtggttcctactggagctggatccgccagcacccagggaagggcctggagtggattggggccatcgtctacagtgggagcacctactacaacccgtccctcaagagtcgagttaccatatcagtagacacgtctaagaaccagttctccctgaagctgagcagtgtgactgccgcggacacggcggtgtactactgcgccagaggggtggaatatcagcatttcgattactggggacagggtacattggtcaccgtctcctca

SEQ ID NO: 10 - protein, 11 aa, synthetic construct
GSISSGGIYWT

SEQ ID NO: 11 - protein, 16 aa, synthetic construct
AIAYSGSTYYNPSLKS

SEQ ID NO: 12 - protein, 119 aa, synthetic construct
QVQLQESGPGLVKPSQTLSLTCTVSGGSISSGGIYWTWIRQHPGKGLEWIGAIAYSGSTYYNPSLKSRVTISVDTSKNQFSLKLSSVTAADTAVYYCARGIEYQHFDYWGQGTLVTVSS

SEQ ID NO: 13 - DNA, 357 bp, synthetic construct
  source 1..357: mol_type = other DNA
caggtgcagctgcaggagtcgggcccaggactggtgaagccttcacagaccctgtccctcacctgtactgtctctggtggctccatcagcagtggtggtatctactggacctggatccgccagcacccagggaagggcctggagtggattggggccatcgcctacagtgggagcacctactacaacccgtccctcaagagtcgagttaccatatcagtagacacgtctaagaaccagttctccctgaagctgagcagtgtgactgccgcggacacggcggtgtactactgcgccagagggattgaatatcagcatttcgattactggggacagggtacattggtcaccgtctcctca

SEQ ID NO: 14 - protein, 11 aa, synthetic construct
GSISSGGYYWS

SEQ ID NO: 15 - protein, 16 aa, synthetic construct
AIVYSGHTYYNPSLKS

SEQ ID NO: 16 - protein, 119 aa, synthetic construct
QVQLQESGPGLVKPSQTLSLTCTVSGGSISSGGYYWSWIRQHPGKGLEWIGAIVYSGHTYYNPSLKSRVTISVDTSKNQFSLKLSSVTAADTAVYYCARGVEYQHFSLWGQGTLVTVSS

SEQ ID NO: 17 - DNA, 357 bp, synthetic construct
  source 1..357: mol_type = other DNA
caggtgcagctgcaggagtcgggcccaggactggtgaagccttcacagaccctgtccctcacctgtactgtctctggtggctccatcagcagtggtggttactactggagctggatccgccagcacccagggaagggcctggagtggattggggccatcgtctacagtgggcacacctactacaacccgtccctcaagagtcgagttaccatatcagtagacacgtctaagaaccagttctccctgaagctgagcagtgtgactgccgcggacacggcggtgtactactgcgccagaggggtggaatatcagcatttcagtctttggggacagggtacattggtcaccgtctcctca

SEQ ID NO: 18 - protein, 11 aa, synthetic construct
GSINSGGYYWT

SEQ ID NO: 19 - protein, 16 aa, synthetic construct
AIYYSGSTVYNPSLKS

SEQ ID NO: 20 - protein, 119 aa, synthetic construct
QVQLQESGPGLVKPSQTLSLTCTVSGGSINSGGYYWTWIRQHPGKGLEWIGAIYYSGSTVYNPSLKSRVTISVDTSKNQFSLKLSSVTAADTAVYYCARGVEYQHFNEWGQGTLVTVSS

SEQ ID NO: 21 - DNA, 357 bp, synthetic construct
  source 1..357: mol_type = other DNA
caggtgcagctgcaggagtcgggcccaggactggtgaagccttcacagaccctgtccctcacctgtactgtctctggtggctccatcaacagtggtggttactactggacctggatccgccagcacccagggaagggcctggagtggattggggccatctattacagtgggagcaccgtctacaacccgtccctcaagagtcgagttaccatatcagtagacacgtctaagaaccagttctccctgaagctgagcagtgtgactgccgcggacacggcggtgtactactgcgccagaggggtggaatatcagcatttcaatgagtggggacagggtacattggtcaccgtctcctca

SEQ ID NO: 22 - protein, 9 aa, synthetic construct
YTFTSYGIN

SEQ ID NO: 23 - protein, 17 aa, synthetic construct
WITAYSGDTNYAQKLQG

SEQ ID NO: 24 - protein, 12 aa, synthetic construct
ARGPGQYADVDV

SEQ ID NO: 25 - protein, 26 aa, synthetic construct
QVQLVQSGAEVKKPGASVKVSCKASG

SEQ ID NO: 26 - protein, 14 aa, synthetic construct
WVRQAPGQGLEWMG

SEQ ID NO: 27 - protein, 30 aa, synthetic construct
RVTMTTDTSTSTAYMELRSLRSDDTAVYYC

SEQ ID NO: 28 - protein, 11 aa, synthetic construct
WGQGTMVTVSS

SEQ ID NO: 29 - protein, 119 aa, synthetic construct
QVQLVQSGAEVKKPGASVKVSCKASGYTFTSYGINWVRQAPGQGLEWMGWITAYSGDTNYAQKLQGRVTMTTDTSTSTAYMELRSLRSDDTAVYYCARGPGQYADVDVWGQGTMVTVSS

SEQ ID NO: 30 - DNA, 357 bp, synthetic construct
  source 1..357: mol_type = other DNA
caggttcagctggtgcagtctggagctgaggtgaagaagcctggggcctcagtgaaggtctcctgcaaggcttctggttacacctttacctcctatggtatcaactgggtgcgacaggcccctggacaagggcttgagtggatgggatggatcaccgcttacagtggtgacacaaactatgcacagaagctccagggcagagtcaccatgaccacagacacatccacgagcacagcctacatggagctgaggagcctgagatctgacgacacggcggtgtactactgcgccagaggacctggacagtatgcagacgtagacgtatggggtcagggtacaatggtcaccgtctcctca

SEQ ID NO: 31 - protein, 9 aa, synthetic construct
GTFGGYAIS

SEQ ID NO: 32 - protein, 17 aa, synthetic construct
GIIPPYATANYAQKFQG

SEQ ID NO: 33 - protein, 11 aa, synthetic construct
ARGVGTTHLDV

SEQ ID NO: 34 - protein, 26 aa, synthetic construct
QVQLVQSGAEVKKPGSSVKVSCKASG

SEQ ID NO: 35 - skipped

SEQ ID NO: 36 - protein, 30 aa, synthetic construct
RVTITADESTSTAYMELSSLRSEDTAVYYC

SEQ ID NO: 37 - skipped

SEQ ID NO: 38 - protein, 118 aa, synthetic construct
QVQLVQSGAEVKKPGSSVKVSCKASGGTFGGYAISWVRQAPGQGLEWMGGIIPPYATANYAQKFQGRVTITADESTSTAYMELSSLRSEDTAVYYCARGVGTTHLDVWGQGTMVTVSS

SEQ ID NO: 39 - DNA, 354 bp, synthetic construct
  source 1..354: mol_type = other DNA
caggtgcagctggtgcagtctggggctgaggtgaagaagcctgggtcctcggtgaaggtctcctgcaaggcttctggaggcaccttcggaggatatgctatcagctgggtgcgacaggcccctggacaagggcttgagtggatgggagggatcatccctccatacgcaacagcaaactacgcacagaagttccagggcagagtcacgattaccgcggacgaatccacgagcacagcctacatggagctgagcagcctgagatctgaggacacggcggtgtactactgcgccagaggggtgggaactacccatctagacgtatggggtcagggtacaatggtcaccgtctcctca

SEQ ID NO: 40 - protein, 9 aa, synthetic construct
GTFQGYAIS

SEQ ID NO: 41 - skipped

SEQ ID NO: 42 - protein, 11 aa, synthetic construct
ARGVGELHLDV

SEQ ID NO: 43 - skipped

SEQ ID NO: 44 - protein, 14 aa, synthetic construct
WARQAPGQGLEWMG

SEQ ID NO: 45-46 - skipped (2 entries)

SEQ ID NO: 47 - protein, 118 aa, synthetic construct
QVQLVQSGAEVKKPGSSVKVSCKASGGTFQGYAISWARQAPGQGLEWMGGIIPPYATANYAQKFQGRVTITADESTSTAYMELSSLRSEDTAVYYCARGVGELHLDVWGQGTMVTVSS

SEQ ID NO: 48 - DNA, 354 bp, synthetic construct
  source 1..354: mol_type = other DNA
caggtgcagctggtgcagtctggggctgaggtgaagaagcctgggtcctcggtgaaggtctcctgcaaggcttctggaggcaccttccaaggatatgctatcagctgggcgcgacaggcccctggacaagggcttgagtggatgggagggatcatccctccatacgcaacagcaaactacgcacagaagttccagggcagagtcacgattaccgcggacgaatccacgagcacagcctacatggagctgagcagcctgagatctgaggacacggcggtgtactactgcgccagaggggtgggagagttgcatctagacgtatggggtcagggtacaatggtcaccgtctcctca

SEQ ID NO: 49 - protein, 9 aa, synthetic construct
GTFSSYYIS

SEQ ID NO: 50 - protein, 17 aa, synthetic construct
GIAPISGTASYAQKFQG

SEQ ID NO: 51 - protein, 12 aa, synthetic construct
ARGLAYDGYFDL

SEQ ID NO: 52 - skipped

SEQ ID NO: 53 - protein, 11 aa, synthetic construct
WGRGTLVTVSS

SEQ ID NO: 54 - protein, 119 aa, synthetic construct
QVQLVQSGAEVKKPGSSVKVSCKASGGTFSSYYISWVRQAPGQGLEWMGGIAPISGTASYAQKFQGRVTITADESTSTAYMELSSLRSEDTAVYYCARGLAYDGYFDLWGRGTLVTVSS

SEQ ID NO: 55 - DNA, 357 bp, synthetic construct
  source 1..357: mol_type = other DNA
caggtgcagctggtgcagtctggggctgaggtgaagaagcctgggtcctcggtgaaggtctcctgcaaggcttctggaggcaccttcagcagctactacatcagctgggtgcgacaggcccctggacaagggcttgagtggatgggagggatcgcccctatctccggtacagcatcctacgcacagaagttccagggcagagtcacgattaccgcggacgaatccacgagcacagcctacatggagctgagcagcctgagatctgaggacacggcggtgtactactgcgccagagggttggcatatgatggctacttcgacctatgggggaggggtaccttggtcaccgtctcctca

SEQ ID NO: 56 - protein, 9 aa, synthetic construct
YTFTDEGIN

SEQ ID NO: 57 - protein, 17 aa, synthetic construct
WITAYSGDTVYAQKLQG

SEQ ID NO: 58 - protein, 119 aa, synthetic construct
QVQLVQSGAEVKKPGASVKVSCKASGYTFTDEGINWVRQAPGQGLEWMGWITAYSGDTVYAQKLQGRVTMTTDTSTSTAYMELRSLRSDDTAVYYCARGPGQYADVDVWGQGTMVTVSS

SEQ ID NO: 59 - DNA, 357 bp, synthetic construct
  source 1..357: mol_type = other DNA
caggttcagctggtgcagtctggagctgaggtgaagaagcctggggcctcagtgaaggtctcctgcaaggcttctggttacacctttaccgacgaaggtatcaactgggtgcgacaggcccctggacaagggcttgagtggatgggatggatcaccgcttacagtggtgacacagtctatgcacagaagctccagggcagagtcaccatgaccacagacacatccacgagcacagcctacatggagctgaggagcctgagatctgacgacacggcggtgtactactgcgccagaggacctggacagtatgcagacgtagacgtatggggtcagggtacaatggtcaccgtctcctca

SEQ ID NO: 60 - protein, 17 aa, synthetic construct
WIDAYSGSTNYAQKLQG

SEQ ID NO: 61 - protein, 119 aa, synthetic construct
QVQLVQSGAEVKKPGASVKVSCKASGYTFTSYGINWVRQAPGQGLEWMGWIDAYSGSTNYAQKLQGRVTMTTDTSTSTAYMELRSLRSDDTAVYYCARGPGQYADVDVWGQGTMVTVSS

SEQ ID NO: 62 - DNA, 357 bp, synthetic construct
  source 1..357: mol_type = other DNA
caggttcagctggtgcagtctggagctgaggtgaagaagcctggggcctcagtgaaggtctcctgcaaggcttctggttacacctttaccagctatggtatcaactgggtgcgacaggcccctggacaagggcttgagtggatgggatggatcgacgcttacagtggttccacaaactatgcacagaagctccagggcagagtcaccatgaccacagacacatccacgagcacagcctacatggagctgaggagcctgagatctgacgacacggcggtgtactactgcgccagaggacctggacagtatgcagacgtagacgtatggggtcagggtacaatggtcaccgtctcctca

SEQ ID NO: 63 - protein, 9 aa, synthetic construct
YTFTNEGIS

SEQ ID NO: 64 - protein, 17 aa, synthetic construct
WISAYTADTFYAQKLQG

SEQ ID NO: 65 - protein, 14 aa, synthetic construct
ARTRHDTSGLRFDY

SEQ ID NO: 66 - protein, 121 aa, synthetic construct
QVQLVQSGAEVKKPGASVKVSCKASGYTFTNEGISWVRQAPGQGLEWMGWISAYTADTFYAQKLQGRVTMTTDTSTSTAYMELRSLRSDDTAVYYCARTRHDTSGLRFDYWGQGTLVTVSS

SEQ ID NO: 67 - DNA, 363 bp, synthetic construct
  source 1..363: mol_type = other DNA
caggttcagctggtgcagtctggagctgaggtgaagaagcctggggcctcagtgaaggtctcctgcaaggcttctggttacacctttaccaacgaaggtatcagctgggtgcgacaggcccctggacaagggcttgagtggatgggatggatctccgcttacaccgcagacacattctatgcacagaagctccagggcagagtcaccatgaccacagacacatccacgagcacagcctacatggagctgaggagcctgagatctgacgacacggcggtgtactactgcgccagaactagacacgacacctccggactaagattcgactactggggacagggtacattggtcaccgtctcctca

SEQ ID NO: 68 - protein, 9 aa, synthetic construct
YTFASYDII

SEQ ID NO: 69 - protein, 17 aa, synthetic construct
WISALSGDTVYAQKLQG

SEQ ID NO: 70 - protein, 14 aa, synthetic construct
ARTRHDTSGIRFDY

SEQ ID NO: 71 - protein, 121 aa, synthetic construct
QVQLVQSGAEVKKPGASVKVSCKASGYTFASYDIIWVRQAPGQGLEWMGWISALSGDTVYAQKLQGRVTMTTDTSTSTAYMELRSLRSDDTAVYYCARTRHDTSGIRFDYWGQGTLVTVSS

SEQ ID NO: 72 - DNA, 363 bp, synthetic construct
  source 1..363: mol_type = other DNA
caggttcagctggtgcagtctggagctgaggtgaagaagcctggggcctcagtgaaggtctcctgcaaggcttctggttacacctttgccagctatgacatcatctgggtgcgacaggcccctggacaagggcttgagtggatgggatggatcagcgctctcagtggtgacacagtctatgcacagaagctccagggcagagtcaccatgaccacagacacatccacgagcacagcctacatggagctgaggagcctgagatctgacgacacggcggtgtactactgcgccagaactagacacgacacctccggaattcggttcgactactggggacagggtacattggtcaccgtctcctca

SEQ ID NO: 73 - protein, 9 aa, synthetic construct
YTFDSYGIN

SEQ ID NO: 74 - protein, 17 aa, synthetic construct
WISAYSGDTAYAQELQG

SEQ ID NO: 75 - protein, 11 aa, synthetic construct
VRDTQYGGSPY

SEQ ID NO: 76 - protein, 118 aa, synthetic construct
QVQLVQSGAEVKKPGASVKVSCKASGYTFDSYGINWVRQAPGQGLEWMGWISAYSGDTAYAQELQGRVTMTTDTSTSTAYMELRSLRSDDTAVYYCVRDTQYGGSPYWGQGTLVTVSS

SEQ ID NO: 77 - DNA, 354 bp, synthetic construct
  source 1..354: mol_type = other DNA
caggttcagctggtgcagtctggagctgaggtgaagaagcctggggcctcagtgaaggtctcctgcaaggcttctggttacacctttgacagctatggtatcaactgggtgcgacaggcccctggacaagggcttgagtggatgggatggatctccgcttacagtggtgacacagcctatgcacaggagctccagggcagagtcaccatgaccacagacacatccacgagcacagcctacatggagctgaggagcctgagatctgacgacacggcggtgtactactgcgttcgggatactcagtatggtggtagcccatactggggacagggtacattggtcaccgtctcctca

SEQ ID NO: 78 - protein, 9 aa, synthetic construct
YTFTDYGIF

SEQ ID NO: 79 - protein, 17 aa, synthetic construct
WITAYSGDTHYAQKLQG

SEQ ID NO: 80 - protein, 118 aa, synthetic construct
QVQLVQSGAEVKKPGASVKVSCKASGYTFTDYGIFWVRQAPGQGLEWMGWITAYSGDTHYAQKLQGRVTMTTDTSTSTAYMELRSLRSDDTAVYYCVRDTQYGGSPYWGQGTLVTVSS

SEQ ID NO: 81 - DNA, 354 bp, synthetic construct
  source 1..354: mol_type = other DNA
caggttcagctggtgcagtctggagctgaggtgaagaagcctggggcctcagtgaaggtctcctgcaaggcttctggttacacctttaccgactatggtatcttctgggtgcgacaggcccctggacaagggcttgagtggatgggatggatcaccgcttacagtggtgacacacactatgcacagaagctccagggcagagtcaccatgaccacagacacatccacgagcacagcctacatggagctgaggagcctgagatctgacgacacggcggtgtactactgcgtgcgtgatactcagtatggtggtagcccatactggggacagggtacattggtcaccgtctcctca

SEQ ID NO: 82 - protein, 9 aa, synthetic construct
YTFDSLGMS

SEQ ID NO: 83 - protein, 17 aa, synthetic construct
YISTYSGDTVYAQKLQG

SEQ ID NO: 84 - protein, 13 aa, synthetic construct
ARESGQQVSALDP

SEQ ID NO: 85 - protein, 26 aa, synthetic construct
QVQLVQSGAEVKKPGATVKVSCKASG

SEQ ID NO: 86 - protein, 120 aa, synthetic construct
QVQLVQSGAEVKKPGATVKVSCKASGYTFDSLGMSWVRQAPGQGLEWMGYISTYSGDTVYAQKLQGRVTMTTDTSTSTAYMELRSLRSDDTAVYYCARESGQQVSALDPWGQGTLVTVSS

SEQ ID NO: 87 - DNA, 360 bp, synthetic construct
  source 1..360: mol_type = other DNA
caggttcagctggtgcagtctggagctgaggtgaagaagcctggggccacggtgaaggtctcctgcaaggcttctggttacacctttgacagcttaggtatgagctgggtgcgacaggcccctggacaagggcttgagtggatgggatatatcagcacgtacagtggtgacacagtatatgcacagaagctccagggcagagtcaccatgaccacagacacatccacgagcacagcctacatggagctgaggagcctgagatctgacgacacggcggtgtactactgcgcacgagagtctggccagcaggtttccgcgctcgacccttggggccagggcaccctggtcaccgtctcctca

SEQ ID NO: 88 - protein, 9 aa, synthetic construct
YTFDSYGIH

SEQ ID NO: 89 - protein, 13 aa, synthetic construct
ARESGQTSSALGP

SEQ ID NO: 90 - protein, 120 aa, synthetic construct
QVQLVQSGAEVKKPGATVKVSCKASGYTFDSYGIHWVRQAPGQGLEWMGWITAYSGDTVYAQKLQGRVTMTTDTSTSTAYMELRSLRSDDTAVYYCARESGQTSSALGPWGQGTLVTVSS

SEQ ID NO: 91 - DNA, 360 bp, synthetic construct
  source 1..360: mol_type = other DNA
caggttcagctggtgcagtctggagctgaggtgaagaagcctggggccacagtgaaggtctcctgcaaggcttctggttacacctttgacagctatggtatccactgggtgcgacaggcccctggacaagggcttgagtggatgggatggatcaccgcttacagtggtgacacagtctatgcacagaagctccagggcagagtcaccatgaccacagacacatccacgagcacagcctacatggagctgaggagcctgagatctgacgacacggcggtgtactactgcgccagagagtctggacaaacaagctctgccctggggccatggggacagggtacattggtcaccgtctcctca

SEQ ID NO: 92 - protein, 16 aa, synthetic construct
RSSQSLLHSNGYNYLD

SEQ ID NO: 93 - protein, 7 aa, synthetic construct
LGSNRAS

SEQ ID NO: 94 - protein, 9 aa, synthetic construct
MQTLEAPPT

SEQ ID NO: 95 - protein, 23 aa, synthetic construct
DIVMTQSPLSLPVTPGEPASISC

SEQ ID NO: 96 - protein, 15 aa, synthetic construct
WYLQKPGQSPQLLIY

SEQ ID NO: 97 - protein, 32 aa, synthetic construct
GVPDRFSGSGSGTDFTLKISRVEAEDVGVYYC

SEQ ID NO: 98 - protein, 10 aa, synthetic construct
FGGGTKVEIK

SEQ ID NO: 99 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQTLEAPPTFGGGTKVEIK

SEQ ID NO: 100 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcagacactcgaagcccctcctacttttggcggagggaccaaggttgagatcaaa

SEQ ID NO: 101 - protein, 11 aa, synthetic construct
RASQGISSWLA

SEQ ID NO: 102 - protein, 7 aa, synthetic construct
AASSLQS

SEQ ID NO: 103 - protein, 9 aa, synthetic construct
QQANSFPIT

SEQ ID NO: 104 - protein, 23 aa, synthetic construct
DIQMTQSPSSVSASVGDRVTITC

SEQ ID NO: 105 - protein, 15 aa, synthetic construct
WYQQKPGKAPKLLIY

SEQ ID NO: 106 - protein, 32 aa, synthetic construct
GVPSRFSGSGSGTDFTLTISSLQPEDFATYYC

SEQ ID NO: 107 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGGGTKVEIK

SEQ ID NO: 108 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctatcacctttggcggagggaccaaggttgagatcaaa

SEQ ID NO: 109 - skipped

SEQ ID NO: 110 - protein, 9 aa, synthetic construct
MQALQTPIT

SEQ ID NO: 111-114 - skipped (4 entries)

SEQ ID NO: 115 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGGGTKVEIK

SEQ ID NO: 116 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctatcacctttggcggagggaccaaggttgagatcaaa

SEQ ID NO: 117 - protein, 16 aa, synthetic construct
RSSQSLLYSNGYNYLD

SEQ ID NO: 118 - protein, 9 aa, synthetic construct
MQAIGVPLT

SEQ ID NO: 119-122 - skipped (4 entries)

SEQ ID NO: 123 - protein, 112 aa, synthetic construct
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQAIGVPLTFGGGTKVEIK

SEQ ID NO: 124 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgtatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaggcaataggcgtccctctcacttttggcggagggaccaaggttgagatcaaa

SEQ ID NO: 125 - protein, 11 aa, synthetic construct
RASQGIDSWLA

SEQ ID NO: 126 - protein, 9 aa, synthetic construct
QQVHSFPIT

SEQ ID NO: 127 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQVHSFPITFGGGTKVEIK

SEQ ID NO: 128 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattgacagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcaggtacacagtttccctatcacttttggcggagggaccaaggttgagatcaaa

SEQ ID NO: 129 - protein, 11 aa, synthetic construct
RASQSVSSNLA

SEQ ID NO: 130 - protein, 7 aa, synthetic construct
GASTRAT

SEQ ID NO: 131 - protein, 9 aa, synthetic construct
QQYNNWPIT

SEQ ID NO: 132 - protein, 23 aa, synthetic construct
EIVMTQSPATLSVSPGERATLSC

SEQ ID NO: 133 - protein, 15 aa, synthetic construct
WYQQKPGQAPRLLIY

SEQ ID NO: 134 - protein, 32 aa, synthetic construct
GIPARFSGSGSGTEFTLTISSLQSEDFAVYYC

SEQ ID NO: 135 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPITFGGGTKVEIK

SEQ ID NO: 136 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctatcacctttggcggagggaccaaggttgagatcaaa

SEQ ID NO: 137 - protein, 11 aa, synthetic construct
RASQGISRWLA

SEQ ID NO: 138 - protein, 9 aa, synthetic construct
QQGASFPLT

SEQ ID NO: 139 - protein, 107 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQGASFPLTFGGGTKVEIK

SEQ ID NO: 140 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcaggtggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcagggagccagtttccctctcacttttggcggagggaccaaggttgagatcaaa

SEQ ID NO: 141-142 - skipped (2 entries)

SEQ ID NO: 143 - protein, 449 aa, synthetic construct
QVQLQESGPGLVKPSQTLSLTCTVSGGSITSGGSYWSWIRQHPGKGLEWIGAIVYSGSTYYNPSLKSRVTISVDTSKNQFSLKLSSVTAADTAVYYCARGVEYQHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 449 aa, synthetic construct
QVQLQESGPGLVKPSQTLSLTCTVSGGSISSGGIYWTWIRQHPGKGLEWIGAIAYSGSTYYNPSLKSRVTISVDTSKNQFSLKLSSVTAADTAVYYCARGIEYQHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 449 aa, synthetic construct
QVQLQESGPGLVKPSQTLSLTCTVSGGSISSGGYYWSWIRQHPGKGLEWIGAIVYSGHTYYNPSLKSRVTISVDTSKNQFSLKLSSVTAADTAVYYCARGVEYQHFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 449 aa, synthetic construct
QVQLQESGPGLVKPSQTLSLTCTVSGGSINSGGYYWTWIRQHPGKGLEWIGAIYYSGSTVYNPSLKSRVTISVDTSKNQFSLKLSSVTAADTAVYYCARGVEYQHFNEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 449 aa, synthetic construct
QVQLVQSGAEVKKPGASVKVSCKASGYTFTSYGINWVRQAPGQGLEWMGWITAYSGDTNYAQKLQGRVTMTTDTSTSTAYMELRSLRSDDTAVYYCARGPGQYADVDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 448 aa, synthetic construct
QVQLVQSGAEVKKPGSSVKVSCKASGGTFGGYAISWVRQAPGQGLEWMGGIIPPYATANYAQKFQGRVTITADESTSTAYMELSSLRSEDTAVYYCARGVGTTHLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 448 aa, synthetic construct
QVQLVQSGAEVKKPGSSVKVSCKASGGTFQGYAISWARQAPGQGLEWMGGIIPPYATANYAQKFQGRVTITADESTSTAYMELSSLRSEDTAVYYCARGVGELHLDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 449 aa, synthetic construct
QVQLVQSGAEVKKPGSSVKVSCKASGGTFSSYYISWVRQAPGQGLEWMGGIAPISGTASYAQKFQGRVTITADESTSTAYMELSSLRSEDTAVYYCARGLAYDGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 449 aa, synthetic construct
QVQLVQSGAEVKKPGASVKVSCKASGYTFTDEGINWVRQAPGQGLEWMGWITAYSGDTVYAQKLQGRVTMTTDTSTSTAYMELRSLRSDDTAVYYCARGPGQYADVDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 449 aa, synthetic construct
QVQLVQSGAEVKKPGASVKVSCKASGYTFTSYGINWVRQAPGQGLEWMGWIDAYSGSTNYAQKLQGRVTMTTDTSTSTAYMELRSLRSDDTAVYYCARGPGQYADVDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 451 aa, synthetic construct
QVQLVQSGAEVKKPGASVKVSCKASGYTFTNEGISWVRQAPGQGLEWMGWISAYTADTFYAQKLQGRVTMTTDTSTSTAYMELRSLRSDDTAVYYCARTRHDTSGLRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 451 aa, synthetic construct
QVQLVQSGAEVKKPGASVKVSCKASGYTFASYDIIWVRQAPGQGLEWMGWISALSGDTVYAQKLQGRVTMTTDTSTSTAYMELRSLRSDDTAVYYCARTRHDTSGIRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 448 aa, synthetic construct
QVQLVQSGAEVKKPGASVKVSCKASGYTFDSYGINWVRQAPGQGLEWMGWISAYSGDTAYAQELQGRVTMTTDTSTSTAYMELRSLRSDDTAVYYCVRDTQYGGSPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 448 aa, synthetic construct
QVQLVQSGAEVKKPGASVKVSCKASGYTFTDYGIFWVRQAPGQGLEWMGWITAYSGDTHYAQKLQGRVTMTTDTSTSTAYMELRSLRSDDTAVYYCVRDTQYGGSPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 450 aa, synthetic construct
QVQLVQSGAEVKKPGATVKVSCKASGYTFDSLGMSWVRQAPGQGLEWMGYISTYSGDTVYAQKLQGRVTMTTDTSTSTAYMELRSLRSDDTAVYYCARESGQQVSALDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 450 aa, synthetic construct
QVQLVQSGAEVKKPGATVKVSCKASGYTFDSYGIHWVRQAPGQGLEWMGWITAYSGDTVYAQKLQGRVTMTTDTSTSTAYMELRSLRSDDTAVYYCARESGQTSSALGPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQTLEAPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160-162 - skipped (3 entries)

SEQ ID NO: 163 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - skipped

SEQ ID NO: 166 - protein, 219 aa, synthetic construct
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQAIGVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167-168 - skipped (2 entries)

SEQ ID NO: 169 - protein, 214 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQVHSFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - skipped

SEQ ID NO: 171 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - skipped

SEQ ID NO: 173 - protein, 214 aa, synthetic construct
DIQMTQSPSSVSASVGDRVTITCRASQGISRWLAWYQQKPGKAPKLLIYAASSLQSGVPSRFSGSGSGTDFTLTISSLQPEDFATYYCQQGASFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - skipped

SEQ ID NO: 175 - protein, 107 aa, synthetic construct [sequence omitted]

SEQ ID NO: 176 - protein, 21 aa, Homo sapiens
CPPCPAPELLGGPSVFLFPPK

SEQ ID NO: 177 - protein, 21 aa, synthetic construct
CPPCPAPELAGAPSVFLFPPK

SEQ ID NO: 178 - protein, 21 aa, synthetic construct
CPPCPAPEFEGGPSVFLFPPK

SEQ ID NO: 179 - protein, 21 aa, synthetic construct
CPPCPAPEAAGGPSVFLFPPK

SEQ ID NO: 180 - protein, 21 aa, synthetic construct
CPPCPAPEAAGAPSVFLFPPK

SEQ ID NO: 181 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 184 - protein, 308 aa, Macaca fascicularis
MPGQELKTLNGSQMLLVLLVLLWPPHGGAVSLAEASRASFPGPSDLHSEDSRFRELRKRYEDLLTRLRANQSWEDSNTDLIQAPEVRILTPEVRLGSGGHLHLRISRAVLPEGLPEACRIHRALFRLSPTASRSRDVTRPLRRQLRLARPQAPALHLRLSPPPSQSDQLLVKSSSSRPQLALHLRPRASRGRRRARARNGDRCPLGPGRCCRLHTVHASLEDLGWADWVLSPREVQVTMCIGACPSQFREANMHAQIKMNLHRLKPDTVPAPCCVPASYNPMVLIQKTDTGVSLQTYDDLLAKDCHCV

SEQ ID NO: 185 - protein, 307 aa, Canis lupus
  source 1..307: note = Canis lupus familiaris
MPGQGPAPAHCSPMLVILVMLSWLPSGGALSLAQEHLPAFPGPSDPHSSTDVSRIQELRKRYEHLQTKLRLNQGWADSNPDLVPATRVRILTPKLRLGPRGHLHLRIARADLTAGLPAASRLHRALLRLSPTEPSSWDVTRPLQRQLSRVGSRTPTLRLRLLPRWDRSRALPSARPQLELHWRPRAARGRRNAHAHARDGCPLGEGRCCRLQSLRASLQDLGWANWVVAPRELDVRMCVGACPSQFRSANTHAQMQARLHGLNPDAAPAPCCVPASYEPVVLMHQDSDGRVSLTPFDDLVAKDCHCV

SEQ ID NO: 186 - protein, 307 aa, Felis catus
SVQNSASTGMPGPGPTPPMLLMLLMLLMLCWLPSGGALSLAQEHLPAFPGPSEARSGTDVSRFEEFRKLYEHLQTRLRLNQSWEDSNPDRVISEAQVRILTPKLRLGLGGHLHLRIARADLTKGLPASFRLHRALLRLSPTELSSWDVTRPLRRQLSLGGSGRDRSPAALPSSARPQLELHWRPRAARGRRNAHARSKDDCPLGAGRCCRLQSLRASLEDLGWASWVVAPRELDVRMCIGACPSQFRSANTHAQMQARLHGLNPDATPAPCCVPARYEPVVLMHQDSDGRVSLTPFDDLVAKDCHCL

SEQ ID NO: 187 - protein, 29 aa, Homo sapiens
MPGQELRTVNGSQMLLVLLVLSWLPHGGA

SEQ ID NO: 188 - protein, 165 aa, Homo sapiens
LSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRR

SEQ ID NO: 189 - protein, 114 aa, Homo sapiens
ARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 190 - DNA, 1417 bp, Homo sapiens
  source 1..1417: mol_type = genomic DNA
ctgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcggaggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttcaaaaaaaaaaaaaaaaaa

SEQ ID NO: 191 - protein, 11 aa, synthetic construct
ARGIEYQHFDY

SEQ ID NO: 192 - protein, 11 aa, synthetic construct
ARGVEYQHFSL

SEQ ID NO: 193 - protein, 11 aa, synthetic construct
ARGVEYQHFNE

SEQ ID NO: 194-199 - skipped (6 entries)

SEQ ID NO: 200 - protein, 16 aa, synthetic construct
AIAYSGSTYYNPSLES

SEQ ID NO: 201 - protein, 119 aa, synthetic construct
QVQLQESGPGLVKPSQTLSLTCTVSGGSISSGGIYWTWIRQHPGKGLEWIGAIAYSGSTYYNPSLESRVTISVDTSKNQFSLKLSSVTAADTAVYYCARGIEYQHFDYWGQGTLVTVSS

SEQ ID NO: 202 - protein, 449 aa, synthetic construct
QVQLQESGPGLVKPSQTLSLTCTVSGGSISSGGIYWTWIRQHPGKGLEWIGAIAYSGSTYYNPSLESRVTISVDTSKNQFSLKLSSVTAADTAVYYCARGIEYQHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - DNA, 357 bp, synthetic construct
  source 1..357: mol_type = other DNA
caggtgcagctgcaggagtcgggcccaggactggtgaagccttcacagaccctgtccctcacctgtactgtctctggtggctccatcagcagtggtggtatctactggacctggatccgccagcacccagggaagggcttggagtggattggggccatcgcctacagtgggagcacctactacaacccgtccctcgagagtcgagttaccatatcagtagacacgtctaagaaccagttctccctgaagctgagcagtgtgactgccgcggacacggcggtgtactactgcgccagagggattgaatatcagcatttcgattactggggacagggtacattggtcaccgtctcctca

SEQ ID NO: 204 - protein, 9 aa, synthetic construct
MQTLELPPT

SEQ ID NO: 205 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQTLELPPTFGGGTKVEIK

SEQ ID NO: 206 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQTLELPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcagacactcgaactgcctcctacttttggcggagggaccaaggttgagatcaaa

SEQ ID NO: 208 - protein, 9 aa, synthetic construct
MSTLELPPT

SEQ ID NO: 209 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMSTLELPPTFGGGTKVEIK

SEQ ID NO: 210 - protein, 219 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMSTLELPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgagtacactcgaattgcctcctacttttggcggagggaccaaggttgagatcaaa

SEQ ID NO: 212 - protein, 11 aa, synthetic construct
RASQGISPWLA

SEQ ID NO: 213 - protein, 23 aa, synthetic construct
DIQLTQSPSSVSASVGDRVTITC

SEQ ID NO: 214 - protein, 107 aa, synthetic construct
DIQLTQSPSSVSASVGDRVTITCRASQGISPWLAWYQQKPGKAPKLLIYAASSLQSGVPSRFSGSGSGTDFTLTISSLQPEDFATYYCQQANSFPITFGGGTKVEIK

SEQ ID NO: 215 - protein, 214 aa, synthetic construct
DIQLTQSPSSVSASVGDRVTITCRASQGISPWLAWYQQKPGKAPKLLIYAASSLQSGVPSRFSGSGSGTDFTLTISSLQPEDFATYYCQQANSFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - DNA, 321 bp, synthetic construct
  source 1..321: mol_type = other DNA
gacatccagttgacccagtctccatcttccgtgtctgcatctgtaggagacagagtcaccatcacttgtcgggcgagtcagggtattagcccctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctatcacctttggcggagggaccaaggttgagatcaaa

SEQ ID NO: 217 - protein, 9 aa, synthetic construct
QQASQFPIT

SEQ ID NO: 218 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQASQFPITFGGGTKVEIK

SEQ ID NO: 219 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQASQFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcaacaggctagtcagttccctatcacctttggcggagggaccaaggttgagatcaaa

SEQ ID NO: 221 - protein, 106 aa, Homo sapiens [sequence omitted]

SEQ ID NO: 222 - protein, 11 aa, Homo sapiens
DLGWADWVLSP

SEQ ID NO: 223 - protein, 62 aa, Homo sapiens
  DISULFID 6..13: note = Intrachain disulfide bond
  DISULFID 14: note = Intrachain disulfide bond with the cysteine at position 25 of SEQ ID NO: 231
  DISULFID 43: note = Intrachain disulfide bond with the cysteine at position 56 of SEQ ID NO: 231
  DISULFID 47: note = Intrachain disulfide bond with the cysteine at position 58 of SEQ ID NO: 231
RNGDHCPLGPGRCCRLHTVRASLEDLGWADWVLSPREVQVTMCIGACPSQFRAANMHAQIKT

SEQ ID NO: 224 - protein, 66 aa, Homo sapiens
GLECDGKVNICCKKQFFVSFKDIGWNDWIIAPSGYHANYCEGECPSHIAGTSGSSLSFHSTVINHY

SEQ ID NO: 225 - protein, 67 aa, Homo sapiens
RGLECDGRTNLCCRQQFFIDFRLIGWNDWIIAPTGYYGNYCEGSCPAYLAGVPGSASSFHTAVVNQY

SEQ ID NO: 226 - protein, 61 aa, Homo sapiens
QWDEPRVCSRRYLKVDFADIGWNEWIISPKSFDAYYCAGACEFPMPKIVRPSNHATIQSIV

SEQ ID NO: 227 - protein, 4 aa, synthetic construct
LAGA

SEQ ID NO: 228 - protein, 4 aa, synthetic construct
FEGG

SEQ ID NO: 229 - protein, 4 aa, synthetic construct
AAGG

SEQ ID NO: 230 - protein, 4 aa, synthetic construct
AAGA

SEQ ID NO: 231 - protein, 59 aa, Homo sapiens
  DISULFID 24: note = Interchain disulfide bond
  DISULFID 25: note = Intrachain disulfide bond with the cysteine at position 14 of SEQ ID NO: 223
  DISULFID 56: note = Intrachain disulfide bond with the cysteine at position 43 of SEQ ID NO: 223
  DISULFID 58: note = Intrachain disulfide bond with the cysteine at position 47 of SEQ ID NO: 223
AANMHAQIKTSLHRLKPDTVPAPCCVPASYNPMVLIQKTDTGVSLQTYDDLLAKDCHCI

SEQ ID NO: 232 - protein, 67 aa, Homo sapiens
GTSGSSLSFHSTVINHYRMRGHSPFANLKSCCVPTKLRPMSMLYYDDGQNIIKKDIQNMIVEECGCS

SEQ ID NO: 233 - protein, 66 aa, Homo sapiens
GVPGSASSFHTAVVNQYRMRGLNPGTVNSCCIPTKLSTMSMLYFDDEYNIVKRDVPNMIVEECGCA

SEQ ID NO: 234 - protein, 64 aa, Homo sapiens
KIVRPSNHATIQSIVRAVGIIPGIPEPCCVPDKMNSLGVLFLDENRNVVLKVYPNMSVDTCACR